(19) United States
(12) Patent Application Publication
(10) Pub. No.: US 2024/0390433 A1
(43) Pub. Date: Nov. 28, 2024

(54) GENETICALLY MODIFIED BACILLUS SUBTILIS STRAIN AND USE AS A LIVE DELIVERY AND PRODUCTION SYSTEM

(71) Applicant: BiomEdit, LLC, Fishers, IN (US)

(72) Inventors: Dharanesh Mahimapura Gangaiah, Fishers, IN (US); Arvind Kumar, Fishers, IN (US); Shrinivasrao Peerajirao Mane, Fishers, IN (US)

(21) Appl. No.: 18/693,585

(22) PCT Filed: Sep. 21, 2022
(86) PCT No.: PCT/US2022/044211

§ 371 Date: Mar. 20, 2024

Related U.S. Application Data
(60) Provisional application No. 63/247,271, filed on Sep. 22, 2021.
(60) Provisional application No. 63/247,273, filed on Sep. 22, 2021.
(60) Provisional application No. 63/247,400, filed on Sep. 23, 2021.

(51) Int. Cl. A61K 35/742; C07K 14/32; C12N 1/20; C12N 15/75; C12R 1/125

(52) U.S. Cl. CPC A61K 35/742; C07K 14/32; C12N 1/205; C12N 15/75; C12R 2001/125

(57) ABSTRACT

The present invention relates to genetically modified Bacillus subtilis, compositions, and uses thereof in production of biomolecules and heterologous proteins and for delivery of biomolecules and heterologous proteins in animals and associated methods for improving animal health.

### CROSS REFERENCE TO RELATED APPLICATIONS

The present application claims priority to U.S. Application Ser. No. 63/247,271, filed Sep. 22, 2021, U.S. Application Ser. No. 63/247,273, filed Sep. 22, 2021, and U.S. Application Ser. No. 63/247,400, filed Sep. 23, 2021, the entire contents of which are incorporated by reference herein.

### SEQUENCE LISTING

This application contains a Sequence Listing, which was submitted in XML format via EFS-Web, and is hereby incorporated by reference in its entirety. The XML copy, created on Sep. 21, 2022, is named “2950-12_ST26.xml” and is 12,483,777 bytes in size.

### FIELD OF THE INVENTION

The present invention relates to genetically modified Bacillus subtilis, compositions, and uses thereof in production and for delivery of biomolecules and heterologous proteins in animals and associated methods for improving animal health.

### BACKGROUND

Direct fed microbials (DFMs), often also called probiotics, are microorganisms which colonize the gastrointestinal tract of an animal and provide some beneficial effect to that animal. The microorganisms can be bacterial species, for example those from the genera Bacillus, Lactobacillus, Lactococcus, and Enterococcus. The microorganisms can be provided to an animal orally or mucosally or, in the case of birds, provided to a fertilized egg, i.e. in ovo. The beneficial activity provided by a DFM can be through the synthesis and secretion of vitamins or other nutritional molecules needed for a healthy metabolism of the host animal. A DFM can also protect the host animal from disease, disorders, or clinical symptoms caused by pathogenic microorganisms or other agents. For example, the DFM may naturally produce factors having inhibitory or cytotoxic activity against certain species of pathogens, such as deleterious or disease-causing bacteria. Probiotics and DFMs provide an attractive alternative or addition to the use and application of antibiotics in animals. Antibiotics can promote resistant or less sensitive bactaeria and can ultimately end up in feed products or foods consumed by other animals or humans. DFMs are characterized as being generally safe (even denoted Generally Regarded as Safe (GRAS) and most are not naturally resistant to antibiotics.

However, the DFM may not be able to produce such factors in sufficient quantity to reduce infection of the host with the pathogen, or the factors may affect only a limited set of pathogens, leaving the host vulnerable to other pathogens. Strains suitable as DFMs can provide an attractive and useful starting point for applications to produce or generate biomolecules and heterologous proteins, including as a live delivery system for synthesis and delivery of molecules or proteins with wide applications including in therapy and in animal health.

Recombinant protein production in microbial cells is an important aspect of the modern biotechnological industry. Intracellular expression of heterologous proteins in host cells is widely utilized and such proteins are isolated from a culture of producing host cells. Biomolecules or heterologous proteins can be expressed from plasmids transfected into bacterial cells or from encoding sequence(s) integrated in the host bacteria genome.

In addition, recent achievements in secretory expression of recombinant proteins have encouraged both the scientific and industrial communities to apply and implement bacteria with a secretory ability for protein production. Using secretory-type host cells, synthesized target biomolecules and proteins are secreted directly and accumulated in the extracellular medium, which provides cost-effective downstream purification processing. Further, this can permit production and isolation of target biomolecules and proteins without the need or requirement for lysing the host cells. Also, secretory expression of recombinant proteins prevents accumulation of target biomolecules heterologous proteins within host cells, which can limit cell growth and production, lead to cell toxicity and result in incorrect protein folding (Mergulhao, F. J.; Summers, D. K.; Monteiro, G. A. (2005) Biotechnol Adv 23(3):177-202; Song, Y.; Nikoloff, J. M.; Zhang, D. (2015) J Microbiol Biotechnol 25(7): 963-77).

Bacillus subtilis is a Gram-positive model bacterium which is widely used for industrial production of recombinant proteins such as alpha-amylase, protease, lipase, and other industrial enzymes. Because of the ability of the bacteria to produce large amounts of a target protein, and also to secrete large amounts of a target protein into the culture medium, and the availability of a low-cost downstream production and purification process, over 60% of commercial industrial enzymes are produced in Bacillus subtilis and relative Bacillus species (Schallmey, M.; Singh, A.; Ward, O. P. (2004) 50 (1): 1-17). In contrast to the frequently used recombinant protein expression host Escherichia coli, Bacillus subtilis has no risk of endotoxin contamination and has been certificated as a GRAS (generally regarded as safe) organism by the FDA, which makes it a choice for food-grade and pharmaceutical protein production.

Provided herein is a Bacillus subtilis expression system which is modified and engineered to produce biomolecules or heterologous proteins. In some instances, the modified Bacillus subtilis is capable of producing high levels of at least one or a multiplicity of biomolecules or heterologous proteins, including in instances as surface-displayed or secreted molecules. Otherwise, the modified Bacillus subtilis is capable of producing or delivering a therapeutic, biomolecule or heterologous protein upon introduction of the bacteria to a host animal. In thus instance, what is provided is a needed delivery system which can constantly deliver useful therapeutic molecules and biomolecules, such as anti-infective molecules, directly to the host, such as to the gastrointestinal tract where pathogenic bacteria are replicating in the host. The gastrointestinal system is also often a point of entry of the pathogen into the host. Preferably, the delivery system is a live genetically-modified microorganism, such as a bacterium, which can reproduce in—and even colonize in some instances—a host and directly deliver therapeutic molecules and biomolecules, such as antiinfective, antipathogenic or antibacterial agents to reduce the number of, or block the entry of, a pathogen.

There is a need in the art for bacterial strains, compositions and methods that provide improved production of beneficial molecules and/or delivery of beneficial molecules to the gastrointestinal tract of an animal and thus improve animal health. There is a need for an improved delivery platform and system, including suitable vectors and nucleic acid-based systems for rapid and effective expression of heterologous proteins or genes of interest and robust generation of numerous vehicles using a single platform. There is a need for strategies to provide intracellular and systemic delivery of therapeutic biomolecules, including antigens, antibodies, proteins and other therapeutic biomolecules.

The citation of references herein shall not be construed as an admission that such is prior art to the present invention.

### SUMMARY OF THE INVENTION

The invention provides compositions and methods for improving animal health and animal production and performance. The invention provides recombinantly manipulated and genetically modified Bacillus subtilis, compositions, and uses thereof in production and/or in direct delivery of biomolecules and heterologous proteins. The production or delivery of biomolecules and heterologous proteins provides materials, agents, compounds and associated methods for improving animal health.

The invention provides a Bacillus subtilis bacterial strain modified to facilitate expression and/or production and/or delivery of a biomolecule or heterologous protein of interest. In embodiments, the Bacillus subtilis bacterial strain is modified to introduce a strong or inducible promoter that drives expression and/or production of a natural or heterologous biomolecule or protein of interest. The strain may me modified to introduce a signal sequence that drives or facilitates secretion of the natural or heterologous biomolecule or protein of interest. In some embodiments, the Bacillus subtilis bacterial strain is modified to introduce nucleic acid encoding a heterologous protein or encoding one or more proteins required or utilized in the production of a heterologous protein. In embodiments, the nucleic acid introduced includes a strong or inducible promoter that drives expression and/or production of the heterologous protein of interest. In embodiments, the nucleic acid introduced includes a signal sequence that drives expression and secretion of the heterologous protein of interest.

The invention provides a production and delivery system which can constantly produce and deliver useful therapeutic molecules and biomolecules, such as anti-infective molecules, in a growth and production capacity and/or directly to the host, such as to the gastrointestinal tract where pathogenic bacteria are replicating in the host. The gastrointestinal system is also often a point of entry of the pathogen into the host. Preferably, the delivery system is a live genetically-modified microorganism, such as a bacterium, which can reproduce in—and even colonize in some instances—a host and directly deliver therapeutic molecules and biomolecules, such as antiinfective, antipathogenic, antibacterial, anti-inflammatory or immunomodulatory peptides, polypeptides, or agents to reduce the number of, or block the entry of, a pathogen. For example, in ovo delivery of a live delivery platform could prevent early colonization of an embryo by pathogens, possibly through competitive exclusion or direct or indirect anti-infective effects. In ovo delivery has the further advantage of bypassing any limitations of colonization by the genetically-modified microorganism due to maternal antibody interference. Preferably, the live bacterial delivery system synthesizes the anti-infective factor in sufficient quantity to have the desired effect on a pathogen. A targeted pathogen may be, without limitation, a bacterium of the genera Salmonella, Clostridium, Campylobacter, Staphylococcus, or Streptococcus, or an E. coli bacterium, or a parasite such as an Eimeria species. Preferably, the live bacterial system persists in the host gastrointestinal tract for a period of time. Preferably, the live bacterial delivery system produces a broad-spectrum anti-infective factor or multiple anti-infective factors, such that a variety of pathogens are targeted. Alternatively, a combination of live delivery systems could be administered to a single animal, with genetically-modified bacteria producing multiple anti-infective factors, immunomodulatory molecules, or growth-promoting biomolecules, or any combination thereof. Thus, more than one disease state is prevented or reduced, or diseases and syndromes having multiple causes can be effectively treated.

The present invention relates to an protein production and intracellular delivery platform which utilizes a genetically modified bacterium to produce or deliver preventative or therapeutic anti-infective activity, immunomodulatory factors, or growth-promoting biomolecules directly to the mucosa of an animal in need thereof.

The invention provides modified Bacillus bacteria, particularly modified Bacillus subtilis strain 105 (ELA191105), as a bacterial strain for production of one or more biomolecules and heterologous proteins. In an embodiment, the modified Bacillus bacteria, particularly modified Bacillus subtilis strain 105 (ELA191105), is a bacterial strain for production and secretion of one or more biomolecules and heterologous proteins.

In embodiments, the Bacillus subtilis strain 105 (ELA191105) is modified to include nucleic acid encoding one or more biomolecule or heterologous protein directly. In embodiments, the Bacillus subtilis strain 105 (ELA191105) is modified to include nucleic acid encoding a protein or proteins which facilitate, induce, enhance or otherwise result in production of one or more biomolecule or heterologous protein. In one such embodiment, the Bacillus subtilis strain 105 (ELA191105) is modified to include nucleic acid encoding one or more protein or enzyme or substrate in a production pathway, synthesis pathway, etc which results in the generation or production of a target biomolecule or heterologous protein.

In an embodiment, a live delivery platform comprising a genetically-modified Bacillus bacteria for production of one or more biomolecules and heterologous proteins in an animal is provided, wherein the modified Bacillus comprises Bacillus subtilis strain 105 (ELA191105) genetically modified to include nucleic acid encoding one or more biomolecule or heterologous protein which is produced and delivered upon administration of the modified Bacillus bacteria to the animal.

In an embodiment, the modified Bacillus comprises Bacillus subtilis strain 105 comprising the nucleic acid sequence set out in SEQ ID NO: 1 or a Bacillus strain having at least 95% identity, 97% identity, 98% identity, 99% identity in genomic sequence to SEQ ID NO: 1.

In an embodiment, the modified Bacillus comprises Bacillus subtilis strain corresponds to ATCC deposit PTA-126786 strain or a Bacillus strain having at least 95% identity, 97% identity, 98% identity, 99% identity in genomic sequence to the sequence of ELA191105 corresponding to ATCC deposit PTA-126786.

Bacillus subtilis strain 105, also denoted ELA191105 and also denoted Bs PTA-86, is an isolated Bacillus subtilis strain that has probiotic capability and characteristics. In an embodiment, Bacillus subtilis strain 105 corresponds to ATCC deposit PTA-126786. In embodiments, the B subtilis strain corresponds to ATCC deposit PTA-126786 strain or a Bacillus strain having at least 90% identity, 95% identity, 97% identity, 98% identity, 99% identity in genomic sequence to the sequence of ELA191105 corresponding to ATCC deposit PTA-126786.

In an embodiment, the B subtilis strain 105 comprises the nucleic acid sequence set out in SEQ ID NO: 1 or a Bacillus strain having at least 90% identity, 95% identity, 97% identity, 98% identity, 99% identity in genomic sequence to SEQ ID NO: 1. In an embodiment, the B subtilis strain 105 comprises the nucleic acid sequence set out in SEQ ID NO: 2, 3, 4, 5 and/or 6 or a Bacillus strain having at least 90% identity, 95% identity, 97% identity, 98% identity, 99% identity in genomic sequence to SEQ ID NO: 2, 3, 4, 5 and/or 6. In an embodiment, the B subtilis strain 105 comprises nucleic acid sequence set out in SEQ ID NO: 1, 2, 3, 4, 5 or 6 or a Bacillus strain having at least 90% identity, 95% identity, 97% identity, 98% identity, 99% identity in genomic sequence to SEQ ID NO: 1, 2, 3, 4 5 or 6. SEQ ID NO: 1 provides the whole genome nucleic acid sequence for ELA191105, deposited as PTA-126786.

The present invention relates to a Bacillus bacteria based production and live delivery system, wherein a genetically modified Bacillus subtilis, particularly a B. subtilis strain which is safe and has probiotic characteristics, is modified to encode and to produce one or more biomolecule or heterologous protein, or is modified to increase production or provide inducible production/expression of one or more biomolecule or heterologous protein. The biomolecule or protein of interest may be a homologous B subtilis protein or may be a heterologous protein. There may be one or more, two or more, three or more, or a complex or gene cassette encoded grouping of proteins or biomolecules. In an embodiment, the biomolecule or heterologous protein is a therapeutic agent. In an embodiment, the biomolecule or heterologous protein is a compound or agent or reagent important in a biological or chemical reaction. In an embodiment, the biomolecule or heterologous protein is an antigen or one or more antigens. In an embodiment, the biomolecule or heterologous protein is an antibody or a fragment thereof, such as a domain antibody or nanobody. In an embodiment, the biomolecule or heterologous protein is an anti-infective, anti-bacterial or anti-pathogen agent. In an embodiment, the biomolecule or heterologous protein is a lytic protein. In an embodiment, the biomolecule is a therapeutic biomolecule, particularly a molecule having preventative or therapeutic anti-infective activity, one or more immunomodulatory factor, or one or more growth-promoting biomolecule. Any of various and known or important biomolecule or protein may be expressed for by the system and modified strain 105 of the invention.

In an embodiment of the Bacillus bacteria based production and live delivery system, a genetically modified Bacillus subtilis is utilized to simultaneously produce one or more biomolecule or heterologous protein. In an embodiment, strain 105 is modified to produce a combination of biomolecules or heterologous proteins. In an embodiment, the combination may result in production of a molecule of interest. In embodiments, the combination may be for utilization as combined agents. In embodiments, the combination may be a set of antigens, such as for a vaccine or an immunogenic composition.

In an embodiment, strain 105 is modified to increase competence. In an embodiment, strain 105 is modified to increase its ability to take up and internalize extracellular nucleic acid or DNA. In an embodiment, strain 105 is modified to express, overexpress, or inducibly express the genes encoding comK and comS. In an embodiment, strain 105 is modified to express, overexpress, or inducibly express the competence comK and comS proteins. In an embodiment, strain 105 is modified inducibly express or overexpress the genes encoding comK and comS. In embodiments, overpress refers to expression of a gene or production of a protein which is greater, particularly significantly greater, than native expression of a gene or production of a protein. In embodiments, overpress refers to expression of a gene or production of a protein which is greater, particularly significantly greater, than the expression by an unmodified or wild type strain. In one embodiment, the promoter is a native promoter of strain 105. In an embodiment the promoter is a native inducible promoter of strain 105. In an embodiment, the promoter is a non-native promoter or a non-native inducible promoter. Exemplary and suitable promoters are provided herein. Alternative promoters are known or can be selected by one skilled in the art.

In an embodiment, nucleic acid encoding or facilitating production of a biomolecule or a homologous protein or heterologous protein is linked to a native strain 105 promoter. In an embodiment, nucleic acid encoding or facilitating production of a biomolecule or a homologous protein or heterologous protein is linked to one or more native strain 105 promoter. In an embodiment, nucleic acid encoding or facilitating production of a biomolecule or a homologous protein or heterologous protein is linked to at least two native strain 105 promoters in tandem. In some embodiments, these promoters facilitate expression and production in strain 105. Exemplary and suitable promoters are provided herein.

In an embodiment, strain 105 is modified to secrete or more effectively secrete a biomolecule, homologous protein or heterologous protein. In an embodiment, strain 105 is modified to include nucleic acid encoding or otherwise capable of producing a biomolecule, homologous protein or heterologous protein, wherein the nucleic acid includes a signal sequence. In one embodiment, the signal sequence is a native signal sequence of strain 105. In an embodiment, the signal sequence is a non-native signal sequence. Exemplary and suitable signal sequence are provided herein. Alternative signal sequences are known or can be selected by one skilled in the art. The signal sequence for secretion may be at least 20 amino acids, at least 25 amino acids, at least 30 amino acids, at least 35 amino acids, at least 40 amino acids, at least 44 amino acids, at least 50 amino acids, at least 55 amino acids, at least 60 amino acids, or at least 65 amino acids. The signal sequence for secretion may also be 20-65 amino acids, 20-60 amino acids; 20-55 amino acids; 20-50 amino acids, 20-45 amino acids, 20-40 amino acids, 20-35 amino acids, 20-30 amino acids, 25-65 amino acids, 25-60 amino acids; 25-55 amino acids; 25-50 amino acids, 25-45 amino acids, 25-40 amino acids, 25-35 amino acids, 25-30 amino acids, 30-65 amino acids, 30-60 amino acids; 30-55 amino acids; 30-50 amino acids, 30-45 amino acids, 30-40 amino acids, or 30-35 amino acids.

In an embodiment, strain 105 is modified to enhance maintenance metabolism and promote more effective growth and growth cycles. In an embodiment, strain 105 is modified to generate non-spore forming bacteria strain. In an embodiment, strain 105 is modified to delete or to otherwise inactivate one or more native sequence responsible for or contributing to spore formation. In an embodiment, one or more of the SpoA and/or SoIVB protein encoding genes are deleted or to otherwise inactivated.

In an embodiment, strain 105 is modified to block production of, delete or otherwise inactivate one or more native protease. In an embodiment, the protease is one or more extracellular prtease. In an embodiment, inactivation or deletion serves to stabilize or increase the half life of one or more excreted biomolecule, protein etc from the strain. In embodiments, one or more of native extracellular proteases NprE, AprE, Epr (Epr1 and Epr2), Bpr, Mpr, NprB, Vpr, and WprA from B. subtilis strain 105 are deleted or otherwise inactivated. In embodiments, one or more of native extracellular proteases NprE, AprE, NprB, Vpr, and WprA from B. subtilis strain 105 are deleted or otherwise inactivated. In an embodiment, one or more of native extracellular proteases NprE, AprE and Epr (Epr1 and Epr2) from B. subtilis strain 105 are deleted or otherwise inactivated. In an embodiment, native extracellular proteases NprE and Vpr from B. subtilis strain 105 are deleted or otherwise inactivated. In and embodiment, native extracellular proteases AprE, NprB and WprA from B. subtilis strain 105 are deleted or otherwise inactivated.

In an embodiment, strain 105 is modified to block production of, delete or otherwise inactivate one or more native lytic enzyme or antibacterial protein. Exemplary strain 105 native lytic enzymes and antibacterial proteins which can be deleted or otherwise inactivated are provided herein.

In embodiments, strain 105 is modified to comprise one or more the self-amplifying nucleic acid encoding one or more biomolecule or protein of interest. In some embodiments, the self-amplifying nucleic acid encodes a biomolecule having a therapeutic effect such as an antibody, an anti-infective peptide, an immunomodulatory protein, and an antigen.

The present invention provides an expression cassette within a genetically-modified strain 105 that includes a heterologous coding region encoding a desired biomolecule or heterologous protein. The desired biomolecule may be a biomolecule having anti-infective activity, a probiotic factor, an immunomodulatory factor, a growth-promoting biomolecule, etc. The biomolecule may have anti-infective activity active against a pathogenic bacterium or a parasite. The expression cassette may be a plasmid or a vector, including a vector for integration in the Bacillus strain genome. The expression cassette, plasmid, vector, may include promoter sequence(s), signal sequence, one or more biomolecule or protein encoding sequence, one or more selection sequence for selection or determination of the growth of the plasmid or vector, and/or for selection or determination of the integration of the plasmid or vector. Suitable promoters, signal sequences are provided herein or would be known and available to one skilled in the art.

The present invention provides a use of any genetically-modified B. subtilis disclosed herein in the manufacture of a medicament. The present invention provides a use of any genetically-modified B. subtilis disclosed herein in the preparation of a feed additive or a component of animal feed.

In an embodiment, the invention provides a probiotic and therapeutic composition comprising the genetically modified B subtilis strain, particularly genetically modified B. subtilis strain 105 as described and detailed herein. In an embodiment, the invention provides a probiotic and therapeutic composition comprising the genetically modified B subtilis strain, particularly genetically modified B. subtilis strain 105 as described and detailed herein and a carrier suitable for animal administration; wherein said composition results in the expression and production of one or more biomolecule or heterologous protein in said animal when an effective amount is administered to an animal, as compared to an animal not administered the composition.

Methods of treating or alleciation a condition, disorder, infection or disease in an animal are provided comprising administering to said animal a genetically modified B subtilis strain, particularly genetically modified B. subtilis strain 105 as described and detailed herein. In an embodiment, the strain is administered with a carrier suitable for animal administration. In an embodiment, the strain is administered orally as part of or a component in feed.

In an embodiment, the invention provides a feed additive comprising the genetically modified B subtilis strain, particularly genetically modified B. subtilis strain 105 as described and detailed herein.

The invention provides a method of manufacturing one or more biomolecule or protein of interest comprising:
(a) modifying B subtilis strain 105 to introduce one or more nucleic acid capable of encoding the one or more biomolecule or protein of interest,
(b) culturing the modified strain 105, and
(c) isolating the one or more biomolecule or protein of interest.

In an embodiment, the B. subtilis strain 105 is modified before step a to improve or otherwise increase the expression and/or production of a biomolecule of interest. In an embodiment, the B. subtilis strain 105 is modified before step a by altering its competence, deleting or inactivating one or more gene such as one or more native protease, lytic enzyme, or deleting or inactivating one or more gene or protein responsible for spore formation.

The invention relates to and provides modified Bacillus bacteria for production or live delivery of one or more biomolecule or heterologous protein, wherein the bacteria comprises Bacillus subtilis strain 105 (ELA191105) genetically modified in one or more aspect selected from the following:
(a) genetically modified to increase competency;
(b) genetically modified to reduce or block spore formation;
(c) genetically modified to delete or inactivate one or more native protease; and
(d) genetically modified to include nucleic acid encoding one or more biomolecule or heterologous protein.

In an embodiment of the modified Bacillus bacteria, the Bacillus subtilis strain 105 comprises the nucleic acid sequence set out in SEQ ID NO: 1 or comprises at least 95% identity, 97% identity, 98% identity, 99% identity in genomic sequence to SEQ ID NO: 1.

In an embodiment of the modified Bacillus bacteria, the Bacillus subtilis strain corresponds to ATCC deposit PTA-126786 strain or has at least 95% identity, 97% identity, 98% identity, 99% identity in genomic sequence to the sequence of ELA 191105 corresponding to ATCC deposit FTA-126786.

In an embodiment of the modified Bacillus bacteria, the B subtilis strain 105 comprises the nucleic acid sequence set out in SEQ ID NO: 1, 2, 3, 4, 5 or 6 or comprises nucleic acid that has at least 90% identity, 95% identity, 97% identity, 98% identity, 99% identity in genomic sequence to SEQ ID NO: 1, 2, 3, 4 5 or 6.

Embodiments are provided wherein in (a) the bacteria is modified to overexpress comK, comS, or comK and comS to increase competency. In an embodiment, a gene cassette encoding comK and comS is integrated in the B subtilis genome.

In an embodiment, competency is increased and transformation efficiency of the strain is increased by at least 20 fold, by 50 fold, by 50 fold or greater, by 60 fold, by 80 fold, by 80 fold or greater, by 90 fold, by 100 fold or by 100 fold or greater. In one embodiment, competency is increased and transformation efficiency of the strain is increased by about 80 fold, by 80 fold or greater, by 90 fold, by 100 fold. In an embodiment, competency is increased and transformation efficiency of the strain is increased by approximately 100 fold.

Embodiments are provided wherein in (b) the bacteria is modified to delete or inactivate one or more native gene encoding SpoOA, SpoIVB or SpoA and SpoIVB.

Embodiments are provided wherein in (c) the bacteria is modified to delete or inactivate one or more native protease or the gene encoding one or more native protease selected from NprE, AprE, Epr1, Epr2, Bpr, Mpr, NprB, Vpr, and WprA. In an embodiment, the bacteria is modified to delete or inactivate the gene encoding native proteases NprE and Vpr. In an embodiment, the bacteria is modified to delete or inactivate the gene encoding native proteases AprE, NprB and WprA.

In other aspects, the modified Bacillus bacteria, particularlu B subtilis strain 105, is further genetically modified to delete or inactivate one or more native lytic enzyme or antibacterial peptide. In an embodiment, one or more native lytic enzyme or antibacterial peptide selected from xpf, lytC1, lytC2 and sdpC are deleted or inactivated.

In other aspects, the modified Bacillus bacteria, particularlu B subtilis strain 105, is further genetically modified to delete or inactivate one or more native gene encoding a virulence factor, toxin or antibacterial resistance (AMR). In embodiments, the one or more virulence factor, toxin or antibacterial resistance (AMR) is selected from macrolide 2′phosphotransferase (mphK), ABC—F type ribosomal protection protein (vmlR), Streptothricin-N-acetyltransferase (satA), tetracyclin efflux protein (tet(L)), aminoglycoside 6-adenylyltransferase (aadK) (29), and rifamycin-inactivating phosphotransferase (rphC), as set out in Table 16.

In some embodiments, the modified Bacillus comprises a B. subtilis isolate having at least at least one gene knockout selected from the following genes: spoOA, spoIIIE, spoIVB, NprE, AprE, NprB, Vpr, WprA; and one or more heterologous gene encoding one or more biomolecule or heterologous protein operatively linked to one or more promoter selected from a tuf promoter, sigx promoter, gros promoter, ftsh promoter, a PxylA promoter, a mannose inducible promoter, and a Physpank promoter.

In some embodiments, the modified Bacillus comprises a B. subtilis strain 105 isolate modified to overexpress comK, comS or comK and comS to increase competency; having at least at least one gene knockout selected from the following genes: spoOA, spoIIIE, spoIVB, NprE, AprE, NprB, Vpr, WprA; and modified to comprise one or more heterologous gene encoding one or more biomolecule or heterologous protein operatively linked to one or more promoter selected from a tuf promoter, sigx promoter, gros promoter, ftsh promoter, a PxylA promoter, a mannose inducible promoter, and a Physpank promoter. In an embodiment, the one or more promoter is selected from SEQ ID NO: 66, SEQ ID NO: 67, SEQ ID NO: 68, SEQ ID NO: 69, SEQ ID NO: 40 and SEQ ID NO: 41.

In other aspects, the one or more heterologous gene encoding one or more biomolecule or heterologous protein is integrated in the host B subtilis strain 105 genome. In some embodiments, the one or more heterologous gene encoding one or more biomolecule or heterologous protein is integrated in the host B subtilis strain 105 genome at one or more gene locations selected from amyE, NprE, AprE, Epr1, Epr2, Bpr, Mpr, NprB, Vpr, and WprA.

In additional embodiments, the one or more biomolecule or heterologous protein is selected from an anti-infective agent, anti-bacterial agent, anti-pathogen agent, immunomodulatory factor or agent, antigen, antibody, growth-promoting biomolecule, a probiotic, and a bio-based chemical.

In an aspect thereof, the one or more biomolecule or heterologous protein is an anti-bacterial agent. In a further such aspect, the one or more anti-bacterial agent is one or more lysin or lytic peptide. In another aspect, the one or more lysin or lytic peptide is PIyCM, CP025C, lysostaphin or a native B. subtilis 105 lytic enzyme.

In some embodiments, the one or more anti-bacterial agent is one or more antimicrobial peptide (AMP). In an embodiment, the one or more antimicrobial peptide (AMP) is a mersacidin or a cathelicidin peptide. In an embodiment, the one or more antimicrobial peptide (AMP) is a CAP18 peptide. In embodiments thereof, the CAP 18 peptide may be rabbit CAP18 or human Cap18 LL37 or a CAP18 peptide from another animal, or variant thereof. In embodiments thereof, the CAP 18 peptide may be SEQ ID NO: 95 or SEQ ID NO: 96, or variant thereof.

In some embodiments, the one or more biomolecule or heterologous protein is one or more antibody or a fragment thereof. In an embodiment, the one or more antibody or fragment thereof is one or more single chain antibody, domain antibody, VHH antibody or nanobody. In other embodiments, the one or more single chain antibody, domain antibody, VHH antibody or nanobody one or more single chain antibody, domain antibody, VHH antibody or nanobody directed against a pathogenic bacteria.

In another embodiment, the one or more antibody is one or more VHH antibody or nanobody directed against Clostridium perfringens. In an embodiment, the one or more antibody is one or more VHH antibody or nanobody directed against Clostridium perfringens alpha toxin and NetB. In some embodiments, the one or more VHH antibody is selected from SEQ ID NO: 97, SEQ ID NO: 98, SEQ ID NO: 101 and SEQ ID NO: 102.

In other aspects, the one or more biomolecule or heterologous protein is one or more antigen and wherein said antigen is capable of stimulating an immune response against a parasite, bacteria, or virus. In an aspect, the one or more biomolecule or heterologous protein is one or more antigen capable of stimulating an immune response against an Eimeria parasite. In an aspect, the one or more antigen is selected from Eimeria tenella elongation factor-1α, EtAMA1, EtAMA2, Eimeria tenella 5401, Eimeria acervuline lactate dehydrogenase antigen gene, Eimeria maxima surface antigen gene, Glyceraldehyde 3-phosphate Dehydrogenase (GAPDH) and Eimeria common antigen 14-3-3. In a particular aspect, the one or more antigen is an Eimeria antigen encoded by one or more of SEQ ID NO: 105, SEQ ID NO: 106, SEQ ID NO: 107, SEQ ID NO: 108, or SEQ ID NO: 109.

In other embodiments, the one or more heterologous gene encoding one or more biomolecule or heterologous protein is provided on a biosynthetic gene cluster (BGC) and wherein the BGC or a portion thereof is integrated in the host B subtilis strain 105 genome.

In an embodiment, the biosynthetic gene cluster (BGC) is a PKS BGC or a mersacidin BGC. In one embodiment, the PKS BGC is capable of producing an AhR-activating metabolite. In another embodiment, the mersacidin BGC is capable of producing one or more mersacidin polypeptide SEQ ID NO: 22 or SEQ ID NO: 23 capable of inhibiting or killing one or more bacteria or virus.

In some embodiments, the PKS BGC comprises the nucleic acid set out in SEQ ID NO: 110 or comprises nucleic acid encoding one or more polypeptide selected from SEQ ID NOs: 7-21. In some embodiments, the mersacidin BGC comprises the nucleic acid set out in SEQ ID NO: 24 or comprises nucleic acid encoding one or more polypeptide selected from SEQ ID NOs: 25-32.

In other aspects, the one or more biomolecule or heterologous protein is a bio-based chemical. Chemicals or agents which are bio-based and are synthesized or capable of being synthesized by an animal, bacteria or fungi host cell are well known to one skilled in the art. These may include enzymes or intermediates in enzymatic reactions. These may include additives for stabilization of other agents. These may include molecules or proteins useful in the food, cosmetic or pharmaceutical industry.

In one embodiment, the bio-based chemical is gamma polyglutamic acid (γ-PGA). In an embodiment, the γ-PGA is encoded by the CapABC locus and the B subtilis strain 105 is modified to produce increased amounts of γ-PGA by integrating at least one additional copy of the CapABC locus in B subtilis strain 105 genome. In one such embodiment, at least one additional copy of the CapABC locus is integrated in B subtilis strain 105 genome at one or more gene locus selected from amyE, nprE, apr and wprA.

In some embodiments of the invention, the one or more heterologous gene encoding one or more biomolecule or heterologous protein includes a native B subtilis 105 strain or other bacterial strain signal sequence for secretion of the one or more biomolecule or heterologous protein by the modified bacteria.

In embodiments, the native B subtilis 105 strain or other bacterial strain signal sequence for secretion is selected from SEQ ID NO: 43, SEQ ID NO: 44, SEQ ID NO: 46, SEQ ID NO: 47, SEQ ID NO: 49, and SEQ ID NOs: 50-64.

In another aspect, a live delivery platform is provided herein comprising a genetically-modified Bacillus bacteria for production of one or more biomolecules or heterologous proteins in an animal, wherein the modified Bacillus comprises Bacillus subtilis strain 105 (ELA191105) genetically modified to include nucleic acid encoding one or more biomolecule or heterologous protein which is produced and delivered upon administration of the modified Bacillus bacteria to the animal.

In some aspects, the bacteria comprises Bacillus subtilis strain 105 (ELA191105) genetically modified in one or more aspect selected from the following:
(a) genetically modified to increase competency;
(b) genetically modified to reduce or block spore formation;
(c) genetically modified to delete or inactivate one or more native protease; and
(d) genetically modified to include nucleic acid encoding one or more biomolecule or heterologous protein.

In an aspect, the Bacillus subtilis strain 105 comprises the nucleic acid sequence set out in SEQ ID NO: 1 or comprises at least 95% identity, 97% identity, 98% identity, 99% identity in genomic sequence to SEQ ID NO: 1.

In an aspect, the Bacillus subtilis strain corresponds to ATCC deposit PTA-126786 strain or has at least 95% identity, 97% identity, 98% identity, 99% identity in genomic sequence to the sequence of ELA191105 corresponding to ATCC deposit PTA-126786.

In other aspects, the Bacillus subtilis bacteria is genetically modified to include nucleic acid encoding one or more biomolecule or heterologous protein and comprises an expression cassette;
wherein the expression cassette comprises one or more of:
a promoter for transcriptional expression,
a nucleic acid sequence encoding a signal sequence for secretion,
at least one heterologous coding region encoding a desired biomolecule or heterologous protein, and
terminators for translation and transcription termination.

In some embodiments, the promoter for transcriptional expression is one or more promoter selected from a tuf promoter, sigx promoter, gros promoter, ftsh promoter, a PxylA promoter, a mannose inducible promoter, and a Physpank promoter. In embodiments, the one or more promoter is selected from SEQ ID NO: 66, SEQ ID NO: 67, SEQ ID NO: 68, SEQ ID NO: 69, SEQ ID NO: 40 and SEQ ID NO: 41.

In embodiments, the nucleic acid sequence encoding a signal sequence for secretion encodes at least 20 amino acids, at least 25 amino acids, at least 30 amino acids, at least 35 amino acids, at least 40 amino acids, at least 44 amino acids, at least 50 amino acids, at least 55 amino acids, at least 60 amino acids, or at least 65 amino acids.

In some embodiments, the nucleic acid sequence encoding a signal sequence for secretion encodes a native B subtilis 105 strain or other bacterial strain signal sequence for secretion comprising a sequence selected from SEQ ID NO: 43, SEQ ID NO: 44, SEQ ID NO: 46, SEQ ID NO: 47, SEQ ID NO: 49, and SEQ ID NOs: 50-64.

In some embodiments, the expression cassette or the at least one heterologous coding region encoding a desired biomolecule or heterologous protein is integrated in the host B subtilis strain 105 genome.

In embodiments, the expression cassette or the at least one heterologous coding region encoding a desired biomolecule or heterologous protein is integrated in the host B subtilis strain 105 genome at one or more gene locations selected from amyE, NprE, AprE, Epr1, Epr2, Bpr, Mpr, NprB, Vpr, and WprA.

In certain embodiments, the desired biomolecule or heterologous protein is selected from an anti-infective agent, anti-bacterial agent, anti-pathogen agent, immunomodulatory factor or agent, antigen, antibody, growth-promoting biomolecule, a probiotic, and a bio-based chemical.

The invention further relates to a method of reducing colonization of an animal by a pathogenic bacterium, parasite or virus, the method comprising treating an animal with the modified Bacillus bacteria provided herein or with the live delivery platform provided herein.

In embodiments thereof, the animal is a bird, a human, or a non-human mammal.

In embodiments thereof, the pathogenic bacterium is selected from the group consisting of Salmonella, Clostridium, Campylobacter, Staphylococcus, Streptococcus, and an E. coli bacterium.

In embodiments, the pathogenic parasite is Eimeria.

In embodiments, the modified Bacillus bacteria or the live delivery platform is administered orally, parentally, nasally, or mucosally.

In some embodiments, the animal is a bird and wherein treatment is administered in ovo.

In aspects hereof a modified Bacillus bacteria and a live delivery platform are provided for use in therapy. In aspects, the modified Bacillus bacteria and alive delivery platform are provided for use in reducing colonization of an animal by a pathogenic bacterium, parasite or virus.

In other aspects, the modified Bacillus bacteria and alive delivery platform are provided for use in the manufacture of a medicament for reducing colonization of an animal by a pathogenic bacterium, parasite or virus. In other aspects, the modified Bacillus bacteria and alive delivery platform are provided for use in the manufacture of a medicament for stimulating an immune response in an animal against a pathogenic bacterium, parasite or virus. In other aspects, the modified Bacillus bacteria and alive delivery platform are provided for use in the manufacture of a medicament for passive immunization in an animal against a pathogenic bacterium, parasite or virus.

While there have been described what are presently believed to be the preferred embodiments of the present invention, those skilled in the art will realize that other and further changes and modifications may be made thereto without departing from the spirit of the invention, and it is intended to claim all such modifications and changes as come within the true scope of the invention.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1 depicts the SpoOA and SpoIVB locus from B. subtilis strain 105 wherein SpoIVb and SpoA are encoded by tandem located sequence.

FIG. 2 provides the gene maps for each of amyE, nprE, apr and wprA on the B. subtilis strain 105 genome.

FIGS. 3A and B. A depicts the pathway for poly-γ-glutamate biosynthesis. The B. subtilis strain 105 native locus for producing PGA is shown in B. The native B. subtilis locus comprises capC, capB and capA encoded from a single promoter.

FIG. 4 depicts engineering and design of the comKS cassette for integration and expression.

FIG. 5 depicts engineering of the PKS biosynthetic gene cluster (BGC) into B subtilis 105.

FIG. 6 depicts the Bacillus BGC expression vector with left and right amyE arms for homologous integration into Bacillus subtilis 105. Genes depicted on the left side of the vector circle are elements needed for single copy replication in E. coli. The lacI and kan genes are elements needed for homologous recombination into Bacillus genome, selection and expression. A PxylA and a Physpank promoter element are also depicted.

FIG. 7 depicts engineering of the Mersacidin biosynthetic gene cluster (BGC) into B subtilis 105. (A) provides the Mersacidin cluster including the lagD coding sequence (LagD CDS). (B) provides the Mersacidin cluster without the lagD coding sequence.

### DETAILED DESCRIPTION

In accordance with the present invention there may be employed conventional molecular biology, microbiology, and recombinant DNA techniques within the skill of the art. Such techniques are explained fully in the literature.

As used herein, “isolated” means that the subject isolate has been separated from at least one of the materials with which it is associated in a particular environment, for example, its natural environment.

Thus, an “isolate” does not exist in its naturally occurring environment; rather, it is through the various techniques known in the art that the microbe has been removed from its natural setting and placed into a non-naturally occurring state of existence. Thus, the isolated strain or isolated microbe may exist as, for example, a biologically pure culture in association with an acceptable carrier.

As used herein, “individual isolates” should be taken to mean a composition, or culture, comprising a predominance of a single species, or strain, of microorganism, following separation from one or more other microorganisms. The phrase should not be taken to indicate the extent to which the microorganism has been isolated or purified. However, “individual isolates” can include substantially only one species, or strain, of microorganism.

In certain aspects of the disclosure, the isolated Bacillus strain exists as isolated and biologically pure cultures. It will be appreciated by one of skill in the art, that an isolated and biologically pure culture of a particular Bacillus strain, denotes that said culture is substantially free (within scientific reason) of other living organisms and contains only the individual Bacillus strain in question. The culture can contain varying concentrations of said isolated Bacillus strain. The present disclosure notes that isolated and biologically pure microbes often necessarily differ from less pure or impure materials.

As used herein, “spore” or “spores” refer to structures produced by bacteria that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single bacterial vegetative cell. Bacterial spores are structures for surviving conditions that may ordinarily be nonconductive to the survival or growth of vegetative cells.

As used herein, the terms “colonize” and “colonization” include “temporarily colonize” and “temporary colonization”.

As used herein, “microbiome” refers to the collection of microorganisms that inhabit the gastrointestinal tract of an animal and the microorganisms' physical environment (i.e., the microbiome has a biotic and physical component). The microbiome is fluid and may be modulated by numerous naturally occurring and artificial conditions (e.g., change in diet, disease, antimicrobial agents, influx of additional microorganisms, etc.). The modulation of the gastrointestinal microbiome can be achieved via administration of the compositions of the disclosure can take the form of: (a) increasing or decreasing a particular Family, Genus, Species, or functional grouping of a microbe (i.e., alteration of the biotic component of the gastrointestinal microbiome) and/or (b) increasing or decreasing gastrointestinal pH, increasing or decreasing volatile fatty acids in the gastrointestinal tract, increasing or decreasing any other physical parameter important for gastrointestinal health (i.e., alteration of the abiotic component of the gut microbiome).

As used herein, “probiotic” refers to a substantially pure microbe (i.e., a single isolate) or a mixture of desired microbes, and may also include any additional components (e.g., carrier) that can be administered to an animal to provide a beneficial health effect. Probiotics or microbial compositions of the invention may be administered with an agent or carrier to allow the microbes to survive the environment of the gastrointestinal tract, i.e., to resist low pH and to grow in the gastrointestinal environment.

The term “growth medium” as used herein, is any medium which is suitable to support growth of a microbe. By way of example, the media may be natural or artificial including gastrin supplemental agar, minimal media, rich media, LB media, blood serum, and tissue culture gels. It should be appreciated that the media may be used alone or in combination with one or more other media. It may also be used with or without the addition of exogenous nutrients.

As used herein, “improved” should be taken broadly to encompass improvement of a characteristic of interest, as compared to a control group, or as compared to a known average quantity associated with the characteristic in question. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.”

As used herein, the term “metabolite” refers to an intermediate or product of metabolism. In some embodiments, a metabolite includes a small molecule. Metabolites have various functions, including in fuel, structural, signaling, stimulatory and inhibitory effects on enzymes, as a cofactor to an enzyme, in defense, and in interactions with other organisms (such as pigments, odorants and pheromones). A primary metabolite is directly involved in normal growth, development and reproduction. A secondary metabolite is not directly involved in these processes but usually has an important ecological function. Examples of metabolites include but are not limited to antibiotics and pigments such as resins and terpenes, etc. Metabolites, as used herein, include small, hydrophilic carbohydrates; large, hydrophobic lipids and complex natural compounds.

As used herein, “carrier”, “acceptable carrier”, or “pharmaceutical carrier” are used interchangeably and refer to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Handbook of Pharmaceutical Excipients, (Sheskey, Cook, and Cable) 2017, 8th edition, Pharmaceutical Press; Remington's Pharmaceutical Sciences, (Remington and Gennaro) 1990, 18th edition, Mack Publishing Company; Development and Formulation of Veterinary Dosage Forms (Hardee and Baggot), 1998, 2nd edition, CRC Press.

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, “animal” includes bird, poultry, a human, or a non-human mammal. Specific examples include chickens, turkey, dogs, cats, cattle, salmon, fish, swine and horse. The chicken may be a broiler chicken, egg-laying, or egg-producing chicken. As used herein, the term “poultry” includes domestic fowl, such as chickens, turkeys, ducks, and geese.

As used herein, “gut” refers to the gastrointestinal tract including stomach, small intestine, and large intestine. The term “gut” may be used interchangeably with “gastrointestinal tract”.

As used herein, a “genetically-modified microorganism” means any microorganism which has been altered from the natural state using molecular biological techniques. A genetic modification could be the deletion of a portion of the bacterial chromosome or a naturally-occurring plasmid. The genetic modification could also be the introduction of an artificial or exogenous nucleic acid into a portion of the chromosome. The introduction may or may not disturb or perturb the expression of a bacterial gene. The genetic modification could also be the introduction of an artificial plasmid. The genetically-modified microorganism may be a bacterium, a virus, a yeast, a mold, or a single-celled organism.

An “artificial nucleic acid” or “artificial plasmid” is any nucleic acid or plasmid which does not occur naturally, but rather has been constructed using molecular biological techniques. Portions of the nucleic acid or plasmid may occur naturally, but the those portions are in an artificial relationship or organization.

As used herein, an “expression cassette” is an artificial nucleic acid constructed to result in the expression of a desired biomolecule by the genetically-modified microorganism. An expression cassette comprises one or more of a promoter for transcriptional expression, a nucleic acid sequence encoding a signal sequence for secretion, a nucleic acid sequence encoding a cell-wall anchor, at least one heterologous coding region encoding a desired biomolecule, a nucleic acid sequence encoding an expressed peptide tag for detection, and terminators for translation and transcription termination. A promoter directs the initiation of transcription of the coding regions into a messenger RNA and the translation of the mRNA into a peptide. A signal sequence for secretion, or a secretion signal sequence, directs the peptide to be located outside the cell membrane. The extracellular peptide could be a soluble, secreted protein or it may be cell-associated, particularly if the expression cassette contains a cell wall anchor sequence which attaches the extracellular peptide to a bacterial cell wall. An expressed peptide tag is any amino acid sequence which may be recognized by an antibody or other binding protein. The expressed peptide tag may also bind an inorganic substance, such as a six-histidine tag which binds to nickel molecules. Terminators for translation may be a stop codon or a spacer open reading frame containing a stop codon.

As used herein, a “heterologous coding region” is a nucleic acid sequence containing an open reading frame which encodes a peptide. The coding region is heterologous to the associated promoter, meaning the coding region and the promoter are not associated in their natural states.

A “heterologous” region of a nucleic acid, RNA or DNA, construct is an identifiable segment of RNA or DNA within a larger RNA or DNA molecule that is not found in association with the larger molecule in nature. Thus, when the heterologous region encodes a gene, the gene will usually be flanked by RNA or DNA that does not flank the genomic RNA or DNA in the genome of the source organism.

As used herein, a “protein” is a sequence of amino acids which assumes a three-dimensional structure. A “peptide” can be used interchangeably with protein, but may also be a short linear sequence of amino acids without a defined three-dimensional structure.

As used herein, a “desired biomolecule” is any molecule or peptide which may be advantageous to a host when administered via a live delivery platform. The desired biomolecule may be a peptide with anti-infective activity, a probiotic factor, an immunomodulatory factor, an anti-antinutritional factor, or a growth-promoting biomolecule. The desired biomolecule may also be an enzyme which produces a substance with anti-infective activity or a probiotic factor such as a vitamin.

As used herein, “anti-infective activity” includes any activity which prevents infection of a host with a pathogenic organism. The following molecules are examples of biomolecules possessing anti-infective activity: an antibacterial peptide; a lysin or lytic enzyme; a prophage, phage or virus; an enzyme, for example one that cleaves or disables a protein made by a pathogen; and an antibody which blocks, inhibits, or clears a pathogenic molecule. An anti-infective may have bactiostatic activity, which slows, reduces, or prevents the growth of a pathogenic species. A non-limiting example of an antibacterial peptide is a member of the mersacidin family or a mersacidin-like molecule, such as those described in EP0700998. A non-limiting example of lysins are lytic molecules produced by phage. Lysins may have specificity for certain pathogenic species of bacteria and have been suggested for use in substitution for traditional antibiotics. V. A. Fischetti, Viruses, vol. 10, no. 310 (2018); and R. Vazquez et al. Frontiers in Immunology, vol. 9, article 2252 (2018).

As used herein, a “probiotic factor” is a substance which, when produced by a genetically-modified microorganism, proves beneficial to a host. The probiotic factor may be an attachment molecule or an agglutinizing molecule which promotes colonization of the host with the genetically-modified microorganism and/or prolongs the period of time where the genetically-modified microorganism colonizes the host. The longer the genetically-modified microorganism persists in the host the longer the beneficial effect is provided.

As used herein, an “immunomodulatory factor” could be a cytokine, lymphokine, chemokine, interleukin, interferon, a colony stimulating factor, or a growth factor. The immunomodulatory factor could provide nonspecific enhancement of an immune response or the immunomodulatory factor could increase the number or tissue distribution of immune cells present in the host. The immunomodulatory factor may also reduce an inappropriate immune response, such as without limitation an autoimmune response.

As used herein, a “growth-promoting biomolecule” could be a growth factor, a transfer factor (such as an iron-chelating molecule), a hormone, or any other factor which promotes healthy metabolic activity.

As used herein, an “anti-nutritional factor” could include protease inhibitors for example, a trypsin inhibitors.

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, the terms “treating”, “to treat”, or “treatment”, include restraining, slowing, stopping, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

As used herein, “subject” includes bird, poultry, fish, a human, or a non-human animal. Specific examples include chickens, turkey, dogs, cats, cattle, and swine. The chicken may be a broiler chicken, egg-laying or egg-producing chicken. As used herein, the term “poultry” includes domestic fowl, such as chickens, turkeys, ducks, quail, and geese.

A “heterologous” region of a nucleic acid, RNA or DNA, construct is an identifiable segment of RNA or DNA within a larger RNA or DNA molecule that is not found in association with the larger molecule in nature. Thus, when the heterologous region encodes a gene, the gene will usually be flanked by RNA or DNA that does not flank the genomic RNA or DNA in the genome of the source organism.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

A “chimeric protein” or “fusion protein” comprises all or (preferably a biologically active) part of a first polypeptide operably linked to a heterologous polypeptide. Chimeric proteins or peptides are produced, for example, by combining two or more proteins having two or more active sites. In a chimeric or fusion protein, a first polypeptide may be covalently attached to an entity which may provide additional function or enhance the use or application of the first polypeptide(s), including for instance a tag, label, targeting moiety or ligand, a cell binding or cell recognizing motif or agent, an antibacterial agent, an antibody, an antibiotic. Exemplary labels include a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. The label may be an enzyme, and detection of the labeled lysin polypeptide may be accomplished by any of the presently utilized or accepted colorimetric, spectrophotometric, fluorospectrophotometric, amperometric or gasometric techniques known in the art. Chimeric protein and peptides can act independently on the same or different molecules or targets, and hence have a potential to provide multiple activities, such as to treat or stimulate immune response against two or more different bacterial infections or infective agents at the same time.

As used herein, the term “mutant”, refers to a variation in a nucleic acid or DNA or RNA sequence or in a chromosome structure from that which is considered a normal or wild-type sequence or chromosome without defect. In the context of a nucleic acid, DNA or RNA sequence, examples of mutations include point mutations, insertions, and deletions. A deletion includes deletion of a part or entire gene. Such mutations may have functional effects such as, for example, a decrease in function of a gene product, ablation of function in a gene product, and/or a new or altered function in a gene product.

As used herein, “mutation” includes any alteration in one or more nucleic acids in a genomic sequence, including one or more base changes, deletions, and/or insertions, that result in silent mutations, non-sense mutations, mis-sense mutations, or any such other mutations that result in reduced function of a gene or result in an inactive or otherwise non-functional protein encoded by a gene. Mutations include but are not limited to mutations that result in premature stop codons, aberrant splicing, altered or failed transcription, or altered or failed translation. A gene comprising a mutation can have more than one mutation. Mutations include deletion of a gene or a significant portion of a gene, particularly such that the gene's protein is not produced or expressed and/or is inactive. Mutations include insertions, such as wherein a foreign or heterologous sequence or nucleic acid is introduced into or otherwise inserted in the gene. Such insertion may block or eliminate translation to active or full length protein, or may result in a significantly altered and distinct protein that is not active as the wild type. An insertion may facilitate isolation, detection, selection of the gene mutant, such as by introduction or insertion of an antibiotic resistance gene or a detectable marker or protein. In particular embodiments of the invention and as described herein, the mutation, including one or more mutation, is a non-natural mutation and is genetically engineered or recombinantly generated. In some embodiments, the mutation is genetically engineered or generated recombinantly in vitro. In some embodiments, the mutation is genetically engineered or generated recombinantly in a cell.

In some embodiments, a mutation is generated whereby a gene, or a large or significant portion of a gene or protein encoding nucleic acid, is deleted. In embodiments, one or more gene or a large or significant portion of a gene or protein encoding nucleic acid is deleted for example via recombination methods. Recombination methods for targeted deletion of genes are known and available to one skilled in the art. Such methods include homologous recombination, such as via an introduced plasmid, phage or nucleic acid such as DNA or linear DNA fragemt(s), recombination enzymes or recombinase enzyme mediated recombination, for example via recombinase recognition or target sequences sequences, transposon mediated recombination and gene replacement.

In accordance with some embodiments of the present invention, deletion or inactivation mutations have been generated whereby one or more gene(s) are deleted or inactivated in the genome of Bacillus subtilis bacteria. Deletion mutants have thus been generated and utilized or have been utilized whereby deletions in each of the genes were constructed to provide new Bacillus subtilis mutant strains of bacteria. In some embodiments, these mutant bacteria are altered in growth.

In embodiments of the invention the gene mutation is a gene deletion mutation. In embodiments the gene mutation is a deletion generated by recombination, including wherein a substantive portion of the encoding region of the gene is deleted. In some embodiments, a substantive portion of the encoding gene is deleted and is replaced by insertion of a tag or marker, such as a detectable tag or a selectable marker.

The therapeutic or biologically active molecule may be any molecule, including a polypeptide or nucleic acid, having a useful or desired activity. A therapeutic biomolecule includes a biomolecule having a therapeutic effect. Examples of therapeutic biomolecules include antibody, a ribonucleic acid (RNA), and antigen. Antibody includes antibody fragments, such as VHH. RNA includes inactivating RNA, such as shRNA and siRNA. Antigen includes a biomolecule that stimulates an immune response. Examples of antigens include a peptide, polypeptide, protein, nucleic acid molecule, and carbohydrate molecule. In some embodiments, the molecule may be selected from an antibody, a ribonucleic acid (RNA), a peptide or protein, and an antigen.

Antibodies in accordance with the present disclosure include an immunoglobulin and particularly any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies. Also included within the meaning of the term “antibody” are any “antibody fragment”. An “antibody fragment” refers to a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv), which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); (xii) a minibody, which is a bivalent molecule comprised of scFv fused to constant immunoglobulin domains, CH3 or CH4, wherein the constant CH3 or CH4 domains serve as dimerization domains (Olafsen T et al (2004) Prot Eng Des Sel 17(4):315-323; Hollinger P and Hudson P J (2005) Nature Biotech 23(9):1126-1136); and (xiii) other non-full length portions of heavy and/or light chains, or mutants, variants, or derivatives thereof, alone or in any combination. Chimeric molecules comprising an immunoglobulin binding domain, or equivalent, fused to another polypeptide are also included.

An “antibody combining site” is that structural portion of an antibody molecule comprised of light chain or heavy and light chain variable and hypervariable regions that specifically binds antigen. Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v). Antibodies may also be bispecific, wherein one binding domain of the antibody has a first binding specificity, and the other binding domain has a different specificity, e.g. to recruit an effector function or the like. The other binding domain may be an antibody that recognizes or targets a particular cell type or to recognize particular cell receptors and/or modulate cells in a particular fashion, as for instance an immune modulator (e.g., interleukin(s)), a growth modulator or cytokine or a toxin (e.g., ricin) or anti-mitotic or apoptotic agent or factor.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. An antigen binding domain may comprise an antibody light chain variable region (VL) and an antibody heavy chain variable region (VH), or may only comprise an antibody heavy chain variable region (VH).

Immunoconjugates or antibody fusion proteins are also contemplated, wherein the antibodies, antibody molecules, or fragments thereof, applicable in the present invention are conjugated or attached to other molecules or agents. Such immunoconjugates or antibody fusion proteins may further include, but are not limited to such antibodies, molecules, or fragments conjugated to a chemical ablation agent, toxin, immunomodulator, cytokine, cytotoxic agent, chemotherapeutic agent, antimicrobial agent or peptide, cell wall and/or cell membrane disrupter, or drug.

Single domain antibodies are included as a particular embodiment of the therapeutic or biologically active molecules delivered in accordance with the intracellular delivery platform provided herein and expressed by the self amplifying nucleic acid. Single domain antibodies were initially isolated from camelid animals and have been designated interchangeably as camelid antibodies, nanobodies or VHH. A VHH antibody corresponds to the variable region of an antibody heavy chain and has a very small size of around 15 kDa—hence the name “nanobody”. The advantage of these antibody-derived molecules is their small size which can enable their binding to hidden epitopes not accessible to whole antibodies. In the context of therapeutic applications, a small molecular weight also means an efficient penetration and fast clearance. Both scFv and VHH nanobodies can he linked to the Fc fragment of the desired species and keep their specificity and binding properties and are then termed minibody.

Delivering nanobodies: Nanobodies are small, low molecular weight, single-domain, heavy-chain only antibody found in camelids. Owing to its smaller size, genes of these proteins are easy to clone inside a plasmid. Therefore, by using molecular cloning techniques, nanobodies against various antigens can be presented in the systemic circulation. The present invention and intracellular delivery platform has been utilized to deliver and express antibody fragments, particularly VHH or nanobodies.

An antigen is a substance, such as a protein or peptide, which induces an immune response, especially the production of antibodies. In immunology, an antigen is a molecule or molecular structure, such as may be present on the outside of a pathogen, that can be bound by an antigen-specific antibody or B-cell antigen receptor. The presence of antigens in the body normally triggers an immune response. Antigens, or peptide or protein sequences, capable of eliciting an immune response, particularly a protective or neutralizing immune response, have been defined in many systems. The basis of vaccines is the presentation of one or more antigen from an infectious agent to an animal or host, such that the animal or host has an immune response and raises antibodies against the infectious agent. This immune response and these reaised antibodies serve to protect the host or animal from further infection, disease or illness by the infectious agent. In embodiments of the present invention, vaccines provided and contemplated herein are capable of and utilized to generate mucosal, systemic and cellular immunity against one or more pathogen(s).

An antigen may include all or a portion of a protein. In particular, an antigen may be an antigenic portion or fragment of a full length protein. An antigen may be a non-natural fragment of a protein. The delivery platform may be utilized to express one or more antigen for a particular pathogen. Multiple antigens may be expressed from a single self-amplifying RNA for example. Multiple antigens of an infectious agent or pathogen may be expressed from a single B. subtilis 105 strain.

There are various peptides or proteins which act independently as therapeutic biolmolecules. Among these are anti-infective or anti-bacterial peptides which can serve to block or treat infection by infectious agents or bacteria.

A wide range of antimicrobial peptides is secreted in plants and animals to challenge attack by foreign viruses, bacteria or fungi (Boman, H. G. (2003) J. Intern. Med. 254 (3):197-215). These form part of the innate immune response to infection, which is short term and fast acting relative to humoral immunity. These peptides are heterogeneous in length, sequence and structure, but most are small, cationic and amphipathic (Zasloff, M. (2002) Nature 415(6870):389-395). Exemplary such known antimicrobial peptides are listed at an antimicorobial database (aps.unmc.edu/AP/main.php; Wang Z and Wang G (2004) NAR 32:D590-D592) and the content and disclosure of this site is incorporated herein by reference in its entirety. While the external cell wall may be the initial target, several lines of evidence suggest that antimicrobial peptides act by lysing bacterial membranes. Cells become permeable following exposure to peptides, and their membrane potential is correspondingly reduced. Protamines or polycationic amino acid peptides containing combinations of one or more recurring units of cationic amino acids, such as arginine (R), tryptophan (W), lysine (K), even synthetic polyarginine, polytryptophan, polylysine, have been shown to be capable of killing microbial cells.

A cell-wall degrading enzyme is an enzyme which degrades components of the cell wall, including peptidoglycans, such as murein and pseudomurein, chitin, and teichoic acid. Cell-wall degrading enzymes can include, but are not limited to amidases, muramidases, endopeptidases, glucosaminidases. Bacteriophage lysins are cell wall degrading anti-bacterial enzymes encoded by phage in bacteria. Lysins are peptidoglycan hydrolases that break bonds in the bacterial wall, rapidly hydrolyzing covalent bonds essential for peptidoglycan integrity, causing bacterial lysis and concomitant progeny phage release. Bacteriophage lytic enzymes have been established as useful in the assessment and specific treatment of various types of infection in subjects through various routes of administration. Phage associated lytic enzymes have been identified and cloned from various bacteriophages, each shown to be effective in killing specific bacterial strains.

The application of bacteria such as Bacillus as a vector to express, produce or deliver immune, prophylactic or any such other therapeutic biomolecules pmvides a number of applicable products and therapies targeting multiple disease conditions across a range of host species. There are multiple ways whereby live bacterial vectors and expression systems can be modified to deliver heterologous antigens, for example, as chromosomal or plasmid integrated genes, or a payload of eukaryotic antigen-expression plasmids (so-called DNA vaccines), but these systems have limitations, including in their means of expressing the heterologous antigens. More recently, RNA-based vaccines, both messenger RNA (mRNA) and self-amplifying replicons (SAM) are emerging as an increasingly promising alternative to traditional plasmid DNA for gene vaccination (DNA vaccines). RNA vaccines have been shown to elicit antigen specific antibody and cellular immune responses against several viral pathogens with some clear advantages over DNA. The present invention provides a novel delivery platform for delivering antigens, immunogens, antibodies, bioactive peptides, RNAs and other biotherapeutics and therapeutic biomolecules. The present invention provides a novel delivery platform for delivering immunogens, antibodies and therapeutic biomolecules as vaccines, including prophylactic and therapeutic vaccines.

The intracellular delivery platform and production system of the present disclosure includes a genetically modified bacterium having a self-amplifying or integrated nucleic acid capable of encoding a biomolecule or heterologous protein.

In an embodiment, a probiotic composition is provided comprising the genetically modified Bacillus subtilis strain 105 herein comprising nucleic acid encoding a biomolecule or heterologous protein for production, for delivery, of interest, or of therapeutic importance.

In some embodiments, the composition includes a genetically modified Bacillus subtilis strain 105 wherein ELA191105 or an active and effective variant thereof has been modified. In some embodiments, the composition includes a genetically modified Bacillus subtilis strain 105 and also, including in combination, another isolated Bacillus strain, particularly a distinct Bacillus strain having probiotic properties or activity, including particularly when combined with strain 105. In some embodiments, the B subtilis strain 105 can be combined with one or more isolated Bacillus amyloliquefaciens strain, particularly selected from ELA191024 (corresponding to ATCC deposit PTA-126784), ELA191036 (corresponding to ATCC deposit PTA-126785), ELA191006 (corresponding to ATCC deposit PTA-127065) and ELA202071 (corresponding to ATCC deposit PTA-127064).

These probiotic strain combinations and compositions and methods thereof are described and provided in PCT/US2021/051973 published as WO2022/067052 Mar. 31, 2022, which is incorporated herein by reference.

In some embodiments, the composition does not include Lactobacillus. An example of a LactoBacillus species includes LactoBacillus reuteri and LactoBacillus crispatus, LactoBacillus vaginalis, LactoBacillus helviticus, and LactoBacillus johnsonii.

In some embodiments, the composition does not include non-Bacillus strains. Examples of non-Bacillus strains include Lactobacillus, Leuconostoc (e.g., Leuconostoc mesenteroides).

The composition may include or comprise live bacteria or bacterial spores, or a combination thereof.

In some embodiments, the composition does not include antibiotics. Exemplary antibiotics include tetracycline, bacitracin, tylosin, salinomycin, virginiamycin and bambermycin.

The compositions described above may include a carrier suitable for animal consumption or use. Examples of suitable carriers include edible food grade material, mineral mixture, gelatin, cellulose, carbohydrate, starch, glycerin, water, glycol, molasses, corn oil, animal feed, such as cereals (barley, maize, oats, and the like), starches (tapioca and the like), oilseed cakes, and vegetable wastes. In some embodiments, the compositions include vitamins, minerals, trace elements, emulsifiers, aromatizing products, binders, colorants, odorants, thickening agents, and the like.

In some embodiments, the compositions include one or more biologically active molecule or therapeutic molecule. Examples of the aforementioned include ionophore; vaccine; antibiotic; antihelmintic; virucide; nematicide; amino acids such as methionine, glycine, and arginine; fish oil; krill oil; and enzymes.

In some embodiments, the compositions or combinations may additionally include one or more prebiotic. In some embodiments, the compositions may be administered along with or may be coadministered with one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

The composition may be formulated as animal feed, feed additive, animal food, food ingredient, water additive, water-mixed additive, consumable solution, consumable spray additive, consumable solid, consumable gel, injection, or combinations thereof. The composition may be formulated and suitable for use as or in one or more of animal feed, feed additive, food ingredient, water additive, water-mixed additive, consumable solution, consumable spray additive, consumable solid, consumable gel, injection, or combinations thereof. The composition may be suitable and prepared for use as animal feed, feed additive, animal food, food ingredient, water additive, water-mixed additive, consumable solution, consumable spray additive, consumable solid, consumable gel, injection, or combinations thereof.

In some embodiments, the disclosure provides for the use of any of the compositions described above in a therapy or treatment or to improve a phenotypic trait in an animal.

In embodiments of the invention, an animal may include a farmed animal or livestock or a domesticated animal. Livestock or farmed animal may include cattle (e.g. cows or bulls (including calves)), poultry (including broilers, chickens and turkeys), pigs (including piglets), birds, aquatic animals such as fish, agastric fish, gastric fish, freshwater fish such as salmon, cod, trout and carp, e.g. koi carp, marine fish such as sea bass, and crustaceans such as shrimps, mussels and scallops), horses (including race horses), sheep (including lambs). A domesticated animal may be a pet or an animal maintained in a zoological environment and may include any relevant animal including canines (e.g. dogs), felines (e.g. cats), rodents (e.g. guinea pigs, rats, mice), birds, fish (including freshwater fish and marine fish), and horses. The animal may be a human.

The animal may be a pregnant or breeding animal, such as a pregnant sow or a pregnant pig.

Examples of improving a phenotypic trait includes decreasing pathogen-associated lesion formation in the gastrointestinal tract or otherwise in the animal, decreasing colonization of pathogens, decreasing transmission of one or more pathogen, promoting immune response or generation of antibodies against a pathogen, and increasing gut health or characteristic (reducing permeability and inflammation).

Examples of pathogens include Eimeria spp., Salmonella Typhimurium, Salmonella Infantis, Salmonella Hadar, Salmonella Enteritidis, Salmonella Newport, Salmonella Kentucky, Clostridium perfringens, Staphylococcus aureus, Streptoccus uberis, Streptococcus suis, Streptococcus pneumoniae, Escherichia coli, Campylobacter jejuni, Clostridium perfringes, Fusobacterium necrophorum, Avian pathogenic Escherichia coli (APEC), Pisciricketsia salmonis, Tenacibaculum spp., Salmonella Lubbock, Trueperella pyogenes, shiga toxin producing E. coli, enterotoxigenic E. coli, Campylobacter coli, and Lawsonia intracellularis.

A pathogen may be a bacteria, a parasite or a virus. The virus may include a pathogenic virus infecting animals, including humans, livestock animals or domesticated animals and may be specific for a particular animal such as a poultry virus or a swine virus.

The compositions may be used to treat an infection particularly a bacterial infection. In some aspects, the compositions described above are used to treat an infection from at least one of Eimeria spp., Salmonella Typhimurium, Salmonella Infantis, Salmonella Hadar, Salmonella Enteritidis, Salmonella Newport, Salmonella Kentucky, Clostridium perfringens, Staphylococcus aureus, Streptoccus uberis, Streptococcus suis, Escherichia coli, Campylobacter jejuni, Fusobacterium necrophorum, Avian pathogenic Escherichia coli (APEC), Salmonella Lubbock, Trueperella pyogenes, shiga toxin producing E. coli, enterotoxigenic E. coli, Campylobacter coli, and Lawsonia intracellularis. The compositions may be used to inhibit infection, particularly a bacterial infection. Infection may be by one or more of Eimeria spp., Salmonella Typhimurium, Salmonella Infantis, Salmonella Hadar, Salmonella Enteritidis, Salmonella Newport, Salmonella Kentucky, Clostridium perfringens, Staphylococcus aureus, Streptoccus uberis, Streptococcus suis, Escherichia coli, Campylobacter jejuni, Fusobacterium necrophorum, Avian pathogenic Escherichia coli (APEC), Salmonella Lubbock, Trueperella pyogenes, shiga toxin producing E. coli, enterotoxigenic E. coli, Campylobacter coli, and Lawsonia intracellularis.

In some aspects, the compositions described above are used to reduce colonization by or inhibit colonization by a bacteria in an animal, particularly in a herd or group of animals, particularly of pathogenic bacteria. In some aspects, the compositions described above are used to reduce colonization by or inhibit colonization of at least one of Eimeria spp., Salmonella Typhimurium, Salmonella Infantis, Salmonella Hadar, Salmonella Enteritidis, Salmonella Newport, Salmonella Kentucky, Clostridium perfringens, Staphylococcus aureus, Streptoccus uberis, Streptococcus suis, Escherichia coli, Campylobacter jejuni, Fusobacterium necrophorum, Avian pathogenic Escherichia coli (APEC), Salmonella Lubbock, Trueperella pyogenes, shiga toxin producing E. coli, enterotoxigenic E. coli, Campylobacter coli, and Lawsonia intracellularis.

In some aspects, the compositions described above are used to reduce transmission of bacteria, particularly pathogenic bacteria, in an animal pen or in a group or herd of animals. In some aspects, the compositions described above are used to reduce transmission in an animal pen or in a group or herd of animals of at least one of Eimeria spp., Salmonella Typhimurium, Salmonella Infantis, Salmonella Hadar, Salmonella Enteritidis, Salmonella Newport, Salmonella Kentucky, Clostridium perfringens, Staphylococcus aureus, Streptoccus uberis, Streptococcus suis, Escherichia coli, Campylobacter jejuni, Fusobacterium necrophorum, Avian pathogenic Escherichia coli (APEC), Salmonella Lubbock, Trueperella pyogenes, shiga toxin producing E. coli, enterotoxigenic E. coli, Campylobacter coli, and Lawsonia intracellularis.

In some aspects, the compositions described above are used to reduce bacterial load, particularly pathogenic bacteria or clinically significant bacteria, including the number or amount of bacteria in the gut or gastrointestinal tract of an animal. The bacteria may be selected from at least one of Eimeria spp., Salmonella Typhimurium, Salmonella Infantis, Salmonella Hadar, Salmonella Enteritidis, Salmonella Newport, Salmonella Kentucky, Clostridium perfringens, Staphylococcus aureus, Streptoccus uberis, Streptococcus suis, Escherichia coli, Campylobacter jejuni, Fusobacterium necrophorum, Avian pathogenic Escherichia coli (APEC), Salmonella Lubbock, Trueperella pyogenes, shiga toxin producing E. coli, enterotoxigenic E. coli, Campylobacter coli, and Lawsonia intracellularis.

In some aspects, the compositions described above are used to treat at least one of inflammatory bowel disease, obesity, liver abscess, ruminal acidosis, leaky gut syndrome, piglet diarrhea, necrotic enteritis, coccidiosis, salmon ricketsial septicemia, and foodborne diseases.

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal, intramammary, topical, mucosally, or inhalation. When the animal is a bird the treatment may be administered in ovo or by spray inhalation.

Compositions may include a carrier in which the bacterium or any such other components is suspended or dissolved. Such carrier(s) may be any solvent or solid or encapsulated in a material that is non-toxic to the inoculated animal and compatible with the organism. Suitable pharmaceutical carriers include liquid carriers, such as normal saline and other non-toxic salts at or near physiological concentrations, and solid carriers, such as talc or sucrose and which can also be incorporated into feed for farm animals. When used for administering via the bronchial tubes, the composition is preferably presented in the form of an aerosol. A dye may be added to the compositions hereof, including to facilitate chacking or confirming whether an animal has ingested or breathed in the composition.

When administering to animals, including farm animals, administration may include orally or by injection. Oral administration can include by bolus, tablet or paste, or as a powder or solution in feed or drinking water. The method of administration will often depend on the species being feed or administered, the numbers of animals being fed or administered, and other factors such as the handling facilities available and the risk of stress for the animal.

The dosages required will vary and need be an amount sufficient to induce an immune response or to effect a biological or phenotypic change or response expected or desired. Routine experimentation will establish the required amount. Increasing amounts or multiple dosages may be implemented and used as needed.

In an embodiment of the invention, the bacterial strains are administered in doses indicated as CFU/g or colony forming units of bacteria per gram. In an embodiment, the dose is in the range of 1×10³ to 1×10⁹ CFU/g. In an embodiment, the dose is in the range of 1×10³ to 1×10⁷. In an embodiment, the dose is in the range of 1×10′ to 1×10⁶. In an embodiment, the dose is in the range of 5×10⁴ to 1×10⁶. In an embodiment, the dose is in the range of 5×10′ to 6×10⁵. In an embodiment, the dose is in the range of 7×10⁴ to 3×10⁵. In an embodiment, the dose is approximately 50K, 75K, 100K, 125K, 150K, 200K, 300K, 400K, 500K, 600K CFU/g.

Administration of the compositions disclosed herein may include co-administration with a vaccine or therapeutic compound. Administration of the vaccine or therapeutic compound includes administration prior to, concurrently, or after the composition disclosed herein.

Suitable vaccines in accordance with this embodiment include a vaccine that aids in the prevention of coccidiosis.

In some embodiments, the methods described above are administered to an animal in the absence of antibiotics.

An antigen is a substance, such as a protein or peptide, which induces an immune response, especially the production of antibodies. In immunology, an antigen is a molecule or molecular structure, such as may be present on the outside of a pathogen, that can be bound by an antigen-specific antibody or B-cell antigen receptor. The presence of antigens in the body normally triggers an immune response. Antigens. or peptide or protein sequences, capable of eliciting an immune response, particularly a protective or neutralizing immune response, have been defined in many systems. The basis of vaccines is the presentation of one or more antigen from an infectious agent to an animal or host, such that the animal or host has an immune response and raises antibodies against the infectious agent. This immune response and these reaised antibodies serve to protect the host or animal from further infection, disease or illness by the infectious agent. In embodiments of the present invention, vaccines provided and contemplated herein are capable of and utilized to generate mucosal, systemic and cellular immunity against one or more pathogen(s).

An antigen may include all or a portion of a protein. In particular, an antigen may be an antigenic portion or fragment of a full length protein. An antigen may be a non-natural fragment of a protein. The delivery platform may be utilized to express one or more antigen for a particular pathogen. Multiple antigens may be expressed from a single self-amplifying RNA for example. Multiple antigens of an infectious agent or pathogen may be expressed from a single modified B subtilis 105 strain.

Avian coccidosis is a common poultry disease caused by Eimeria. Control of coccidosis has been approached by medicating feed with anticocciddial drugs or administering vaccines containing low doses of virulent or attenuated Eimeria oocysts. Problems of drug resistance and nonuniform administration of these Eimeria resulting in variable immunity prompt efforts to develop improved and recombinant Eimeria vaccines and other approaches to stimulate immunity and address cocciosis disease.

Eimeria is a genus of parasites that includes various species capable of causing the disease coccidiosis in animals such as cattle, poultry, dogs (especially puppies), cats (especially kittens), and smaller ruminants including sheep and goats. Species of this genus infect a wide variety of hosts. The most prevalent species of Eimeria that cause coccidiosis in cattle are E. bovis, E. zuernii, and E. auburnensis.

Delivery of an antigen capable of generating an immune response via the live intraceullular delivery platform and using the SAM vectors of the present invention has been demonstrated herein. Coccidial vaccine (Poultry): Salmonella Typhimurium was modified to deliver cross-protective antigens covering Eimeria tenella, E. maxima and E. acervulina as a part of SAM payload. Eimeria tenalla elongation factor-1α; EtAMA1; EtAMA2; Eimeria tenella 5401; Eimeria acervuline lactate dehydrogenase antigen gene; Eimeria maxima surface antigen gene; Glyceraldehyde 3-phosphate Dehydrogenase (GAPDH); Eimeria common antigen 14-3-3 antigens were delivered and expressed in an applicable system.

There are various peptides or proteins which act independently as therapeutic biolmolecules. Among these are anti-infective or anti-bacterial peptides which can serve to block or treat infection by infectious agents or bacteria.

A wide range of antimicrobial peptides is secreted in plants and animals to challenge attack by foreign viruses, bacteria or fungi (Boman, H. G. (2003) J. Intern. Med. 254 (3):197-215). These form part of the innate immune response to infection, which is short term and fast acting relative to humoral immunity. These peptides are heterogeneous in length, sequence and structure, but most are small, cationic and amphipathic (Zasloff, M. (2002) Nature 415(6870):389-395). Exemplary such known antimicrobial peptides are listed at an antimicorobial database (aps.unmc.edu/AP/main.php; Wang Z and Wang G (2004) NAR 32:D590-D592) and the content and disclosure of this site is incorporated herein by reference in its entirety. While the external cell wall may be the initial target, several lines of evidence suggest that antimicrobial peptides act by lysing bacterial membranes. Cells become permeable following exposure to peptides, and their membrane potential is correspondingly reduced. Protamines or polycationic amino acid peptides containing combinations of one or more recurring units of cationic amino acids, such as arginine (R), tryptophan (W), lysine (K), even synthetic polyarginine, polytryptophan, polylysine, have been shown to be capable of killing microbial cells.

A cell-wall degrading enzyme is an enzyme which degrades components of the cell wall, including peptidoglycans, such as murein and pseudomurein, chitin, and teichoic acid. Cell-wall degrading enzymes can include, but are not limited to amidases, muramidases, endopeptidases, glucosaminidases. Bacteriophage lysins are cell wall degrading anti-bacterial enzymes encoded by phage in bacteria. Lysins are peptidoglycan hydrolases that break bonds in the bacterial wall, rapidly hydrolyzing covalent bonds essential for peptidoglycan integrity, causing bacterial lysis and concomitant progeny phage release. Bacteriophage lytic enzymes have been established as useful in the assessment and specific treatment of various types of infection in subjects through various routes of administration. Phage associated lytic enzymes have been identified and cloned from various bacteriophages, each shown to be effective in killing specific bacterial strains.

The present invention has wide applicability to the development of effective immune stimulating compositions, immune promotion compositions, and vaccines against bacterial, fungal, parasite or viral disease agents where local immunity is important and might be a first line of defense. Such vaccines may be applicable to hatchery or field vaccine programs, particularly in farm and feed animals. Viral vaccines can be produced against either DNA or RNA viruses. Vaccines to protect against infection by pathogenic fungi, protozoa and parasites are also contemplated by this invention. The invention provides both therapeutic vaccines, such as wherein an antibody or portion thereof is administered and expressed via the delivery platform, for example to an animal with a disease or infection, and prophylactic vaccines, wherein a protein or antigen is administered and expressed via the delivery platform and serves to stimulate immunity in an animal.

Any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.” In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

Throughout this specification, quantities are defined by ranges, and by lower and upper boundaries of ranges. Each lower boundary can be combined with each upper boundary to define a range. The lower and upper boundaries should each be taken as a separate element. Two lower boundaries or two upper boundaries may be combined to define a range.

### Deposit Information

Bacillus subtillis strain “ELA191105” was deposited on 19 Jun. 2020 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-126786.

Access to the deposit will be available during the pendency of this application to persons determined by the Commissioner of Patents and Trademarks to be entitled thereto under 37 C.F.R. § 1.14 and 35 U.S.C. § 122. Upon allowance of any embodiments in this application, all restrictions on the availability to the public of the variety will be irrevocably removed.

The deposit will be maintained in the ATCC depository, which is a public depository, for a period of 30 years, or 5 years after the most recent request, or for the effective life of the patent, whichever is longer, and will be replaced if a deposit becomes nonviable during that period.

The present disclosure may be better understood with reference to the examples, set forth below. The following examples are put forth so as to provide those of ordinary skill in the art with a complete disclosure and description of how the compounds, compositions, and/or methods claimed herein are made and evaluated, and are intended to be purely exemplary and are not intended to limit the disclosure. It will be appreciated that other embodiments and uses will be apparent to those skilled in the art and that the invention is not limited to these specific illustrative examples or preferred embodiments.

### Example 1

### Isolation of Bacillus Subtilis Strain and Strain Characterization

Samples are isolated from chicken cecal samples. The samples are either heated to 90° C. for 10 minutes or treated with ethanol to a final concentration of 50% for 1 hour for spore isolation. The treated samples are plated on LB medium and the resulting colonies are purified by three sequential transferred onto LB agar plates. Identity of isolates is determined by amplification of 16 S-rRNA gene followed by DNA Sanger sequencing of the PCR amplicon.

Inhibition of bacterial strains by ELA191105 was tested. Table 1 summarizes the results of inhibition of other isolated bacterial strains by B. subtilis strain 105.

TABLE 1

| Strain | | | APEC | APEC | APEC | | Salmonella | C. |
|---|---|---|---|---|---|---|---|---|
| ID | Origin | ID | O2 | O78 | O18 | CP15* | Thypimurium | jejuni |
| 105 | Poultry | Bacillus | +++ | + | +++ | ++ | + | − |
| | cecum, | subtilis | | | | | | |
| | BRRS | | | | | | | |

Note:
*Clostridium perfringens strain 15

Antibiotic Susceptibility: Antibiotic susceptibility of Strain ELA191105 was tested. ELA191105 is susceptible to chloramphenicol, gentamicin, tetracycline, erythromycin, clindamycin, streptomycin, kanamycin, and vancomycin.

Growth Media: Growth on arbinoxylan and banana starch as the sole growth media was tested. ELA191105 is capable of growth on the aforementioned as the sole growth substrates.

Sporulation: Sporulation of ELA191105 was tested. ELA191105 formed spores in tested sporulation medium (Difco Sporulation Medium, DSM) and the culture is grown at 37° C. for 72 h.

Digestive Enzyme Analysis: Amylase and protease activities of ELA191105 was tested following protocol as described by Latorre, J D, 2016. Briefly, overnight culture of Bacillus isolate was spotted onto agar plate containing soluble starch and skim milk for amylase and protease assay, respectively. The plates were incubated at 37° C. for 48 h. The zone of clearance due to protease activity is observed directly whereas zone of clearance from amylase activity was visualized by flooding the surface of the plates with 5 mL of Gram's iodine solution. Protease activity of ELA191105 was tested by way of protease assay and amylase and protease activity are observed. Beta-mannanase activity for ELA191105 was tested and it is demonstrated that the strain is capable of digesting galactomannan.

Cytotoxicity Assay: Cytotoxicity of ELA191105 was tested against Vero cells. Cytotoxicity is measured by LDH cytotoxicity test. Positive control: Bacillus cereus DSM 31 (ATCC 14579) (78.6% cytotoxicity); Negative control: Bacillus licheniformis ATCC 14580 (−0.1% cytotoxicity); Test control: Subtilis 747 (CorrelinkTM strain) (8.7% cytotoxicity; non-toxic). ELA191105 strain is not cytotoxic to Vero cells. The percent cytotoxicity is less than 10.

Genomic Analysis: The genome of strain ELA191105 was sequenced and some genomic features are as follows: Contigs: 3; Coverage: 117×; % GC: 43%; Length (Mbp): 4.089.

ELA191105 possesses genes that are absent in other Bacillus strains used for genome comparison. Some of the unique genes include Metabolic enzymes (Phosphosulfolactate synthase, ethanolamine/propanediol utilization, Malate/lactate dehydrogenase); Antioxidant (Prokaryotic glutathione synthetase); Transporters (Organic Anion Transporter Polypeptide (OATP) family); and Digestive enzymes (alpha-amylase). Details regarding unique genes and metabolic analysis as well as exemplary antimicrobial peptides, secondary metabolite genes of ELA191105, including in comparison with other Bacillus strains is provided in U.S. Ser. No. 63/083,697 filed Sep. 25, 2020 and in U.S. Ser. No. 63/241,369 filed Sep. 7, 2021, each of which are incorporated by reference herein.

The genome nucleic acid sequence for strain 105 (ELA191105) is provided in SEQ ID NO:1 as a full genome sequence and in SEQ ID NOs: 2-6.

Table 2 summarizes some of the digestive enzyme identified in genomic analysis of the B. subtilis strain 105.

TABLE 2

| Digestive enzymes | ELA191105 |
|---|---|
| Lipase | Present |
| 3-Phytase | Present |
| Endo-1,4-beta-mannosidase (Beta-D-mannanase) | Present |
| 1,4-α-glucan branching enzyme GlgB | Present |
| 6-phospho-beta-galactosidase | Absent |
| Alpha-amylase | Present |
| Alpha-galactosidase | Present |
| Beta-glucanase | Present |
| Beta-hexosaminidase | Present |
| Endo-1,4-beta-xylanase A | Present |
| Endoglucanase | Present |
| L-Ala--D-Glu endopeptidase | Present |
| Maltose-6′ phosphate glucosidase | Present |
| Oligo-1,6-glucosidase | Present |
| Oligo-1,6-glucosidase 1 | Present |
| Pectate lyase | Present |
| Pectate lyase C | Present |
| Pectin lyase | Present |
| Pullulanase | Present |
| putative 6-phospho-beta- glucosidase | Present |
| putative oligo-1,6-glucosidase2 | Present |

Strain ELA191105 includes genes encoding bacteriocins, particularly SubtilosinA, Plipastatin, Surfactin, Bacillibactin and Bacilysin. In addition 2 clusters of Terpene-derived metabolites and 1 cluster of Polyketide-derived metabolites are present in ELA191105 strain.

### Example 2

### Global Metabolomics Analysis

A global metabolomics analysis of strain B. subtilis (ELA191105) was conducted. The strain was grown individually and the resulting cell pellet and supernatant analyzed to identify metabolites. Strains are grown at 37° C. for 24 hours in minimal media or rich media. Fresh media (no cells) were used as control samples. The metabolites in the supernatant represent molecules that are secreted by the cell. Minimal medium: M9 salts with 0.5 g casamino acids/L and 1% glucose. M9 salts contains Disodium Phosphate (anhydrous) 6.78 g/L, Monopotassium Phosphate 3 g/L, Sodium Chloride 0.5 g/L, Ammonium Chloride 1 g/L. Rich medium: Bacillus broth (per liter): Peptone 30 g; Sucrose 30 g; Yeast extract 8 g; KH2PO4 4 g; MgSO4 1.0 g; MnSO4 25 mg.

Samples are prepared using the automated MicroLab STAR® system from Hamilton Company. Several recovery standards are added prior to the first step in the extraction process for QC purposes. Samples are extracted with methanol under vigorous shaking for 2 min (Glen Mills GenoGrinder 2000) to precipitate protein and dissociate small molecules bound to protein or trapped in the precipitated protein matrix, followed by centrifugation to recover chemically diverse metabolites. The resulting extract is divided into five fractions: two for analysis by two separate reverse phase (RP)/UPLC-MS/MS methods using positive ion mode electrospray ionization (ESI), one for analysis by RP/UPLC-MS/MS using negative ion mode ESI, one for analysis by HILIC/UPLC-MS/MS using negative ion mode ESI, and one reserved for backup. Samples are placed briefly on a TurboVap® (Zymark) to remove the organic solvent. The sample extracts are stored overnight under nitrogen before preparation for analysis.

Ultrahigh Performance Liquid Chromatography-Tandem Mass Spectroscopy (UPLC-MS/MS): All methods utilize a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract is dried then reconstituted in solvents compatible to each of the four methods. Each reconstitution solvent contains a series of standards at fixed concentrations to ensure injection and chromatographic consistency. One aliquot is analyzed using acidic positive ion conditions, chromatographically optimized for more hydrophilic compounds. In this method, the extract is gradient-eluted from a C18 column (Waters UPLC BEH C18-2.1×100 mm, 1.7 m) using water and methanol, containing 0.05% perfluoropentanoic acid (PFPA) and 0.1% formic acid (FA). A second aliquot is also analyzed using acidic positive ion conditions, but is chromatographically optimized for more hydrophobic compounds. In this method, the extract is gradient eluted from the aforementioned C18 column using methanol, acetonitrile, water, 0.05% PFPA, and 0.01% FA, and is operated at an overall higher organic content. A third aliquot is analyzed using basic negative ion optimized conditions using a separate dedicated C18 column. The basic extracts are gradient-eluted from the column using methanol and water, however with 6.5 mM Ammonium Bicarbonate at pH 8. The fourth aliquot is analyzed via negative ionization following elution from a HILIC column (Waters UPLC BEH Amide 2.1×150 mm, 1.7 sm) using a gradient consisting of water and acetonitrile with 10 mM Ammonium Formate, pH 10.8. The MS analysis alternates between MS and data-dependent MSn scans using dynamic exclusion. The scan range covers approximately 70-1000 m/z.

Data are subject to global untargeted metabolic profiling. Welch t-test and Principal Component Analysis (PCA) are used to analyze the data. Principal component analysis (PCA) is a mathematical procedure that reduces the dimensionality of the data while retaining most of the variation in a dataset. This approach allows visual assessment of the similarities and differences between samples (growth conditions, including media type and strains present). Populations that differ are expected to group separately and vice versa.

Metabolite Quantification and Block Correction: Peaks are quantified as area-under-the-curve detector ion counts. For studies spanning multiple days, a data adjustment step is performed to correct block variation resulting from instrument inter-day tuning differences, while preserving intra-day variance. Essentially, each compound is corrected in balanced run-day blocks by registering the daily medians to equal one (1.00), and adjusting each data point proportionately (termed the “block correction”). For studies that do not require more than one day of analysis, no adjustment of raw data is necessary, other than scaling for purposes of data visualization.

Metabolite is identified as unique to a strain if the value for the secreted metabolite is at least 1.5-fold greater than those of other strains or control strain single isolates. Unique metabolites for strain consortia are determined using >1.5-fold cut off compared to values of respective metabolites secreted by single isolates of the consortium. In rich medium, 231 metabolites were identified for strain ELA 191105, while 111 metabolites were identified in minimal medium, for a total of 272 metabolites. Overall, strain ELA 191105 had 77 unique metabolites, 45 which were at values above a 2-fold threshold, compared to other Bacillus strains used in the analysis.

Strain ELA191105 was cultured individually in minimal media or in rich media and the supernatant analyzed for secreted metabolites. Table 3 provides an exemplary list of metabolites secreted by the strain. Unless otherwise noted, the metabolite is at least 1.5 fold greater than the media control.

TABLE 3

| | Minimal media | Rich media |
|---|---|---|
| METABOLITE | ELA191105 | ELA191105 |
| N-acetyl-cadaverine | No | Yes^{A, B} |
| isovalerate (C5) | Yes^{A} | Yes^{A} |
| N-acetylisoleucine | Yes^{A, B, C} | Yes^{A, B, C} |
| Homocysteine | Yes^{A, B, C} | Yes^{A, B, C} |
| Homocystine | Yes^{A, B, C} | No |
| N-acetylcitrulline | Yes^{A, B, C} | No |
| Glucoronate | No | Yes^{A, B, C} |
| alpha-ketoglutarate | Yes^{A, B, C} | No |
| 5-hydroxyhexanoate | Yes^{A, B} | No |
| glycerol 3-phosphate | Yes^{A} | Yes |
| 5-aminoimidazole-4-carboxamide | No | Yes^{A, B, C} |
| Hypoxanthine | Yes^{A, B, C} | No |
| Guanine | Yes^{A, B, C} | No |
| Orotate | Yes^{A, B, C} | No |
| Orotidine | No | No |
| 5,6-dihydrouridine | Yes^{A, B} | Yes^{A, B, C} |
| 3-dehydroshikimate | Yes^{A, B} | No |
| Kynurenate | No | — |
| Indolactate | Yes | Yes^{A, B, C} |
| Cyclo (gly-pro) | Yes | |
| Cyclo (his-phe) | Yes^{A, B, C} | — |
| Cyclo(phe-pro) (L, D) | Yes | |
| 1-kestose | Yes^{A, B} | Yes^{A, B, C} |
| Thioproline | Yes^{A, B, C} | Yes^{A, B, C} |
| N-acetylaspartate (NAA) | Yes^{A, B} | Yes^{A} |
| Glutamine | Yes | Yes^{A, B, C} |
| Tryptophan | Yes^{A, B, C} | — |
| Cysteine | Yes^{A, B, C} | Yes^{A, B, C} |
| Pyridoxamine | Yes^{A, B, C} | Yes^{A} |
| Pyridoxamine phosphate | Yes^{A} | Yes^{A} |
| pantothenate (Vitamin B5) | Yes^{A, B, C} | — |
| pyridoxine (Vitamin B6) | Yes^{A} | — |
| 2R,3R-dihydroxybutyrate | Yes^{A, B, C} | — |
| Choline | Yes^{A, B, C} | — |
| 5-aminoimidazole-4-carboxamide | No | Yes^{A, B, C} |
| trigonelline (N′-methylnicotinate) | Yes^{A, B, C} | — |
| Mevalonolactone | Yes^{A, B, C} | — |
| Tricarballylate | No | Yes^{A} |

A—metabolite is secreted at least 2 fold greater than media control;
B—metabolite is secreted at least 3 fold greater than media control;
C—metabolite is secreted at least 5 fold greater than media control.

Strain ELA191105 was cultured individually in rich media and the supernatant analyzed for secreted metabolites. Table 4 provides an exemplary list of metabolites secreted by the strain. Unless otherwise noted, the metabolite is at least 1.5 fold greater than the media control.

TABLE 4

| METABOLITE | ELA191105 |
|---|---|
| N-acetyl-cadaverine | Yes^{A, B} |
| isovalerate (C5) | Yes^{A} |
| N-acetylisoleucine | Yes^{A, B, C} |
| Homocystine | No |
| Homocysteine | Yes^{A, B, C} |
| N-acetylcitrulline | No |
| Glucoronate | Yes^{A, B, C} |
| alpha-ketoglutarate | No |
| 5-hydroxyhexanoate | No |
| 2R,3R-dihydroxybutyrate | Yes^{A, B} |
| glycerol 3-phosphate | Yes |
| 5-aminoimidazole-4-carboxamide | Yes^{A, B, C} |
| Hypoxanthine | No |
| Guanine | No |
| Orotate | No |
| Orotidine | No |
| 5,6-dihydrouridine | Yes^{A, B, C} |
| 3-dehydroshikimate | No |
| Indolactate | Yes^{A, B, C} |
| Indoleacetate | Yes^{A, B} |
| 1-kestose | Yes^{A, B, C} |
| Thioproline | Yes^{A, B, C} |
| N-acetylaspartate (NAA) | Yes^{A} |
| Glutamine | Yes^{A, B, C} |
| Cysteine | Yes^{A, B, C} |
| Pyridoxamine | Yes^{A} |
| Pyridoxamine phosphate | Yes^{A} |
| 5-aminoimidazole-4- | Yes^{A, B, C} |
| carboxamide | |
| Tricarballylate | Yes |

A-metabolite is at least 2 fold greater than media control;
B-metabolite is at least 3 fold greater than media control;
C-metabolite is at least 5 fold greater than media control.

Strain ELA191105 was cultured individually in minimal media and rich media, and the supernatants are analyzed for secreted metabolites. An exemplary list of metabolites uniquely secreted by strain 105 is as follows: betaine^{A}, carboxyethyl-GABA^{A}, 3-methylhistidine^{A}, saccharopine, pipecolate, N,N-dimethyl-5-aminovalerate^{A,B}, N-butyryl-phenylalanine^{A}, tryptophanA, N-butyryl-leucine, 2-hydroxy-4-(methylthio)butanoic acid^{A}, S-methylcysteine^{A}, ornithine, N-methylproline^{A}, N,N,N-trimethyl-alanylproline betaine (TMAP)^{A}, N-monomethylarginine^{A}, guanidinoacetate, putrescine, cysteinylglycine^{A,B,C}, cyclo(gly-phe), tryptophylglycine, pymvate^{A,B}, mannose, N-acetylmuramateA, eicosenamide (20:1), deoxycarnitineA, 2 S,3R-dihydroxybutyrate, chiro-inositol^{A,B}, choline, glycerophosphorylcholine (GPC)^{A}, 1-palmitoyl-GPE (16:0)^{A}, 1-linoleoylglycerol (18:2), 3-hydroxy-3-methylglutarate, 3-ureidopropionate, (3′-5′)-uridylyluridine, nicotinamide riboside, trigonelline (N′-methylnicotinate), oxalate (ethanedioate)^{A}, pyridoxine (Vitamin B6), maltol, histidine betaine (hercynine), 2,6-dihydroxybenzoic acid, pentose acid, N-acetylserine^{R,A}, N-acetylthreonine^{R}, N-acetylglutamine^{R,A}, 1-methylhistidine^{R,A,B}, N-acetylhistidine^{R,A}, trans-urocanate^{R,A}, N6-acetyllysine^{R}, N-acetyl-cadaverine^{R,A,B}, N-acetylphenylalanine^{R,A}, phenyllactate (PLA)^{R,A}, 3-(4-hydroxyphenyl)lactate (HPLA)^{R,A,B}, isovalerate (C5)^{R,A,B}, N-acetylisoleucine^{R,A,B,C}, N-acetylvaline^{R,A}, N-acetylmethionine^{R}, S-adenosylmethionine (SAM)^{R}, 2-hydroxy-4-(methylthio)butanoic acid^{R}, S-methylcysteine^{R,A}, N-acetylarginine^{R}, acetylagmatine^{R,A}, glutathione, oxidized (GSSG)^{R,A}, 2-hydroxybutyrate/2—hydroxyisobutyrate^{R,A}gamma-glutamylhistidine^{R,A}, glucuronate^{R,A,B}, aconitate [cis or trans]^{R}, 2-methylcitrate^{R}, 2R,3R-dihydroxybutyrate^{R,A,B}, 5-aminoimidazole-4-carboxamide^{R,A,B,C}, N-carbamoylaspartate^{R,A}, dihydroorotate^{R}, orotidine^{R,A,B,C}, thymine^{R,A,B}, (3′-5′)-adenylylguanosine^{R,A,B,C}, nicotinamide riboside^{R}, NAD+^{R,A}, Pyridoxamine, pyridoxamine phosphate^{A} and homocitrate.

R-metabolite secreted when grown in rich media; A-metabolite is at least 2 fold greater than the two other strains; B-metabolite is at least 3 fold greater than the two other strains; C-metabolite is at least 5 fold greater than the two other strains.

The 16 S rRNA sequence of ELA191105 bacteria strain is provided below:

Full 16S-rRNA sequences
>Bacillus subtilis ELA191105 (BSUB_00009)
(SEQ ID NO: 34)
tcggagagtttgatcctggctcaggacgaacgctggcggcgtgcctaat
acatgcaagtcgagcggacagatgggagcttgctccctgatgttagcgg
cggacgggtgagtaacacgtgggtaacctgcctgtaagactgggataac
tccgggaaaccggggctaataccggatggttgtttgaaccgcatggttc
aaacataaaaggtggcttcggctaccacttacagatggacccgcggcgc
attagctagttggtgaggtaacggctcaccaaggcaacgatgcgtagc
cgacctgagagggtgatcggccacactgggactgaggcacggcccagac
tcctacgggaggcagcagtagggaatcttccgcaatggacgaaagtc
tgacggagcaacgccgcgtgagtgatgaaggttttcggatcgtaaagct
ctgttgttagggaagaacaagtaccgttcgaatagggcggtaccttgac
ggtacctaaccagaaagccacggctaactacgtgccagcagccgcggta
atacgtaggtggcaagcgttgtccggaattattgggcgtaaagggctc
gcaggcggtttcttaagtctgatgtgaaagcccccggctcaaccgggga
gggtcattggaaactggggaacttgagtgcagaagaggagagtggaat
tccacgtgtagcggtgaaatgcgtagagatgtggaggaacaccagtggc
gaaggcgactctctggtctgtaactgacgctgaggagcgaaagcgtgg
ggagcgaacaggattagataccctggtagtccacgccgtaaacgatgag
tgctaagtgttagggggtttccgccccttagtgctgcagctaacgcatt
aagcactccgcctggggagtacggtcgcaagactgaaactcaaaggaat
tgacgggggcccgcacaagcggtggagcatgtggtttaattcgaagc
aacgcgaagaaccttaccaggtcttgacatcctctgacaatcctagaga
taggacgtccccttcgggggcagagtgacaggtggtgcatggttgtcgt
cagctcgtgtcgtgagatgttgggttaagtcccgcaacgagcgcaaccc
ttgatcttagttgccagcattcagttgggcactctaaggtgactgccgg
tgacaaaccggaggaaggtggggatgacgtcaaatcatcatgcccctta
tgacctgggctacacacgtgctacaatggacagaacaaagggcagcgaa
accgcgaggttaagccaatcccacaaatctgttctcagttcggatcgca
gtctgcaactcgactgcgtgaagctggaatcgctagtaatcgcggatca
gcatgccgcggtgaatacgttcccgggccttgtacacaccgcccgtcac
accacgagagtttgtaacacccgaagtcggtgaggtaaccttttaggag
ccagccgccgaaggtgggacagatgattggggtgaagtcgtaacaaggt
agccgtatcggaaggtgcggctggatcacctccttt

### EXAMPLE 3

### Metabolite and Genome Analysis of Bacillus Strains

Metabolite analysis was conducted on strains ELA1901105 (also denoted strain 105). TABLE 5 provides analysis of the presence or absence of certain natural antibiotics/antibacterials or bacteriocins in the 105 (ELA1901105) strain.

TABLE 5

| Class | ELA191105 |
|---|---|
| 132.2; LCI | Absent |
| Lanthipeptide_class_II | Absent |
| 266.1; Amylocyclicin | Absent |
| 318.1; ComX1 | Absent |
| 216.2; Subtilosin_(Sbox) | Present |
| 492.1; Competence | Present |
| 490.1; Pumilarin | Absent |
| 225.2; UviB | Absent |
| 294.1; Plantathiazolicin_(Plantazolicin) | Absent |
| 54.1; LichenicidinVK21A1_(Lichenicidin_A1) | Absent |
| 121.1; Sporulation-killingfactor_skfA | Absent |
| 321.1; ComX4 | Absent |
| 145.1; Subtilosin_A | Absent |
| 127.1; Sublancin_168 | Absent |

Small peptides have powerful biological activities ranging from antibiotic to immune suppression. Some of these peptides are synthesized by Non Ribosomal Peptide Synthetases (NRPS) (Challis G L and Naismith J H (2004) Cur Opin Struct Biol 14(6):748-756). While the vast majority of peptide bond formation is catalyzed by ribosomes, the catalysis of peptide bond formation by NRPS is of importance and relevance. Some of the most well known examples of molecules made by NRPS illustrate the importance of NRPS systems. The antibiotic vancomycin and its analogues have very complex structures made by NRPS and associated enzymes. Indeed, almost all peptide based antibiotics are made by NRPS. Chelation of iron by bacteria is vital for their survival and is often a virulence determinant in pathogens. NRPS synthesize macrocycles such as enterobactin, which have an extraordinary high iron affinity. Cyclosporin, an immune suppressor and the potent anti tumour compound bleomycin are both made by NRPS. The molecules made by NRPS are often cyclic, have a high density of non-proteinogenic amino acids, and often contain amino acids connected by bonds other than peptide or disulfide bonds. NRPS are now known to be very large proteins and, despite the obvious complexity of the products, consist of a series of repeating enzymes fused together.

The non-ribosomal peptide synthetases are modular enzymes that catalyze synthesis of important peptide products from a variety of standard and non-proteinogenic amino acid substrates. Within a single module are multiple catalytic domains that are responsible for incorporation of a single residue. After the amino acid is activated and covalently attached to an integrated carrier protein domain, the substrates and intermediates are delivered to neighboring catalytic domains for peptide bond formation or, in some modules, chemical modification. In the final module, the peptide is delivered to a terminal thioesterase domain that catalyzes release of the peptide product. (Miller B R and Gulick A M (2016) Methods Mol Biol 1401:3-29).

The Bacillus strain 105 of use in the invention includes numerous NRPS and also predicted proteins which are expected to be synthesized by NRPS. Certain such proteins are as follows: NRPS; NRPS; NRPS,betalactone; CDPS; head_to_tail,sactipeptide; transAT-PKS,PKS-like,T3PKS,transAT-PKS-like,NRPS; terpene; terpene; T3PKS.

The presence of certain predicted proteins and secondary metabolites is indicated with the number of predicted such type proteins provided in parenthesis below in TABLE 6.

TABLE 6

| | ELA191105 |
|---|---|
| | (strain 105) |
| CDPS | Present (1) |
| LAP | — |
| NRPS | Present (2) |
| NRPS, PKS-like, T3PKS, transAT-PKS, transAT-PKS-like | Present (1) |
| NRPS, T3PKS, transAT-PKS, transAT-PKS-like | — |
| NRPS, bacteriocin | — |
| NRPS, betalactone | Present (1) |
| NRPS, betalactone, transAT-PKS | — |
| NRPS, transAT-PKS | — |
| PKS-like | — |
| T3PKS | Present (1) |
| head_to_tail, sactipeptide | Present (1) |
| lanthipeptide | — |
| terpene | Present (2) |
| transAT-PKS | — |
| transAT-PKS,transAT-PKS-like | — |
| other | Present (1) |

No plasmids were identified in the strain ELA1901105 (also denoted strain 105) by analysis of the whole genome sequences.

Further analysis of predicted antioxidant proteins from the sequence analysis of the Bacillus strains was conducted. Certain results are provided below in TABLE 7.

TABLE 7
Antioxidant prediction. Putative genes encoding
antioxidant in the genome of Bacillus subtilis strain 105

| | | B. subtilis |
|---|---|---|
| | | PTA-86 |
| | | (strain 105; |
| Description | Accession Number | BSUB105) |
| Alkyl hydroperoxide reductase C | UniRef100_P80239 | NA |
| | UniRef100_O34564 | PRESENT |
| | UniRef100_P80239 | PRESENT |
| Glutathione peroxidase BsaA | UniRef100_P40581 | NA |
| Hydroperoxy fatty acid reductase gpx1 | UniRef100_P40581 | NA |
| | UniRef100_P40581 | PRESENT |
| Organic hydroperoxide resistance | UniRef100_O34777 | NA |
| transcriptional regulator | UniRef100_O34777 | NA |
| | UniRef100_O34777 | PRESENT |
| Peroxide operon regulator | Q2G282 | NA |
| | Q2G282 | PRESENT |
| Sporulation thiol-disulfide | UniRef100_O31687 | NA |
| oxidoreductase A | UniRef100_O31687 | PRESENT |
| Superoxide dismutase [Mn] | UniRef100_P49114 | NA |
| | UniRef100_P49114 | PRESENT |
| Thiol peroxidase | UniRef100_P80864 | NA |
| | UniRef100_P80864 | PRESENT |
| Thiol-disulfide oxidoreductase ResA | UniRef100_P35160 | NA |
| | UniRef100_O31820 | NA |
| | UniRef100_O31820 | PRESENT |
| | UniRef100_P35160 | PRESENT |
| Thiol-disulfide oxidoreductase YkuV | UniRef100_O31699 | NA |
| | UniRef100_O31699 | PRESENT |
| Vegetative catalase | UniRef100_Q64405 | NA |
| | UniRef100_Q64405 | PRESENT |

Toxin or Antitoxin prediction analysis indicated that strain ELA191105 (strain 105) includes an Antitoxin EndoAI corresponding to Uniprot ID P96621 and a Endoribonuclease EndoA corresponding to Uniprot ID P96622.

Digestive enzymes include enzymes that cleave cell wall or cell membrane components, particularly of bacteria. Among these are for instance lysins which are cell wall hydrolases and often are found on and encoded by bacteriophages. The activities of lysins can be classified into two groups based on bond specificity within the peptidoglycan: glycosidases that hydrolyze linkages within the aminosugar moieties and amidases that hydrolyze amide bonds of cross-linking stem peptides. (Fischetti V A et al (2006) Nat Biotechnol 24(12):1508-11). Predicted digestive enzymes in the Bacillus strain 105 based on sequence analysis are provided in TABLE 8 below.

Strain 105 was evaluated for various other components and particularly antimicrobial resistance genes as shown below in TABLE 9.

TABLE 8
Predicted Digestive Enzymes identified in the genome of B. subtilis
PTA-86 (percent identity >50 and alignment length percent >90)

| | | B. subtilis |
|---|---|---|
| | | PTA-86 |
| | | (ELA191105/ |
| | Accession | strain |
| Gene Description | Number | 105) |
| 1,4-alpha-glucan branching enzyme GlgB | AGA22754.1 | Present |
| 50S ribosomal protein L1 | QFI56506.1 | NA |
| | QFI56506.1 | Present |
| 6-phospho-beta-glucosidase GmuD | CDG27774.1 | NA |
| | QAS06813.1 | Present |
| | QDK91913.1 | NA |
| Alpha-amylase | AIW36239.1 | NA |
| | QAV82864.1 | Present |
| | BAT21551.1 | NA |
| Alpha-galactosidase | QEK97784.1 | NA |
| | AIC99339.1 | Present |
| | QDK91116.1 | NA |
| Alpha-galacturonidase | ASB68722.1 | Present |
| Arabinoxylan arabinofuranohydrolase | AAD30363.1 | NA |
| | AAD30363.1 | Present |
| Aryl-phospho-beta-D-glucosidase BglA | CDG26179.1 | NA |
| | QAW06324.1 | Present |
| | QDK90194.1 | NA |
| Aryl-phospho-beta-D-glucosidase BglC | CDG24623.1 | NA |
| | ARV97270.1 | Present |
| Aryl-phospho-beta-D-glucosidase BglH | CDG27827.1 | NA |
| | QAS10023.1 | Present |
| ATP synthase subunit alpha | QHB16940.1 | NA |
| | QHB16940.1 | Present |
| Beta-glucanase | ATV02530.1 | NA |
| | AID00207.1 | Present |
| | AYL88759.1 | NA |
| Beta-hexosaminidase | ANB82474.1 | NA |
| | QAR91196.1 | Present |
| | QDK88534.1 | NA |
| Cephalosporin-C deacetylase | AHZ14331.1 | NA |
| | QAS06568.1 | Present |
| | QDK88655.1 | NA |
| Cortical fragment-lytic enzyme | CCP20089.1 | NA |
| | QBJ80577.1 | Present |
| | ATC51419.1 | NA |
| Cysteine synthase | AYA39814.1 | NA |
| | AYA39814.1 | Present |
| Demethyllactenocin | CDG24864.1 | NA |
| mycarosyltransferase | ARV97526.1 | Present |
| | QDK88875.1 | NA |
| Endo-1,4-beta-xylanase A | AQQ16389.1 | NA |
| | ANJ02848.1 | Present |
| | QDK91715.1 | NA |
| Endoglucanase | CDG26057.1 | NA |
| | NP_389695.1 | Present |
| | QDK90074.1 | NA |
| General stress protein A | AKF74877.1 | NA |
| | AGA22706.1 | Present |
| | QDK91887.1 | NA |
| Glucose-1-phosphate adenylyltransferase | QHA28835.1 | Present |
| Glucuronoxylanase XynC | CBL17903.1 | NA |
| | CBL17903.1 | Present |
| Glycogen phosphorylase | AIX08721.1 | Present |
| Glycogen synthase | QAW13524.1 | Present |
| Intracellular maltogenic amylase | AMR45682.1 | Present |
| L-Ala--D-Glu endopeptidase | QGH57794.1 | NA |
| | QGI53236.1 | Present |
| | QGT57119.1 | NA |
| Maltose-6′-phosphate glucosidase | QGH55793.1 | NA |
| | QGU25462.1 | Present |
| | QDK89133.1 | NA |
| Melibiose/raffinose/stachyose import | QEK97784.1 | NA |
| permease protein MelC | QEK97784.1 | Present |
| Membrane-bound lytic murein | CCP21108.1 | NA |
| transglycosylase F | ASB92721.1 | Present |
| | QDK90428.1 | NA |
| | QDK89432.1 | NA |
| N-acetyl-alpha-D-glucosaminyl | ATV01518.1 | NA |
| L-malate deacetylase 1 | AYE64850.1 | Present |
| | QEQ03843.1 | NA |
| N-acetyl-alpha-D-glucosaminyl | ASS91714.1 | NA |
| L-malate synthase | ASS91714.1 | Present |
| N-acetylglucosamine-6- | QGH58054.1 | NA |
| phosphate deacetylase | QAS09601.1 | Present |
| | QDK91560.1 | NA |
| N-acetylglucosaminyl- | ATV02313.1 | NA |
| diphosphoundecaprenol N-acetyl- | AYE66149.1 | Present |
| beta-D-mannosaminyltransferase | QDK91629.1 | NA |
| Oleandomycin glycosyltransferase | CDG26160.1 | NA |
| | CDG29153.1 | NA |
| | AIC97767.1 | Present |
| | AKN14112.1 | Present |
| | QDK90176.1 | NA |
| | QDK89494.1 | NA |
| Oligo-1,6-glucosidase | AJK66530.1 | NA |
| | ASZ62584.1 | Present |
| | AKD24292.1 | NA |
| Oligo-1,6-glucosidase 1 | QAW18252.1 | Present |
| Pectate lyase | AWG39450.1 | NA |
| | AFP43210.1 | Present |
| | QDK89071.1 | NA |
| Pectate lyase C | AIC99792.1 | Present |
| Pectin lyase | AWG40278.1 | NA |
| | AKE23786.1 | Present |
| | QDK91950.1 | NA |
| Penicillin-binding protein 1A/1B | AWG38114.1 | NA |
| | QCU15370.1 | Present |
| | QDK90466.1 | NA |
| Penicillin-binding protein 1F | CDG25272.1 | NA |
| | QCU14315.1 | Present |
| | QDK89310.1 | NA |
| Penicillin-binding protein 2D | CDG27654.1 | NA |
| | QGU22813.1 | Present |
| | QDK91796.1 | NA |
| Penicillin-binding protein 4 | AWG37355.1 | NA |
| | ASB94693.1 | Present |
| | QDK91217.1 | NA |
| | CDG30505.1 | NA |
| | CDG25227.1 | NA |
| | QAW40980.1 | Present |
| | QDK90813.1 | NA |
| | QDK89265.1 | NA |
| Peptidoglycan-N-acetylmuramic | QEQ05952.1 | NA |
| acid deacetylase PdaA | AFQ56715.1 | Present |
| Peptidoglycan-N-acetylmuramic | AIX06967.1 | Present |
| acid deacetylase PdaC | | |
| Processive diacylglycerol beta- | AKF76366.1 | NA |
| glucosyltransferase | QAV83859.1 | Present |
| | AYE64796.1 | Present |
| | QDK90264.1 | NA |
| PTS system maltose-specific | VEC47756.1 | NA |
| EIICB component | VEC47756.1 | Present |
| Pullulanase | AYA43052.1 | Present |
| putative 6-phospho-beta- | CCG51779.1 | NA |
| glucosidase | NP_391735.1 | Present |
| | QDK91899.1 | NA |
| putative esterase YxiM | AOY05484.1 | Present |
| putative glycosyltransferase | QCV96123.1 | NA |
| | QCV96123.1 | Present |
| putative glycosyltransferase YkoT | ASB41833.1 | Present |
| putative oligo-1,6-glucosidase 2 | AJK64068.1 | NA |
| | QAW02849.1 | Present |
| | AMP32821.1 | NA |
| | AHC40869.1 | NA |
| putative protein YqbO | QAW41282.1 | Present |
| putative rhamnogalacturonan | ASZ60459.1 | Present |
| acetylesterase YesY | | |
| Putative sporulation-specific | AKF76978.1 | NA |
| glycosylase YdhD | CAB12390.2 | Present |
| | NP_391282.1 | Present |
| | QDK89600.1 | NA |
| Rhamnogalacturonan acetylesterase RhgT | QFY87628.1 | Present |
| Rhamnogalacturonan endolyase YesW | QAT44941.1 | Present |
| Rhamnogalacturonan exolyase YesX | AIX06475.1 | Present |
| Trehalose-6-phosphate hydrolase | AIW36656.1 | NA |
| | NP_388662.1 | Present |
| | AVX18210.1 | NA |
| UDP-N-acetylglucosamine--N- | AMR47346.1 | Present |
| acetylmuramyl-(pentapeptide) | QDK89788.1 | NA |
| pyrophosphoryl-undecaprenol N- | | |
| acetylglucosamine transferase | | |

TABLE 9
Putative antimicrobial resistance genes identified in B. subtilis PTA-86 (strain ELA191105)
through genome analysis (percent identity >80 and percent coverage >95)

| | | % | % | Accession | |
|---|---|---|---|---|---|
| Gene product | Gene id | Coverage | Identity | number | Antibiotic Resistance |
| macrolide 2′- | mphK | 100 | 97.72 | NG_065846.1 | MACROLIDE |
| phosphotransferase | | | | | |
| MphK | | | | | |
| ABC-F type | vmlR | 100 | 98.66 | NG_063831.1 | LINCOSAMIDE; |
| ribosomal | | | | | STREPTOGRAMIN; |
| protection protein | | | | | TIAMULIN |
| VmlR | | | | | |
| rifamycin- | rphC | 99.39 | 82.18 | NG_063825.1 | RIFAMYCIN |
| inactivating | | | | | |
| phosphotransferase | | | | | |
| RphC | | | | | |
| aminoglycoside 6- | aadK | 99.77 | 98.12 | NG_047379.1 | STREPTOMYCIN |
| adenylyltransferase | | | | | |
| AadK | | | | | |
| streptothricin N- | satA_Bs | 100 | 95.78 | NG_064662.1 | STREPTOTHRICIN |
| acetyltransferase | | | | | |
| SatA | | | | | |
| tetracycline efflux | tet(L) | 100 | 100 | NG_048204.1 | TETRACYCLINE |
| MFS transporter | | | | | |
| Tet(L) | | | | | |

### Example 4

### Safety and Multi-Omics Characterization of Host-Derived Bacillus Species Strain as Potential Probiotic, Manufacturing and Live Delivery Strain

Host-derived Bacillus strains were isolated and screened for desirable probiotic properties and safety and stability as a production or live delivery strain. The phenotypic, genomic and metabolomic analyses of a B. subtilis bacteria (Bs ATCC PTA126786 (ELA191105, strain 105)), showed that the strain has promising probiotic traits and safety and stability profiles.

Microbial feed ingredients, also called direct-fed microorganisms (DFMs) or probiotics, have attracted tremendous interest as an alternative to AGPs to support improved production efficiency. Probiotics are defined as “live microorganisms that, when administered in adequate amounts, confer a health benefit on the host” (5). Probiotics are believed to exert their benefits through mechanisms such as: assisting with nutrition and digestion, competitive exclusion of pathogens, modulating the immune system and gut microbiota, improving epithelial integrity, and/or producing small molecule metabolites that are beneficial to the host (6, 7). In addition to the above probiotic effects, microorganisms used as probiotics or ingested by an animal must survive environmental and processing challenges prior to reaching their target site in the animal. This includes low acidity of the upper gastrointestinal tract (GIT), bile acid toxicity, and heat exposure during manufacture of bacteria containing feed and feed pelleting application.

Endospore-forming Bacillus spp. can offer advantages over traditional probiotic strains due to the ability of Bacillus spores to withstand hostile environments such as high temperature, desiccation, and acidic pH, resulting in increased viability during the manufacturing and feed-pelleting process, increased stability inside animals' GIT and extended product shelf-life. Bacillus strains have been widely used to support improved production parameters (8-11). Once inside the GIT, the spores germinate into metabolically active vegetative cells (12-15). Within the Bacillus genus, species commonly used are B. subtilis, B. coagulans, B. clausii, B. amyloliquefaciens, and B. licheniformis (16). Bacillus strains are also utilized and known to produce commercial enzymes, antimicrobial peptides, and small metabolites that may confer health benefits to the host by supporting improved feed digestion, suppressing undesirable organisms, and by maintaining a healthy gut microbiota and immune system (reviewed in (17)).

To fill the knowledge gap in the genomic and phenotypic characterization of Bacillus spp. DFMs, we take advantage of DNA sequencing and omics technologies for the comprehensive identification, screening, and characterization of Bacillus spp. to assess their safety and efficacy as probiotic candidates. Detailed strain characterization employing multi-omics approaches could uncover correlations between strain properties and the effects of administration on the host, underpin possible mechanisms of action of probiotic strains, identify biomolecules that could be used in place of live bacteria (i.e. peptides, enzymes, metabolites), and help to rationally design strain to maximize positive effects on the host and/or delivery of biomolecules to the host.

### Materials and Methods

Microbial strains and growth conditions—The Bacillus spp. strains were routinely grown in Lysogeny Broth (LB) and incubated at 37° C. overnight while shaking at 200 rpm. Avian pathogenic Escherichia coli (APEC) serotypes O2, O18, O78 and Clostridium perfringens NAH 1314-JP1011 were obtained from the Elanco pathogen library. Salmonella enteritica serovar Typhimurium ATCC 14028 was purchased from the American Type Culture Collection (ATCC, Manassas, VA). E. coli strains and S. Typhimurium, were routinely grown in LB, and C. perfringens was grown in anaerobic Brain Heart Infusion (BHI) broth supplemented with yeast extract (5.0 g/L) and L-cysteine (0.5 g/L). For growth in liquid culture, a colony from the respective agar plate was inoculated into a 10 mL tube containing liquid media and the tube was incubated in a shaker incubator at 37° C. and 200 rpm for E. coli and S. Typhimurium, and statically at 39° C. for C. perfringens inside a Bactron anaerobic chamber (Sheldon Manufacturing, Inc., Cornellius, OR). The anaerobic chamber contained a mixture of N2:CO2:H2 (87.5:10:2.5, v/v/v).

Vero cells growth condition—Vero cells were obtained from Elanco cell culture collection and were maintained in Opti-MEM® I reduced serum media containing 5% Fetal Bovine Serum (FBS) (Cytiva, Marlborough, MA) and Gentamicin (Opti-5-Gent) (Life Technologies, Carlsbad, CA). The serum-free cell culture medium was similarly prepared with Minimal Essential Medium with Earle's Balanced Salt Solution (MEM/EBSS), 10% fetal bovine serum (FBS), 1% non-essential amino acids and 1% L-glutamine in place of FBS. To generate wells containing 100% confluent cells for the cytotoxicity assay, Vero cells grown for two to three days were divided into a 96-well flat bottom tissue culture plate (Fisher Scientific, Waltham, MA) where each well contained 1×104 cells. The cells were then incubated on the plate for 48-72 hours inside a CO2 incubator (37° C.; % CO2 was maintained at 5±1%).

Bacillus spp. isolation and identification

Bacillus isolation—Bacillus spp. were isolated from cecal contents of healthy 30-42 day old chickens raised at poultry research farms in Arkansas, Georgia, and Indiana, USA employing a combination of a high-throughput isolation platform employing Prospector® (GALT, Inc, San Carlos, CA) following the manufacture's protocol, and a classical isolation method as described previously (22). For both approaches, isolation protocols were preceded by selection of Bacillus spores from the starting cecal contents by applying heat at 95° C. for 5 min or treatment with ethanol. For the latter, frozen cecal samples from the Elanco library preserved in BHI containing 20% glycerol were thawed and equal amounts of Tryptic Soy Broth (TSB) medium were added and mixed. An equal amount of absolute ethanol was added to the sample to a final concentration of 50% and the mixture was incubated at 30° C. for an hour. The ethanol-treated samples were then used for isolation. For Bacillus spp. isolation employing conventional methods, 10-fold serial-dilution was applied to the treated cecal samples to ensure separate colonies recovered on agar plates. Each colony was purified by three sequential passages onto agar plates.

Strain identification—For an initial strain identification, Bacillus cell lysates were sent to the TACGen genomic sequencing facility (Richmond, CA) for strain identification. The strain identities were determined by Sanger sequencing of amplified regions of a partial length of 16 S ribosomal RNA (rRNA) gene employing primers 27F (5′ AGA GTT TGA TCM TGG CTC AG 3′) and 1492R (5′ CGG TTA CCT TGT TAC GAC TT3′). The resulting 16 S rRNA sequences were then searched against the NCBI 16 S rRNA database using BLAST searches with an e-value cutoff of <10-20 and a percent sequence identity value of >95%. Strain identification of select isolates were further confirmed by ortholog analyses as described in the following section: Genome-based strain identification and comparative genomic analyses.

In vitro microorganism inhibition assay—Bacillus spp. strains were screened for their antimicrobial activity against five microorganisms, namely APEC serotypes O2, O18, O78, Salmonella Typhimurium ATCC 14028, and Clostridium perfringens NAH 1314-JP1011. The assays were modified from a protocol described in (23) and performed in duplicate.

The assays were modified from a protocol described in (113) and performed in duplicate. Briefly, 10 μl of Bacillus freezer stock was inoculated into 2 mL of 0.5× LB in a 15 mL round bottom shaker tube. The cultures were incubated at 37° C. for 48 hours while shaking at 200 rpm. For APEC strains and S. Typhimurium, 50 μl of freezer stock was inoculated into 5 mL of LB in a 15 mL round bottom shaker tube. The cultures were incubated at 37° C. overnight while shaking at 200 rpm. Once pathogens had grown overnight in liquid culture, 1.0×105 cfu/ml of the overnight culture were inoculated into freshly prepared LB soft agar (0.8% w/v) that was cooled in a water bath set to 45° C. after autoclave sterilization. 5 mL of the molten agar was aliquoted into each well of a 6-well cell culture plate (2 wells per Bacillus strain plus the negative control). The soft agar was solidified and air-dried for 3-4 hours. Onto this agar, 5 μl of 48-hour Bacillus culture were applied to the center of each well. The plates were inverted and allowed to incubate overnight at 37° C. for 24 hours and zones of inhibition were observed and recorded.

For Clostridium perfringens screening, 5 mL of molten LB agar (1.5%, w/v) were aliquoted into each well of a 6-well cell culture plate and allowed to solidify overnight. Then 5 μl of 48-hour Bacillus culture were spotted onto the center of each well. The plates were inverted and allowed to incubate overnight aerobically at 37° C. A colony of Clostridium perfringens NAH 1314-JP1011 was inoculated in liquid BYC broth an incubated overnight at 39° C. in the anaerobic chamber. Freshly prepared BYC soft agar (0.8%, w/v) was autoclaved and allowed to cool in a water bath set to 45° C. Once cooled, the overnight C. perfringens culture was inoculated into molten soft agar at 1.0×105 cfu/ml and mixed on a stir plate. 5 mL of the molten agar was aliquoted on top of each well of the 6-well cell culture plates containing Bacillus spots. As a negative control, C. perfringens-containing molten agar was poured onto LB agar without Bacillus. Once solidified, plates were inverted and allowed to incubate anaerobically overnight at 39° C. for 24 hours. Then, zones of inhibition were observed and recorded.

Enzyme activities—The β-mannanase assay was adapted from a protocol as described by Cleary, B., et. al. (24). Assays for amylase and protease activities were done following protocols in (23). β-mannanase assay was adapted from a protocol as described by Cleary, B., et. al. (114). Assays for amylase and protease followed protocols in (113). For testing β-mannanase activity, Bacillus strains were grown in 5 milliliters of LB medium in a 15 mL culture tube overnight at 37° C. while shaking at 200 rpm. Then 5 μl of 24 hour Bacillus culture were spotted in duplicate onto the center of an LB agar plate containing 100 mM CaCl2). The agar plates were incubated overnight at 37° C. Fresh soft agar containing Azo-carob Galactomannan (0.5%, w/v), agar (0.7%, w/v), dissolved in 50 mM Tris-HCl pH 7.0 buffer was autoclaved and allowed to cool in a water bath set to 45° C. Once cooled, the soft agar substrate was overlayed on to agar plates containing Bacillus colonies until each colony was surrounded by substrate. The plates were incubated overnight at 37° C. and allowed to incubate for 48 hours. The zone of clearance due to β-mannanase activity could be directly visualized and recorded.

For the amylase assay, agar plates containing the following ingredients were used (entity, g/L): Tryptone, 10, Soluble starch, 3, KH2PO4, 5, Yeast extract, 10, Noble Agar, 15. An overnight culture of Bacillus isolates in 0.5× LB was used as an inoculum. The Bacillus culture was spotted onto the above plate containing soluble starch and the inoculated plates were incubated at 37° C. for 48 hours. The zone of clearance due to amylase activity was visualized by flooding the surface of the plates with 5 mL of Gram's iodine solution.

For testing protease activity, agar plates containing the following ingredients were used (entity, g/L): skim milk, 25, noble agar, 25. An overnight culture of Bacillus isolates in 0.5× LB was used as inoculum. The Bacillus culture was spotted onto the above plate containing soluble starch and the inoculated plates were incubated at 37° C. for 24 hours. The zone of clearance due to protease activity could be directly visualized.

Cytotoxicity assay—Cytotoxicity assays of Bacillus culture supernatants were performed following the protocol described in EFSA guidelines (25). Culture supernatant of B. cereus ATCC 14579 and B. licheniformis ATCC14580 were used as positive and negative controls, respectively. Bacillus spp. strains were grown in 5 mL Brain Heart Infusion (BHI) liquid medium at 30° C. overnight. This overnight culture served as an inoculum for 5 mL fresh LB, the inoculated medium was then incubated at 30° C. for 6 hours without shaking. The expected cell density was at least 108 CFU/mL. The culture was then centrifuged at 1,700×g for 1 hour to generate cell-free culture supernatant.

200 μL serum-free medium were added to the 100% confluent Vero cells grown on 96-well plates generated following the protocol described in Materials and Methods. The cells were then exposed to 100 μL of cell-free culture supernatant of Bacillus spp. and the mixture was incubated inside a CO2 incubator (5% v/v headspace of C02, Thermo Scientific, Waltham, MA) at 37° C. for 3 hour. The corresponding cell-free culture supernatant was used in the control wells. B. cereus and B. licheniformis were used as positive and negative controls, respectively, and 0.1% Triton-X, 100 μL was used as a positive cytotoxicity control. The assay was performed in three technical replicates with three biological replicates.

At the end of the incubation period, culture supernatants were collected by centrifugation at 300×g for 5 min. Culture supernatants from technical replicate wells were combined. Four micro liters of the culture supernatant were used for a lactate dehydrogenase assay (Sigma Aldrich, St. Louis, MO) with a total volume of 100 μL, following the protocol as described in (115). The reaction was monitored at an absorbance of 450 nm at 37° C. for 10 minutes measuring the generation of NADH from NAD+ as products from lactate dehydrogenase reaction. The percent cytotoxicity level was calculated by the following formula. % Cytotoxicity=(A460 nm sample—A460 nm media control)

$$\% Cytotoxicity = \frac{( A 460 nm sample - A 460 nm media control )}{( A 460 nm \_Triton X - A 460 nm\_ media control )}$$

(A460 nm_Triton X—A460 nm_media control)

The A450 nm value is an average of three biological replicates. A cytotoxicity percentage value higher than 20 was considered cytotoxic. The assays were repeated if cytotoxicity percentage of B. cereus, a positive control, was less than 40 or that of B. licheniformis, a negative control, was higher than 20.

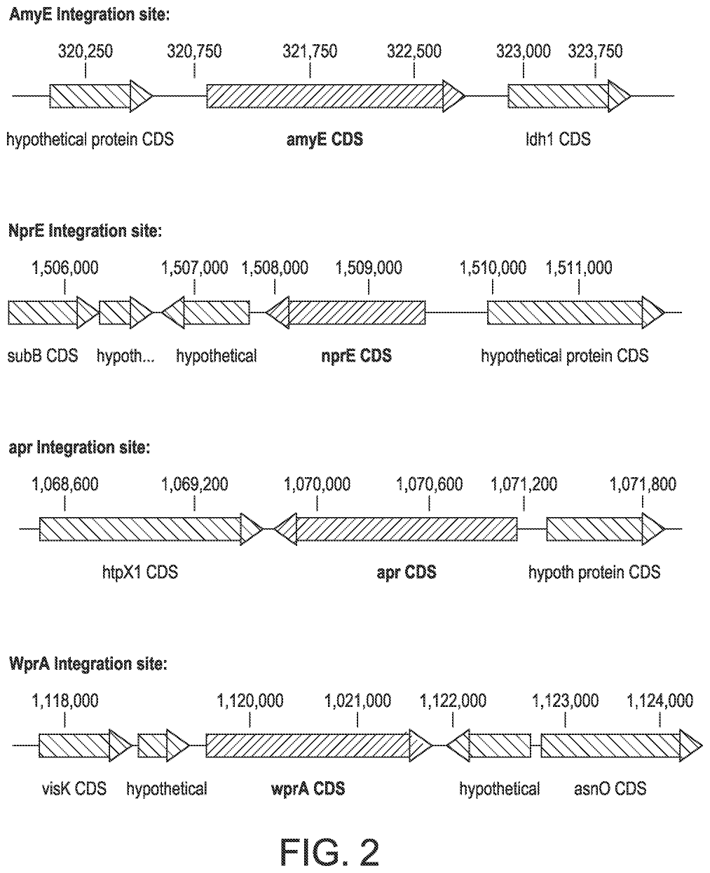

Antimicrobial susceptibility assessment—Antibiotic susceptibility assays of Bacillus spp. for tetracycline, chloramphenicol, streptomycin, kanamycin, erythromycin, vancomycin, gentamycin, ampicillin, and clindamycin were performed and assessed according to an EFSA guideline for Antimicrobial resistance of the Bacillus spp. as direct fed microbials (25). Bacillus spp. strains on LB agar plates were sent to Microbial Research, Inc. (Fort Collins, CO) for analysis following protocols in compliance with Clinical Laboratory Standard Institute (CLSI) document VET01 (26). Briefly, MIC plates were prepared using cation-adjusted Mueller Hinton Broth (MHB) and the antimicrobials were 2-fold serially diluted to obtain a final concentration range of 0.06-32 μg/mL. Growth of Bacillus spp. in the presence of each of nine antimicrobials with different dilutions was monitored. Susceptibility was interpreted as the lack of Bacillus spp. growth in the presence of antimicrobial at a concentration that was lower that the cut-off values of the respective antimicrobials described in the EFSA guideline (FIG. 2A). For quality control, the following organisms were used as controls, Escherichia coli ATCC 25922, Enterococcus faecalis ATCC 29212, Pseudomonas aeruginosa ATCC 27853 and Staphylococcus aureus ATCC 29213.

### Whole Genome Sequencing, Assembly and Annotation

Genomic DNA isolation—High molecular weight genomic DNA of Bacillus spp. were extracted employing a Phenol:Chloroform:Isoamyl alcohol (PCI) method as described previously (27). Bacterial cells were harvested by centrifugation at 7,000×g for 10 min from an overnight culture of Bacillus spp. grown in 25 mL LB supplemented with 0.005% Tween 80 in 50 mL sterile Falcon tube (Fisher Scientific, Waltham, MA). The resulting cell pellet was resuspended in 0.75 mL of 1× Tris-EDTA (TE) buffer (Life Technologies, Carlsbad, CA), pH 8, containing Tris-HCl and EDTA at final concentrations of 10 and 1 mM, respectively, in a 2 mL Eppendorf tube (Fisher Scientific, Waltham, MA). To lyse the cells, Lysozyme (Sigma Aldrich, St. Louis, MO) was added at a final concentration of 7 mg/mL and the mixture was incubated at 37° C. for an hour. Then, SDS and Proteinase K (Sigma Aldrich, St. Louis, MO) were added to the mixture at final concentrations of 2% and 400 μg/mL, respectively, and the lysate was incubated at 60° C. for 1 hour. To remove RNA from the cell lysate, 10 μL of RNase (ThermoFisher Scientific, Waltham, MA) were added and the mixture was incubated at 37° C. for 30 min. An equal volume of a mixture of PCI (25:24:1, v/v/v) was added to the supernatant and was mixed by carefully inverting tubes 5-10 times rigorously. The aqueous phase containing DNA was separated from the organic phase by centrifugation at 12,000×g for 15 min, and the top aqueous layer was collected into a fresh 2 mL Eppendorf tube. An equal volume of a mixture of Chloroform:Isoamyl alcohol (24:1, v/v) was added to this aqueous phase containing DNA, and mixed by carefully inverting the tube. The mixture was centrifuged at 12,000×g for 10 min. DNA from the aqueous layer was precipitated by an addition of one tenth volume of sodium acetate (3M, pH 5.2) followed by centrifugation at 16,000×g for 20 min. The DNA pellet was washed three times with ice-cold 70% ethanol, air-dried, and resuspended in 0.5 mL 1× TE buffer.

PacBio long read genome sequencing—The bacterial genomic DNA samples were shipped on dry-ice to DNA Link, Inc. (San Diego, CA) for whole genome sequencing using PacBio RSII platform.

Briefly, 20 kb DNA fragments were generated by shearing genomic DNA using the covaris G-tube according to the manufacturer's recommended protocol (Covaris, Woburn, MA). Smaller fragments were purified by the AMpureXP bead purification system (Beckman Coulter, Brea, CA). For library preparation, 5 μg of genomic DNA were used. The SMRTbell library was constructed using SMRTbell^{T}M Template Prep Kit 1.0 (PacBio®, Menlo Park, CA). Small fragments were removed using the BluePippin Size selection system (Sage Science, Beverly, MA). The remaining DNA sample was used for large-insert library preparation. A sequencing primer was annealed to the SMRTbell template and DNA polymerase was bound to the complex using DNA/Polymerase Binding kit P6 (PacBio®, Menlo Park, CA).

Following the polymerase binding reaction, the MagBead was bound to the library complex with MagBeads Kit (PacBio®, Menlo Park, CA). This polymerase-SMRTbell-adaptor complex was loaded into zero-mode waveguides. The SMRTbell library was sequenced by 2 PacBio® SMRT cells (PacBio®, Menlo Park, CA) using the DNA sequencing kit 4.0 with C4 chemistry (PacBio®, Menlo Park, CA). A 1×240-minute movie was captured for each SMRT cell using the PacBio® RS sequencing platform.

Genome Assembly, Annotation and Features Prediction—The genome was assembled by DNA link, Inc. with HGAP.3. Genome annotation was carried out using a custom annotation pipeline by combining several prediction tools. Coding sequences, transfer RNA and transmembrane RNA were predicted and annotated using Prokka (28-30). Ribosomal binding site (RBS) prediction was carried out using RBSFinder (31). TranstermHP was used to predict Rho-independent transcription terminators (TTS) (32). Ribosomal RNA and other functional RNAs such as riboswitches and non-coding RNA was annotated with Infernal (33). Operons were predicted based on primary genome sequence information with Rockhopper v2.0.3 using default parameters (34). Insertion sequence prediction was done using ISEscan v.1.7.2.1 (40). Prophage prediction was done using PhiSpy v4.2.6 which combines similarity- and composition-based strategies (41).

Genome-based strain identification and comparative genomic analyses—Taxonomic labelling of the assembled microbial genomes was carried out using CAMITAX (35). CAMITAX is a scalable workflow that combines genome distance-, 16 S ribosomal RNA gene-, and gene homology-based taxonomic assignments with phylogenetic placement. OrthoFinder v2.3.1 (36) was used to determine orthologous relationships (37).

Phylogenetic analysis—Phylogenetic relationships of the genomes were explored with UBCG v3.0 using default settings (38). This software tool employs a set of 92 single-copy core genes commonly present in all bacterial genomes. These genes then were aligned and concatenated within UBCG using default parameters. The estimation of robustness of the nodes is done through the gene support index (GSI), defined as the number of individual gene trees, out of the total genes used, that present the same node. A maximum-likelihood phylogenetic tree was inferred using FastTree v.2.1.10 with the GTR+CAT model (39).

Patent depository of Bacillus amyloliquefaciens ATCC PTA-126784 and PTA-126785, and B. subtilis ATCC PTA-126786-Bacillus amyloliquefaciens ATCC PTA-126784 and PTA-126785, and B. subtilis ATCC PTA-126786 strains were deposited in the ATCC culture collection (Manassas, VA). For simplicity, Bacillus amyloliquefaciens ATCC PTA-126784 and PTA-126785, and B. subtilis ATCC PTA-126786 strains are referred to as Ba PTA84 and Ba PTA85, and Bs PTA86, respectively.

Global untargeted metabolomic analysis—Bacillus strains Bs PTA86, Ba PTA84, and Ba PTA85 were grown as three single strain cultures, and then were analyzed as a two-strain (Ba-PTA84 and PTA85) or three-strain (Bs-PTA86, Ba-PTA84, and Ba-PTA85) consortia in 5 mL of minimal or rich liquid media. For growth in minimal media, medium containing 1× M9 salts, and glucose at a final concentration of 0.5% (w/v) was used. Rich medium contained the following entities (g/L): peptone 30; sucrose 30; yeast extract 8; KH2PO4 4; MgSO4 1; and MnSO4 0.025. The culture was grown at 37° C. overnight. Bacillus cells were pelleted by centrifugation at 10,000×g for 10 min, cell pellets were washed three times with ice-cold PBS. The resulting cell pellets and cell-free supernatants were stored at −80C and sent to metabolon Inc. (Durham, NC) for global untargeted metabolomic profiling. Detailed description of metabolomic analysis is presented in Supplementary Methods.

In-vivo assessment of Bacillus DFM for improvement of growth performance in broiler chickens

Spore generation—Bacillus spores were generated employing a modified protocol as described in (42). Bacillus spp. was grown in a liquid Difco sporulation medium containing Nutrient Broth (BD Difco, Franklin Lakes, NJ, USA), 8.0 g/L; KCl, 1 g/L, and MgSO4·7H2 O, 0.12 g/L. The mixture was adjusted to pH 7.6 with additions of NaOH. After adjusting the pH and sterilizing the media by the use of an autoclave at 121° C., 1 mL of each of the following mineral sterile stock solutions were added to broth media, 1.0 M CaCl2), 0.01 M MnSO4, 1.0 mM FeSO4. A sterile glucose solution was also added to the medium mixture to a final concentration of 5.0 g/L. A single colony was taken from an agar plate and was inoculated into 100 mL of the sporulation medium. The culture was incubated overnight at 37° C. with shaking at 200 rpm. This culture served as a seeding culture for 1 L of liquid culture. All growth were done employing vented baffled flasks. This culture was incubated at 37° C. while shaking at 200 rpm for at least 72 hours. The presence of spores was monitored with a brightfield microscope. The spores were harvested at 17,000 rpm and washed three times with pre-chilled sterile distilled water. The spores were then resuspended in 30 mL of pre-chilled sterile distilled water and the spore suspension was mixed with irradiated ground rice hulls (Rice Hull Specialty Products, Stuttgart, AR), dried at 60° C. for 3-4 hours to eliminate vegetative cells. To determine spore inclusion in the rice hulls, 0.25 g of the material containing spores was heat treated at 90° C. for 5 min. One milliliter of water was added to the material and allowed to soak for 15-30 min. The suspension was vortexed for 30 sec and serially diluted 10-fold for colony counts on agar plates.

### Study Design

A total of 2,500 one-day-old male broiler chicks (Cobb 500) were randomly allocated to two treatment groups on Study Day (SD) 0. The control group received only the basal diet, while the treated group received the basal diet plus 1.5×105 CFU of Ba PTA84 per gram of final feed. The control group consisted of 30 pens of 50 birds per pen, and the Ba PTA84 group consisted of 20 pens of 50 birds per pen.

Birds were housed in floor pens in a single environmentally controlled room with ad libitum access to treatment diets and water. Basal diets were formulated to be iso-nutritive, and to meet or exceed the nutrient requirements recommended for broilers. Feed was issued in four study phases: Starter Phase I (SD 0-12); Grower Phase II (SD 12-26); Finisher Phase III (SD 26-35), Withdrawal Phase IV (SD 35-42). Diets did not contain antibiotics, anticoccidials or growth promoters and were fed to the birds as a mash in all phases.

Bird weights (pen weight) were measured and recorded at SD 0, 12, 26, 35 and 42. Feed issued and weighed back were recorded for each feeding phase. Bird general health, mortality and environmental temperature were recorded daily.

### Statistical Analysis

The experimental unit was the pen. All statistical analysis was conducted using the SAS® system version 9.4 (SAS Institute, Cary, NC)) and all tests were performed comparing the control group to the treated group using a one-sided test at P<0.05 level of significance.

Performance variables of interest for each feeding period and overall included: live final body weight (LFBW), average daily gain (ADG), average daily feed intake (ADFI), gain to feed efficiency (GF), feed to gain efficiency (FCR), mortality, and the European Broiler Index (EBI). These variables were calculated and evaluated for each study phase (Starter, Grower, Finisher, Withdrawal and Overall (SD 0-42)) and both adjusted for mortality and unadjusted.

Microbiome Profiling of Cecal Content from Birds Treated with Ba PTA84

DNA Extraction, Library Preparation and Sequencing—Total DNA from cecal content samples were extracted employing the Lysis and Purity kit (Shoreline Biome, Farmington, CT) following manufacturer's protocol. The resulting DNA was used as template for library preparation using Shoreline Biome's V4 16 S DNA Purification and Library Prep Kit (Shoreline Biome, Farmington, CT). Briefly, PCR amplification of the V4 region of the 16 S rRNA gene was performed using the extracted DNA and the primers 515F (5′GTGGCCAGCMGCCGCGGTAA (SEQ ID NO: 35)) and 806R (5′ GGACTACHVHHHTWTCTAAT (SEQ ID NO: 36)). The resulting amplicons were then sequenced using 2×150 bp paired-end kits on the Illumina iSeq platform. To increase diversity, PhiX 50 μM was added to a final concentration of 5% into the amplicon library.

Bioinformatic analysis—Forward and reverse reads were processed with cutadapt (v 2.5) (43) to remove primer sequences. Read pairs without primer sequences present or more than 15% primer mismatches were discarded. The DADA2 pipeline (v. 1.12.1) (44) was used to generate a count matrix of amplicon sequence variants (ASVs) across samples. Due to the short length of iSeq reads, forward and reverse reads were trimmed to a length of 110 bp and merged with DADA2's justConcatenate option. The DADA2 parameters parameters maxN=O, truncQ=2, rm.phix=TRUE and maxEE=2 were used. Taxonomic labels were assigned to each ASV using the DADA2 assignTaxonomy method and the Silva v. 138 database (45). Diversity and richness per sample were quantified from the ASV matrix using the Simpson, Shannon and Chao indices (46-48) and compared across treatments with the Mann-Whitney U test. Comparison of microbiome structures across treatments was performed using PERMANOVA and ANOSIM analysis based on the Bray-Curtis dissimilarity between samples. PERMANOVA and ANOSIM were performed using code in the scikit-bio python package (49). Principal component analysis of the Bray-Curtis dissimilarity matrix was used to analyze sample clustering according to treatment group.

Global untargeted metabolomic analysis—Metabolite analysis was performed at Metabolon, Inc. utilizing non-targeted UPLC-MS/MS approach employing a Waters ACQUITY ultra-performance liquid chromatography (Waters, Milford, MA) and a Q-Extractive high resolution/accurate mass spectrometer (Thermo Scientific, Waltham, MA) interfaced with a heated electrospray ionization (HESI-II) source and Orbitrap mass analyzer operated at 35,000 mass resolution. The samples were dried, reconstituted and aliquoted into four samples for the following analyses, a) Analysis of hydrophilic compounds employing acidic positive ion conditions with a C18 column (Waters UPLC BEH C18-2.1×100 mm, 1.7 μm) using water and methanol, containing 0.05% perfluoro pentanoic acid (PFPA) and 0.1% formic acid (FA), b) Analysis of more hydrophobic compounds employing a similar system as mentioned above except the mobile phase used was methanol, acetonitrile, water, 0.05% PFPA and 0.01% FA and was operated at an overall organic content. c) Analysis of basic negative ion employing a C18 column with methanol and water as mobile phase that contained 6.5 mM Ammonium Bicarbonate at pH 8. d) negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis covered approximately 70-1000 m/z.

Metabolic compounds were identified by comparison to the Metabolon libraries of purified standards and recurrent unknown metabolites. The identification was based on retention index within a narrow RI window of the proposed identification, accurate mass match to the library+/−10 ppm, and the MS/MS forward and reverse scores.

Data from cell pellets and culture supernatants were analyzed separately. Raw intensity values were re-scaled for each identified metabolite by dividing them by the median intensity across samples. Missing values for a given metabolite and sample were imputed by assigning the minimum value for the metabolite across samples. The scaled and imputed data were Log10 transformed for subsequent analyses. Principal component analysis (PCA) was used to analyze the similarity of metabolic profiles between samples. For supernatant samples, secreted metabolites were identified by comparing the scaled and imputed intensities to the respective metabolites in media controls. A 1.5-fold increase in scaled intensities over media was used to define metabolites secreted. A similar 1.5-fold increase between an individual strain and the remaining 2 strains, or between strain consortia and the corresponding individual strains, was used to define uniquely secreted metabolites.

### Results

Isolation and Identification Bacillus Spp. from Healthy Animals

Bacillus spp. strains were isolated from the cecal contents and fecal materials of healthy chickens. The taxonomic identities of the isolates were determined by 16 S-rRNA amplicon sequencing. These isolates belonged to 30 different Bacillus species with the top hits of B. velezensis, B. amyloliquefaciens, B. haynesii, B. pumilus, B. subtilis, and B. licheniformis.

Due to safety considerations, Bacillus spp. isolates chosen for further screening included only those that belong to the species listed as DFMs in the Association of American Feed Control Officials, Inc. (AAFCO) Official Publication since they “were reviewed by FDA Center for Veterinary Medicine and found to present no safety concerns when used in direct-fed microbial products”(50), and to the species listed as Qualified Presumption of Safety (QPS) status according to the European Food Safety Authority (EFSA) BIOHAZ Panel (3). These were B. subtilis, B. amyloliquefaciens, B. pumilus, and B. licheniformis.

In-Vitro Screening for Probiotic Properties of Bacillus Spp. Strains

Bacillus spp. strains were tested to determine their effect on selected microorganisms and their ability to secrete selected enzymes (23). For the former, Gram-negative and Gram-positive microorganisms (E. coli O2, O18, and O78, and Clostridium perfringens NAH 1314-JP1011) and Salmonella enterica serovar Typhimurium ATCC 14028, were used. For the latter, plate-based assays for determining the secretion of amylase, protease, and β-mannanase were performed.

A total of 266 Bacillus strains were first screened against E. coli 02, and 71% of the strains showing positive E. coli 02 inhibition were selected for a second-round of assays targeting E. coli 018, then E. coli O78, S. Typhimurium and lastly C. perfringens JP1011. The top 8 Bacillus strain candidates were selected according to their cumulative inhibition scores, and selected data for included B. subtilis (Bs) isolate Bs PTA86 (ELA191105, also designated as strain 105) is provided in TABLE 10.

TABLE 10
In Vitro Pathogen Inhibition and Digestive Enzyme Activities of
Bacillus subtilis PTA86

| Pathogen inhibition and | Bs-PTA86 |
|---|---|
| digestive enzyme activities | (ELA191105/strain 105) |
| Pathogen inhibition^{a} | |
| Salmonella Typhimurium | 0 |
| ATCC 14028 | |
| Clostridium perfringens | 3 |
| JP1011 | |
| APEC O78 | 0 |
| APEC O2 | 1 |
| APEC O18 | 1 |
| Cumulative pathogen | 5 |
| inhibition Score | |
| Digestive enzymes^{b} | |
| Amylase | 2.04 |
| Protease | 1.95 |
| Beta-mannanase | 2 |
| Cumulative REA Score | 5.99 |

^{a}Pathogen inhibiton scores were assigned based on the size of clearance zone as follows, 0, no inhibition; 1, 2, 3, 4, clearance zone values of 0-0.9, 1.0-1.9, 2.0-2.9, and 3.0-4.0 mm, respectively. A clearance zone value is defined as the distance from the outer part of Bacillus colony to the end of pathogen growth inhibition zone.
^{b}Relative digestive enzyme activities were measured in Relative Enzyme Activity values (REA) that were calculated as a ratio between a diameter of clearance zone from enzyme activity and the diameter of Bacillus colony.

The cumulative inhibition score was calculated as the sum of the inhibition score values of a Bacillus strain against the five microorganisms tested. The average cumulative inhibition score was 5.5 for Bs PTA86.

The Bacillus strain candidate was evaluated for the ability to secrete enzymes. Bacillus strains are known to produce a variety of enzymes (51, 52). In vitro plate-based assays for protease, amylase, and β-mannanase activities showed that Bs PTA86 demonstrated amylase, protease, and β-mannanase activities.

Safety Assessment of Bacillus Spp. Strains

To evaluate the safety of Bacillus spp. as microbial feed ingredients, the Bacillus candidates were tested for antimicrobial susceptibility to medically relevant antimicrobials. Microbial feed ingredients should not carry or be capable of transferring antimicrobial resistance genes to other gut microbes. This is especially important in the case of medically relevant antimicrobials that are used in humans, given the rise of multidrug resistant bacteria. Antimicrobial susceptibility tests for Bacillus strain BS PTA86 showed that was susceptible to all of the tested antibiotics, specifically to each of clindamycin, chloramphenicol, erythromycin, gentamicin, kanamycin, streptomycin, tetracycline, vancomycin and ampicillin (data not shown).

To determine the potential toxicity of Bacillus strains on host cells, culture supernatants of Bacillus spp. were tested for cytotoxicity toward Vero cells according to (25). The cytotoxicity assay was performed by monitoring the lactate dehydrogenase (LDH) enzyme originated from compromised Vero cells as described in (53). The results suggested that the tested Bacillus strains were non-cytotoxic with toxicity levels far below 20%, a percentage that is considered cytotoxic according to the EFSA guidelines (data not shown). The cytotoxicity level of Bs PTA86 was the lowest among strains evaluated, 5%.

### Selection of Bacillus Spp. as Direct Fed Microbial Candidates

Based on performance on microorganism inhibition, enzymatic activities, antimicrobial susceptibility, and low toxicity against Vero cells, strain Bs PTA86 was chosen for more detailed characterization employing genomic and metabolomic approaches described in the following sections.

Untargeted Global Metabolomic Analysis of Cell Pellets and Culture Supernatants Bs PTA86

Untargeted metabolomics analysis of cell pellets and culture supernatants of Bs PTA86 was performed to assess differences in metabolite profiles. Cells were cultured in both rich and minimal media as individual strains. Named metabolites were identified in the supernatant and pellet samples, respectively. Thus, strain Bs PTA86 (ELA191105) secretes metabolites and includes intracellular metabolites that are unique versus other Bacillus strains. Details and specifics, including tablulated listings, regarding unique metabolites of ELA191105, including in comparison with other Bacillus strains is provided in U.S. Ser. No. 63/083,697 filed Sep. 25, 2020 and in U.S. Ser. No. 63/241,369 filed Sep. 7, 2021, each of which are incorporated by reference herein.

### Genome Properties of Bs PTA86

The genome of Bs PTA86 was sequenced by PacBio sequencing. The genome properties and annotation of different features are summarized in TABLE 11. The whole-genome sequences were deposited at DDBJ/ENA/GenBank under BioProject numbers PRJNA701126 and PRJNA701127. The genome sequence of strain Bs PTA 86 is included and provided in U.S. Ser. No. 63/083,697 filed Sep. 25, 2020 and in U.S. Ser. No. 63/241,369 filed Sep. 7, 2021, each of which are incorporated by reference herein. The BsPTA86 strain (ELA191105) genome nucleic acid sequence is also provided in SEQ ID NO: 1 and in SEQ ID NOs:2-6.

TABLE 11
Genome Assembly and Annotation Summary of Bacillus spp.

| | PTA-86 |
|---|---|
| | (ELA191105/strain |
| Feature | 105) |
| No. sequences | 1 |
| Total genome size (bp) | 4,089,676 |
| CDS | 4,027 |
| Miscellaneous feature | 8 |
| Mobile Elements | 2 |
| Non-coding RNA | 11 |
| Operons | 747 |
| Ribosomal RNA | 30 |
| Ribosomal binding sites | 4,026 |
| Transcription Terminators | 2,196 |
| Riboswitch | 48 |
| Transfer RNA | 86 |
| Transfer-messenger RNA | 1 |

Phylogenetic analysis of Bs PTA86—Phylogenetic relationships of the genome was explored with UBCG v3.0 which employs a set of 92 single-copy core genes commonly present in all bacterial genomes. The Bs PTA86 genome was compared against the genomes of B. amyloliquifaciens, B. velezensis and B. subtilis strains along with LactoBacillus reuterii as an outgroup (Accession numbers: AL009126, CP000560, CP002627, CP002634, CP002927, HE617159, HG514499, JMEFO1000001, CP005997, CP009748, CP009749, CP011115, LHCCO1000001, CP014471 and QVMXO1000001). Bs PTA86 showed closest relationship to Bacillus subsp. Subtilis 168 (ATCC 23857, DSM 23788)

Genome analysis Bs PTA86—The assembled genome sequence of Bacillus strain 105 was annotated for the potential probiotic properties such as enzymes, antioxidants, bacteriocins, and secondary metabolites, and for the presence of genes of potential safety concerns such as genes encoding toxins, virulence factors, and antimicrobial resistance genes. A detailed description of each of the above-mentioned features is described below.

Selected enzymes analyses—TABLE 12 illustrates the presence genes encoding selected digestive enzymes identified in the Bacillus Bs PTA86 genome. All three Bacillus genomes encode lipase, 3-phytase, alpha-amylase, endo-1,4-β xylanase A, p glucanase, β-glucanase, β-mannanase, pectin lyase, and alpha galctosidase. Bs PTA86 carried two copies of β-mannanase genes. β-mannanase catalyzes the hydrolysis of β-1,4-linkage of glucomannan releasing mannan oligosaccharide (24, 54). This enzyme along with phytase, xylanase, amylase are added as feed ingredients to improve feed digestibility (55-57). Bs PTA86 possessed pullulanase, oligo-1,6-glucosidase, and glycogen degradating enzymes such as 1,4-alpha-glucan branching enzyme.

TABLE 12

| | Bs PTA86 |
|---|---|
| Enzyme | (strain 105) |
| Lipase | Present |
| 3-phytase | Present |
| Alpha-amylase | Present |
| Endo-1,4-beta-zylanase A | Present |
| Beta-glucanase | Present |
| Beta-mannanase | Present |
| | (Multicopy) |
| Pectin lyase | Present |
| Alpha-galactosidase | Present |
| 1,4-Alpha-glucan branching | Present |
| enzyme | |
| Pullulanase | Present |
| Oligo-1,6-glucosidase 1 | Present |

Secondary metabolites—Secondary metabolite clusters accounted 12% of the genome of Bacillus Bs PTA86. TABLE 13 illustrates the respective clusters for the Bacillus Bs PTA86 genome, which encodes for 10 clusters. More than half of the clusters were contributed by biosynthetic genes for antimicrobial peptides (AMPs) (TABLE 14). The Bs PTA86 genome possessed subtilosin A, a cyclic antimicrobial peptide potent against some Gram positive and Gram negative bacteria such as Listeria monocytogenes, Enterococcus faecalis, Porphyromonas gingivalis, Klebsiella rhizophila, Streptococcus pyogenes and Shigella sonnei, Pseudomonas aeruginosa and Staphylococcus aureus (58-60). For non-ribosomally synthesized AMPs, Bs PTA 86 carries plipastatin, surfactin, bacillibactin, and bacilysin.

TABLE 14 provides a tabulation and comparison of some antimicrobial peptides and TABLE 15 provides digestive enzymes provided by the strain Bs PTA 86.

TABLE 13
Secondary Metabolites Gene Clusters of Bs PTA86

| Type | PTA |
|---|---|
| Class/Cluster* | 86 |

Bacteriocin and RiPPs class

| | |
|---|---|
| Amylocyclicin | 0 |
| ComX1 | 0 |
| LCI | 0 |
| Lanthipeptide_class_II | 0 |
| Competence | 1 |
| Subtilosin_(SboX) | 1 |

Secondary metabolite biosynthesis gene cluster

| | |
|---|---|
| CDPS | 1 |
| NRPS | 2 |
| NRPS, PKS-like, T3PKS, transAT-PKS, transAT-PKS- | 1 |
| like | |
| NRPS, T3PKS, transAT-PKS, transAT-PKS-like | 0 |
| NRPS, bacteriocin | 0 |
| NRPS, betalactone | 1 |
| NRPS, betalactone, transAT-PKS | 0 |
| NRPS, transAT-PKS | 0 |
| PKS-like | 0 |
| T3PKS | 1 |
| head_to_tail, sactipeptide | 1 |
| lanthipeptide | 0 |
| terpene | 2 |
| transAT-PKS | 0 |
| transAT-PKS, transAT-PKS-like | 0 |
| other | 1 |

*Abbreviations, RiPP, ribosomally synthesized and post-translationally modified peptides; NRPS, Non-Ribosomal Peptide Synthase; PKS, polyketide synthasePolyketide Synthase; T3PKS, type III polyketide synthase; trans-Type 3-PKS; AT-PKS, trans-acyltransferase polyketide synthase. Acyltransferase PKS.

TABLE 14
Antimicrobial peptides

| | Bs PTA86 |
|---|---|
| PEPTIDE | (strain 105) |
| Ribosomally-synthesized | |
| antimicrobial peptides | |
| Lichenicidin A | Absent |
| Circularin | Absent |
| LCI | Absent |
| Subtilosin A | Present |
| Salicylate containing AMPs | Absent |
| Non-Ribosomally-synthesized | |
| antimicrobial peptides | |
| Plipastatin | Present |
| Surfactin | Present |
| Bacillibactin | Present |
| Bacilysin | Present |
| Gramicidin/Tyrocidin | Absent |

TABLE 15
Putative digestive enzymes identified in the genome of B. subtilis PTA-85

| | Accession | B. subtilis |
|---|---|---|
| Gene Description | Number | PTA-86 |
| 1,4-alpha-glucan branching enzyme GlgB | AGA22754.1 | Present |
| 6-phospho-beta-galactosidase | QDK89482.1 | None |
| 6-phospho-beta-glucosidase GmuD | QDK91913.1 | Present |
| Alpha-amylase | BAT21551.1 | Present |
| Alpha-galactosidase | QDK91116.1 | Present |
| Alpha-galacturonidase | ASB68722.1 | Present |
| Aryl-phospho-beta-D-glucosidase BglA | QDK90194.1 | Present |
| Aryl-phospho-beta-D-glucosidase BglC | ARV97270.1 | Present |
| Aryl-phospho-beta-D-glucosidase BgIH | QAS10023.1 | Present |
| Beta-glucanase | AYL88759.1 | Present |
| Beta-glucanase | AYL88759.1 | None |
| Beta-hexosaminidase | QDK88534.1 | Present |
| Beta-mannanase | | Present |
| Cephalosporin-C deacetylase | QDK88655.1 | Present |
| Cortical fragment-lytic enzyme | ATC51419.1 | Present |
| Demethyllactenocin mycarosyltransferase | QDK88875.1 | Present |
| Endo-1,4-beta-xylanase A | QDK91715.1 | Present |
| Endoglucanase | QDK90074.1 | Present |
| Endoglucanase | CCF05300.1 | None |
| General stress protein A | QDK91887.1 | Present |
| GlcNAc-binding protein A | QEQ03549.1 | None |
| Glycogen phosphorylase | AIX08721.1 | Present |
| Glycogen synthase | QAW13524.1 | Present |
| Intracellular maltogenic amylase | AMR45682.1 | Present |
| L-Ala--D-Glu endopeptidase | QGT57119.1 | Present |
| Maltose-6′-phosphate glucosidase | QDK89133.1 | Present |
| Melibiose/raffinose/stachyose import permease protein MelC | QEK97784.1 | Present |
| Membrane-bound lytic murein transglycosylase F | QDK89432.1 | Present |
| Membrane-bound lytic murein transglycosylase F | QDK90428.1 | None |
| N-acetyl-alpha-D-glucosaminyl L-malate deacetylase 1 | QEQ03843.1 | Present |
| N-acetylglucosamine-6-phosphate deacetylase | ASB54801.1 | Present |
| N-acetylglucosamine-6-phosphate deacetylase | ASB54800.1 | None |
| N-acetylglucosaminyldiphosphoundecaprenol N-acetyl-beta-D- | QDK91629.1 | Present |
| mannosaminyltransferase | | |
| Oleandomycin glycosyltransferase | QDK89494.1 | Present |
| Oleandomycin glycosyltransferase | QDK90176.1 | Present |
| Oligo-1,6-glucosidase | AKD24292.1 | Present |
| Oligo-1,6-glucosidase 1 | QAW18252.1 | Present |
| 3-phytase | | Present |
| Pectate lyase | QDK89071.1 | Present |
| Pectate lyase C | AIC99792.1 | Present |
| Pectin lyase | QDK91950.1 | Present |
| Penicillin-binding protein 1A/1B | QDK90466.1 | Present |
| Penicillin-binding protein 1A/1B | QDK90466.1 | None |
| Penicillin-binding protein 1F | QDK89310.1 | Present |
| Penicillin-binding protein 2D | QDK91796.1 | Present |
| Penicillin-binding protein 4 | QDK91217.1 | Present |
| Peptidoglycan-N-acetylglucosamine deacetylase | QDK89265.1 | Present |
| Peptidoglycan-N-acetylglucosamine deacetylase | QDK90813.1 | None |
| Peptidoglycan-N-acetylmuramic acid deacetylase PdaA | QEQ05952.1 | Present |
| Peptidoglycan-N-acetylmuramic acid deacetylase PdaC | AIX06967.1 | Present |
| Processive diacylglycerol beta-glucosyltransferase | QAV83859.1 | Present |
| Processive diacylglycerol beta-glucosyltransferase | QDK90264.1 | Present |
| Pullulanase | AYA43052.1 | Present |
| putative 6-phospho-beta-glucosidase | QDK91899.1 | Present |
| putative esterase YxiM | AOY05484.1 | Present |
| putative oligo-1,6-glucosidase 2 | AHC40869.1 | Present |
| putative protein YqbO | QDK89544.1 | Present |
| putative rhamnogalacturonan acetylesterase YesY | ASZ60459.1 | Present |
| Putative sporulation-specific glycosylase YdhD | CAB12390.2 | Present |
| Putative sporulation-specific glycosylase YdhD | QDK89600.1 | Present |
| Rhamnogalacturonan acetylesterase RhgT | QFY87628.1 | Present |
| Rhamnogalacturonan endolyase YesW | QAT44941.1 | Present |
| Rhamnogalacturonan exolyase YesX | AIX06475.1 | Present |
| Trehalose-6-phosphate hydrolase | AVX18210.1 | Present |
| UDP-N-acetylglucosamine--N-acetylmuramyl- | QDK89788.1 | Present (2) |
| (pentapeptide)pyrophosphoryl-undecaprenol N- | | |
| acetylglucosamine transferase | | |

### Genes of Safety Concern

To search for genes encoding known virulence factors, toxins, and antimicrobial resistance (AMR), we applied a screening approach using cutoff values according to an EFSA guideline (61), sequence identity and coverage values higher than 80 and 70%, respectively. According to the analysis, genes for known virulence factors or toxins were not identified in the Bacillus strain Bs PTA86.

TABLE 16 presents genes for putative genes encoding for antimicrobial resistance (AMR). The Bs PTA86 genome carried putative genes that encoded macrolide 2′phosphotransferase (mphK), ABC—F type ribosomal protection protein (vmlR), Streptothricin-N-acetyltransferase (satA), tetracyclin efflux protein (tet(L)), aminoglycoside 6-adenylyltransferase (aadK) (29), and rifamycin-inactivating phosphotransferase (rphC). The aadK gene from B. subtilis was originally found in susceptible derivatives of Marburg 168 strains. Heterologous expression of the gene in a plasmid in E. coli resulted in resistance phenotype toward rifamycin suggesting the need for high gene copies to confer resistance (30).

TABLE 16
Putative antimicrobial resistance genes identified in B. subtilis PTA-86 (strain ELA191105)

| | Gene | % | % | Accession | |
|---|---|---|---|---|---|
| Gene product | id | Coverage | Identity | number | Antibiotic Resistance |
| macrolide 2′- | mphK | 100 | 97.72 | NG_065846.1 | Macrolide |
| phosphotransferase | | | | | |
| MphK | | | | | |
| ABC-F type ribosomal | vmlR | 100 | 98.66 | NG_063831.1 | Lincosamide; |
| protection protein | | | | | Streptogramin; |
| VmlR | | | | | Tiamulin |
| streptothricin N- | satA_Bs | 100 | 95.78 | NG_064662.1 | Streptothricin |
| acetyltransferase SatA | | | | | |
| tetracycline efflux MFS | tet(L) | 100 | 100 | NG_048204.1 | Tetracycline |
| transporter Tet(L) | | | | | |
| aminoglycoside 6- | aadK | 99.77 | 98.12 | NG_047379.1 | Streptomycin |
| adenylyltransferase | | | | | |
| AadK | | | | | |
| rifamycin-inactivating | rphC | 99.39 | 82.18 | NG_063825.1 | Rifamycin |
| phosphotransferase | | | | | |
| RphC | | | | | |

### Antioxidants, Adhesion, and Folate Biosynthesis

Genes encoding primary redox enzymes such as superoxide dismutase and catalase that scavenge reactive oxygen species were found in the three Bacillus genomes, TABLE 17. A thioredoxin system and genes for bacillithiol biosynthesis were also identified. The Bs PTA86 genome encoded for a thioredoxin reductase and a Trx for Bs PTA86. Thioredoxin systems maintain cellular redox homeostasis (62). Interestingly, despite lacking glutathione-glutaredoxin system, several genes for glutathione transport were found suggesting the potential transport of redox proteins, possibly bacillithiol, to the extracellular environment maintaining redox potential of the surroundings. Two genes for bacillithiol biosynthesis (63), bshA and B, were identified in the genome of Bs PTA86, TABLE 17.

TABLE 17
Putative genes encoding antioxidant in the genomes of Bacillus strain 105

| | | B. subtilis |
|---|---|---|
| Description | Gene id | PTA-86 |
| Superoxide dismutase [Mn] | sodA | Present |
| Superoxide dismutase-like protein | yojM | Present |
| Thiol peroxidase | tpx | Present |
| Thiol-disulfide oxidoreductase | resA_1 | Present (2) |
| Thiol-disulfide oxidoreductase YkuV | ykuV | Present |
| Thioredoxin | trxA_1 | NA |
| | trxA_2 | Present |
| Thioredoxin reductase | trxB | Present |
| Thioredoxin-like protein YdbP | ydbP | Present |
| Thioredoxin-like protein YtpP | ytpP | Present |
| Catalase | katA_1 | Present |
| Catalase HPII | katE | NA |
| | katE_2 | Present |
| Putative deferrochelatase/peroxidase EfeN | efeN | Present |
| Sporulation thiol-disulfide oxidoreductase A | stoA | NA |
| Glutathione hydrolase proenzyme | ggt | Present |
| | ggt_2 | Present |
| Glutathione hydrolase-like YwrD proenzyme | ywrD | Present |
| Glutathione transport system permease protein GsiD | gsiD | Present |
| Glutathione-independent formaldehyde | fdhA | Present |
| dehydrogenase | | |
| Glutathione-regulated potassium-efflux system | kefB | Present |
| protein KefB | | |
| Putative peroxiredoxin bcp | bcp | Present |
| Bacillithiol biosynthesis gene | BshA | Present |
| | BshB | Present (2) |
| Free methionine-R-sulfoxide reductase | msrC | Present |
| Peptide methionine sulfoxide reductase MsrA | msrA | Present |

One of the key desirable traits in a probiotic candidate is the ability to adhere to epithelial cells. The two genes identified in all three strains putatively encode proteins involved in adhesion to mucus, epithelial cells and are known to be involved in host immunomodulation and unwanted microorganism aggregation, providing stability to the strains and the ability to compete with other undesirable resident gut bacteria, thereby enabling effective colonization of the gut and exclusion of pathogens (64, 65). Two genes each encoding for elongation factor Tu and 60 kDa chaperonin involved in adhesion of Bacillus species to intestinal epithelium were identified in all three genomes.

Probiotic bacteria confer several health benefits to the host, including vitamin production. We searched for key components of folate production pathways in Bacillus strains using the Enzyme Commission (EC) numbers associated with folate biosynthetic pathway. The analysis of genome sequences of Bacillus strains identified genes involved para-aminobenzoic acid (PABA) synthesis in all three strains (TABLE 18). However, strain Ba PTA84 has a frameshift mutation in pabB gene. The enzymes necessary for chorismate conversion into PABA are present in all three Bacillus probiotic strains. Bacillus probiotic strains also contain the genes of DHPPP de novo biosynthetic pathway. Previous studies have shown that B. subtilis genome harbor all the pathways components and have been engineered for folate production (66-68).

TABLE 18
Genes Involved in Folate Biosynthetic Pathway in Probiotic Bacillus spp.

| | | EC | |
|---|---|---|---|
| Annotation | Gene | Number | PTA-86 |
| Protein AroA(G) | aroA | 5.4.99.5 | Present |
| 3-phosphoshikimate 1- | aroA1 | 2.5.1.19 | Present |
| carboxyvinyltransferase 1 | | | |
| 3-dehydroquinate synthase | aroB | 4.2.3.4 | Present |
| Chorismate synthase | aroC | 4.2.3.5 | Present |
| 3-dehydroquinate dehydratase | aroD | 4.2.1.10 | Present |
| Shikimate dehydrogenase | aroE | 1.1.1.25 | Present |
| (NADP(+)) | | | |
| Shikimate dehydrogenase | aroE_1 | 1.1.1.25 | — |
| (NADP(+)) | | | |
| Shikimate dehydrogenase | aroE_2 | 1.1.1.25 | — |
| (NADP(+)) | | | |
| Chorismate mutase AroH | aroH | 5.4.99.5 | Present |
| Shikimate kinase | aroK | 2.7.1.71 | Present |
| Aromatic amino acid transport | aroP | — | Present |
| protein AroP | | | |
| Dihydroneopterin aldolase | folB | 4.1.2.25 | Present |
| Bifunctional protein FolD protein | folD | 1.5.1.5 | Present |
| GTP cyclohydrolase 1 | folE | 3.5.4.16 | Present |
| GTP cyclohydrolase FolE2 | folE2 | 3.5.4.16 | Present |
| 2-amino-4-hydroxy-6- | | | |
| hydroxymethyldihydropteridine | folk | 2.7.6.3 | Present |
| pyrophosphokinase | | | |
| Dihydropteroate synthase | folP | 2.5.1.15 | Present |
| Dihydropteroate synthase | folP1 | 2.5.1.15 | — |
| Aminodeoxychorismate/anthranilate | | | |
| synthase component 2 | pabA | 2.6.1.85 | Present |
| Aminodeoxychorismate synthase | pabB | 2.6.1.85 | Present |
| component 1 | | | |
| Alkaline phosphatase D | phoD | 3.1.3.1 | Present |
| Alkaline phosphatase 3 | phoB | 3.1.3.2 | Present |
| Alkaline phosphatase 4 | phoA | 3.1.3.3 | Present |
| Dihydrofolate reductase type 3 | dhfrIII | 1.5.1.3 | — |

Screening for prophages, insertion sequences and transposases—Strain Bs PTA-86 was scanned for presence of mobile genetic elements such as prophages, insertion sequences (IS) and transposases. BsPTA86 has 4 transposases and 2 copies of IS21 insertion sequence.

### DISCUSSION

A clear understanding of the physiology and safety of probiotic or live delivery strains as well as their interactions with target host, and hosts' gut microbiota are essential to rationally develop the next generation of probiotics or live delivery strains with improved safety and efficacy, and increased reproducibility. Here, we employed comprehensive multi-omics, biochemical, and microbiological approaches for the selection and characterization of Bacillus spp. strains to improve growth performance in poultry.

Bacillus spp. isolates were screened for their activities to inhibit certain pathogens and ability to secrete digestive enzymes in-vitro. The best candidates were further selected based on their safety profiles (i.e. antimicrobial resistance profile and cytotoxicity level). Genomic and metabolomic analyses were performed on the select isolates to further investigate potential host-benefit properties and possible health/safety concerns. This bottom-up approach ensures selection of the best candidates at each screening step. Strains that did not meet safety criteria were not selected. Only the best candidates that met phenotypic selection criteria moved forward to the next screening step. Genomic analysis of the top Bacillus strains helped to create a link between phenotypic observations with genomic traits.

Host-adapted Bacillus strains. We expected that host-adapted Bacillus strains to exert better probiotic effects in the host environment than those isolated from other sources, thus, we targeted our isolation to those Bacillus spp. from animal GIT content or fecal samples of healthy animals (8). A higher diversity of isolates was obtained from the ethanol-treated samples compared to heat-treated samples, as reported previously (8, 22). Despite the general heat resistance feature of Bacillus spores, spore core, cortex, coat, and membrane composition determines the degree of the spores' heat resistance (10, 69, 70) resulting in different responses of spores toward heat stresses.

Desirable probiotic properties. With the continuing reduction in use of antibiotics in poultry farms, as driven by regulations, and some customer preferences, the development of microbial feed additives that support maintenance of poultry health in the face of undesirable organisms would be beneficial. Our screening results showed that Bacillus spp. controlled the growth of undesirable E. coli O2, O18, and O78, C. perfringens—and Salmonella Typhimurium. APEC strains cause collibacillosis, which is a major problem in commercial production (74, 75). Collibacillosis occurs when APEC originating from fecal materials translocate into the lung epithelium during fecal aerosolization. Thus, reducing the APEC load in feces as a potential effect of Bacillus spp. in the feed could help reduce the incidence of collibacillosis (76, 77). C. perfringens is a pathogen that causes necrotic enteritis in poultry (78) by the production of alpha oxin and NetB (79, 80). Necrotic enteritis is a multi-factorial disease that cost poultry farmers 6 billion dollar annually (81). Salmonella Typhimurium, a poultry gut commensal, is the major cause of salmonellosis in humans. This infection is facilitated by the consumption of Salmonella-containing poultry products (82, 83). The ability of Bacillus spp to supress growth of these undesirable organisms might be due to the production of AMPs (bacteriocins). Genome analysis of BsPTA86 suggested that the genome encoded distinct AMPs (TABLE 14).

Bacillus species are known to secrete host beneficial enzymes such cellulase, xylanase, amylase, protease, β-mannanase, phytase (23, 51, 84). These enzymes, when fed to animals, improve digestion of low-calorie diets or reduce intestinal inflammation by breaking down non-starch polysaccharides (NSPs). Some NSPs are anti-nutritional factors, and increase the gut content viscosity, slow down feed retention time in the gut, and thus reduce nutrient absorption (85). An accumulation of undigested NSPs can lead to the growth of pathogens that cause subclinical infection challenges (86, 87). Production of pro-inflammatory cytokines as a response to NSPs demands a significant amount of energy, which otherwise could be preserved for growth, lowering food efficiency and growth performance (reviewed in (88)). Bs PTA-86 showed comparable protease, amylase, and β-mannanase activities. These activities were supported by our genomic analysis showing that Bs possesses genes encoding for amylase, protease, β-mannanase, and phytase.

It is noteworthy that genome analyses revealed other potential benefits the Bacillus candidate for animals. Genes encoding a wide array of antioxidant proteins were identified, superoxide dismutase, catalase, thioredoxin, and methionine sulfoxide, and bacillithiol. These proteins when expressed and secreted in the GIT could provide protection toward oxidative stress (89-91). Oxidative stress occurs in the GIT when the level of free radicals generated by reactive oxygen/nitrogen species (RO/NS) is much higher than the level of antioxidant proteins for neutralization of these toxic compounds (57). This event is triggered by various factors including nutritional or environmental heat stress, or pathological factors which ultimately decrease growth performance and quality of meat and eggs (57).

Among proposed functions of probiotic bacteria are the reduction of potential pathogenic bacteria, immune modulation, removal of harmful metabolites in the intestine and/or providing bioactive or otherwise regulatory metabolites. Folate-producing probiotic bacteria enable better nutrient digestion and energy recovery. Folate-producing probiotic strains could potentially confer protection against cancer, inflammation, stress, and digestive disturbances (66, 92-95). Several studies exploring the commercial utility of probiotic strains for folate production have been reported (92, 96, 97). Genes encoding essential enzymes in the biosynthetic pathways of folate were also found in the genome of three Bacillus strains. The products of these pathways supply important cofactors which once secreted would be absorbed by the host improving health status ((92, 96, 97).

Safety profiles. In addition, Bacillus DFM candidates must have acceptable safety profiles as expected by regulatory authorities. Some Bacillus spp. are known to produce AMPs and enterotoxins that might exert deleterious effects on the host cells (25). Cytotoxicity assessment of Bacillus spp. strains suggested that Bacillus spp. did not cause cytotoxicity of Vero cells. Moreover, genome analysis of Bs PTA 86 suggested that enterotoxins and other known virulence factors were absent in the subject Bacillus spp. Another important safety criterion is that Bacillus genomes must be devoid of transferable antimicrobial resistance genotypes (100). The data showed that the tested Bacillus isolates were sensitive to the antimicrobials tested and the apparent MIC values were below the recommended cut-off values. Genomic analysis of three Bacillus spp. identified putative genes for antimicrobial resistance to tetracycline, lincosamide, and strepthrothricine. In the genome of Bs PTA86, putative genes conferring resistance to rifampicin and macrolides were found. However, these genes have been reported present in Ba and Bs isolates from the environment (101, 102), suggesting these genes may be intrinsic properties of Ba and Bs strains. Furthermore, transferable mobile genetic elements such as transposons, insertion sequences were absent in the proximity of these genes indicating the very low risk of these genes being horizontally transferred to other gut microbes pose little to no risk to public health safety.

Metabolomic analysis. Probiotic strains are also known to secrete beneficial metabolites as microbial fermentation by-products such as short chain fatty acids (SCFAs) that help with mucus secretion, mucosal epithelial integrity, immune cell regulation, and serve as energy sources for colonocytes (103, 104). To investigate the potential host beneficial metabolites secreted, we performed global untargeted metabolomic analyses of Bs PTA86. A metabolite of particular interest was 1-kestose that was identified in the culture supernatants of the strains. 1-Kestose, the smallest fructooligosaccharide (FOS), is a trisaccharide molecule composed of a glucose and two fructose residues linked by glycosidic bonds. Kestose is a prebiotic that, when consumed, enriches the growth of gut commensals such as Bifidobacteria, Lactobacillus, and Faecalibacterium prausnitzii promoting gut health (105). Thioproline, an antioxidant molecule, was identified in the culture supernatant of Bs PTA86. Thioproline was reported to inhibit carcinogenesis in humans, and is expected to act as a nitrite scavenger (106). Pyridoxine (Vitamin B6) was found in the culture supernatant of Bs PTA86. Betaine and choline were possibly secreted by Bs PTA86. These molecules are methyl donors required for the biosynthesis of acetylcholine and phosphatidylcholine, for neural transmission and cell membrane integrity, respectively (107). Betaine, when supplemented in feed, has shown improved growth performance of birds during heat stresses (108, 109). Inclusion of choline has been associated with reduced FCR in broiler chickens (110).

### REFERENCES

1. FAO-UN (2018) The future of food and agriculture, Alternative pathways to 2050. in Supplementar Material (Food and Agriculture Organization of the United Nations).
2. US-FDA (2013) New animal drugs and new animal drug combination products administered in or on medicated feed or drinking water of food-producing animals: recommendations for drug sponsors for voluntarily aligning product use conditions with GFI #209. (Department of Health and Human Services).
3. EFSA Panel on Biological Hazards (BIOHAZ) et al. (2020) Update of the list of QPS-recommended biological agents intentionally added to food or feed as notified to EFSA 13: suitability of taxonomic units notified to EFSA until September 2020.
4. P. R. Moore, Evenson, A., Luckey, T. D., McCoy, E., Elvehjem, C. A. Hart, E. B., Use of sulfasuxidine, streptothricin, and streptomycin in nutritional studies with the chick. Journal of Biological Chemistry 2, 437-441 (1946).
5. C. Hill et al., The International Scientific Association for Probiotics and Prebiotics consensus statement on the scope and appropriate use of the term probiotic. Nature Reviews Gastroenterology & Hepatology 11, 506-514 (2014).
6. R. Jha, R. Das, S. Oak, P. Mishra, Probiotics (Direct-Fed Microbials) in Poultry Nutrition and Their Effects on Nutrient Utilization, Growth and Laying Performance, and Gut Health: A Systematic Review. Animals (Basel) 10 (2020).
7. A. Grant, C. G. Gay, H. S. Lillehoj, Bacillus spp. as direct-fed microbial antibiotic alternatives to enhance growth, immunity, and gut health in poultry. Avian Pathol 47, 339-351 (2018).
8. T. M. Barbosa, C. R. Serra, R. M. La Ragione, M. J. Woodward, A. O. Henriques, Screening for Bacillus isolates in the broiler gastrointestinal tract. Appl Environ Microbiol 71, 968-978 (2005).
9. X. Guo, D. Li, W. Lu, X. Piao, X. Chen, Screening of Bacillus strains as potential probiotics and subsequent confirmation of the in vivo effectiveness of Bacillus subtilis MA139 in pigs. Antonie Van Leeuwenhoek 90, 139-146 (2006).
10. P. Setlow, Spores of Bacillus subtilis: their resistance to and killing by radiation, heat and chemicals. Journal of Applied Microbiology 101, 514-525 (2006).
11. S. Shivaramaiah et al., Evaluation of Bacillus species as potential candidates for direct-fed microbials in commercial poultry. Poult Sci 90, 1574-1580 (2011).
12. M. Bernardeau, M. J. Lehtinen, S. D. Forssten, P. Nurminen, Importance of the gastrointestinal life cycle of Bacillus for probiotic functionality. J Food Sci Technol 54, 2570-2584 (2017).
13. J. D. Latorre et al., Evaluation of germination, distribution, and persistence of Bacillus subtilis spores through the gastrointestinal tract of chickens. Poult Sci 93, 1793-1800 (2014).
14. T. T. Hoa et al., Fate and dissemination of Bacillus subtilis spores in a murine model. Applied and environmental microbiology 67, 3819-3823 (2001).
15. G. Casula, S. M. Cutting, Bacillus probiotics: spore germination in the gastrointestinal tract.

Applied and environmental microbiology 68, 2344-2352 (2002).
16. S. Mingmongkolchai, W. Panbangred, Bacillus probiotics: an alternative to antibiotics for livestock production. J Appl Microbiol 124, 1334-1346 (2018). 17. A. Q. Grant, C. G. Gay, H. S. Lillehoj, Bacillus spp. as direct-fed microbial antibiotic alternatives to enhance growth, immunity, and gut health in poultry. Avian Pathology 47, 339-351 (2018).
18. Y. Ma et al., Supplemental Bacillus subtilis DSM 32315 manipulates intestinal structure and microbial composition in broiler chickens. Sci Rep 8, 15358 (2018).
19. J. S. Weese, H. Martin, Assessment of commercial probiotic bacterial contents and label accuracy. Can Vet J 52, 43-46 (2011).
20. J. S. Weese, Microbiologic evaluation of commercial probiotics. J Am Vet Med Assoc 220, 794-797 (2002).
21. D. H. Green et al., Characterization of two Bacillus probiotics. Appl Environ Microbiol 65, 4288-4291 (1999).
22. J. R. Koransky, S. D. Allen, V. R. Dowell, Use of ethanol for selective isolation of sporeforming microorganisms. Applied and Environmental Microbiology 35, 762-765 (1978).
23. J. D. Latorre et al., Evaluation and Selection of Bacillus Species Based on Enzyme Production, Antimicrobial Activity, and Biofilm Synthesis as Direct-Fed Microbial Candidates for Poultry. Front Vet Sci 3, 95 (2016).
24. B. V. McCleary, A simple assay procedure for β-d-mannanase. Carbohydrate Research 67, 213-221 (1978).
25. G. R. EFSA Panel on Additives and Products or Substances used in Animal Feed (FEEDAP), Gabriele Aquilina, Giovanna Azimonti, Vasileios Bampidis, Maria de Lourdes Bastos, Georges Bories, Andrew Chesson, Pier Sandro Cocconcelli, Gerhard Flachowsky, Jürgen Gropp, Boris Kolar, Maryline Kouba, Marta López-Alonso, Secundino López Puente, Alberto Mantovani, Baltasar Mayo, Fernando Ramos, Maria Saarela, Roberto Edoardo Villa, Robert John Wallace, Pieter Wester, Boet Glandorf, Lieve Herman, Sirpa KArenlampi, Jaime Aguilera, Montserrat Anguita, Rosella Brozzi, Jaume Galobart (2018) Guidance on the characterisation of microorganisms used as feed additives or as production organisms. (EFSA (European Food Safety Authority)).
26. G. L. S. f. a. H. World (2021) Performance Standards for Antimicrobial Susceptibility Testing. (CLSI), p 352.
27. J. Rao et al., Tn2008-driven carbapenem resistance in Acinetobacter baumannii isolates from a period of increased incidence of infections in a Southwest Virginia hospital (USA). J Glob Antimicrob Resist 12, 79-87 (2018).
28. D. Hyatt et al., Prodigal: prokaryotic gene recognition and translation initiation site identification. BMC Bioinformatics 11, 119 (2010).
29. D. Laslett, B. Canback, ARAGORN, a program to detect tRNA genes and tmRNA genes in nucleotide sequences. Nucleic Acids Res 32, 11-16 (2004).
30. T. Seemann, Prokka: rapid prokaryotic genome annotation. Bioinformatics 30, 2068-2069 (2014).
31. B. E. Suzek, M. D. Ermolaeva, M. Schreiber, S. L. Salzberg, A probabilistic method for identifying start codons in bacterial genomes. Bioinformatics 17, 1123-1130 (2001).
32. C. L. Kingsford, K. Ayanbule, S. L. Salzberg, Rapid, accurate, computational discovery of Rho-independent transcription terminators illuminates their relationship to DNA uptake. Genome Biol 8, R22 (2007).
33. E. P. Nawrocki, D. L. Kolbe, S. R. Eddy, Infernal 1.0: inference of RNA alignments. Bioinformatics 25, 1335-1337 (2009).
34. B. Tjaden, A computational system for identifying operons based on RNA-seq data. Methods. 176, 62-70 (2020).
35. A. Bremges, A. Fritz, A. C. McHardy, CAMITAX: Taxon labels for microbial genomes. GigaScience 9 (2020).
36. D. M. Emms, S. Kelly, OrthoFinder: phylogenetic orthology inference for comparative genomics. Genome Biol 20, 238 (2019).
37. E. W. Sayers et al., Database resources of the National Center for Biotechnology Information. Nucleic Acids Res 49, D10-d17 (2021).
38. S. I. Na et al., UBCG: Up-to-date bacterial core gene set and pipeline for phylogenomic tree reconstruction. J Microbiol 56, 280-285 (2018).
39. M. N. Price, P. S. Dehal, A. P. Arkin, FastTree 2—approximately maximum-likelihood trees for large alignments. PLoS One 5, e9490 (2010).
40. Z. Xie, H. Tang, ISEScan: automated identification of insertion sequence elements in prokaryotic genomes. Bioinformatics 33, 3340-3347 (2017).
41. S. Akhter, R. K. Aziz, R. A. Edwards, PhiSpy: a novel algorithm for finding prophages in bacterial genomes that combines similarity- and composition-based strategies. Nucleic Acids Res 40, e126 (2012).
42. W. N. P. Setlow, Molecular Biological Methods for Bacillus. C. H. S. Cutting, Ed. (John Wiley, New York, 1990).
43. M. Martin, Cutadapt removes adapter sequences from high-throughput sequencing reads. 2011 17, 3 (2011).
44. B. J. Callahan et al., DADA2: High-resolution sample inference from Illumina amplicon data. Nat Methods 13, 581-583 (2016).
45. P. Yilmaz et al., The SILVA and “All-species Living Tree Project (LTP)” taxonomic frameworks. Nucleic acids research 42, D643-D648 (2014).
46. E. H. Simpson, Measurement of Diversity. Nature 163, 688-688 (1949).
47. C. E. Shannon, The mathematical theory of communication. 1963. MD Comput 14, 306-317 (1997).
48. A. Chao, Nonparametric Estimation of the Number of Classes in a Population. Scandinavian Journal of Statistics 11, 265-270 (1984).
49. Anonymous (http://scikit-bio.org/.
50. The Association of American Feed Control Officials, Inc., 2021 Official Publication (FASS Inc., Champaign, IL).
51. I. Danilova, M. Sharipova, The Practical Potential of Bacilli and Their Enzymes for Industrial Production. Frontiers in Microbiology 11 (2020).
52. F. J. Contesini, R. R. d. Melo, H. H. Sato, An overview of Bacillus proteases: from production to application. Critical Reviews in Biotechnology 38, 321-334 (2018).
53. A. Fagerlund, T. Lindbdck, A. K. Storset, P. E. Granum, S. P. Hardy, Bacillus cereus Nhe is a pore-forming toxin with structural and functional properties similar to the ClyA (HlyE, SheA) family of haemolysins, able to induce osmotic lysis in epithelia. Microbiology (Reading) 154, 693-704 (2008).
54. M. Saeed et al., The Role of 8-Mannanase (Hemicell) in Improving Poultry Productivity, Health and Environment. Brazilian Journal of Poultry Science 21 (2019).
55. H. Fayek, M. Abou El-Ella, M. Attia, Y. Mady (1995) Evaluation of using some feed additives in layer diets. in Sci. Conf. Anim. Nutr, pp 313-321.
56. Y. Zhou, Z. Jiang, D. Lv, T. Wang, Improved energy-utilizing efficiency by enzyme preparation supplement in broiler diets with different metabolizable energy levels. Poult Sci 88, 316-322 (2009).
57. K. Sherif, Performance of broiler chicks fed plant protein diets supplemented with commercial enzymes. Journal of Animal and Poultry Production 34, 2819-2834 (2009).
58. K. Babasaki, T. Takao, Y. Shimonishi, K. Kurahashi, Subtilosin A, a new antibiotic peptide produced by Bacillus subtilis 168: isolation, structural analysis, and biogenesis. J Biochem 98, 585-603 (1985).
59. C. E. Shelburne et al., The spectrum of antimicrobial activity of the bacteriocin subtilosin A. Journal of Antimicrobial Chemotherapy 59, 297-300 (2007).
60. T. Stein, S. Diisterhus, A. Stroh, K. D. Entian, Subtilosin production by two Bacillus subtilis subspecies and variance of the sbo-alb cluster. Appl Environ Microbiol 70, 2349-2353 (2004).
61. E. F. S. A. (EFSA), EFSA statement on the requirements for whole genome sequence analysis of microorganisms intentionally used in the food chain. EFSA Journal, 1-15 (2021).
62. Y. Meyer, B. B. Buchanan, F. Vignols, J.-P. Reichheld, Thioredoxins and Glutaredoxins: Unifying Elements in Redox Biology. Annual Review of Genetics 43, 335-367 (2009).
63. A. Gaballa et al., Biosynthesis and functions of bacillithiol, a major low-molecular-weight thiol in Bacilli. Proc Natl Acad Sci USA 107, 6482-6486 (2010).
64. H. Antelmann, C. Scharf, M. Hecker, Phosphate starvation-inducible proteins of Bacillus subtilis: proteomics and transcriptional analysis. J Bacteriol 182, 4478-4490 (2000).
65. B. Sfnchez, P. Bressollier, M. C. Urdaci, Exported proteins in probiotic bacteria: adhesion to intestinal surfaces, host immunomodulation and molecular cross-talking with the host. FEMS Immunology & Medical Microbiology 54, 1-17 (2008).
66. T. Zhu et al., Engineering of Bacillus subtilis for enhanced total synthesis of folic acid. Appl Environ Microbiol 71, 7122-7129 (2005).
67. V. de Crecy-Lagard, Variations in metabolic pathways create challenges for automated metabolic reconstructions: Examples from the tetrahydrofolate synthesis pathway. Comput Struct Biotechnol J 10, 41-50 (2014).
68. A. R. Salem, J. R. Pattison, M. A. Foster, Folic acid and the methylation of homocysteine by Bacillus subtilis. Biochem J 126, 993-1004 (1972).
69. S. Brul et al., Challenges and advances in systems biology analysis of Bacillus spore physiology; molecular differences between an extreme heat resistant spore forming Bacillus subtilis food isolate and a laboratory strain. Food Microbiology 28, 221-227 (2011).
70. E. M. Berendsen, M. H. Zwietering, O. P. Kuipers, M. H. J. Wells-Bennik, Two distinct groups within the Bacillus subtilis group display significantly different spore heat resistance properties. Food Microbiology 45, 18-25 (2015).
71. F. Van Immerseel, J. I. Rood, R. J. Moore, R. W. Titball, Rethinking our understanding of the pathogenesis of necrotic enteritis in chickens. Trends Microbiol 17, 32-36 (2009).
72. K. Grave, M. C. Kaldhusdal, H. Kruse, L. M. Harr, K. Flatlandsmo, What has happened in norway after the ban of avoparcin? Consumption of antimicrobials by poultry. Prev Vet Med 62, 59-72 (2004).
73. L. Hughes, P. Hermans, K. Morgan, Risk factors for the use of prescription antibiotics on UK broiler farms. J Antimicrob Chemother 61, 947-952 (2008).
74. F. Dziva, M. P. Stevens, Colibacillosis in poultry: unravelling the molecular basis of virulence of avian pathogenic Escherichia coli in their natural hosts. Avian Pathol 37, 355-366 (2008).
75. M. Mellata, Human and avian extraintestinal pathogenic Escherichia coli: infections, zoonotic risks, and antibiotic resistance trends. Foodborne Pathog Dis 10, 916-932 (2013).
76. H. Duan et al., Transmission identification of Escherichia coli aerosol in chicken houses to their environments using ERIC-PCR. Sci China C Life Sci 51, 164-173 (2008).
77. Y. C. Chien, C. J. Chen, T. H. Lin, S. H. Chen, Y. C. Chien, Characteristics of microbial aerosols released from chicken and swine feces. J Air Waste Manag Assoc 61, 882-889 (2011).
78. K. K. Cooper, J. G. Songer, Necrotic enteritis in chickens: A paradigm of enteric infection by Clostridium perfringens type A. Anaerobe 15, 55-60 (2009).
79. A. L. Keyburn et al., NetB, a new toxin that is associated with avian necrotic enteritis caused by Clostridium perfringens. PLoS Pathog 4, e26 (2008).
80. A. L. Keyburn et al., Alpha-toxin of Clostridium perfringens is not an essential virulence factor in necrotic enteritis in chickens. Infect Immun 74, 6496-6500 (2006).
81. B. Wade, A. Keyburn, The true cost of necrotic enteritis. World Poult 31, 16-17 (2015).
82. M. B. Batz, S. Hoffmann, J. G. Morris, Jr., Ranking the disease burden of 14 pathogens in food sources in the United States using attribution data from outbreak investigations and expert elicitation. J Food Prot 75, 1278-1291 (2012).
83. S. L. Foley, A. M. Lynne, Food animal-associated Salmonella challenges: pathogenicity and antimicrobial resistance. J Anim Sci 86, E173-187 (2008). 84. S. Singh, B. K. Bajaj, Bioprocess optimization for production of thermoalkali-stable protease from Bacillus subtilis K-1 under solid-state fermentation. Preparative Biochemistry & Biotechnology 46, 717-724 (2016).
85. A. Raza, S. Bashir, R. Tabassum, An update on carbohydrases: growth performance and intestinal health of poultry. Heliyon 5, e01437-e01437 (2019).
86. M. Kaldhusdal, Necrotic enteritis as affected by dietary ingredients. World Poultry 16, 42-43 (2000).
87. F. Van Immerseel et al., Clostridium perfringens in poultry: an emerging threat for animal and public health. Avian Pathol 33, 537-549 (2004).
88. G. Cardoso Dal Pont, M. Farnell, Y. Farnell, M. H. Kogut, Dietary Factors as Triggers of Low-Grade Chronic Intestinal Inflammation in Poultry. Microorganisms 8, 139 (2020).
89. Y. Wang et al., Bacillus amyloliquefaciens SC06 alleviates the oxidative stress of IPEC-1 via modulating Nrf2/KeapI signaling pathway and decreasing ROS production. Appl Microbiol Biotechnol 101, 3015-3026 (2017).
90. A. Abudabos, A. Alyemni, H. Zakaria, Effect of Two Strains of Probiotics on the Antioxidant Capacity, Oxidative Stress, and Immune Responses of Salmonella-Challenged Broilers. Brazilian Journal of Poultry Science 18, 175-180 (2016).
91. T. Inatomi, K. Otomaru, Effect of dietary probiotics on the semen traits and antioxidative activity of male broiler breeders. Scientific Reports 8, 5874 (2018).
92. M. Rossi, A. Amaretti, S. Raimondi, Folate production by probiotic bacteria. Nutrients 3, 118-134 (2011).
93. J. Zhang et al., Screening of folate-producing lactic acid bacteria and modulatory effects of folate-biofortified yogurt on gut dysbacteriosis of folate-deficient rats. Food Funct 11, 6308-6318 (2020).
94. C. S. Fuchs et al., The Influence of Folate and Multivitamin Use on the Familial Risk of Colon Cancer in Women. Cancer Epidemiology Biomarkers &amp; Prevention 11, 227-234 (2002).
95. E. White, J. S. Shannon, R. E. Patterson, Relationship between vitamin and calcium supplement use and colon cancer. Cancer Epidemiol Biomarkers Prev 6, 769-774 (1997).
96. M. Schallmey, A. Singh, O. P. Ward, Developments in the use of Bacillus species for industrial production. Can J Microbiol 50, 1-17 (2004).
97. U. Sauer, D. C. Cameron, J. E. Bailey, Metabolic capacity of Bacillus subtilis for the production of purine nucleosides, riboflavin, and folic acid. Biotechnology and Bioengineering 59, 227-238 (1998).
98. F. M. Asrar, D. L. O'Connor, Bacterially synthesized folate and supplemental folic acid are absorbed across the large intestine of piglets. J Nutr Biochem 16, 587-593 (2005).
99. L. H. Duc, H. A. Hong, T. M. Barbosa, A. O. Henriques, S. M. Cutting, Characterization of Bacillus probiotics available for human use. Applied and environmental microbiology 70, 2161-2171 (2004).
100. V. Ciorba, A. Odone, L. Veronesi, C. Pasquarella, C. Signorelli, Antibiotic resistance as a major public health concern: epidemiology and economic impact. Ann Ig 27, 562-579 (2015).
101. D. B. Adimpong et al., Antimicrobial susceptibility of Bacillus strains isolated from primary starters for African traditional bread production and characterization of the bacitracin operon and bacitracin biosynthesis. Appl Environ Microbiol 78, 7903-7914 (2012).
102. R. W. Phelan et al., Tetracycline resistance-encoding plasmid from Bacillus sp. strain #24, isolated from the marine sponge Haliclona simulans. Appl Environ Microbiol 77, 327-329 (2011).
103. L. R. Lopetuso, F. Scaldaferri, V. Petito, A. Gasbarrini, Commensal Clostridia: leading players in the maintenance of gut homeostasis. Gut pathogens 5, 1-8 (2013).
104. H. M. Hamer et al., Review article: the role of butyrate on colonic function. Aliment Pharmacol Ther 27, 104-119 (2008).
105. J. A. Patterson, J. I. Orban, A. L. Sutton, G. N. Richards, Selective enrichment of Bifidobacteria in the intestinal tract of broilers by thermally produced kestoses and effect on broiler performance. Poult Sci 76, 497-500 (1997).
106. J. A. Patterson et al., Thioproline formation as a driver of formaldehyde toxicity in Escherichia coli. BiochemJ 477, 1745-1757 (2020).
107. R. R. Zempleni J, McCormick D B, Suttie J W, Handbook of vitamins. (CRC Press, Boca Raton (FL), ed. 4th, 2007).
108. W. Liu et al., Effects of Dietary Betaine on Growth Performance, Digestive Function, Carcass Traits, and Meat Quality in Indigenous Yellow-Feathered Broilers under Long-Term Heat Stress. Animals (Basel) 9 (2019).
109. S. He, S. Zhao, S. Dai, D. Liu, S. G. Bokhari, Effects of dietary betaine on growth performance, fat deposition and serum lipids in broilers subjected to chronic heat stress. Anim Sci J 86, 897-903 (2015).
110. G. S. Santiago et al., Dietary choline affects field performance and broiler leg deviations. Livestock Science 240, 104127 (2020).
111. Y. Shang, S. Kumar, B. Oakley, W. K. Kim, Chicken Gut Microbiota: Importance and Detection Technology. Front Vet Sci 5, 254 (2018).
112. A.-R. Al-Fataftah, A. Abdelgader, Effects of dietary Bacillus subtilis on heat-stressed broilers performance, intestinal morphology and microflora composition. Animal Feed Science and Technology 198, 279-285 (2014).
113. J. D. Latorre et al., Evaluation and Selection of Bacillus Species Based on Enzyme Production, Antimicrobial Activity, and Biofilm Synthesis as Direct-Fed Microbial Candidates for Poultry. Front Vet Sci 3, 95 (2016).
114. B. V. McCleary, A simple assay procedure for β-d-mannanase. Carbohydrate Research 67, 213-221 (1978).
115. G. R. EFSA Panel on Additives and Products or Substances used in Animal Feed (FEEDAP), Gabriele Aquilina, Giovanna Azimonti, Vasileios Bampidis, Maria de Lourdes Bastos, Georges Bories, Andrew Chesson, Pier Sandro Cocconcelli, Gerhard Flachowsky, Jürgen Gropp, Boris Kolar, Maryline Kouba, Marta López-Alonso, Secundino López Puente, Alberto Mantovani, Baltasar Mayo, Fernando Ramos, Maria Saarela, Roberto Edoardo Villa, Robert John Wallace, Pieter Wester, Boet Glandorf, Lieve Herman, Sirpa KArenlampi, Jaime Aguilera, Montserrat Anguita, Rosella Brozzi, Jaume Galobart (2018) Guidance on the characterisation of microorganisms used as feed additives or as production organisms. (EFSA (European Food Safety Authority)).

### Example 5

Bacillus strain 105 (BSUB105; PTA-126786 or PTA-86) was analysed and certain classes of genes or secondary metabolite pathways unique to the strain identified. Some results are provided in the earlier examples and tables, such as bacteriocin predictions, secondary metabolites, carbohydrate metabolizing ezymes. Unique proteins (predicted proteins for which an equivalent or homologous protein encoding gene is not identified by identity searches in other compared Bacillus strains) are predicted based on strain sequence comparisons and assessment of gene protein sequences for Bacillus subtilis 105 (BSUB105; PTA-126786 or PTA-86). Strain 105 includes 4 subtilosin genes, pullulanase (which helps break down branched chain carbohydrates to simple carbohydrates), cyclodextrin-binding protein, 9 sporulation related genes, beta-galactosidase YesZ and GanA genes, oxidoreductase YjmC. Unique genes encoded based on the genome sequence of strain Bs PTA 86 are included and provided in U.S. Ser. No. 63/083,697 filed Sep. 25, 2020 and in U.S. Ser. No. 63/241,369 filed Sep. 7, 2021, each of which are incorporated by reference herein.

### Example 6

### Application of B. Subtilis Strain 105 as a Live Delivery Platform and Production System

Bacillus subtilis strain 105, also denoted ELA19105 (PTA-126786) has been selected and implemented as a useful and applicable strain for development and use in food-grade and pharmaceutical protein production.

A comparison between Bacillus subtilis strain 105, also denoted ELA19105 (PTA-126786) and B. subtilis strain 168 was conducted. Genome analysis and comparison showed that the S. sub 168 genome includes 1109 genes, relative to 1681 reactions, 1376 metabolites, 243 exchanges and 2 compartments. The B. subtilis strain 105 (ELA191105) genome includes 1077 genes, relative to 1462 reactions, 1253 metabolites, 153 exchanges and 2 compartments.

Metabolic genes unique to strain 105 versus strain 168, particularly their encoded proteins, are indicated below in TABLE 19.

TABLE 19
metabolic genes unique to strain B. subtilis 105

| ID Number | | |
|---|---|---|
| Internal | PROTEIN | EC NUMBER |
| JS609_00401 | 4′′′-phosphopantetheinyl transferase Sfp | EC:2.7.8.7 |
| JS609_00409 | Protein BsdD | EC:4.1.4.61 |
| JS609_00458 | 5-oxoprolinase subunit | EC:3.5.2.9 |
| JS609_00545 | Fatty acid desaturase | EC:1.14.19.- |
| JS609_00559 | Ornithine lipid N-methyltransferase | EC:2.1.1.344 |
| JS609_00561 | Sensor histidine kinase RscC | EC:2.7.13.3 |
| JS609_00883 | Phosphatidylglycerol lysyltransferase | EC:2.3.2.3 |
| JS609_01398 | Putative phosphoserine phosphatase | EC:3.1.3.3 |
| JS609_02073 | Putative N-acetyl-alpha-D-glucosaminyl | EC:3.5.1.- |
| | L-malate deacetylase | |
| JS609_02535 | Putative quinone oxidoreductase YhfP | EC:1.6.5.- |
| JS609_02536 | L-threonine 3-dehydrogenase | EC:1.1.1.103 |
| JS609_02539 | N-ethylmaleimide reductase | EC:1.3.1.- |
| JS609_02540 | Putative non-heme bromoperoxidase | EC:1.11.1.18 |
| | BpoC | |
| JS609_02543 | 3-oxoacyl-[acyl-carrier-protein] synthase | EC:2.3.1.179 |
| JS609_02569 | Mannan endo-1,4-beta-mannosidase | EC:3.2.1.78 |
| JS609_02702 | Holliday junction ATP_dependent DNA | EC:3.6.4.12 |
| | helicase RuvB | |
| JS609_03194 | Sensor histidine kinase ComP | EC:2.7.13.3 |
| JS609_03320 | Oligoendopeptidase F, plasmid | EC:3.4.24.- |
| JS609_03553 | N(G), N(G)-dimethylarginine | EC:3.5.3.18 |
| | dimethylaminohydrolase | |
| JS609_03556 | ADP-ribose pyrophophatase | EC:3.6.1.13 |
| JS609_03557 | Pyrimidine 5′-nucleotidase YjjG | EC:3.1.3.5 |
| JS609_03563 | Vitamin B12 import ATP-binding protein | EC:7.6.2.8 |
| | BtuD | |
| JS609_03610 | Teichoic acid poly(glycerol phosphate) | EC:2.7.8.12 |
| | polymerase | |
| JS609_03830 | dTDP-4-dehydrorhamnose 3,5-epimerase | EC:5.1.3.13 |
| JS609_03973 | Dihydroanticapsin 7-dehydrogenase | EC:1.1.1.385 |
| JS609_03980 | Ribonuclease YxiD | EC:3.1.-.- |
| JS609_04080 | Sensor histidine kinase RscC | EC:2.7.13.3 |

The Bacillus subtilis strain 105, also denoted ELA19105 (PTA-126786) provides a useful and applicable strain for development and use in food-grade and pharmaceutical protein production and as a live delivery platform to deliver and produce useful biomolecules and proteins, including homologous and heterologous proteins in an animal host.

TABLE 20
Developing and Applying Bacillus subtilis #105 as a production and live delivery strain

| | Requirement | Status | Remarks |
|---|---|---|---|
| SAFETY | Antimicrobial | Susceptible to all EFSA | The genome is free of antimicrobial |
| | resistance | recommended, clinically | resistance genes |
| | | relevant antimicrobials | |
| | Cytotoxicity | No cytotoxicity was detected | |
| | | using Vero cells | |
| | Virulence | No virulence factors and | This consistent with the cytotoxicity |
| | factors & | toxins were identified | data |
| | toxins | | |
| | Biogenic | No biogenic amines were | Bacillus species generally do not |
| | amines | identified. | produce biogenic amines. |
| GROWTH | Growth- | Grows well under laboratory | Minimal media available for Bacillus |
| & SCALE- | laboratory | conditions; grows in M9 | subtilis #168 and works for B. subtilis |
| UP | scale | media; does not | #105 growth as well. |
| | | demomnstrate complex | M9 media which is a minimal media is |
| | | nutritional requirements | suitable. For growth in minimal media, |
| | | | medium containing 1X M9 salts, and |
| | | | glucose at a final concentration of 0.5% |
| | | | (w/v) was used. |
| | | | Rich medium for growth contains the |
| | | | following entities (g/L): peptone 30; |
| | | | sucrose 30; yeast extract 8; KH₂PO₄ 4; |
| | | | MgSO₄ 1; and MnSO₄ 0.025. |
| | | | 1X M9 salts (per liter): |
| | | | 7 g Na₂HPO₄•7H₂O |
| | | | 3 g KH₂PO₄ |
| | | | 0.5 g NaCl |
| | | | 1 g NH₄Cl |
| | Growth- | The strain grows well in | |
| | fermentation | fermenters | |
| | Manufacturing | The strain is being | No issues with scale up for |
| | scale | manufactured in large | manufacturing have been identified in |
| | | amounts for various studies | evaluation runs |
| | | including in vivo and animal | |
| | | studies | |
| | Naturally | Higher transformation and | Strain 105 is competent. |
| | competent | relative ease of genetic | Transformation using electroporation |
| | | manipulation to increase | has been confimed. |
| | | competency. | Strain 105 naturally produces ComK. |
| | | | Competency increased by genetic |
| | | | modification to overexpress ComK. |
| | | | Competency increased by |
| | | | overexpression of ComK under an |
| | | | inducible promoter. |
| | | | SEE EXAMPLE 7 |
| | Protease | Potentially produces | Strain 105 is genetically modified to |
| | production | proteases which can reduce | delete or inactivate genes encoding one |
| | | amount of one or more | or more proteases. |
| | | secreted proteins of interest. | SEE EXAMPLE 11 |
| | Plasmids | Strain 105 is free of any | The strain has been screened for |
| | | plasmids | potential mobile genetic elements |
| | | | (MGEs) and risk analysis of MGEs in |
| | | | the context of AMRs, toxins or |
| | | | virulence factors has been completed |
| | | | with no immediate risks identified. |
| | Prophages | No prophages were identified | |
| | | in the genome | |
| | Native | Strain 105 does not contain | |
| | CRISPR-Cas9 | any native Cas9 or CRISPR | |
| | | elements | |
| | Single | Similar to other bacterial | Kanamycin, chloramphenicol and |
| | crossovers, | species, B. subtilis undergoes | spectinomycin are commonly used |
| | double | single croosovers, which then | antibiotic markers for selection of |
| | crossovers, | resolve into double | initial site integration. Antibiotic |
| | markers for | crossovers | marker is used to select for single |
| | selection | | crossovers, then a non-antibiotic |
| | | | marker (such as manAP, upp, PheS etc) |
| | | | flanking the integration cassette is then |
| | | | used to select for double crossovers. |
| | Spores | Strain 105 is naturally spore | Spore forms can be used for |
| | | forming. | administration such as in a feed |
| | | | additive or other food type product, |
| | | | including for oral ingestion. |
| | | | Strain 105 can be genetically modified |
| | | | to delete or inactivate spore-related |
| | | | genes to generate a non-spore forming |
| | | | strain. Non-spore forming 105 mutant |
| | | | strain may increase production of one |
| | | | or more biomolecule or protein product |
| | | | of interest. |
| | | | SEE EXAMPLE 9 |
| | Genome | Predict metabolic pathways | Researchers have reduced the B. |
| | reduction & | based on the sequence data, | subtilis genome from 4 MB to 2.7 MB |
| | metabolic | metabolomics data and using | with enhanced productivity and with no |
| | pathway | B. subtilis #168 as a | issues (Reuß DR et al (2017) Genome |
| | prediction | reference strain. Identify | Res 27:289-299) |
| | | non-essential pathways/genes | ALSO SEE EXAMPLE 13 for lytic |
| | | and delete to reduce genome | enzyme and antibacterial peptide |
| | | size and improve genome | deletions |
| | | insertion and integration size | |
| | | capacity and | |
| | | production/expression | |
| | | efficiency. | |

### Example 7

### Modification of Strain 105 to Increase Competence

Competence is a physiological state that enables celis, including bacterial cells, to take up and internalize extracellular DNA. In practice, only a smali subpopulation of bacterial cells, such as B. subtilis celis, becomes competent when they enter stationary phase. Specifically, B. subtilis becomes competent when the competence transcription factor, ComK, reaches a certain threshold level (Maamar and Dubnau, 2005; Smits et al., 2005). ComK is the competence master regulator which activates about 100 genes for DNA-recombination, -repair, -binding, -uptake (Berka et al., 2002; Hamoen et al., 2002), celi division (Hamoen, 2011), as well as its own promoter in a positive feedback loop (van Sinderen and Venema, 1994). When B. subtilis cells enter stationary phase due to nutrient deprivation and high celi density, they start to differentiate into various subpopulations. Some of them become motile (Nishihara and Freese, 1975), while the others form biofilm (Vlamakis et al., 2008), secrete degradative enzymes and antibiotics (GonzAlez-Pastor et al., 2003), or finally sporulate (Rudner and Losick, 2001; Piggot and Hilbert, 2004). Another small subpopulation differentiates into competent cells able to take up extracellular DNA (Dubnau, 1991a; Dubnau and Provvedi, 2000).

It would be beneficial to have improved transformation efficiency of B. subtilis, including in rich media. Construction of a super-competent Bacillus strain has been reported using a genetically modified comKS encoding gene cassette, particularly under the control of a mannitol-inducible PmtlA promoter (Rahmer R et al (2015) Front Microbiol 6:1431; doi: 10.3389/fmicb.2015.01431). This cassette resulted in overexpression of both comK and comS and increased the transformation efficiency of B. subtilis with plasmid DNA by over 6-fold compared to wild type B. subtilis without the cassette.

B. subtilis strain 105 is genetically modified to increase competency by generating a modified 105 strain overexpressing comK and comS. In one approach, an expression cassette comprising the PxylA promoter from B. subtilis strain 105 linked to comK encoding sequence and followed by in frame comS encoding sequence. ComK and comS are produced under the control of the PxylA promoter. The PxlA promoter is a xylose inducible promoter.

The following provides an exemplary expression cassette including a promoter (PxlA promoter from B. subtilis strain 105, ComK encoding sequence, and ComS encoding sequence (SEQ ID NO: 37)

AGCGATATCCACTTCATCCACTCCATTTGTTTAATCTTTAAATTAAGTAT
AAACATAGTACATAGCGAATCTTCCCTTTATTATATCTAATGTGTTCATA
AAAAACTAAAAAAAATATTGAAAATACTGATGAGGTTATATAAGATGAAA
GTAAGTTAGTTTGTTTAAACAACAAACTAATAGGTAACTTACAATATGAA
ATAAAATGCATTTGTATTTGAATGATCAGGTTTTGAATTTATTTTTAAGG
GGGAAATCACATGAGTCAGAAAACAGACGCACCTTTAGAATCGTATGAAG
TGAACGGCGCAACAATTGCCGTGCTGCCAGAAGAAATAGACGGCAAAATC
TGTTCCAGAATTATTGAAAAAGATTGCGTGTTTTATGTAAACATGAAGCC
GCTGCAAATTGTCGACAGAAGCTGCCGATTTTTTGGATCAAGCTATGCGG
GAAGAAAAGCAGGAACTTATGAAGTGACAAAAATTTCACACAAGCCGCCG
ATCATGGTGGACCCTTCGAACCAAATCTTTTTATTCCCTACACTTTCTTC
GACAAGACCCCAATGCGGCTGGATTTCCCATGTGCATGTAAAAGAATTCA
AAGCGACTGAATTCGACGATACGGAAGTGACGTTTTCGAATGGGAAAACG
ATGGAGCTGCCGATCTCTTATAATTCGTTCGAGAACCAGGTATATCGAAC
AGCGTGGCTCAGAACCAAATTCCAAGACAGAATCGACCACCGCGTGCCGA
AAAGGCAGGAATTTATGCTGTACCCGAAGGAAGAGCGGACGAAGATGATT
TATGATTTTATTTTGCGTGAGCTCGGGGAACGGTATTAGAATTTATTTTT

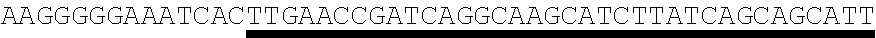

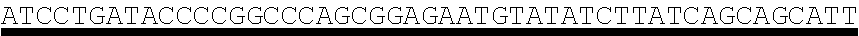

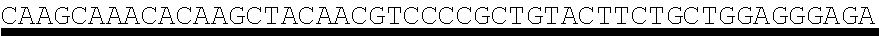

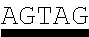

BOLD: PxylA promoter from B. subtilis #105
DOUBLE UNDERLINED: ComK encoding sequence
BOLD UNDERLINED: ComS encoding sequence

ComK encoding sequence of strain 105 is provided below (SEQ ID NO: 38):

ATGAGTCAGAAAACAGACGCACCTTTAGAATCGTATGAAGTGAACGGCG
CAACAATTGCCGTGCTGCCAGAAGAAATAGACGGCAAAATCTGTTCCAG
AATTATTGAAAAAGATTGCGTGTTTTATGTAAACATGAAGCCGCTGCAA
ATTGTCGACAGAAGCTGCCGATTTTTTGGATCAAGCTATGCGGGAAGAA
AAGCAGGAACTTATGAAGTGACAAAAATTTCACACAAGCCGCCGATCAT
GGTGGACCCTTCGAACCAAATCTTTTTATTCCCTACACTTTCTTCGACA
AGACCCCAATGCGGCTGGATTTCCCATGTGCATGTAAAAGAATTCAAAG
CGACTGAATTCGACGATACGGAAGTGACGTTTTCGAATGGGAAAACGAT
GGAGCTGCCGATCTCTTATAATTCGTTCGAGAACCAGGTATATCGAACA
GCGTGGCTCAGAACCAAATTCCAAGACAGAATCGACCACCGCGTGCCGA
AAAGGCAGGAATTTATGCTGTACCCGAAGGAAGAGCGGACGAAGATGAT
TTATGATTTTATTTTGCGTGAGCTCGGGGAACGGTATTAG

ComS encoding sequence of strain 105 is provided below (SEQ ID NO: 39): PGP-45 DNA

TTGAACCGATCAGGCAAGCATCTTATCAGCAGCATTATCCTGTATCCCC
GGCCCAGCGGAGAATGTATATCCTCAATCAGCTTGGACAAGCAAACACA
AGCTACAACGTCCCCGCTGTACTTCTGCTGGAGGGAGAAGTAG

Sequence of PxylA_Bs105, a xylose inducible promoter from strain 105 is provided below (SEQ ID NO: 40):

AGCGATATCCACTTCATCCACTCCATTTGTTTAATCTTTAAATTAAGTA
TAAACATAGTACATAGCGAATCTTCCCTTTATTATATCTAATGTGTTCA
TAAAAAACTAAAAAAAATATTGAAAATACTGATGAGGTTATATAAGATG
AAAGTAAGTTAGTTTGTTTAAACAACAAACTAATAGGTAACTTACAATA
TGAAATAAAATGCATTTGTATTTGAATGATCAGGTTTTGAATTTATTTT
TAAGGGGGAAATCAC

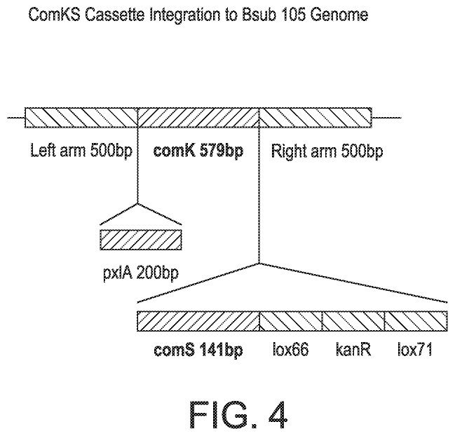

In order to enhance the transformation efficiency of B. subtilis #105, the CDS of native ComK gene was deleted and replaced with ComKS under a xylose inducible promoter (xylA). A diagram of the intergration and replacement strategy is provided in FIG. 4. PCR and sequencing confirmed that the ComKS expression cassette was integrated correctly in the genome. Junction PCT first confirmed the correct integration of the ComKS expression cassette and then colony PCR showed the entire ComKS insert in the genome (data not shown).

Evaluation of the ComKS system demonstrated that it resulted in improvement in competency. Engineering of this inducible ComKS system improved the transformation efficiency by approximately 100-fold (from 2-3 colonies to 200-400 colonies/500 ng of DNA).

Other native or non-native promoters may be utilized in a comKcomS inducible expression cassette. In some embodiments the native or non-native promoter is inducible and permits controlled and timed competency, including under specific growth conditiond, with particular media additions, or under distinct or specified bacterial cell growth phases (such as growth phase vs stationary phase etc). Other exemplary indicible promoters include a strain 105 mannitol inducible promoter. The sequence of the mannose inducible promoter from B. subtilis strain 105 is as follows (SEQ ID NO: 41):

AAGCAGGGATTATTCCTTGCTTTTTTTTGTTATAGGGAAAAATGCCTTT
ATTACCGGAACCTATGGTAAAAAAAAGCGATTTTAATGAGCTGATTTCG
GTATACAGTTGAGACAAGATCTTATAATTCACACTTTAAAGGAGGGTTC
CT

### Example 8

### Signal Peptides for Secretory Production of Heterologous Proteins in Bacillus Subtilis

In instances where one or more relevant target biomolecule or protein is a secreted protein or must be secreted by the bacterial delivery cells to be either active or to reach the relevant location in the host system or organ(s) or tissues, a secretion signal or signal sequence can be incorporated to promote secretion of the molecule(s) or protein(s). In B. subtilis, the export of protein is generally accomplished by the Sec-type secretion pathway, which governs over 90 percent of the secretory proteins found in B. subtilis.

The N-terminal sequence of a secreted protein carries a specific secretion signal known as signal peptide. After the nascent peptide with the signal peptide is synthesized, it can be recognized and translocated by the components of the Sec-type secretory pathway through the membrane into the extracellular medium. The signal peptide can be a key factor determining the best pathway for the target protein and how it is secreted across the membrane.

Exemplary and suitable secretion signal peptides of strain 105 were identified through analysis of the global proteomics data. Several secretion signals are provided below. These can be fused to existing or encoding protein sequences, including in combination with a high expression or inducible promoter. These signal sequences can be utilized in expression cassettes and/or integrated with the encoding nucleic acid sequence to provide effective and efficient secretion of a biomolecule or heterologous protein. The signal sequence is fused in frame to coding sequence.

Bacillus subtilis strain 105 beta mannanase secretion signal is as follows (SEQ ID NO: 42):

TTGTTTAAGAAACATACGATCTCTTTGCTCATTATATTTTTACTTGCGT
CTGCTGTTTTAGCAAAACCAATTGAAGCGCATACTGTGTCGCCT

This sequence encodes a secretion signal peptide of amino acid sequence of:

(SEQ ID NO: 43)
LFKKHTISLLIIFLLASAVLAKPIEAHTVSP
(SEQ ID NO: 44)
LFKKHTISLLIIFLLASAVLAKPIEA

Bacillus subtilis strain 105 pel secretion signal is as follows (SEQ ID NO: 45):

ATGAAAAAAGTGATGTTAGCTACGGCTCTGTTTTTAGGATTGACTCCAG
CTGGCGCGAACGCAGCTGATTTAGGCCAC

This sequence encodes a secretion signal peptide of amino acid sequence of:

(SEQ ID NO: 46)
MKKVMLATALFLGLTPAGANAADLGH
(SEQ ID NO: 47)
MKKVMLATALFLGLTPAGANA

Bacillus subtilis strain 105 dacC secretion signal is as follows (SEQ ID NO: 48):

ATGAAAAAAAGCATAAAGCTTTATGTTGCTGTTTTACTGCTTTTCGTCG
TTGCTTCGGTGCCTTATATGCATCAGGCTGCGCTTGCC

This sequence encodes a secretion signal peptide of amino acid sequence of (SEQ ID NO: 49):

MKKSIKLYVAVLLLFVVASVPYMHQAALA

Alternative secretion signal sequences are known and available in the art. For example, Fu et al evaluated the extracellular production of alpha-amylase (AmyS) from GeoBacillus stearothermophilus by generating and screening a high-capacity signal peptide library in Bacillus subtilis (Fu, G et al (2018) J Agric Food Chem 66:13141-13151). A total of 173 Sec-type signal peptides from B. subtilis were fused to the target protein by a sequence-independent, PCR-based cloning method without using a restriction endonuclease or ligase (You, C.; Zhang, X. Z.; Zhang, Y. H. (2012) Appl Environ Microbiol 78 (5): 1593-5). The resulting multimeric plasmid library DNA harboring different signal peptides was transformed into B. subtilis and screened for constructs with high extracellular alpha-amylase activity utilizing a starch-iodine based high-throughput method. Signal peptides optimized for the secretory expression of AmyS were identified and validated by high-density fermentation. Numerous signal peptides were identified as candidates for improving the secretory expression of the alpha-amylase AmyS in B. subtilis. These signal peptides are listed below in TABLE 21 and may be utilized in strain 105. In fact, the above strain 105 pel secretion signal amino acid sequence corresponds in sequence to the pel signal sequence in the below table.

TABLE 21
Signal Peptides for Secretion of Protein as assessed based on AmyS Amylase Secretion

| name | length (aa) | signal peptide sequence |
|---|---|---|
| yvcE | 30 | MRKSLITLGLASVIGTSSFLIPFTSKTASA (SEQ ID NO: 50) |
| yoqM | 25 | MKLRKVLTGSVLSLGLLVSASPAFA (SEQ ID NO: 51) |
| yuaB | 30 | MKRKLLSSLAISALSLGLLVSAPTASFAAE (SEQ ID NO: 52) |
| pel | 21 | MKKVMLATALFLGLTPAGANA (SEQ ID NO: 53) |
| pelB | 24 | MKRLCLWFTVFSLFLVLLPGKALG (SEQ ID NO: 54) |
| yoaW | 24 | MKKMLMLAFTFLLALTIHVGEASA (SEQ ID NO: 55) |
| yqxI | 28 | MFKKLLLATSALTFSLSLVLPLDGHAKA (SEQ ID NO: 56) |
| lipA | 32 | MKFVKRRIIALVTILMLSVTSLFALQPSAKAA (SEQ ID NO: 57) |
| lipB | 28 | MKKVLMAFIICLSLILSVLAAPPSGAKA (SEQ ID NO: 58) |
| yoqH | 23 | MKRFILVLSFLSIIVAYPIQTNA (SEQ ID NO: 59) |
| ybfO 2 | 8 | MKRMIVRMTLPLLIVCLAFSSFSASARA (SEQ ID NO: 60) |
| sacB | 29 | MNIKKFAKQTVLTFTTALLAGGATQAFA (SEQ ID NO: 61) |
| bglS | 28 | MPYLKRVLLLLVTGLFMSLFAVTATASA (SEQ ID NO: 62) |
| yddT | 28 | MRKKRVITCVMAASLTLGSLLPAGYASA (SEQ ID NO: 63) |
| yobB | 25 | MKIRKILLSSALSFGMLISAVPALA (SEQ ID NO: 64) |

A person of skill in the art would recognize that, because of the redundancy of the genetic code, multiple nucleic acid sequences could encode the above peptides.

### Example 9

### Modification of Strain 105 to Generate Non-Spore Forming Strain

Bacillus subtilis is a gram-positive endospore-forming microorganism and holds a qualified presumption of safety (QPS) status from the European Food Safety Authority (Hohmann H P et al (2016) Industrial Biotechnology: Microorganisms pp 221-297) based on its non-pathogenicity and lack of exotoxins and endotoxins production. However, the B. subtilis strain frequently sporulates in response to physical and chemical stimuli, thus halting growth and causing nutrient wastage and reduced yield. Sporulation occurs naturally in B. subtilis culture and helps the bacterium to resist physical and chemical stimuli, supporting its terrestrial life. Spore is the dormant state, during which the synthesis and secretion of enzymes or chemical products cease. The spore is better equipped to resist extreme environments than a vegetative cell and can germinate, resuming vegetative growth in response to appropriate nutrients. Several reports have described varied approaches to prevent B. subtilis sporulation during fermentation for different target products. For example, deletion of the initial regulatory sporulation gene spoOA results in enhanced maintenance metabolism (Tannler S et al (2008) Microb Cell Factories 7:9-19), increased glucose consumption and acetate formation rates (Fischer and Sauer, 2005 Nat. Genet. 37, 636-640), and abolished polymyxin production in B. subtilis BSK4-OA (Park et al. (2012) Appl. Environ. Microbiol. 78, 4194-4199).

Wang et al engineered several non-sporulating B. subtilis strains by knocking out single sporulation-related genes involved in various stages of sporulation (spoOA, spoIIIE, and spoIVB) (Wang M et al (2020) Metabolic Engineering 62:235-248). The SpoOA-null non-spore forming mutant was especially efficient in producing secondary metabolites, such as surfactin.

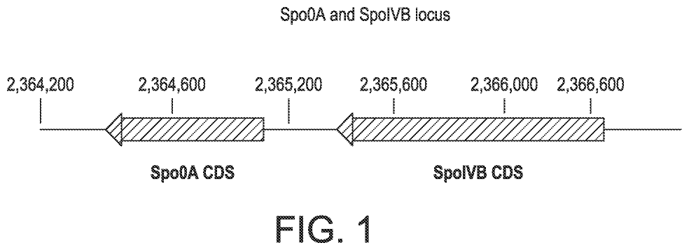

Bacillus subtilis strain 105 (ELA191105) is modified and engineered to delete or otherwise inactivate SpoOA and/or SpoIVB encoding sequence. FIG. 1 depicts the SpoOA and SpoIVB locus wherein SpoIVb and SpoA are encoded by tandem located sequence. Both genes can be deleted by single deletion of the encoding region using overlapping sequences at the ends of or outside of the SpoIVb and SpoOA encoding sequence. The SpoOA and SpoIVB sequence for deletion to generate non-spore forming and modified B. subtilis strain 105 is as follows:

>Spo0A and SpoIVB sequence for deletion to generate non-spore forming B. subtilis
#105
(SEQ ID NO: 65)
TTAAGAAGCCTTATGTTCTAATCTCAGCTTATCCGCAACCATTGCAATGAATTCCGAATTTGTCGGTTTAGCTTTTGT
CATGCTGACAGTATAACCGAACAACGAGGAAATGGAATCAATGTTTCCTCTGCTCCATGCCACTTCAATTGCATGG
CGGATCGCTCTTTCTACACGGCTTGCGGTTGTGTTGAATTTTTTGGCGATGTCCGGATAGAGGACTTTTGTAATGCT
GCCGAGCAATTCGATGTCATTGTATACCATTGAGATTGCTTCGCGCAGATAGAGATAGCCTTTAATATGGGCTGGG
ACGCCGATTTCATGGATAATGCTTGTGATGCTCGCGTCGAGATTTTTCTTCTTTGGTTCAGGCTGGCTGCTGCGTAT
AATACTGCTTTGCGATGATGGCGCACGATGCGTCACACTGCTGGCATTTCCGCTGACCTGGCGGATATGGCCGACA
AGGTTTTCCATATCAAACGGTTTGAGAATAAAGTAGGACGCGCCTAAATCGACGGCCTTTTTCGTGACATCTTCCT
GTCCAAAGGCTGTCAGCATAATGACATTCGGCTGTTTTTTCAGATCTGATTCCCTCAGCCTCTCTAAAACCGCAAGT
CCGTCTAAATGCGGCATAATAATATCTAATACGAGCACATCGGGATCTTTTTCTTTAAACAGCGACAGGCATTCCTG
TCCGTTATAAGCAACGCCGATCACTTCCATGTCTTCCTGTCCTTCTATATATTCACTTAACAGGCTTACCAGCTCTCG
ATTATCATCAGCAACACAAACTTTAATTTTCTCCACGTTTCTTCCTCCCCAAATGTAGTTAACAGGATTCACCCTTGC
TACATGTTTACATTCGACAAAACCGCTATATACCCTCTAAAAAAATCATAATCACCAATATTAGTGGCATATCTTTGT
TATTCTATGTTTTTCTCTGTTTTTCGACTAAAAATGAAATTTGACAAACAAGGAAACGTGAATTTGTCGAAAAATCTT
CTTTTGTATATTTTACCGTATGTATTCTGAGAAGTGAAGAGGGATCAAAATAAAAAAACTGCCGGAAACTCCGGCA
GTCAGCTTGCTTTTTCTTTTCCATAAATATCGATTCCTGCTTCTGACAGCATCCATTCAATATGAACACCGTAGCCGC
TTGTCGGGTCATTTACAAATACATGGGTGACAGCACCGATCACTTTTCCATTTTGAATGATCGGGCTTCCGCTCATC
CCCTGTACGATGCCTCCTGTTTCTTTCAACAGTCTTGGATCGGTAATTTTCAACACCATTCCTTTTGTCGCAGGGAAT
TTTTGCGGCGTTGTGCTGACGATTTCAATATCGAATTTTTCTACTTTGTCATCATCAATAACGGTTAAAATTTCAGCC
GGCCCTTTTTTGACTTCGGTAGAAAACGCAACCGGCAATGCTTGATCTGATATGTTGTTTTGAATCGGCTGATGCA
GTGTGCCGAAAATCCCAAACGGGCTGTTTCTGTTAATATCCCCGATCGTTTTGCGTTCTGAGGAAAATCGCGCCAG
TTTTTCTCCCGGATTACCGCCTGTCCCTTTTTCAATTGATGTTACAGTGGATTTAACGATTTCTCCATTCTCCACTACA
ATTGGTTTCTTTGTGTCCATATCGGAAATCACGTGGCCAAGTGCTCCGTATTTTTTTGTTTTCGGTTCATAAAAGGTC
ATAGTGCCGATGCCGGCAGCAGAATCTCTGATATATAACCCGATTCTGTATTTGCCTTCTCCTTCATCCTTTTCTGGG
ATCAGCTTCGTTTTGATTTTCTGTTTATCACGTTTGATCAGTAAGTCTAAAGATTCACCAGTTTTCCCAGCCTTTTGA
ATAAATGGGGCTACATCATTCATTTTTTCAATTTTCTGTCCATTCATCTCAATAATGATGTCGCCCGCCTCAATTCCT
GCCGTTTCTCCCGGAGATTTTTTGCCTTCACTTGTATTGATTTGATGAAATCCGACGACAAGAACACCGACGGAATG
AAGTTTTACACCGATTGATTGTCCGCCAGGTATAACTTTTAAATCAGGAAGAACATGCACTTTTGTTTTTTTAATTG
GAAATCCGGCAAGATCATATACCAATTCTGACTCACCTGATTTTTTGCCCGTCACCTTGATTTCATGCGGATCTTTCT
TTACTGTAAACGCTTCTGAGGATTCTGATGTCTGAGCGTTTACCGATAAACTCGTTTCAATCGCTTGTGTTTGGGTT
TCAAATACTCTCATTTGCGTTGGAATCAGTAAATATTCTTTTAGCGGTTTGCATAAACCTACACTTAATAACGAAAC
AAGGAGAATTAAACCTACTGCTTTTCTGATGTTATCGGGCAT

Using the ComKS inducible strain described in Example 7 as a basis (this genetically modified strain having increased competence), a non-sporulating version of the B. subtilis #105 was generated.

A non-sporulating version of B. subtilis #105 was generated by deleting SpoOA and SpoIVB coding sequences and confirmed by PCR and sequencing. Junction PCR confirmed the correct deletion of sporulation genes in B. subtilis strain 105 (data not shown).

### Example 10

### Strain 105 Promoters for Expression of Heterologous Proteins or Sequences

Suitable B subtilis strain 105 promoters are identified through analysis of the genome and global proteomics data. The promoters are engineered upstream of nucleic acid encoding one or more biomolecules or heterologous proteins. The promoters are used in expression cassettes suitable to generate a genetically-modified bacterium which can produce biomolecules or heterologous proteins and/or express desired biomolecules or heterologous proteins to deliver them to host animals in need thereof. Expression cassettes would comprise a suitable promoter, a heterologous coding sequence encoding a desired biomolecule or heterologous protein, and a transcription terminator. The biomolecule or heterologous coding sequence may also comprise a signal sequence for secretion, a cell-wall anchor sequence, and/or a detectable peptide tag. Notably, multiple promoters in tandem are utilized and applicable in some constructs. Several copies of these promoters may be used in tandem to further increase expression. Selected and suitable promoter sequences from strain 105 include:

Bacillus subtilis #105 tuf promoter
(SEQ ID NO: 66)
AAGTGCCGAAGAGCGTCGCAGAAGAAATTATCAAAAAAAATAAAGGCGA
ATAATTGATTTTGCCGCTTAACTCAAGTATAACTACTATTGTAAGATGA
GGAAGTGAAAGCTTTCTTTCACTTCCTATCACTCTATACATTACTAATT
AAAAGCTCTTAAGGAGGATTTTAGA
Bacillus subtilis #105 SigX promoter
(SEQ ID NO: 67)
ATCGAGTCTGAATTTGCCGAAGAATCTTGTTCCATAAGAAACACCCGCT
GACTGAGCGGGTGTTTTTTTAATAGCCAACATTAATAAAATTTAAGGAT
ATGTTAATATAAATTCCCTTCCAAATTCCAGTTACTCGTAATATAGTTG
TAATGTAACTTTTCAAGCTATTCATACGACAAAAAAGTGAACGGAGGGG
TTTCAA
Bacillus subtilis #105 groS promoter
(SEQ ID NO: 68)
ATGGTATGTACTCCTTTGTTAAGTGGGTTTCGTTCATCTACAGCTATTG
TAACATAATCGGTACGGGGGTGAAAAAGCTAACGGAAAAGCGAGCGGAA
AAGAATGATGTAAGCGTGAAAAAATTTTTATCTTATCACTTGAAATTGG
AAGGGAGATTCTTTATTATAAGAATTGTGTTAGCACTCTTTAGTGCTGA
GTGCTAAAATTACATATTCATACTATTGAGGAGGTTATTACA
Bacillus subtilis #105 ftsH promoter
(SEQ ID NO: 69)
TCGGCAGCCTGCTTCCGAGATGGTTATTGTTTGTATTGGAATGATTTTC
TATGGTACTATTGAACATAGTTGTGCTTACTGTGGGAGGAGGTAAGGA

### Example 11

### Modification of Strain 105 to Inactivate or Delete Proteases

In instances where one or more relevant target biomolecule or protein must be secreted by bacterial host cells, inherent environmental proteases, including native bacterial proteases produced by the delivery bacteria, can reduce the amount and extent of active and full length biomolecule or protein available. The expression of recombinant secretory proteins in B. subtilis can be less efficient, of lower than desired yield, or even unsuccessful due to the degradation of secreted proteins by extracellular proteases (Westers L, Westers H, Quax W J. (2004) Biochim Biophys Acta 1694:299-310; https://doi.org/10 0.1016/j.bbamcr.2004.02.011). B. subtilis has eight native extracellular proteases, known as NprE, AprE, Epr, Bpr, Mpr, NprB, Vpr, and WprA (Jeong H et al (2018) Microbiol Resour Announc 7:e01380-18; doi.org/10.1128/MRA.01380-18). To increase the stability and/or systemic activity of secreted proteins, extracellular-protease-deficient mutants are constructed.

Although a factor limiting the application of Bacillus subtilis as an expression host has been its production of at least eight extracellular proteases, researchers have also reported that some proteases benefited the secretion of foreign proteins at times. Therefore, to maximize the yield of a foreign protein, the proteases can be selectively inactivated. Accordingly, the optimal protease-deficient host is constructed through inactivating the most unfavorable proteases.

Zhao and colleagues have conducted studies with various protease inactivations and combinations in B. subtilis and assessing the production of non-native proteins, particularly α-amylase (AmyM) (Corallociccus sp.), methyl parathion hydrolase (MPH) (Plesiomonas sp.) and chlorothalonil hydrolytic dehalogenase (Chd) (Pseudomonas sp.) by the protease mutants (Zhao L. et al (2019) Biotechnology and Engineering 116:2052-2060) This study showed that B. subtilis proteases AprE and NprE contribute the majority of extracellular protease activity. The remaining significant protease activity is fulfilled by Epr, NprB, Bpr, Vpr, WprA, and Mpr which is in agreement with previous reports (Ferrari, Jarnagin, & Schmidt (1993) in Bacillus subtilis and other Gram-positive bacteria 263:917-937)). For secreted AmyM and Chd protein production, mutant strains deficient in NprE, AprE and Epr proteases and mutant strains deficient in NprE, AprE, NprB, Vpr and WprA, respectively, were shown to provide optimal production. Overall, it is evident that the secretion level of a target protein can be improved through inactivation of extracellular proteases.

The sequences of the eight native extracellular proteases NprE, AprE, Epr (Epr1 and Epr2), Bpr, Mpr, NprB, Vpr, and WprA from B. subtilis strain 105 are provided below. These sequences are targeted by inactivation or deletion using recombinant techniques and genetic manipulation of the 105 genome. Deletion can be accomplished for example by targeting one or more gene in the genome using n-terminal region and C terminal region genomic sequence from that provided below and/or using flanking nucleic acid sequence to the N-terminus or C-terminus sequence linked to heterologous or selectable sequence for insertion to replace the selected and targeted protease sequence. Recombination and gene replacement can be selected and/or detected using skilled artisan known and recognized means and methods in the art.

Genes encoding proteases in Bacillus #105
>nprE
(SEQ ID NO: 70)
GTGGGTTTAGGTAAGAAATTGTCTGTTGCTGTCGCTGCTTCGTTTATGAGTTTATCAATCAGCCTGCCAGGTGTTCA
GGCTGCTGAAGGTCATCAGCTTAAAGAGAATCAAACAAATTTCCTCTCCAAAAATGCGATTGCGCAATCAGAACTC
TCTGCACCAAATGACAAGGCTGTCAAGCAGTTTTTGAAAAAGAACAGCAACATTTTTAAAGGTGATCCTTCCAAAA
GGCTGAAGCTTGTTGAAAGCACGACTGATGCCCTTGGATACAAGCACTTTCGATATGCGCCTGTCGTTAACGGAGT
GCCAATTAAAGATTCGCAAGTGATCGTTCACGTCGATAAATCCGATAATGTCTATGCGGTCAATGGTGAATTACAC
AATCAATCTGCTGCAAAAACAGATAACAGCCAAAAAGTCTCTTCTGAAAAAGCGCTGGCACTCGCTTTCAAAGCTA
TCGGCAAATCACCAGACGCTGTTTCTAACGGAGCGGCCAAAAACAGCAATAAAGCCGAATTGAAAGCCATAGAAA
CAAAAGACGGCAGCTATCGTCTTGCTTACGACGTGACGATTCGCTATGTCGAGCCTGAACCTGCAAACTGGGAAG
TCTTAGTTGACGCCGAAACAGGCAGCATTTTAAAACAGCAAAATAAAGTAGAACATGCCGCCGCCACTGGAAGCG
GAACAACTCTAAAGGGCGCAACTGTTCCTTTGAACATCTCTTATGAAGGCGGAAAATATGTTCTAAGAGATCTTTC
AAAACCAACAGGCACCCAAATCATCACATATGATTTGCAAAACAGACAAAGCCGCCTTCCGGGAACGCTTGTCTCA
AGCACAACGAAAACATTTACATCTTCATCACAGCGGGCAGCCGTTGACGCACACTATAACCTCGGTAAAGTGTACG
ATTATTTTTATTCAAACTTTAAACGAAACAGCTATGATAACAAAGGCAGTAAAATCGTTTCTTCCGTTCACTATGGC
ACTCAATACAATAACGCTGCATGGACAGGAGACCAGATGATTTACGGTGATGGCGACGGTTCATTCTTCTCTCCGC
TTTCCGGCTCATTAGATGTGACAGCGCATGAAATGACACATGGCGTCACCCAAGAAACAGCCAACTTGATTTATGA
AAATCAGCCAGGTGCATTAAACGAGTCTTTCTCTGACGTATTCGGGTATTTTAACGATACAGAAGACTGGGACATC
GGTGAAGACATTACGGTCAGCCAGCCTGCTCTTCGCAGCCTGTCCAACCCTACAAAATACAACCAGCCTGACAATT
ACGCCAATTACCGAAACCTTCCAAACACAGATGAAGGCGATTATGGCGGTGTACACACAAACAGCGGAATTCCAA
ACAAAGCCGCTTACAACACCATCACAAAACTTGGTGTATCTAAATCACAGCAAATCTATTACCGTGCGTTAACAAC
GTACCTCACGCCTTCTTCCACGTTCAAAGATGCCAAGGCAGCTCTCATTCAGTCTGCCCGTGACCTCTACGGCTCAA
CTGATGCCGCTAAAGTTGAAGCAGCCTGGAATGCTGTTGGATTGTAA
The encoded nprE protease is (SEQ ID NO: 71):
VGLGKKLSVAVAASFMSLSISLPGVQAAEGHQLKENQTNFLSKNAIAQSELSAPNDKAVKQFLKKNSNIF
KGDPSKRLKLVESTTDALGYKHFRYAPVVNGVPIKDSQVIVHVDKSDNVYAVNGELHNQSAAKTDNSQKV
SSEKALALAFKAIGKSPDAVSNGAAKNSNKAELKAIETKDGSYRLAYDVTIRYVEPEPANWEVLVDAETG
SILKQQNKVEHAAATGSGTTLKGATVPLNISYEGGKYVLRDLSKPTGTQIITYDLQNRQSRLPGTLVSST
TKTFTSSSQRAAVDAHYNLGKVYDYFYSNFKRNSYDNKGSKIVSSVHYGTQYNNAAWTGDQMIYGDGDGS
FFSPLSGSLDVTAHEMTHGVTQETANLIYENQPGALNESFSDVFGYENDTEDWDIGEDITVSQPALRSLS
NPTKYNQPDNYANYRNLPNTDEGDYGGVHTNSGIPNKAAYNTITKLGVSKSQQIYYRALTTYLTPSSTFK
DAKAALIQSARDLYGSTDAAKVEAAWNAVGL
>bpr
(SEQ ID NO: 72)
ATGAAGAAAAAAACGAAAAACAGACTCATCAGCTCTGTTTTAAGTACAGTTGTCATCAGTTCACTGCTGTTTCCGG
GAGCAGCCGGGGCAAGCAGTAAAGTCACCTCACCTTCTGTTAAAAAGGAGCTTCAATCTGCGGAATCCATTCAAA
ACAAGATTTCGAGTTCATTAAAGAAAAGCTTTAAAAAGAAAGAAAAAACGACTTTTCTGATTAAATTTAAAGATCA
GGCTAACACAGAAAAAGCGGCAAAAGCGGCTGTTAAAAAAGCGAAATCGAAGAAGCTGTCTGCCGCTAAGACGG
AATATCAAAAGCGTTCTGCTGTTGTGTCATCTTTAAAAGTCACAGCCGATGAATCCCAGCAAGATGTCCTAAAATAT
TTGAACGCCCAGAAAGATAAAGGAAATGCAGACCAAATTCATTCTTATTATGTGGTGAACGGGATTGCTGTTCATG
CCTCAAAAGAGGTTATGGAAAAAGTGGCGCAGTTTCCCGAAGTGGAAAAGGTGCTTCCTAATGAGAAACGGCAG
CTTTTTAAGTCATCCTCCCCATTTAATATGAAAAAAGCACAGAAAGCTATTAAAGCAACTGACGGTGTGGAATGGA
ATGTAGACCAAATCGATGCTCCAAAAGCTTGGGCACTTGGATATGATGGAACTGGCACGGTTGTTGCGTCCATTG
ATACCGGGGTGGAATGGAATCATCCGGCATTAAAAGAGAAATATCGCGGATATAATCCGGAAAATCCTAATGAGC
CTGAAAATGAAATGAACTGGTATGATGCCGTAGCAGGCGAGGCAAGCCCTTATGATGATTTGGCTCATGGAACCC
ATGTGACAGGCACGATGGTGGGCTCTGAACCTGATGGAACAAATCAAATCGGTGTAGCGCCTGGCGCAAAATGG
ATTGCTGTTAAAGCGTTCTCTGAAGATGGCGGCACTGATGCTGACATTTTGGAAGCTGGTGAATGGGTTTTAGCAC
CAAAGGACGCGGAAGGAAATCCCCACCCGGAAATGGCTCCTGATGTTGTCAATAACTCATGGGGAGGGGGCTCT
GGACTTGATGAATGGTACAGAGACATGGTCAACGCCTGGCGTGCGGCCGATATTTTCCCTGAGTTTTCAGCGGGG
AATACGGATCTCTTTATTCCCGGCGGGCCTGGTTCTATCGCAAATCCGGCAAACTATCCAGAATCGTTTGCAACTG
GAGCGACTGATATCAATAAAAAGCTCGCTGACTTTTCTCTTCAAGGGCCATCTCCATATGATGAGATAAAGCCGGA
AATATCTGCACCGGGCGTTAATATTCGTTCATCCGTTCCCGGTCAGACATATGAGGATGGTTGGGACGGCACATCA
ATGGCAGGGCCGCATGTATCCGCTGTTGCTGCACTGCTGAAACAGGCGAATGCCTCACTTTCTGTTGATGAGATGG
AGGATATATTAACCAGCACGGCTGAACCGCTCACGGATTCAACATTTCCTGATTCACCGAATAACGGATATGGCCA
TGGTCTGGTGAATGCTTTTGATGCTGTATCCGCTGTTACAGATGGTTTAGGGAAAGCGGAAGGACAAGTTTCTGTA
GAGGGGGATGACCAAGAGCCTCCTGTCTATCAGCATGAGAAAGTAACTGAAGCTTATGAAGGTGGTAGCCTGCC
ACTGACTTTGACAGCTGAAGACAATGTGAGTGTGACATCTGTAAAGCTGTCCTACAAGCTTGATCAAGGTGAATG
GACAGAAATAACGGCTAAACGAATCAGCGGTGATCATCTAAAAGGAACGTATCAGGCAGAGATCCCAGATATAA
AAGGAACTAAACTAAGCTATAAGTGGATGATTCACGATTTTGGCGGTCATGTCGTTTCGTCTGACGTATACGATGT
AACAGTGAAACCAAGCATCACGGCGGGATATAAGCAGGACTTTGAAACTGCACCCGGCGGCTGGGTTGCGAGCG
GAACAAATAATAACTGGGAATGGGGAGTTCCGTCAACTGGCCCAAATACAGCAGCATCCGGAGAAAAAGTATAT
GGAACGAATTTGACAGGAAATTATGCCAACTCAGCAAACATGAACCTTGTTATGCCTCCTATTAAAGCACCTGATT
CAGGAAGTCTGTTCCTTCAATTTAAAAGCTGGCACAATTTAGAGGATGATTTTGATTACGGCTACGTTTTTGTTCTT
CCGGAAGGTGAAAAGAATTGGGAGCAGGCTGGTGTCTATAACGGAAAGACATCAAGCTGGACGGACGAAGAAA
TTGATTTATCGGCTTATAAAGGCCAAAACATTCAAGTGATGTTTAACCTTCAATCTGATGAAAGCATTGCAAAAGA
GGGCTGGTATATTGATGATGTAGTGCTTTCTGACAAATCAGCCGGAAAAACAGTCAAAAAGAATAAGCTGGGCGT
CGAAAAGCCGTCTGGAAAGCAAAAGAAAAAGCCAGTAAATCCGAAAAAGGCTAAGCCATCTGCAAACACAGCGG
TAAAACATCAGAACAAGGCTATACAGCCTCAAGTTTTGCCGCTCAGGGCACAAGTCAGTGTAGTGGAAACAGGAA
AATCAACATATTCAGATCAATCCACAGGGCAGTACACGCTGAAGCACAAAGCGGGAGACTATACGCTTATGGCAG
AAGCATATGGTTATCAGTCGAAAACACAAAAAGTATCTTTAAAGACGGATCAGACGACACAAGCTAATTTTACGTT
AGAAGAGATGAAGAAGGGCACATTAAAAGGCATGGTCATCAATAAAACGACAGGAGAGCCGGTAACAGGCGCTT
CCGTTTATGTTGTAGAGGATGCTGCTGTGGAACCGGCTATGACAAACGACAAAGGTGAATATACGCTGGAGGCCT
ATGAAGGCGCTTATACGATTAAAGTCGCTGCACCGGGTTATTACAGTGATGAATTTTCCGTTGAGTTAAAAGGTGA
TGTTACAAAAGAAACTGCATTGAAGCCTTTCGTCGGTTATCCGGGTGAAATTGCATATGATGACGGAACAGCGGA
GAATGCCAATTCCTATTTTGCTGCCGGTAACGGATGGGCGGTAAAAATGACGCTCGCTGACGGCAAGGACAAAGG
CATGCTTACAGGAGGGCTGTTCAGATTCTGGGATACAGAGTTCCCAGATCCGGGCGGCACAGAGTTTAAGGTTGA
GGTATACGATGCCACAGGAAAAGACGGAGCACCGGGCAAGAAAATTGCAGGGCCATTTAACGCTGAAGCTCTTC
GCAATGGCGAGTGGACTAAGGTAGATCTCAGTTCAAAAGGGATTATGGTCGATAGAGACTTTTATCTCGTATATAT
CCAGTCAAAACCTGACCCGTATTCACCTGGACTGGCAATGGATGAAACCGGCAAGAATTCCGGCCGCAACTGGCA
GTATATAGGTGGACAATGGGAGCCGGGTGACGAAGCAGACGGCAACTATATGATTCGCGCATTAGTTAATTATGA
AGCTGCTGTACCTGAGATTACTTCACCGACAGACAAATCATACACAAATAAGGATAGCGTCACTGTAAAAGGAAA
CGCTTCTCCTGGCACAACGGTACACATTTATAATGGAGAGAAAGAAGCAGGAGAAACGAAAGCTGCTGCGGATG
GCACGTTCCATGCAGGCATCATACTCAACAAGGGTGAAAATGAGCTGACGGCAACTGCATCAACTGACAACGGAA
CAACAGATGCCTCCAGCCCAATCACGGTCACGCTTGATCAAGAAAAGCCTGAATTAACACTGGACAATCCAAAGG
ATGGCGGGAAAACAAATAAAGAAACGCTGACTGTCAAAGGGGCTGTATCCGATGACAATCTGAAAGACGTCAAG
GTGAATGGCAAAAAAGCAACAGTAGCTGATGGTTCATACTCAGCCCGTATTCTTTTGGAAAATGGAAGAAATGAA
ATCAAGGTAATTGCTACAGACTTGGCAGGCAACAAAACGACGAAAAAGACAGTCATTGATGTGAACTTTGACAAG
CCTGTCATTTCCGGCTTAATTCCGGGAGAGGATAAAAACTTAAAAGCCGGTGAATCTGTGAAAATCGCTTTCTCAA
GCGCTGAGGATTTAGATGCAACGTTTACCATTCGTATGCCGCTGACGAATGCAAGAGCGAGTGTGCAAAATGCCA
CCGAACTCCCGTTAAGAGAAATCTCTCCGGGGAGATATGAAGGCTATTGGACTGCCACTTCTTCTATTAAAGCAAA
AGGAGCAAAAGTAGAAGTGATCGTCCGAGATGATTATGGAAATGAAACAAGAAAAACTGCGAATGGAAAACTTA
ATATCAACACAGAAAATTAA
>vpr
(SEQ ID NO: 73)
TTGAAAAAGGGGATCATTCGCTTTCTGCTTGTAAGTTTCGTCTTATTTTTTGCGTTATCCACAGGTATTACGGGCGT
CCAGGCAGCTCCGGCTTCTTCAAAAACGTCGGCTGATCTGGAAAAAGCCGAGGTATTCGGTGATATCGACATGAC
GACAAGCAAAAAAACAACCGTTATAGTGGAATTAAAAGAAAAATCCTTGGCAGAAGCGAAGGAAGCGGGAGAAA
GCCAATCGAAAAGCAAGCTGAAAACCGCTCGCACCAAAGCAAAAAATAAAGCAATCAAAGCGGTGAAAAACGGA
AAAGTAAACCGGGAATATGAGCAGGTATTCTCAGGCTTCTCTATGAAGCTTCCAGCTAATGAGATTCCAAAACTTC
TCGCGGTAAAAGACGTTAAGGCGGTGTACCCGAACGTCACATATAAAACAGACAATATGAAGGATAAAGACGTCA
CAATCTCCGAAGACGCCGTATCTCCGCAAATGGATGACAGTGCGCCTTATATCGGAGCAAACGATGCATGGGATT
TAGGCTACACAGGAAAAGGCATTAAGGTGGCGATTATTGACACTGGGGTTGAATACAATCACCCAGATCTGAAGA
AAAACTTTGGACAATATAAAGGATACGATTTTGTGGACAATGATTACGATCCAAAAGAAACACCAACCGGCGATC
CGAGGGGCGAGGCAACTGACCATGGCACACACGTAGCCGGAACTGTGGCTGCAAACGGAACAATTAAAGGCGTA
GCGCCTGATGCCACACTTCTTGCTTATCGTGTGTTAGGGCCTGGCGGAAGCGGCACAACGGAAAACGTCATCGCG
GGCGTGGAACGTGCAGTGCAGGACGGGGCAGATGTGATGAACCTGTCTCTCGGAAACTCTTTAAACAACCCGGA
CTGGGCGACAAGCACAGCGCTTGACTGGGCCATGTCAGAAGGCGTTGTCGCTGTTACCTCAAACGGCAACAGCGG
ACCGAACGGCTGGACAGTCGGATCGCCGGGCACATCAAGAGAAGCGATTTCTGTCGGTGCGACTCAGCTGCCGCT
CAATGAATACGCCGTCACTTTCGGCTCCTATTCTTCAGCAAAAGTGATGGGCTATAACAAAGAGGACGACGTCAAA
GCGCTCAATAACAAAGAAGTTGAGCTTGTCGAAGCGGGAATCGGCGAAGAAAAGGATTTTCAAGGGAAAGACCT
GACAGGCAAAGTCGCCGTTGTCAAACGAGGTAGCATTGCATTTGTGGATAAAGCGGATAACGCTAAAAAAGCCG
GTGCAATCGGCATGGTTGTGTATAACAACCTCTCTGGAGAAATTGAAGCCAATGTGCCAGGCATGTCTGTCCCAAC
GATTAAGCTTTCATTAGAAGACGGCGAAAAACTCGTCAGCGCCCTGAAAGCTGGTGAGACAAAAACAACATTCAA
GTTGACGGTCTCAAAAGCGCTCGGTGAACAAGTGGCTGATTTCTCATCACGCGGCCCTGTTATGGATACGTGGAT
GATTAAGCCTGATATTTCCGCGCCAGGGGTCAATATCGTGAGCACGATCCCAACACACGATCCTGACCATCCATAC
GGCTACGGATCAAAACAAGGAACAAGCATGGCATCGCCTCATATTGCCGGAGCGGTTGCCGTTATTAAACAAGCC
AAGCCAAAGTGGAGCGTTGAACAGATTAAAACCGCCATCATGAATACCGCTGTCACTTTAAAGGATAGCGATGGG
GAAGTATATCCGCATAACGCTCAAGGCGCAGGCAGCGCAAGAATCATGAACGCAATCAAAGCAGATTCGCTCGTC
TCACCTGGAAGCTATTCATACGGCACGTTCTTGAAGGAAAACGGAAACGAAACGAAAAAAGAAACGTTTACGATT
GAAAATCAATCTTCCATTAGAAAATCATACACACTGGAATACTCATTTAATGGCAGCGGCATCTCCACATCCGGCAC
AAGCCGTGTTGTGATTCCGGCACATCAAACCGGGAAAGCCACTGCAAAAGTAAAGGTCAATACGAAGAAAACAA
AAGCTGGCACCTATGAAGGAACGGTTATCGTCAGAGAAGGCGGAAAAACGGTCGCTAAGGTACCTACATTGCTG
ATTGTGAAAGAGCCCGATTATCCGAGAGTCACATCTGTCTCTGTCAGCGAAGGGTCTGTACAAGGTACCTATCAAA
TTGAAACCTACCTTCCTGCGGGAGCGGAAGAGCTAGCGTTCCTCGTCTATGACAGCAACCTTGATTTCGCAGGCCA
AGCCGGCATTTATAAAAACCAAGATAAAGGTTACCAGTACTTTGACTGGGACGGCACGATTAATGGCGGAACCAA
ACTTCCGGCCGGAGAGTATTACTTGCTCGCATATGCCGGAAACAAAGGCAAGTCAAGCCAGGTTTTGACCGAAGA
ACCTTTCACTGTTGAATAA
The encoded vpr protease is:
(SEQ ID NO: 74)
LKKGIIRFLLVSFVLFFALSTGITGVQAAPASSKTSADLEKAEVFGDIDMTTSKKTTVIVELKEKSLAEA
KEAGESQSKSKLKTARTKAKNKAIKAVKNGKVNREYEQVFSGFSMKLPANEIPKLLAVKDVKAVYPNVTY
KTDNMKDKDVTISEDAVSPQMDDSAPYIGANDAWDLGYTGKGIKVAIIDTGVEYNHPDLKKNFGQYKGYD
FVDNDYDPKETPTGDPRGEATDHGTHVAGTVAANGTIKGVAPDATLLAYRVLGPGGSGTTENVIAGVERA
VQDGADVMNLSLGNSLNNPDWATSTALDWAMSEGVVAVTSNGNSGPNGWTVGSPGTSREAISVGATQLPL
NEYAVTFGSYSSAKVMGYNKEDDVKALNNKEVELVEAGIGEEKDFQGKDLTGKVAVVKRGSIAFVDKADN
AKKAGAIGMVVYNNLSGEIEANVPGMSVPTIKLSLEDGEKLVSALKAGETKTTFKLTVSKALGEQVADFS
SRGPVMDTWMIKPDISAPGVNIVSTIPTHDPDHPYGYGSKQGTSMASPHIAGAVAVIKQAKPKWSVEQIK
TAIMNTAVTLKDSDGEVYPHNAQGAGSARIMNAIKADSLVSPGSYSYGTFLKENGNETKKETFTIENQSS
IRKSYTLEYSFNGSGISTSGTSRVVIPAHQTGKATAKVKVNTKKTKAGTYEGTVIVREGGKTVAKVPTLL
IVKEPDYPRVTSVSVSEGSVQGTYQIETYLPAGAEELAFLVYDSNLDFAGQAGIYKNQDKGYQYFDWDGT
INGGTKLPAGEYYLLAYAGNKGKSSQVLTEEPFTVE
>epr1
(SEQ ID NO: 75)
ATGAAAAACATGTCTTGCAAACTTGTTGTATCAGTCACTCTGTTTTTCAGTTTTCTCGCCATAGGCCCTCTCGCTCAT
GCGCAAAACAGCAGCGAGAAAGAGGTTATTGTGGTTTATAAAAACAAGGCCGGAAAGGAAACCATCCTGGATAG
TGATGCTGATGTTGAACAGCAGTATAAGCATCTTCCCGCGGTAGCGGTCACAGCAGACCAGGAGACAGTAAAAGA
ATTAAAGCAGGATCCTGATATTTTGTATGTAGAAAACAACGTATCATTTACCGCAGCAGACAGCACGGATTTCAAA
GTGCTGTCAGACGGCACTGACACCTCTGACAACTTTGAGCAATGGAACCTTGAGCCCATTCAGGTGAAACAGGCTT
GGAAGGCAGGACTGACAGGAAAAAATATCAAAATTGCCGTCATTGACAGCGGGATCTCCCCCCACGATGACCTGT
CGATTGCCGGCGGGTATTCAGCTGTCAGTTATACCTCTTCTTACAAAGATGATAACGGCCACGGAACACATGTCGC
AGGGATTATCGGAGCCAAGCATAACGGCTACGGAATTGACGGCATCGCACCGGAAGCACAAATATACGCGGTTA
AAGCGCTTGATCAGAACGGCTCGGGGATCTTCAAAGTCTTCTCCAAGGAATTGACTGGTCGATCGCAAACGGGAT
GGACATCGTCAATATGA
>epr2
(SEQ ID NO: 76)
ATGAGCCTTGGCACGACGTCAGACAGCAAAATCCTTCATGAGGCCGTGAACAAAGCATATGAACAAGGTGTTCTG
CTTGTTGCCGCAAGCGGTAACGACGGAAACGGCAAGCCAGTGAATTATCCGGCGGCATACAGCAGTGTCGTTGCT
GTTTCAGCAACAAACGAAAAGAATCAGCTTGCCTCCTTCTCAACAACTGGAGATGAAGTTGAATTTTCAGCGCCGG
GGACAAACATCACAAGCACTTACTTAAACCAGTATTATGCAACAGGAAGCGGAACATCCCAAGCGACACCGCACG
CCGCTGCCATGTTTGCTTTGTTAAAACAGCGTGATCCTGCCGAGACAAACGTCCAGCTTCGCGAGGAAATGCGGA
GAAATATCGTTGATCTTGGTACCGCAGGCCGCGATCAGCAATTTGGCTACGGCTTAATCCAATATAAAGCACAGGT
AACAGATTCAGCGTACGCGGCAGCAGAGCAAGCGGTGAAAAAAGCGGAACAAACAAAAGCACAAACCGAAATCA
ACAAAGCGCGAGAACTCATCAGCCAGCTGCCGGGCTCCGACGCCAAAACTGCCCTGCAAAAAAGACTGGATAAA
GTACAGTCATACAGAAATGTAAAAGATGCGAAAGACAAAGTCGCAAAGGCAGAAAAATATAAAACACAGCAAAC
CGTTGATACAGCACAAACTGCCATCAACAAGCTGCCAAACGGAACAGACAAAAAGAACCTTCAAAAACGCTTAGA
CCAAGTAAAACGCTACATCGCGTCAAAGCAAGCGAAAGACAAAGTTGCGAAAGCGGAAAAAAGCAAAAAGAAAA
CAGATGTGGACAGCGCACAATCAGCAATTGGCAAGCTGCCTGCAAGTTCAGAAAAAACGTCCCTGCAAAAACGCC
TTAACAAAGTGAAGAGCACCAATTTGAAGACGGCACAGCAATCCGTATCTGCGGCTGAAAAGAAATCAACTGATG
CAAATGCGGCAAAAGCACAATCAGCCGTCAATCAGCTTCAAGCAGGCAAGGACAAAACGGCATTGCAAAAACGA
TTAGACAAGGTGAAGAAAAAGGTGGCGGCGGCTGAAGCAAAAAAAGTCGAAACTGCCAAGGCAAAAGTGAAGA
AAGCGGAAAAAGACAAAACAAAGAAATCAAAGACATCCGCTCAGTCTGCAGTGAATCAATTAAAAGCATCCCATG
AAAAAACAAAGCTGCAAAAACGGCTGAACGCCGTCAAACCGAAAAAGTAA
>wprA
(SEQ ID NO: 77)
ATGAAACGCAGAAAATTCAGCTCGGTTGTGGCGGCAGTGCTTATTTTTGCACTGATTTTCAGCCTTTTTTCTCCGGG
AACCAAAGCTGCAGCGGCCGGCGCGATCGATCAGGCGGCTGCTCTGGAAAACGGCAAAGAGCAGACAGGCGCC
ATGAAGGAGCCGGAACAGGTGAAATGGTACAAAGTGACCCCGGGAGCAACGGATATTCAGAAAAACTCACATAT
GGCACTGACCGTAAAGAGTGATTCAGTACTGAATGTATCTGTATATCCAAGTAAGGAAAAAGCGCTTAAAGATGA
GACGTTTGAGATGTACCGTTCTTTCACAGCGGAGGATGGAAAAAGCGAAGTCATTTTTCCATACGCGTGGAGCGG
CCCTTACTATGTAAAAGTTGAATACCTCGGAGAAGAAGAGCCAGAGGACGGCGGAACGGCAGAAGCAGCTGCAG
AAGCCAAGTATACGATTGGGTATAAAGGCACCAAAAAACAGCCGTCAGATTTAGAAGAGGAAGAAGCTTGTCCG
GTTGAAATGAGTGTCGATCAGAAGAAATCAGGAAAAGGCATCCTGGATAAGCTGAGAGCGATTCGTGATGAGCA
GCTGAGCCAAACAGCAGAAGGCAAAGAACTGACAAGCCTTTATTACAAAGCAGCACCGTTTATCGTTGCAAAGCT
CGCACTCAATAAAACAGCAAGAAATGAAATCTATCAGGATCTTGTGACATTAAAGCCGTTATTTGACGATGTGTCA
GAAAACGGAGCATCATCTTCGCATAAGGTCACTGAAAAGGATCAAAAAGCAATCAACCGGCTATATGATAAAGCT
TTACAATCAGTCCCGTCATTCCTTAAAGAGGAGATAAAGAAACAAGCGGACCGACTAAATATGAAGCAGCTGCAA
GGCAAAACAGCCGGAGCCATTTTAACAGAAAACCATATTGCAGCAAAAAGTGAAGTTCAGACAACAAAGGTTATT
TTCAAGGTGAAGGACAATAAAAGCCTCTCATCCGTACATAATGAAATGAAGGGCTTTTCTGCAAGCGCGCAATCG
AAAAAAGACATATCCAATGTGAAAAAGGCAAAGAAACTGTTTGACAATCTGTATTCATTTGAACTTCCGAAAGATG
AGAAACAGAACGGCGCATATACGGCAAGCGCCAAAAGGGTCAAAAGCGCTGCTGCGACACTATCCAAGATGTCC
AATGTAGAGTTTGCGGAACCCGTGCAGGAATACAAAAGCCTGGCAAACGACATTCAGTACCCTTATCAATGGCCG
CTTAAAAACAACGGTGAAAACGGGGGTGTCAAAAATGCGGATGTGAAATATGAGCCTGCCAACACATTGCTGTCC
AAACGCAAGCTTAACGATACACTCATTGCAGTAGTAGACACAGGCGTAGACAGCACGCTTGCTGATTTAAAAGGA
AAAGTAAGAACAGATCTCGGACACAATTTTGTCGGACGGAATAACAATGCAATGGATGATCAGGGGCATGGGAC
GCATGTCGCAGGCATTATTGCGGCCCAAAGCGATAACGGCTATTCAATGACTGGATTGAATGCCAAAGCAAAAAT
CATCCCGGTAAAAGTGCTTGATTCCGCAGGTTCCGGAGATACTGAACAAATTGCTCTCGGCATCAAATATGCTGCT
GACAAAGGAGCAAAGGTGATTAATTTAAGTTTAGGCGGAGGCTACAGCCGCGTGCTTGAATTTGCTTTGAAGTAC
GCAGCTGACAAAAATGTCTTGATTGCCGCAGCCAGCGGGAATGATGGAGAAAATGCCTTATCTTATCCAGCATCTT
CTAAATATGTGATGTCAGTCGGCGCAACGAACCGCATGGATATGACCGCTGATTTCTCTAACTATGGAAAAGGTCT
GGACATCTCTGCTCCAGGGTCTGATATCCCGAGCTTAGTACCGAACGGAAATGTCACGTACATGAGCGGAACGTC
TATGGCGACGCCGTATGCTGCCGCTGCTGCAGGATTGCTGTTTGCTCAAAATCCTAAGCTGAAAAGAACAGAAGTT
GAGGATATGTTGAAAAAGACGGCAGATGACATTTCCTTTGAAAGTGTCGATGGCGGAGAAGAAGAGTTGTATGA
CGATTATGGCGATCCGATTGAAATTCCGAAGACACCTGGTGTAGACTGGCATTCAGGCTACGGGCGGCTGAATGT
CATGAAGGCTGTCAGCGCAGCTGATTTACAGCTTAAGGTCAACAAGCTGGAAAGCACTCAAACAGCTGTCAGAGG
AAACGCGAAGGAAGGCACACTTATCGAGGTGATGAACGGCAAAAAGAAACTCGGCAGCGCCAAAGCCGGAAAA
GACAATGCGTTCAAGGTCAATATCGCGACTCAAAAACAGGATCAAGTACTGTATGTGAAAGCAACAAAAGGCGAT
GCGAAAACATCGTATAAAGTTGTCGTCGTCAAAGGAAAACCTTCCGGCACACCGAAAGTAAACGCGGTGAAAACG
AAGGATACGGCAGTAAAAGGGAAGGCAAACAGCAAAGCGATGATCAGAGTGAAAAACAAATCAAAGAAAGTCA
TTGCTTCTGCCAAAGCTGACGCAAAAGGAACGTTTTCGGTGAAAATCAAAAAACAAAAAGCCGGAACGGTGCTGT
ACGTCACGGCTGCGGATACAGATAAAAAAGAAAGCAAGGAAGCAAAAGTGGTTGTTGAAAAGTAA
>nprB
(SEQ ID NO: 78)
TTGCGCAACCTGACCAAGACATCTCTATTACTGGCCGGCTTATGCACGGCGGCCCAAATGGTTTTTGTAACACATG
CCTCAGCTGATCAAAGCATCAAATACGACCATACGTATCAAACCCCTTCATACATCATCGAAAAGTCACCGCAGAA
GCCGGTACAAAACACAACCCAGAAAGAATCGCTATTTTCCTATCTTGACAAGCATCAAACGCAGTTTAAGCTCAAA
GGGAATGCGAACAGCCATTTTCGTGTTTCGAAAACCATAAAGGATCCAAAGACAAAACAAACGTTTTTTAAATTAA
CGGAGGTTTACAAAGGAATTCCGATTTACGGCTTTGAACAAGCGGTCGCGATGAAGGAAAACAAACAAGTGAAA
AGTTTCTTTGGAAAGGTGCATCCGCAAATCAAGGACGTCTCCATCACACCGTCTATTTCTGAGAAAAAAGCAATAC
ATGCAGCAAGGCGTGAGCTCGAGGCTTCCATTGGAAAAATCGAATATCTTGATGGGGAACCAAAAGGCGAATTAT
ATATCTATCCACACGACGGTGAATATGATCTCGCCTACCTTGTGAGACTCTCGACATCTGAACCTGAGCCTGGCTAT
TGGCATTATTTCATCGATGCCAAAAACGGAAAGGTCATTGAGTCCTTTAATGCCATTCATGAAACGGCAGGTACAG
GAATCGGCGTATCAGGTGATGAAAAAAGCTTTGACGTCAGAGAACAAAATGGGCGCTTTTATTTGGCTGACGAAA
CAAGGGGAAAAGGGATCAATACATTTGACGCGAAGAACCTGAACGAAACCTTGTTTACGCTTTTGTCTCAACTGAT
CGGGTATACGGGCAAAGAAATAGTCAGCAGCACGTCCGTATTTAATGAACCTGCGGCTGTAGACGCACACGCAAA
TGCGCAAGCCGTTTACGATTATTACAGCAAGACATTTGGCCGTGATTCTTTTGATCAAAACGGAGCAAGGATTACG
TCTACCGTGCATGTCGGCAAACAATGGAACAATGCTGCGTGGAACGGTGTCCAGATGGTATACGGGGATGGAGA
CGGTTCGAAATTTAAGCCGCTGTCTGGATCGCTCGACATTGTCGCGCATGAAATCACACACGCAGTCACACAGTAT
TCCGCCGGTCTTTTATATCAAGGAGAACCCGGTGCATTAAATGAGTCCATTTCTGACATTATGGGCGCGATGGCTG
ACCGTGATGATTGGGAGATCGGCGAAGATGTCTATACTCCTGGTATTGCAGGAGATTCATTGCGGTCATTGGAGG
ACCCATCTAAGCAGGGAAATCCAGATCATTACTCGAACCGCTACACAGGAACAGAGGATTATGGCGGAGTCCATA
TCAATTCGTCCATTCACAATAAAGCAGCTTATCTTCTTGCAGAAGGAGGCGTGCACCACGGTGTACAGGTTGAAGG
GATTGGGCGTGAAGCAAGTGAACAAATTTACTATCGGGCTTTAACATATTATGTAACGGCATCTACAGATTTCAGC
ATGATGAAGCAAGCGGCGATTGAAGCTGCCAATGATTTATACGGTGAAGGCTCGAAGCAATCAGCTTCAGTCGAA
AAGGCGTATGAGGCTGTCGGCATTCTATGA
The encoded nprB protease is (SEQ ID NO: 79):
LRNLTKTSLLLAGLCTAAQMVFVTHASADQSIKYDHTYQTPSYIIEKSPQKPVQNTTQKESLFSYLDKHQ
TQFKLKGNANSHFRVSKTIKDPKTKQTFFKLTEVYKGIPIYGFEQAVAMKENKQVKSFFGKVHPQIKDVS
ITPSISEKKAIHAARRELEASIGKIEYLDGEPKGELYIYPHDGEYDLAYLVRLSTSEPEPGYWHYFIDAK
NGKVIESFNAIHETAGTGIGVSGDEKSFDVREQNGRFYLADETRGKGINTFDAKNLNETLFTLLSQLIGY
TGKEIVSSTSVFNEPAAVDAHANAQAVYDYYSKTFGRDSFDQNGARITSTVHVGKQWNNAAWNGVQMVYG
DGDGSKFKPLSGSLDIVAHEITHAVTQYSAGLLYQGEPGALNESISDIMGAMADRDDWEIGEDVYTPGIA
GDSLRSLEDPSKQGNPDHYSNRYTGTEDYGGVHINSSIHNKAAYLLAEGGVHHGVQVEGIGREASEQIYY
RALTYYVTASTDFSMMKQAAIEAANDLYGEGSKQSASVEKAYEAVGIL
>aprE
(SEQ ID NO: 80)
GTGAGAAGCAAAAAATTGTGGATCAGCTTGTTGTTTGCGTTAACGTTAATCTTTACGATGGCGTTCAGCAACATGT
CTGCGCAGGCTGCCGGAAAAAGCAGTACAGAAAAGAAATACATTGTCGGATTTAAACAGACAATGAGTGCCATG
AGTTCCGCCAAGAAAAAGGATGTTATTTCTGAAAAAGGCGGAAAGGTTCAAAAGCAATTTAAGTATGTTAACGCG
GCCGCAGCAACATTGGATGAAAAAGCTGTAAAAGAATTGAAAAAAGATCCGAGCGTTGCATATGTGGAAGAAGA
TCATATTGCACATGAATATGCGCAATCTGTTCCTTATGGCATTTCTCAAATTAAAGCGCCGGCTCTTCACTCTCAAG
GCTACACAGGCTCTAACGTAAAAGTAGCTGTTATCGACAGCGGAATTGACTCTTCTCATCCTGACTTAAACGTCAG
AGGCGGAGCAAGCTTCGTACCTTCTGAAACAAACCCATACCAGGACGGCAGTTCTCACGGTACGCATGTAGCCGG
TACGATTGCCGCTCTTAATAACTCAATCGGTGTTCTGGGCGTAGCGCCAAGCGCATCATTATATGCAGTAAAAGTG
CTTGATTCAACAGGAAGCGGCCAATATAGCTGGATTATTAACGGCATTGAATGGGCCATTTCCAACAATATGGATG
TTATTAACATGAGCCTTGGCGGACCTTCTGGTTCTACAGCGCTGAAAACAGTCGTTGATAAAGCCGTTTCCAGCGG
TATCGTCGTTGCTGCCGCTGCCGGAAACGAAGGTTCGTCCGGAAGCTCAAGCACAGTCGGCTACCCTGCAAAATA
TCCTTCTACTATTGCGGTAGGTGCGGTAAACAGCAGCAACCAAAGAGCTTCATTCTCAAGCGCAGGTTCTGAGCTT
GATGTGATGGCTCCTGGCGTATCCATCCAAAGCACACTTCCTGGAGGCACTTACGGTGCTTACAACGGCACGTCCA
TGGCGACTCCTCACGTTGCCGGAGCAGCAGCGCTAATTCTTTCTAAGCACCCGACTTGGACAAACGCGCAAGTCCG
TGATCGTTTAGAAAGCACTGCAACATATCTTGGAAACTCTTTCTACTATGGAAAAGGGTTAATCAACGTACAAGCA
GCTGCACAATAA
The encoded aprE protease is:
(SEQ ID NO: 81)
VRSKKIWISLLFALTLIFTMAFSNMSAQAAGKSSTEKKYIVGFKQTMSAMSSAKKKDVISEKGGKVQKQF
KYVNAAAATLDEKAVKELKKDPSVAYVEEDHIAHEYAQSVPYGISQIKAPALHSQGYTGSNVKVAVIDSG
IDSSHPDLNVRGGASFVPSETNPYQDGSSHGTHVAGTIAALNNSIGVLGVAPSASLYAVKVLDSTGSGQY
SWIINGIEWAISNNMDVINMSLGGPSGSTALKTVVDKAVSSGIVVAAAAGNEGSSGSSSTVGYPAKYPST
IAVGAVNSSNQRASFSSAGSELDVMAPGVSIQSTLPGGTYGAYNGTSMATPHVAGAAALILSKHPTWTNA
QVRDRLESTATYLGNSFYYGKGLINVQAAAQ
>mpr
(SEQ ID NO: 82)
ATGAAATTAGTTCCAAGATTCAGAAAACAATGGTTCGCTTACTTAACGGTTTTGTGTTTGGCTTTGGCAGCAGCGG
TATCTTTTGGCGTACCGGCAAAAGCGGCAGAGAACCCGCAAACTTCTGTATCGAATACCGGTAAAGAAGCTGATG
CTACGAAAAACCAAACGTCAAAAGCAGATCAGGTTTCCGCCCCTTATGAGGGAACCGGAAAAACAAGTAAATCGT
TATACGGCGGCCAAACGGAACTGGAAAAAAACATTCAAACCTTACAGCCTTCGAGCATTATCGGAACTGATGAAC
GCACCAGAATCTCCAGCACGACATCTTTTCCATATAGAGCAACCGTTCAACTGTCAATCAAGTATCCCAACACTTCA
AGCACTTATGGATGTACCGGATTTTTAGTCAATCCAAATACAGTCGTCACGGCTGGACATTGTGTGTACAGCCAGG
ATCATGGATGGGCTTCGACGATAACCGCCGCGCCGGGCCGCAATGGTTCGTCATATCCGTACGGTACTTATTCAGG
CACGATGTTTTACTCCGTCAAAGGATGGACGGAAAGCAAAGACACCAACTATGATTACGGAGCTATTAAATTAAAC
GGTTCTCCTGGAAACACGGTTGGCTGGTACGGCTACCGGACTACAAACAGCAGCAGTCCCGTGGGCCTTTCCTCGT
CAGTGACAGGATTCCCGTGTGACAAAACCTTTGGCACGATGTGGTCTGATACAAAGCCGATTCGCTCCGCTGAAAC
GTATAAGCTGACCTATACAACCGATACGTACGGCTGCCAAAGCGGCTCGCCTGTTTATCGAAACTACAGTGATACA
GGGCAGACAGCTATTGCCATTCACACGAACGGAGGATCGTCATATAACTTGGGAACAAGGGTGACGAACGATGT
ATTCAACAATATTCAATATTGGGCAAATCAATAA

Using the ComKS inducible strain described in Example 7 as a basis (this genetically modified strain having increased competence), protease encoding genes such as nprE and vpr were deleted from B. subtilis #105. Deletion of wprA, nprB and aprE genes from B. subtilis #105 was also undertaken.

Protease encoding genes nprE and vpr were deleted from B. subtilis #105 and confirmed by PCR and sequencing (data not shown).

### EXAMPLE 12

### Integration of Nucleic Acids or Expression Casettes Encoding Biomolecules or Heterologous Proteins in the B. Subtilis Strain 105 Genome

A sequence or coding region encoding a desired biomolecule or heterologous protein can be integrated into the chromosome of the genetically-modified microorganism of B. subtilis strain 105. This is an alternative to expression of one or more biomolecule or heterologous protein on a plasmid, which can lead to stability issues and copy number issues that could limit applicability for delivery of the biomolecule or heterologous protein.

One of the strategies to introduce new genes into bacterial host is based on the homologous recombination between identical sequences of double stranded DNA. The frequency of recombination can depend on the length of homology and on host factors. Antibiotic resistance genes are utilized in the construction of integration vectors and in the integration steps to promote and select for recombination events and integration. If plasmid (integration vector) containing two DNA fragments (fragment A and fragment B) which are homologous to some parts of the chromosome (A and B sequences on the chromosome) and a gene of interest (X gene) is placed between these two fragments, a double crossover event will result in the integration of the gene X into the chromosome between fragments A and B. The original DNA sequence of the chromosome between A and B will be substituted for by the X gene. The integration site is determined by the sequences of A and B. The precision of recombination is achieved by pairing complementary strands of DNA from the plasmid and chromosome.

In the case of a single crossover event, the whole plasmid will integrate into either A or B. Then, in the case of the second crossover the plasmid sequences will be eliminated from the chromosome and the X gene will integrate between sequences A and B. Homologous recombination is utilized in step (1) integration of the whole plasmid (integration vector) into the chromosome (single crossover), and then in step (2) a further recombination event removes all foreign DNA including the plasmid replication origins and antibiotic resistance genes from the chromosome (double crossover). The initial integration step (1) can be monitored by gain of antibiotic resistance and the second step removal of plasmid/vector sequences can be monitores by loss of antibiotic resistance. PCR of the target integration site region of the strain chromosome and sequencing across the region is utilized to confirm full and proper integration.

Chromosomal integration can be accomplished with a suicide vector. A suicide vector comprises an origin of replication for replication in E. coli, a drug resistance marker for selection, and an expression cassette flanked by nucleic acids homologous to a specific region of the chromosome.

For chromosomal integration one or more B. subtilis gene may be interrupted by the insertion of the expression cassette (notably this can also be used to inactivate the B. subtilis gene and simultaneously replace it with a gene or nucleic acid encoding a biomolecule or heterologous protein of interest). Heterologous sequences can be integrated in the strain 105 genome. For example, nonessential gene locations can be selected or chosen for integration. Integration can be achieved by replacing the nonessential gene with another sequence of interest, such as a sequence encoding a biothereapeutic molecule, polypeptide, antigen, thereapeutic molecule, immunomodulatory molecule, antibody or fragment thereof including a VHH antibody or nanoboy etc.

Genes suitable as appropriate and applicable integration sites include alpha amylase (amyE), nprE, apr and wprA. The nprE, apr and wprA genes encode proteases and integration at these gene sites to replace the respective genes serves to integrate the heterologous sequence of interest while also inactivating the protease. The gene maps for each of amyE, nprE, apr and wprA on the B. subtilis strain 105 genome are shown in FIG. 2. Native strain 105 sequences for each of nprE, apr and wprA on the B. subtilis strain 105 genome are provided above in Example 11.

Alpha amylase is an enzyme that hydrolyses a bonds of large, α-linked polysaccharides. Deletion or inactivation of the amyE gene encoding alpha amylase in Bacillus subtilis is well tolerated and not detrimental to the growth of the bacteria. Gene integration at the amyE site is utilized and further described and provided herein in the examples. The amyE gene sequence from B sub strain 105 is provided below:

>ELA191105_amyE
(SEQ ID NO: 111)
ATGTTTGCAAAACGATTCAAAACCTCTTTACTGCCGTTATTCGCTGGATTTTTATTGCTGTTTCATTTGGTTCTGGCA
GGACCGGCGGCTGCGAGTGCAGAAACGGCGAACAAATCGAATGAGCTAACAGCACCGTCGATCAAAAGCGGAAC
CATTCTTCATGCATGGAATTGGTCGTTCAATACGTTAAAACATAATATGAAGGATATTCATGATGCAGGATATACA
GCCATTCAGACATCTCCGATTAACCAAGTAAAGGAAGGGAACCAAGGAAATAAAAGCATGTCGAACTGGTACTGG
CTCTATCAGCCGACATCGTACCAAATTGGCAACCGTTACTTAGGAACTGAACAAGAATTTAAAGAAATGTGTGCAG
CCGCTGAAGAATATGGCATAAAGGTCATTGTTGACGCGGTCATCAATCATACCACCAGTGATTATGCCGCGATTTC
CAATGAGGTTAAGAGTATTCCAAACTGGACACATGGAAACACACAAATTAAAAACTGGTCTGATCGATGGGATGT
CACGCAGAATTCATTGCTCGGGCTGTATGACTGGAATACACAAAATACACAAGTACAGTCCTATCTGAAACGGTTC
TTAGAAAGGGCTTTGAATGACGGGGCAGACGGTTTTCGATTTGATGCCGCCAAACATATAGAGCTTCCGGATGAT
GGGAGTTACGGCAGTCAATTTTGGCCGAATATCACAAATACATCTGCAGAGTTCCAATACGGAGAAATCCTGCAG
GATAGTGCCTCCAGAGATGCTGCATATGCGAATTATATGGATGTGACAGCGTCTAACTATGGGCATTCCATAAGGT
CCGCTTTAAAGAATCGCAATCTGGGCGTGTCGAATATCTCCCACTATGCATCTGATGTGTCTGCGGACAAGCTAGT
GACATGGGTAGAGTCGCATGATACGTATGCCAATGATGATGAAGAGTCGACATGGATGAGCGATGATGATATCC
GTTTAGGCTGGGCGGTGATAGCTTCTCGTTCAGGCAGTACGCCTCTTTTCTTTTCCAGACCTGAGGGAGGCGGAAA
TGGTGTGAGATTCCCGGGGAAAAGCCAAATAGGTGATCGCGGGAGTGCTTTATTTGAAGATCAGGCTATCACAGC
GGTCAATAGATTTCACAATGTGATGGCTGGACAGCCTGAGGAGCTCTCGAATCCAAATGGAAACAACCAGATATT
TATGAATCAGCGCGGCTCACATGGCGTTGTGCTGGCAAATGCAGGTTCATCCTCTGTTTCTATCAATACGCCAACA
AAATTGCCTGATGGCAGATATGACAATAAAGCTGGGGCAGGTTCATTTCAAGTGAATGATGGTAAACTGACAGGC
ACGATCAATGCCAGGTCTGTGGCTGTGCTTTATCCTGATGATATTGCAAAAGCGCCTCATGTTTTCCTTGAGAATTA
CAAAACAGGTGTAACACATTCTTTCAATGATCAACTGACGATTACCTTGCGTGCAGATGCGAATACAACAAAAGCC
GTTTATCAAATCAATAATGGACCAGAGACAGCGTTTAAGGATGGAGATCAATTCACAATCGGAAAAGGAGATCCA
TTTGGCAAAACATACACCATCATGTTAAAAGGAACGAACAGTGATGGTGTAACGAGGACCGAGGAATACAGCTTT
GTTAAAAGAGATCCAGCTTCGGCCAAAACCATCGGCTATCAAAATCCGAATCATTGGAGCCAGGTAAATGCTTATA
TCTATAAACATGATGGGGGCGGGGCAATTGAATTGACCGGATCTTGGCCTGGAAAACCAATGACTAAAAATGCAG
ACGGAATTTACACGCTGACGCTACCTGCGGATACGGATACAACCAACGCCAAAGTGATTTTTAATAATGGCAGCGC
CCAAGTGCCTGGCCAGAATCAGCCTGGCTTTGATTACGTGCAAAATGGTTTATATAATGACGCGGGCTTAAGCGGT
TCTCTTCCTCATTGA

One or more biomolecule or heterologous protein may be integrated in the B subtilis 105 strain genome for production or delivery via a modified B subtilis 105 strain. Integration may be at one site or at more than one site in the B subtilis 105 strain genome. For example, a first construct providing one or more first set of one or more biomolecule or heterologous protein may be integrated at a site selected from amyE, nprE, apr and wprA and a second construct providing one or more second set of one or more biomolecule or heterologous protein may be integrated at a site selected from amyE, nprE, apr and wprA. A first construct providing one or more first set of one or more biomolecule or heterologous protein may be integrated at the amyE site and a second construct providing one or more second set of one or more biomolecule or heterologous protein may be integrated at a site selected from nprE, apr and wprA. Other suitable genes and sites for integration are also contemplated and may be selected from one or more native lytic enzymes and/or antibacterial peptides, for example as provided in Example 15.

### Example 13

### Deletion of Native Lytic Enzymes and/or Antibacterial Peptides

B. sutilis strain 105 is modified to delete one or more native lytic enzymes and/or antibacterial peptides. These one or more deletions serve to reduce the genome size of the B. subtilis strain. It also serves to remove potential antibacterial activity of the strains, to the extent that this might be detrimental to their growth or colonization. The reduced genome serves to enable insertion of larger encoding cassettes or heterologous sequence for encoding or producing biomolecules or homologous or heterologous sequences. Also, the reduced genome size can facilitate improved and/or faster or more efficient growth of the bacteria. This further serves to improve expression and production of the biomolecule or heterologous protein of interest by the modified bacteria.

For example, native B. subtilis strain 105 lytic enzymes for deletion include one or more of the following:

>xpf
(SEQ ID NO: 83)
ATGCAAGACTTACTATTTGAATATAAACGAACGCTCAAACAAACAAGAAACCAATATAAACCGCTCGCTG
AGGCAGATGAATCCGTGCTCTCAGCTGAAGAGCTGAAGGATAAAAAAATCATCAGAAATATGATTACTGA
TCTTGAATATGTAACAGAATGGCTTGAAAAAGGAAGGCAGCCCGGCATCAGACGGGCGATTGACCGGCGT
GATGCTTACCAGCGGCTGATGATCAAGGACCCGAGAATCATCGAATCATTTTCCAGCGCTATGATGTTTG
AGCCGGACGGACAGGTATCAGAAGAAGACAGAGATAGAATTCGAGAAGCATTAGCCCTGTTAACGGACAG
AGAAAAGGAAATGTTTTTGCTGCATAAGGTAGAATGTTTTTCTTATGAACGGATCGCCGATCTTCTCGGC
GTAAAAAAATCGACAGTGCAAACGACGATTAAACGGGCGAGTTTAAAGATGCAAAGACAGCAGGAAGACA
TGAATCGATCACTTGCCTGA
>lytC1
(SEQ ID NO: 84)
ATGTTATGGAGGAGCTTGGCGCTGTGCGGATTGGCTTTGACGCTCGCGCCTTGTGCACAGGCAGCAGAGC
CGATAGAAGGGAAAACAGTGTATATTGATGCCGGACACGGCGGTGAAGATAGTGGAGCGGTCGGAAACGG
GCTCTTTGAAAAGGATATTAATTTGGCTGTCTCAGAGCATGTGACAGACAAACTAAAAGAGGAGGTAGCC
AATCCTGTTGCATCCCGGTCTGACGATCATTTTCTGACCTTGGAGGAAAGAGTGGCTAAGGCAAGTGCCA
ATCAGGCTGACCTTTTTGTCAGCATTCATGTGAATTCAGGGGTTGCTTCAGCTTCAGGAACAGAAACGTA
TTTTCAATCTGATTATGAAGGGGAGAACAGCCGGCGTCTCGCATCCGATATTCAGTCAGAGCTCGTTTCT
TCCTTACAAACGAGAGACAGGGGCGTGAAAGAATCAGACTTTTACGTCATTACATATTCACAAATGCCAA
GTGTGTTGGCGGAGCTTGGGTTTATCACGAACAGCTCTGACGCGGACAAGCTGGGAAGTGAAGAATACCA
GCAAAAGGCCGCAGATGCGATTGTCAACGGCATTGATTCTTATTATGATCAGTAG
>lytC2
(SEQ ID NO: 85)
TTGCGTTCTTATATAAAAGTCCTAACAATGTGTTTTCTGGGGCTCATACTTTTTGTGCCAACAGCTTTGG
CCGATAATTCAGTGAAAAGAGTCGGGGGAAGCAATAGATACGGCACTGCTGTACAAATATCAAAGCAAAT
GTATTCAACAGCAAGTACAGCTGTAATTGTTGGTGGGAGTTCCTATGCAGATGCTATTTCAGCAGCACCC
CTTGCTTACCAGAAGAATGCGCCATTGCTTTACACTAATTCTGATAAGCTTTCATATGAAACGAAAACAA
GATTGAAAGAGATGCAGACTAAAAATGTAATTATTGTAGGCGGAACACCTGCTGTTTCATCTAACACTGC
TAACCAGATTAAAAGTTTGGGGATAAGTATTAAACGAATTGCAGGAAGCAACCGTTATGATACGGCTGCA
CGGGTGGCAAAAGCGATGGGTGCGACTTCAAAAGCTGTTATTTTGAATGGCTTCTTATATGCAGACGCTC
CGGCCGTCATTCCTTATGCAGCGAAAAACGGGTATCCAATTCTTTTTACAAATAAAACATCTATAAATAG
TGCGACAACGTCTGTGATAAAAGATAAGGGAATTTCGAGTACCGTTGTTGTAGGAGGCACTGGAAGTATC
AGCAATACGGTATACAACAAGTTACCTTCTCCTACAAGAATTAGTGGTTCAAACAGATATGAGCTTGCTG
CAAATATCGTACAAAAACTTAATTTATCAACAAGCATCGTATATGTAAGCAATGGATTCAGCTACCCTGA
CTCTATTGCAGGAGCTACACTGGCAGCTAAGAAGAAGCAATCTCTTATCCTTACAAATGGTGAAAATTTA
TCTACAGGAGCCCGTAAAATTATTGGAAGTAAAAACATGTCAAACTTTACGATTATCGGAAACACTCCTG
CCGTAAGCACCAAGGTTGCTAATCAGCTAAAGAATCCAGTTGTAGGTGAAACAATCTTTATTGATCCGGG
TCACGGTGATCAAGATTCAGGAGCAATCGGCAATGGACTCCTTGAGAAAGAAGTCAACCTTGATATAGCG
AAAAGAGTCAATACAAAGCTAAATACTTCAGGTGCTCTTCCAGTACTGTCAAGATCTAATGATACTTTTT
ATTCTTTACAGGAGAGAGTAAATAAAGCAGCTTCTGCACAAGCAGATTTATTTATCAGTATACATGCAAA
TGCTAATGATAGCTCATCACCAAATGGAAGTGAGACGTACTACGATACAACATATCAAGCTGCAAATAGC
AAGAGACTGGCTGAACAAATTCAACCAAAGTTAGCGGCTAATCTTGGAACGAGAGACCGGGGAGTAAAAA
CAGCCGCTTTCTATGTTATTAAATATTCTAAAATGCCGAGTGTTTTAGTTGAAACTGCCTTTATCACTAA
TGCATCAGATGCAAGTAAATTGAAGCAAGCGGTTTATAAAGATAAAGCTGCACAAGCTATTCATGACGGC
ACAGTATCTTATTACAGATAA
>sdpC
(SEQ ID NO: 86)
TTGAAGAAGGCGTTTCTAGTATTTTTGTCGGTTGTATTGGTGACGACTGTGTTTTTGGTGAAGCAGCAAG
AAAGTGTGGCACAGGCAAAGCAGCTTGAGTATTCAGGTGAGGAAATTTTTAAAGGCTTTGTGTTTGCCCA
AGGGGAAGTTGGCAAACAGCTTCCAGAGGTCTTTAATAAAGCGATGACAGACAAACTCAACACAAAACAG
GCAAAGGCTTTTGCCAATCAGGTTGTCGCTGATATTAAAAAAGAGGATGCTGACTTTTTTGATAACCTGA
AGAAAGCTGTTTACAGCAAAGATGCATTGAAAGTGGATGAACTGCTGAAAAAAGCAGGTCAAATCGTTGA
AGAAAAAGTAGAAGCTGCAAAGGAAATCGCCGCTTCTAAGGATGATACATCCAGAGTTCAGGCCGAGCTT
GTCAACACTGTTGATACCGCTAACTATTTCTATTATGTTTCTTATGTTGCTGCAGCAGGTGCCCTGATTC
TGATCATTCTCGCCATTGATATTACGCCGATTGCGATTTCAGACAATGTGGATCGCGAAATGGCAATCAG
AACGCTGGTTGATGAATTAAACTAG

### EXAMPLE 14

### Production of Gamma Polyglutamic Acid

Gamma polyglutamic acid (poly-γ-glutamic acid; (γ-PGA) is a naturally occurring biopolymer made from repeating units of L-glutamic acid, D-glutamic acid, or both. Since some bacteria are capable of vigorous γ-PGA biosynthesis from renewable biomass, γ-PGA is considered a promising bio-based chemical and is already widely used in the food, medical, and wastewater industries due to its biodegradable, non-toxic, and non-immunogenic properties. As a biodegradable, water-soluble, edible, and non-toxic biopolymer, γ-PGA and its derivatives can be used safely in a wide range of applications including as thickeners, humectants, bitterness-relieving agents, cryoprotectants, sustained release materials, drug carriers, heavy metal absorbers, and animal feed additives. Peptidoglycan bound γ-PGA may protect bacterial cells against phage infections and prevent antibodies from gaining access to the bacterium. Also, γ-PGA can be utilized as an oral therapeutic for diabetes in dogs and cats. Dietary γ-PGA has been shown to have plasma glucose-lowering effects.

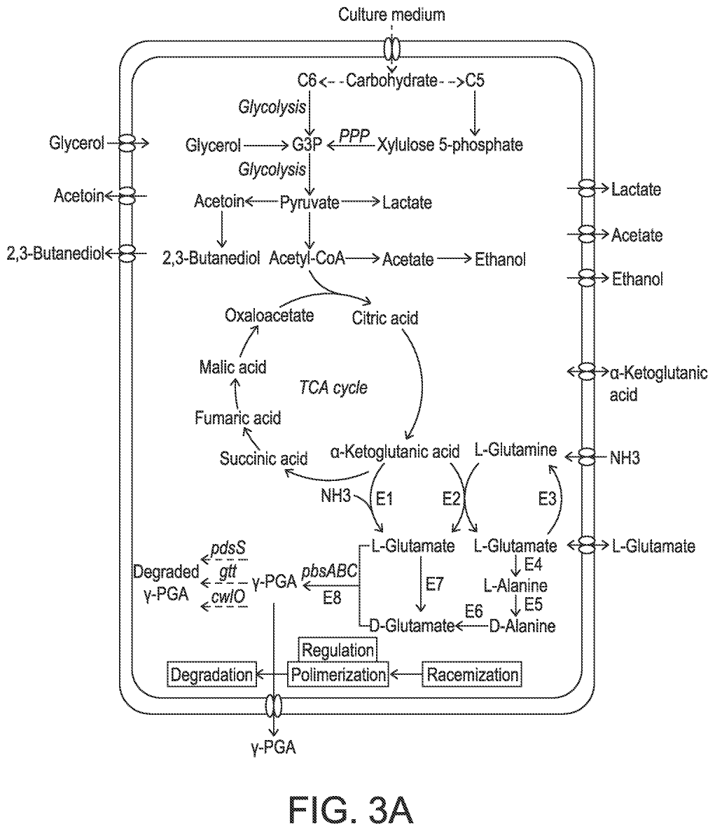

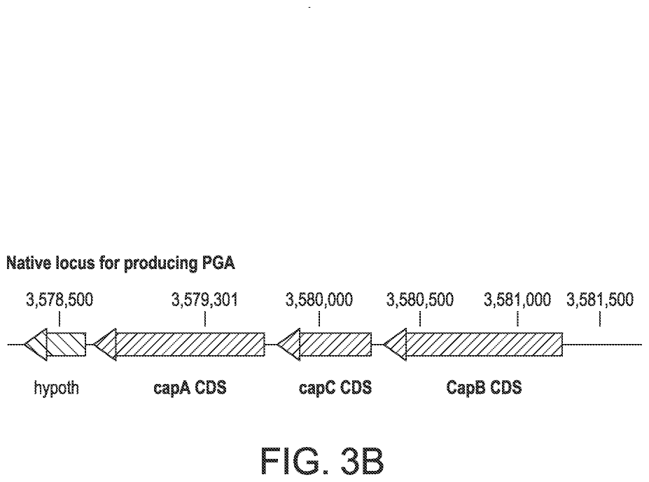

FIG. 3A depicts the pathway for poly-γ-glutamate biosynthesis. The B. subtilis strain 105 native locus for producing PGA is shown in FIG. 3B. The native B. subtilis locus comprises capC, capB and capA encoded from a single promoter. The CapABC locus sequence is as follows:

CapABC locus sequence
(SEQ ID NO: 87)
GCTGGGCAAATAAAAAGAGAGTGTCTCCTCAGACACTCTCTTTTTTGAATTATTTATTGGCGTTTACCGG
TTCTTCCTGCTGCTGGATTTTATTCATTTCCATATCAATCTTCTGTTTTTCCTGATCTGTCAGCTGATCA
TTGAATTTAAAAGCTGACATAAACATGAACACGATGGCTACGGCAACAAACGGCCAGATGGCTTTGACAA
ATTTCATCTTTATCACTCCGTTTATTTAGATTTTAGTTTGTCACTATGATCAATATCAAACGTCAGTTTT
CCGTCTTCTACTTTCCAAGCGAAATTAGAGTCTTTCGTCAGTTCGCGAATAATGGTTTTCTGTTTAAGGC
TGTCTTTTTTCACAGGTGCAGGTGTCGCTTCATGGATATCGATCGGTGTCACTTCAAAGCGGCCTGTTCC
ATTTTTCTTCAGGTGATACTGAACCAGTGCACTGTCTCTTGTTCTCGTCCAGCCTTGGTCAAAGACAAAG
TTGCCGAGGCTGTAGAAAATGACGGTTCCGTTATATACTTCAATCGGTTCTAAGACGTGCGGATGATGGC
CGACGATGATGTCAGCTCCCGCATCAGACATGGCTCTTGCAAGCTGGCGCTGGCGGTCGTTTGGATCATT
GTCATACTCTTGGCCCCAGTGTGACTGCACAACAACGATGTCCGCATGTTTTTTCGCTTCTGAAATCATA
GGGATGAAGATTTCAGGATCTGCGGGCAGCACGCCCGGCGTATTCTTTTTAGCCGCGAAACCTTTCCCGG
ACACATCGGTAAAGCCAAGCGTTGCAATTGTTACCCCGTTGACTTTCTGGTACGAAATTTTCTTTTTCGC
ATCACTTAAGCTGTATCCCGCTCCAACGATATCAAGGTTTTGCTTCGCAAATTCTCCAAGCGTATCTTTC
ATGCCCTGAACGCCGTAATCCATTGCGTGGTTGTTTGCGCTGTTGAGAACTGTGAAATTCATGTCCTTCA
AGACTTTCACTGATTCCTTATTCGTCTGCAGATGAATCTCTTTATCTGCTTGTTTATAATTCTTTTGATA
GGTTACCGGGTTTTCAAAGTTTCCTGCTACATAATCCGAAGCTCTAAAGATCGGTTCAACATATTGAAAA
ATACTGTCTGCCCCTTTTTGCTCCGTTACTTTTTCAACATAGCGTCCCATCATAATATCGCCTACAAATG
AGGCTGAAAGTACGTCGTCAGAATACGTTTTGACCTTCGGCGTTTCCGCTTTTCCCGCCCACATGAAAGC
GAACATAAGGACAAAAACGATCGGAATGGCAATAAATACGTGCTTATTGGTTTTCTTTTTTTGCTGTTTT
GTCAGCTTTAGCAGCTTTTCATGAAAGCTCAGTTCTTTTTTCATCGTTTGACACACCTTACATTAAATTA
AGTAGTAAACAAACATGATAGCAAAGGTCGCTCCGCTCAATAGCAGCGTGCTTCCGAACGTAATGGTTAA
ACCTTGTTTCTGAATGGTATTGGCAATTAAACCTGGCACGATGATGCCGATTCCTCGAAATTCTGCGATT
TCAAATGGTACAATCGGGTATAGAAAATCAAACGCGATTTTTAGGACGATCCCTGTTATCAGCATGGCAG
CGAATTTTCTGCGTCCGTACAAAATCATAAATTTGGATAAACCGTATTTCACGATAACATAAGTGAGCAA
GCTCACTAGCAAAACAAGTAAAATAAAGACCGGCTGATTAAACACAAGTCCTAAATATCCCGGTACAACA
AGTCCTGCCGGCACGATCCCTGTTTTTTCCGCAAAAATTAAACTGAGTAGTACACCTAAAATTAGTGCGA
TGTATAAATCTGATCCGAACATGTTTGCATTTCCCCCTAGCTTACGAGCTGCTTAACCTTGTATTCGTGG
ATTTTTTCAATTAAAGGCTCTGCGGCACCATGAATATTGCCGACGCCATATATGACACGGTTGTGCATTC
TTTTCTTTAACAATTCCATAATTTCATCTGTTGACTTATACTCTAGATCATGCAGTTTGTCTGCAGGAAT
TTTGCCTTCTTCGTAGGCTTTTACGACCGGTTCTGTTGTTTCACCGATTAAGATCAGTTCACTTGCTTCA
ATATAAGGCAATACGTCATTTGCGAATTGCTGTGTCCGATCGACACGGTCTGCGCGGCAGTTCATGATGA
TGATCGGATCATCGGTCGGGTAACCGATTTCTTTTACACGTTTCCATATATTCAAAGTAGAAGAAGCGTC
GTTTGCGGCAAACCCATTAACAAAGTGCCCAGGCTCGCTCGGACTGATCAGCGGAAGAATTCTCATTGCT
CCCGGATCTGGCGGCGCATTCAGCATTCCCTTAAATGCTGTTTCTTCGTCAATGCCGAGTGCTTGAGCCA
CACCCAGCGCCAGAGAAGCGTTATCAGGGAATACCATGTATTCAAATTTACGTAAATACTCATCTGTAAT
TTTTGAGTTATCAGCAATGATGACTTTTGTGTTTCGTTCTTTTGCTTTTTGTTTAAAGAACTCGGTATAT
TCACTATCTGTAATGACAAGATGGCCATTATAAGGAATTGTAGCAGTAAACGCTTCTGCAATTTCATCAA
GCGTCGGCCCCATGACATCCATATGGTCTTCCAAAACATTCACAATGACGCCGATATTGGCCTGCAGAAG
TTCTTCCTGAAAGATGATTTGATAATCTGGGTTAACAGCCATGCATTCACTGACAATCGCGTTAGCCCCT
CTTTCTACTGTTTCTCTCATGACTTCTTTTTGCTCTCCGATATTCGGCCCCTGAGGTTTCCGTTTAATCG
GCTTTTCCTCCGGTGTGTCCCAGTAAATCATTCTTGCATCTGTTCCTGTTGTTTTTCCAACAGTCTTGTA
ACCGGCTTCTATTAATATTCCGGTTGTCAGCCTTGTCACAGTCGATTTTCCGCGGATGCCGTTAATATTC
ACCCGAACAGGGAGGGCATCAATGTTTTTCTGATGTCGTCGTTTTTCTAATATTCCGATGACCAGTATGA
CAGCACAGGCTATAATGAGTAACCACATTGCTTTTCGACATCTCCTTCTATATTGTTGTAAATTCATTCA
TCAGTATATAGAATCACTATGAATTCTCAATCGGCATTATGTAACATAATCTTCCCTAATCGACTAAGCC
AAACTTCTCTTTTGAAAGAGATTTTATACCTACAAACTGTTTTCTATTTTTGGAAAACAGCAGGACTTAT
ACCTTATCACTTTTTTAAAAAATTGAAGAGAATGCGCTTTCATTTTTCTCCTGATATCCTTCTGGACGTA
TAGTCACACCCTGCTGTTTGTATTTAATATGCATTTCAAATCAATCTTTTTTTCAGCCTGTGTTATCAAG
CATTTCCTGCTATCATGCGTGTTTCCGGTTCTTATGAACACAAAATAATTTTATCCTGTTTCTTAAGGAC
AAGGTCATAATGTCATCCTTTCTGCATGGAATAAGCAATATGAGGCCCATTCCAAAATGACTAAGGAGGT
TCGAGATGAAAACGCTAAACATACGAGTCCGAATTCTCTAACATAATTAAACATTTTCTGGGATGATAGT
CTTTTCTGTTTCTCACCATTTACAGGTCTAAACGCATGA

B. subtilis strain 105 is modified to produce increased amounts of poly-γ-glutamate. B. subtilis strain 105 is modified to produce inducible poly-γ-glutamate. Strain 105 capABC locus is modified to add an inducible promoter in place of the native promoter. The strain 105 capABC locus is modified to replace the native promoter with one or more promoter, including tandem promoters. Exemplary promoters are provided in Example 10. In an alternative approach, an additional capABC locus, including with an alternative, inducible or tandem promoters to provide enhanced or increased or inducible production of the capABC locus encoded proteins is integrated in strain 105 genome sequence.

Genes suitable as appropriate and applicable integration sites include amyE, nprE, apr and wprA. The nprE, apr and wprA genes encode proteases and integration at these gene sites to replace the respective genes serves to integrate the heterologous sequence of interest while also inactivating the protease. The gene maps for each of amyE, nprE, apr and wprA on the B. subtilis strain 105 genome are shown in FIG. 2. Native strain 105 sequences for each of nprE, apr and wprA on the B. subtilis strain 105 genome are provided above in Example 11. The amyE sequence is provided in Example 12 and as SEQ ID NO: 111.

### EXAMPLE 15

### Production of Other Biomolecules and/or Heterologous Proteins

Native B. subtilis strain 105 is genetically modified to express a number of biomolecules and heterologous proteins. Several classes of biolomelcules and heterologous proteins are provided and described below.

### I. Lysins

The desired biomolecule may be a biomolecule with anti-infective activity. The anti-infective activity could be lysis of pathogenic bacteria by a lytic enzyme, for example from a bacteriophage, with specificity to a certain genus of pathogenic bacteria. Suitable and exemplary lysins are known to one skilled in the art and available. Phage associated lytic enzymes have been identified and cloned from various bacteriophages, each shown to be effective in killing specific bacterial strains. U.S. Pat. Nos. 7,402,309, 7,638,600 and published PCT Application WO2008/018854 provides distinct phage-associated lytic enzymes useful as antibacterial agents for treatment or reduction of Bacillus anthracis infections. U.S. Pat. No. 7,569,223 describes lytic enzymes for Streptococcus pneumoniae. Lysin useful for Enterococcus (E. faecalis and E. faecium, including vancomycin resistant strains) are described in U.S. Pat. No. 7,582,291. Lysins are unique in that they are generally bacterial species specific and do not effect or kill normal gut etc bacterial flora, thus it is likely that the normal flora will remain essentially intact (M. J. Loessner et al (1995) Mol Microbiol 16, 1231-41). Targeting bacterial pathogens that colonize the gastrointestinal tract with Bacillus strain 105 modified to produce one or more lysins directed against these gut or intestinal tract pathogens is an application of the system and methods.

Lytic enzymes for expression by genetically modified B. subtilis strain 105 may include PlyCM, a lytic enzyme targeting Clostridium perfringens, encoded by a sequence of: (SEQ ID NO: 88)

| | |
|---|---|
| 1 | ATGGAAAGCC GTAATAACAA TAACCTGAAG GGCATCGATG TGAGCAACTG GAAGGGCAAC |
| 61 | ATCAATTTTC AAAGCGTCAA AAATGACGGT GTTGAAGTTG TTTACATTAA GGCAACCGAA |
| 121 | GGCAACTACT TCAAAGACAA ATATGCTAAG CAAAACTACG AGCGCGCTAA AGAACAGGGT |
| 181 | CTGCGTGTGG GCTTCTACCA CTTTTTCCGC GCAAACAAAG GTGCCAAAGA TCAGGCGAAC |
| 241 | TTCTTCGTGA ATTACCTGAA CGAAATCGGT GCGGTCAATT ATGACTGTAA ACTGGCACTG |
| 301 | GACATCGAGA CTACCGAAGG CGTCGGTGCG CGTGACCTGA CCTCTATGTG CATCGAGTTC |
| 361 | CTGGAAGAGG TGAAGCGTAT TACGGGTAAG GAAGTTGTCG TGTACACCTA TACCAGCTTC |
| 421 | GCGAACAATA ATCTGGATTC CCGTCTGTCT AGCTATCCGG TGTGGATTGC GCACTATGGC |
| 481 | GTCAACACCC CGGGTGCGAA CAATATCTGG AGCGAGTGGG TGGGTTTCCA GTACAGCGAG |
| 541 | AATGGCTCCG TCGCCGGTGT CAGCGGTGGC TGCGATATGA ACGAATTTAC CAATGGTATC |
| 601 | TTTATTGACT CGAACAATTT CACGTTGGAC AATGCAACGA CCAAAAATGT TAGCATTAAG |
| 661 | CTGAACATTC GCGCCAAGGG TACGACCAAC AGCAAAGTTA TTGGTAGCAT TCCGGCGAAC |
| 721 | GAAAAGTTTA AGATCAAATG GGTTGATGAA GATTACCTGG GTTGGTATTA CGTTGAGTAT |
| 781 | AACGGTATCG TGGGTTACGT TAACGCCGAT TACGTCGAGA AACTGCAAAT GGCGACCACG |
| 841 | CATAATGTTA GCACCTTTCT GAATGTACGC GAGGAGGGTT CCTTGAATAG CCGTATTGTG |
| 901 | GACAAGATCA ACACTGGCGA CATCTTTCGT ATTGACTGGG TTGATAGCGA TTTCATTGGT |
| 961 | TGGTATCGTG TGACGACGAA AAACGGCAAG GTCGGCTTTG TTAATGCAGA GITTGTGAAA |
| 1021 | AAGTTGTAA |

CP025C, a lytic enzyme targeting Clostridium perfringens, encoded by a sequence of: (SEQ ID NO: 89)

| | |
|---|---|
| 1 | ATGTCGAAGA TTTTTGGTTT AGATGCGGGT CATTGTACGA GCGGCGCAGA TACGGGTGCG |
| 61 | CAGGGCAATG GTTACAAAGA ACAAGACTTG ACCCGTCAAG TTGTTACCTA TCTGAGCGAA |
| 121 | TACTTGGAGA AAGAGGGCCA CACTACCAAG TACTGCCATT GCAATAGCGC GAGCACGGTT |
| 181 | AACGAATCCC TGCGCTATCG TGTGAACAAA GCCAACTCCA TCGGTGTCGA CTACTTCGTG |
| 241 | AGCATCCACC TGAACGCCGG TGGCGGCGTT GGTACCGAAA CGTACATCTG CGCGCGTGGC |
| 301 | GGCGAGGCCG AGCGCGTGGC GAAACGCGTC AATTCTAAAC TGGTGCAGTA CGGTTATCGT |
| 361 | GACCGTGGTG TCAAGGTTGG TAATCTGTAT GTGATTAAGA ACACCAATGC ACCGGCTATC |
| 421 | CTGGTTGAGA TCTGTTTCAT TGACAGCAGC AGCGATGTGG CAAAGTTTAA CGCGAAGGCA |
| 481 | ATCGCGAAAG CGATTGCTGA GGGTCTGCTG GATAAAACCA TTGGTGAAGT CGAGAATAAG |
| 541 | TAA |

Lysostaphin is an antimicrobial lytic peptide originally isolated from Staphylococcus simulans and function as a bacteriocin (bacterial killing) against various bacteria, particularly Staplyococcus bacteria (Kumar, J. K. (2008) Appl. Microbiol. Biotechnol. 80:555-561.; do Carmo de Freire Bastos, M et al (2010) Pharmaceuticals 3: 39-1161; doi:10.3390/ph3041139). The cell-wall degrading activity of lysostaphin is primarily due to a glycylglycine endopeptidase activity, which lyses many staphylococcal strains. Like many lysins and antimicrobial lytic peptides, the lysostaphin molecule consists of two distinct domains: (i) an N-terminal peptidase domain responsible for the catalytic activity of the protein and (ii) a C-terminal targeting domain (CWT) involved in binding to the peptidoglycan substrate. The C-terminal 92 amino acid residues of lysostaphin are dispensable for enzymatic activity but necessary and sufficient for directing lysostaphin to the cell wall envelope of S. aureus. The amino acid sequence of mature lysostaphin is as follows:

(SEQ ID NO: 90)
AATHEHSAQWLNNYKKGYGYGPYPLGINGGMHYGVDFFMNIGTPVKAIS
SGKIVEAGWSNYGGGNQIGLIENDGVHRQWYMHLSKYNVKVGDYV
KAGQIIGWSGSTGYSTAPHLHFQRMVNSFSNSTAQDPMPFLKS
AGYGKAGGTVTPTPNTGWKINKYGTLYKSESASFTPNTDIITR
TTGPFRSMPQSGVLKAGQTIHYDEVMKQDGHVWVGYTGNSG
QRIYLPVRTWNKSTNTLGVLWGTIK

### II. Antibacterial Peptides

Numerous peptides having inherent antibacterial activity have been described. Antimicrobial peptides (AMPs) present an alternative to classical antibiotics. Bacteriocins are a group of antimicrobial peptides produced by bacteria, capable of controlling clinically relevant susceptible and drug-resistant bacteria. Bacteriocins are proteinaceous or peptidic toxins produced by bacteria to inhibit the growth of similar or closely related bacterial strain. They are structurally, functionally. and ecologically diverse. A wide range of antimicrobial peptides is secreted in plants and animals to challenge attack by foreign viruses, bacteria or fungi (Boman, H. G. (2003) J. Intern. Med. 254 (3):197-215). These form part of the innate immune response to infection, which is short term and fast acting relative to humoral immunity.

AMPs have been found in all kingdoms of life, not just in bacteria, are part of the innate immunity and represent the first line of defense in an infection (Zasloff M. (2002) Nature 415: 389-95). Despite their diversity in origin and sequence, many AMPs, particularly cationic antimicrobial peptides, generally have a substantial proportion of hydrophobic amino acids (=>30%), an overall positive charge (+2 to +11), and are relatively short consisting of 10-50 amino acids (Hancock R E W, Sahl H-G (2006) Nat Biotech 24:1551-1557). Protamines or polycationic amino acid peptides containing combinations of one or more recurring units of cationic amino acids, such as arginine (R), tryptophan (W), lysine (K), even synthetic polyarginine, polytryptophan, polylysine, have been shown to be capable of killing microbial cells. These peptides cross the plasma membrane to facilitate uptake of various biopolymers or small molecules (Mitchell D J et al (2002) J Peptide Res 56(5):318-325). Based on these properties, AMPs are able to fold into amphiphilic three-dimensional structures and are often based on their secondary structure categorized into α-helical, β-sheet or peptides with extended/random coil structure. Most of the so far characterized AMPs belong to the family of the α-helical or β-sheet peptides (Takahashi D, Shukla S K, Prakash O, Zhang G. (2010) Biochimie pp. 1236±1241; Nguyen L T, Haney E F, Vogel H J. (2011) Trends in Biotechnology pp. 464-472.

### A. Mersacidin

Mersacidin is a peptide having antibacterial activity and is a bacteriocin. Some mersacidin peptides have been identified and characterized from Lactobacillus, particularly LactoBacillus reuteri. The probiotic and direct feed microbials L reuteri strains 3630 and 3632, and mersacidin peptides therefrom, have been described and detailed in WO 2020/163398, published Aug. 13, 2020, US 2022/0088094 published Mar. 24, 2022, and US 2022/0125860 published Apr. 28, 2022.

B subtilis strain 105 is modified to produce an antibacterial peptide mersacidin, partiocularly mersacidin identified from L. reuteri strain 3632. Nucleic acid encoding a mersacidin (mersacidin-E1) is: (SEQ ID NO: 91)

| | |
|---|---|
| 1 | atggacaaag aagaattaga aaaaattgta ggtaataact ttgaggaaat gagtttacaa |
| 61 | aaaatgacag aaattcaagg tatgggtgaa taccaagtgg attcaacacc agcagcttct |
| 121 | gcgatttcac gggcaacaat tcaagtatca cgtgcatctt ctggaaaatg tctaagttgg |
| 181 | ggtagtggtg cagcatttag tgcttatttt actcataaaa gatggtgcta g |

This nucleic acid encodes a polypeptide: (SEQ ID NO: 92)

MDKEELEKIVGNNFEEMSLQKMTEIQGMGEYQVDSTPAASAISRATIQV
SRASSGKCLSWGSGAAFSAYFTHKRWC

Nucleic acid encoding another mersacidin (mersacidin-E2) is: (SEQ ID NO: 93)

| | |
|---|---|
| 1 | atggaagaaa aagaattaga aggtgtaata gggaattcgt ttgaaagtat gactgtagag |
| 61 | gaaatgacaa aaattcaagg tatgggtgaa tatcaagtag attcgacgcc tggatatttt |
| 121 | atggaaagtg ctgccttttc agctcttaca gccaatataa caagacatgc tatgcatcat |
| 181 | cattaa |

This nucleic acid encodes a polypeptide: (SEQ ID NO: 94)

MEEKELEGVIGNSFESMTVEEMTKIQGMGEYQVDSTPGYFMESAAFSAL
TANITRHAMHHH

### B. Cathelicidins AND CAP18

Cathelicidins represent a novel family of gene-encoded antimicrobial peptides in vertebrates, and play key roles in host immune response to microbial infections (Reddy, K et al (2004) Int J Antimicrob Agents 24:536-547, doi:10.1016/j.ijantimicag.2004.09.005). Due to their potent antimicrobial activities and bacterial resistance, cathelicidin-derived peptides are regarded as potential alternatives to traditional antibiotics (Hancock R E and Sahl H G (2006) Nat Biotechnol 24:1551-1557, doi:10.1038/nbt1267). They usually possess a broad spectrum antimicrobial activity against bacteria including clinical isolated drug-resistant strains, enveloped viruses, fungi, and even parasites (Giacometti, A et al (2003) J Antimicrob Chemother 51:843-847, doi:10.1093/jac/dkg149; Rapala-Kozik M et al (2015) Infect Immun 83:2518-2530, doi:10.1128/IAI.00023-15; Tripathi S et al (2014) J Leukoc Biol 96:931-938, doi:10.1189/jlb.4A1113-604RR)

Cathelicidins are generally characterized by a N-terminal signal peptide, a highly conserved cathelin domain followed by a C-terminal mature peptide with remarkable structural variety (Zanetti, M et al (2000) Adv Exp Med Biol 479:203-218, doi:10.1007/b112037). Most cathelicidins display hydrophobic and cationic traits, which bestow these small peptides a unique antimicrobial mechanism different from the traditional antibiotics, that is, cathelicidins readily adhere to the negatively charged bacterial membranes and form a lipophilic anchor inducing membrane disruption and cell death within several minutes, limiting the opportunity for development of drug resistance through bacterial gene mutation (Reddy, K et al (2004) Int J Antimicrob Agents 24:536-547, doi:10.1016/j.ijantimicag.2004.09.005; Ling G et al (2014) PloS ONE 9,e93216, doi:10.1371/journal.pone.0093216)

More recent evidence suggests that in addition to their antimicrobial effect, cathelicidins also possess anti-inflammatory activities in the process of pathogen infections (Bowdish, D M et al (2005) J Leukocyte Biol 77:451-459, doi:10.1189/jlb.0704380; Finlay B B and Hancock R E (2004) Nat Rev Microbiol 2:497-504, doi:10.1038/nrmicro908). Cathelicidin-derived peptides have great potential to be exploited as medical coating materials and antimicrobial agents for controlling various infections (Ong Z Y et al (2013) Adv Funct Mater 23:3682-3692, doi:10.1002/marc.201300538; Shukla A et al (2010) Biomaterials 31:2348-2357, doi:10.1016/j.biomaterials.2009.11.082).

A cathelicidin (Hc-CATH) showing potent bactericidal activity from the sea snake, Hydrophis cyanocinctus, has been described, consisting of 30 residues and mainly adopting an alpha-helical conformation (Wei L et al (2015) J Biol Chem 290:16633-16652, doi:10.1074/jbc.M115.642645). Peptide variants and hybrid peptides of the Hc-CATH have been described (Yu H et al (2017) Nature Scientific Reports 7:2600; DOI:10.1038/s41598-017-02050-2).

CAP18, originally isolated from rabbit neutrophils, demonstrates antimicrobial activity against a broad range of pathogenic bacteria, is highly thermostable and showed no hemolytic activity in vitro (Ebbensgaard A, Mordhorst H, Overgaard M T, Nielsen C G, Aarestrup F M, Hansen E B. (2015) PLoS One 10:e0144611). In addition, a recent study evaluated a potential therapeutic effect of CAP18 against red mouth disease caused by Y. ruckeri in juvenile rainbow trout either by oral administration or intraperitoneal injection, and injection of CAP18 into juvenile rainbow trout before exposure to Y. ruckeri was associated with lower mortality compared to non-treated fish (Chettri J K, Mehrdana F, Hansen E B, Ebbensgaard A, Overgaard M T, Lauritsen A H, et al. (2017) J Fish Dis. 40:97±104). CAP18 has the potential to act as lead peptide for further development and optimization.

Antibacterial activity of the cathelicidin, cationic antimicrobial protein of 18 kDa (CAP18), was originally isolated from rabbit granulocytes. The C-terminal 37 amino acids of rabbit CAP18 make up the lipopolysaccharide-binding domain and synthetic CAP18₁₀₆₋₁₄₂ has been shown to have broad antimicrobial activity against both gram-positive and gram-negative bacteria, including Staphylococcus aureus, Streptococcus pneumoniae, Escherichia coli, Pseudomonas aeruginosa and Salmonella typhimurium (Larrick J W et al (1993) Antimicrobial Agents Chemotherapy 37(12):2534-2539).

The rabbit CAP8 37 amino acid peptide has the sequence: (SEQ ID NO: 95)

GLRKRLRKFRNKIKEKLKKIGQKIQGLLPKLAPRTDY

Antimicrobial activity of human CAP18 peptides has been assessed (Larrick J W et al (1995) Immunotechnology 1:65-72). Human CAP 18, or cathelicidin peptide, is also denoted LL37 and has been shown to modulate immunity during bacterial infections by recruiting neutrophils, monocytes and T-cells (Ciornei C D et al (2005) Agents Chemother 49:2845-2850, doi:10.1128/AAC.49.7.2845-2850.2005; De Y et al (2000) J Exp Med 192:1069-1074, doi:10.1084/jem.192.7.1069). The Human CAP18 peptide LL37 has the following sequence: LLGDFFRKSKEKIGKEFKRIVQRIKDFLRNLVPRTES (SEQ ID NO: 96)

Other antimicrobial cathelicidins of different species include BMAP28 (CATHL5; bovine), Bac7 (CATHL3; bovine rumen), k9Cath (canine) and PMAP36 (porcine).

### III. Antibodies—Including Single Chain or Single Domain Antibodies

Nanobodies (also denoted VHH) are small, low molecular weight, single-domain, heavy-chain only antibody found in camelids. Owing to its smaller size, genes of these proteins are easy to clone inside a plasmid. Therefore, by using molecular cloning techniques, nanobodies against various antigens can be presented in the systemic circulation. B. subtilis bacteria strain 105 is modified to include a heterologous coding region encoding a desired biomolecule which can be a nanobody, or can encode one or more nanobodies. The desired biomolecule may be a biomolecule with anti-infective activity. The anti-infective activity can be inhibition or neutralization of toxins produced by pathogens. The inhibition or neutralization can be accomplished with single chain antibodies.

For example, LactoBacillus has been described as an expression system for single chain antibodies directed against host attachment factors (WO2012/019054). The L reuteri strains 3630 and 3632 are described and detailed as probiotic strains in WO 2020/163398 published Aug. 13, 2020, and in corresponding US 2022/0088094 published Mar. 24, 2022 and US 2022/0125860 published Apr. 28, 2022. A live delivery system based on L reuteri strain 3630 or 3632 is described and detailed in PCT/US2020/016522 filed Feb. 4, 2020, published as WO 2020/163284 Aug. 13, 2020. This application describes native bacterial promoters, signal sequences suitable for expression and vectors and bacterial genome sites/genes for integration to generate stable modified strains. Recombinant LactoBacillus (L reuteri strain 3630 and L reuteri strain 3632) delivering nanobodies directed against Clostridium perfringes NetB and alpha toxin have been described and shown to confer protection against necrotic enteritis in poultry (Gangaiah D et al MicrobiologyOpen 2022; 11:e1270,doi.org/10.1002/mbo3.1270).

### A. Clostridium Perfringes Toxin Antibodies

Toxins to be targeted by single chain antibodies include Clostridium perfringens alpha toxin and NetB. Camelid heavy-chain only (VHH) antibodies against C. perfringens alpha toxin and NetB are generated. Briefly, two llama calves each are immunized with either recombinant alpha toxin or NetB variant W262A. Neither of these immunogens are haemolytic. The immunized llamas are boosted twice with toxin peptides. On days 44 and 72 after the primary immunization, blood samples are taken and RNA isolated for phage library construction. Phage libraries are screened for binding activity towards each of the two toxins. The candidate antibodies are sequenced and further screened in bioassays.

### Alpha Toxin Antibodies

Alpha toxin causes membrane damage to a variety of erythrocytes and cultured cells. It is preferentially active towards phosphatidylcholine (PC or lecithin) and sphingomyelin (SM), two major components of the outer leaflet of eukaryotic cell membranes. The N-terminal domain possesses full activity towards phosphatidylcholine but lacks the sphingomyelinase activity and is not haemolytic or cytotoxic. The C-terminal domain is devoid of enzymatic activity, but interaction between the N- and C-terminal domain is essential to confer sphingomyelinase activity, haemolytic activity and cytotoxicity to the toxin. Although alpha toxin is a potent haemolysin, the lysis of erythrocytes is only seen after intravenous administration of toxin in experimental animals or in cases of clostridial septicaemia.

The inhibitory capacity of the VHH antibodies directed towards alpha toxin on the alpha toxin lecithinase activity is determined by measuring its effect on egg yolk lipoproteins. Fresh egg yolk is centrifuged (10,000×g for 20 min at 4° C.) and diluted 1:10 in PBS. The ability of the VHHs to neutralize the alpha toxin activity is assessed by pre-incubating a two-fold dilution series of the VHHs (two wells per dilution, 5 μM starting concentration) with a constant amount of alpha toxin (either 5 μg/ml recombinant alpha toxin or 3.33×10⁻⁴U/μl alpha toxin from Sigma, P7633) for 30 minutes at 37° C. prior to the addition of 10% egg yolk emulsion. As a positive control, serum from calves immunized with the recombinant alpha toxin is used, starting from a 1:4 dilution. After incubation at 37° C. for 1 hour, the absorbance at 650 nm (A₆₅₀) was determined. Alpha toxin activity is indicated by the development of turbidity which results in an increase in absorbance.

Control serum is able to neutralize the lecithinase activity of both the commercial and the recombinant alpha toxin. An eight-fold dilution of the antiserum (corresponding to 3.12% serum) is able to completely neutralize the alpha toxin lectihinase activity of the recombinant alpha toxin, whereas only the highest dilution of the antiserum (corresponding to 25% serum) is able to completely neutralize the lecithinase activity of the commercial alpha toxin. Difference in inhibitory capacity is observed between five candidate VHH antibodies. VHH EAT-1F3 had no effect on the lecithinase activity of either of the alpha toxins. The neutralizing capacity of EAT-1A2 and EAT-1C8 is very similar and is the same for both the recombinant and commercial alpha toxin. The maximal inhibitory capacity is preserved until a 32-fold dilution (0.16 μM VHH) of the VHHs. However, both EAT-1A2 and EAT-1C8 are unable to completely neutralize the lecithinase activity, resulting in 40% to 50% residual lecithinase activity. Two other VHHs, EAT-1F2 and EAT-1G4 show a difference in neutralizing capacity towards the recombinant and the commercial alpha toxin. EAT-1F2 has a high neutralizing capacity towards the recombinant alpha toxin but is unable to completely neutralize the commercial alpha toxin, resulting in about 25% residual lecithinase activity. In contrast to EAT-1F2, EAT-1G4 neutralizes 100% of the lecithinase activity of the commercial alpha toxin, but is less capable of neutralizing the recombinant alpha toxin.

Neutralization of the alpha toxin haemolytic activity by the VHH antibodies directed towards alpha toxin is determined by measuring its effect on sheep erythrocytes. Similar to the inhibition of the alpha toxin lecithinase activity, the ability to neutralize the haemolytic activity is assessed by pre-incubating a two-fold dilution series of the VHH antibodies (two wells per dilution, 5 μM starting concentration) with a constant amount of alpha toxin (6.25×10⁻⁵ U/μl alpha toxin from Sigma, P7633) for 30 minutes at 37° C. prior to the addition of 1% sheep erythrocytes. As a positive control, serum from calves immunized with the recombinant alpha toxin is used, starting from a 1:4 dilution. After incubation at 37° C. for 1 hour, the plates are centrifuged to pellet intact red blood cells. The supernatant is transferred to a new 96 well plate and the A₅₅₀ is determined. Alpha toxin activity is indicated by the increase in absorbance due to release of haemoglobin from the erythrocytes.

The inhibitory capacity of the VHH antibodies towards the alpha toxin haemolytic activity is determined using the commercial alpha toxin only, as the recombinant alpha toxin shows no haemolytic activity. Up to a 16-fold dilution of the control serum (corresponding to 1.56% serum) completely inhibits the alpha toxin haemolysis. To the contrary, none of the candidate VHHs has an effect on the haemolytic activity of alpha toxin. Because the control serum contains polyclonal antibodies, whereas the VHHs are monoclonal, the combined effect of all 5 VHHs towards alpha toxin is determined (1 μM of each VHH in the highest dilution, corresponding to 5 μM VHHs in total). Combining the VHHs has no effect on the alpha toxin haemolysis.

Based on the above results, VHH antibodies EAT-1F2 and EAT-1G4 are selected for further characterization and expression. The peptide sequence of EAT-1F2 is: (SEQ ID NO: 97)

EVQLVESGGGLVQAGGSLRLSCAGSGRTGSLYSMGWFRQAPGKEREFVA
AITWRPSSTYYADSVKGRFTISRDDAKNTVYLQMNSLKPEDTAVYFCAA
RPRGGLSPTPQAYDYWGQGTQVTVSSAAASGSLEQKLISEEDLNGAAHH
HHHHGAA

The peptide sequence of EAT-1G4 is: (SEQ ID NO: 98)

EVQLVESGGGLVQPGGSLRLSCAASGSIATINDMGWFRQAPGKQRDWVA
TIVSDGSTAYADSVKGRFTISRDNAKNTVYLQMNSLKPEDTAVYYCSAR
RHYGQGTQVTVSSAAASGSLEQKLISEEDLNGAAHHHHHHGAA

A person of skill in the art would recognize that, because of the redundancy of the genetic code, multiple nucleic acid sequences could encode the above peptides. However, an exemplary sequence encoding EAT-1F2 is: (SEQ ID NO: 99)

| | |
|---|---|
| 1 | GAGGTGCAGC TCGTGGAAAG TGGCGGAGGT CTTGTTCAGG CTGGGGGATC GCTCCGTCTG |
| 61 | AGCTGTGCGG GGTCTGGCAG AACAGGTAGT CTCTATTCCA TGGGTTGGTT TCGGCAGGCC |
| 121 | CCGGGTAAGG AGCGGGAGTT CGTTGCAGCG ATTACGTGGA GGCCCAGCTC TACCTACTAC |
| 181 | GCGGACAGCG TAAAGGGACG ATTCACCATT AGTAGAGACG ACGCAAAGAA TACTGTATAT |
| 241 | TTGCAGATGA ATTCGTTGAA GCCTGAGGAC ACCGCTGTCT ATTTTTGCGC GGCGCGACCG |
| 301 | AGGGGCGGTC TCTCCCCGAC ACCTCAAGCA TATGATTACT GGGGACAAGG GACCCAAGTC |
| 361 | ACTGTATCCA GTGCGGCCGC GAGCGGCAGC CTTGAACAAA AGCTGATAAG CGAGGAGGAT |
| 421 | CTCAATGGTG CTGCACATCA TCATCACCAT CACGGGGCAG CG |

Exemplary sequence encoding EAT-1G4 is: (SEQ ID NO: 100)

| | |
|---|---|
| 1 | GAAGTTCAGC TTGTAGAGTC CGGTGGGGGT CTTGTACAGC CCGGCGGGAG CTTGCGACTC |
| 61 | TCATGCGCTG CTTCCGGAAG CATTGCGACA ATAAATGATA TGGGTTGGTT TAGACAAGCC |
| 121 | CCCGGGAAGC AGCGTGACTG GGTCGCGACT ATTGTGAGTG ACGGCAGCAC GGCTTATGCG |
| 181 | GACTCAGTGA AAGGGAGATT TACGATTTCG CGAGATAACG CGAAAAACAC TGTATACCTG |
| 241 | CAGATGAATT CACTCAAGCC GGAAGATACA GCTGTGTATT ATTGTTCTGC CCGACGGCAC |
| 301 | TACGGACAGG GGACCCAGGT CACAGTCTCG AGCGCTGCCG CCAGTGGGTC ACTCGAGCAG |
| 361 | AAGCTGATAT CAGAGGAGGA CCTTAACGGT GCGGCGCACC ATCACCACCA TCATGGTGCG |
| 421 | GCG |

### NetB Antibodies

NetB is a heptameric beta-pore-forming toxin that forms single channels in planar phospholipid bilayers. The NetB activity is influenced by membrane fluidity and by cholesterol, which enhances the oligomerization of NetB and plays an important role in pore formation. NetB has high haemolytic activity towards avian red blood cells.

Neutralization of the NetB haemolytic activity by camelid VHH antibodies directed towards NetB is determined by measuring NetB-mediated lysis of chicken erythrocytes. The ability to neutralize NetB haemolytic activity is assessed by pre-incubating a two-fold dilution series of the VHH antibodies (two wells per dilution, 5 μM starting concentration) with a constant amount of NetB toxin (20 μg recombinant NetB) for 30 minutes at 37° C. prior to the addition of 1% chicken erythrocytes. The non-toxic NetB variant W262A is included as a negative control as this variant displays no haemolysitic activity. Positive control serum from rabbits immunized with the recombinant NetB (wild type NetB) is used, starting from a 1:4 dilution. After incubation at 37° C. for 1 hour, the plates are centrifuged to pellet intact red blood cells. The supernatants is transferred to a new 96 well plate and the A₅₅₀ is determined. NetB activity is indicated by the increase in absorbance due to release of haemoglobin from the erythrocytes.

The control serum is able to neutralize the haemolytic activity of NetB. VHH antibodies ENB-1F4 and ENB-1F10 have no effect on the NetB haemolysis. ENB-1B9 has intermediate inhibitory capacity, while ENB-1D 11 and ENB-1A4 are able to neutralize the NetB haemolysis up to a 4- to 8-fold dilution (1.25 μM-0.625 μM VHHs).

Based on the above results, VHH antibodies ENB-1A4 and ENB-1D11 are selected for further characterization and bacterial expression. The peptide sequence of ENB-1A4 is: (SEQ ID NO: 101)

EVQLVESGGGLVQAGGSLRLSCAASGSIFSTNVMGWYRQAPGKQREFVA
GITIGGTARYPDSVKGRFTISRDNTQNTVYLQMNNLKPEDTAVYYCNAV
LPSDQRRWSWGQGTQVTVSSAAASGSLEQKLISEEDLNGAAHHHHHHGA
A

The peptide sequence of ENB-1D11 is: (SEQ ID NO: 102)

EVQLVESGGGLVQTGGSLRLSCTASGTIDMTYGLIWYRQAPGKERELVA
SIRRDGRTNYADSVKGRFTISIDNAKNSIHLQMNSLKPDDTARYYCNSP
YHALWGQGTQVTVSSAAASGSLEQKLISEEDLNGAAHHHHHHGAA.

A person of skill in the art would recognize that, because of the redundancy of the genetic code, multiple nucleic acid sequences could encode the above peptides. However, an exemplary sequence encoding ENB-1A4 is: (SEQ ID NO: 103)

| | |
|---|---|
| 1 | GAGGTACAAC TGGTTGAGAG TGGGGGTGGT TTGGTGCAAG CCGGAGGTTC CTTACGTTTG |
| 61 | TCTTGCGCGG CTAGTGGGAG CATCTTTTCA ACAAACGTAA TGGGGTGGTA CCGCCAAGCC |
| 121 | CCAGGTAAGC AGCGGGAATT TGTGGCCGGG ATAACGATCG GAGGAACTGC GAGGTATCCT |
| 181 | GATAGTGTGA AAGGGCGTTT CACAATTAGT CGAGATAATA CACAGAATAC TGTCTATCTC |
| 241 | CAAATGAATA ATCTCAAGCC CGAAGACACA GCAGTTTATT ATTGTAATGC CGTTCTCCCC |
| 301 | TCTGATCAGC GTCGATGGAG CTGGGGACAA GGCACCCAGG TTACGGTTAG CAGCGCGGCA |
| 361 | GCGTCTGGTT CGCTCGAGCA AAAGCTCATA TCTGAGGAGG ACCTGAACGG GGCAGCCCAC |
| 421 | CATCACCACC ATCACGGAGC AGCT |

An exemplary sequence encoding ENB-1G4 is: (SEQ ID NO: 104)

| | |
|---|---|
| 1 | GAGGTACAGC TGGTGGAGTC CGGCGGTGGT TTGGTGCAAA CCGGGGGTAG TCTGCGGCTT |
| 61 | AGTTGCACGG CGTCTGGGAC AATAGACATG ACTTATGGTC TCATATGGTA CAGGCAAGCG |
| 121 | CCTGGGAAAG AGAGGGAACT CGTTGCGAGT ATCAGAAGGG ACGGCCGCAC AAATTACGCT |
| 181 | GATTCAGTGA AAGGGCGCTT CACTATCTCG ATCGATAATG CGAAAAACAG TATTCACCTT |
| 241 | CAAATGAACT CCCTTAAGCC CGATGATACC GCCAGGTATT ATTGCAACAG CCCATATCAC |
| 301 | GCACTTTGGG GTCAGGGTAC GCAGGTAACA GTGTCTAGTG CGGCAGCCTC TGGTAGTTTG |
| 361 | GAGCAAAAGT TGATAAGTGA GGAGGACTTA AATGGGGCGG CACATCACCA CCACCATCAT |
| 421 | GGGGCGGCT |

### IV. Delivering Antigens as a Therapeutic Vaccine

Delivery of antigens in an immunomodulating, immune stimulation or vaccine strategy is an important and viable application of the platform of the present invention. B. subtilis strain 105 is modified and utilized to produce antigens which can serve as immunogenic polypeptides to stimulate an immune reaction and promote immunity, such as immunity against infection by an infectious agent or pathogen in an animal. In some embodiments, B. subttilis strain 105 is modified and utilized to produce one or more or multiple relevant antigens which serve individually or collectively as immunogenic polypeptides to stimulate an immune reaction and promote immunity, particularly enhanced immunity or a broader more effective immune response against an infectious agent or pathogen, including in applications as an immunogen, immune stimulator or vaccine.

Avian coccidosis is a common poultry disease caused by Eimeria. Eimeria is a genus of parasites that includes various species capable of causing the disease coccidiosis in animals such as cattle, poultry, dogs (especially puppies), cats (especially kittens), and smaller ruminants including sheep and goats. Eimeria species infect a wide variety of hosts. The most prevalent species of Eimeria causing coccidiosis in cattle are E. bovis, E. zuernii, and E. auburnensis. In a young, susceptible calf it is estimated that as few as 50,000 infective oocysts can cause severe disease. Eimeria infections are particularly damaging to the poultry industry and costs the United States more than $1.5 billion in annual loses. The most economically important species among poultry are E. tenella. E. acervulina, and E. maxima.

To generate and provide a immunogenic composition or coccidial vaccine for use and application in poultry, B. subtilis strain 105 is modified to deliver cross-protective antigens covering Eimeria parasites including Eimeria tenella, E. maxima and E. acervulina. Eimeria antigens including Eimeria tenalla elongation factor-1a; EtAMA1; EtAMA2; Eimeria tenella 5401; Eimeria acervuline lactate dehydrogenase antigen gene; Eimeria maxima surface antigen gene; Glyceraldehyde 3-phosphate Dehydrogenase (GAPDH); Eimeria common antigen 14-3-3 are cloned in a plasmid or integrated in the genome of strain 105 as the applicable gene of interest. Expression of the Eimeria antigens delivered by the B subtilis 105 in poultry provides vectored delivery of an immunogen to stimulate immune response and provise protection or immunity against Eimeria in the animals.

Cocci antigen sequences:

EmGAPDH
(SEQ ID NO: 105)
ATGGTGTGCCGCATGGGCATCAACGGATTCGGGAGAATTGGAAGGCTGGTGTTTCGGGCCGCTATGGCAAACCC
CGAAGTGGAGGTGGTGGCCGTGAATGATCCTTTCATGGACGTGCAGTACATGGCCTACCAGCTGAAGTACGATAG
CGTGCACGGCAAATTTCCTGGAGAGGTGTCCGTGAAGGACGGGAATCTGGTGGTGGAAGGCAAGACTATCCAGG
TGTTCGCTGAGAAAGATCCTGCAGCCATTCCATGGGGGAAAGTGGGCGCACACTACGTGTGCGAAAGCACAGGG
GTGTTTACTAACAAGGAGAAAGCTGGCCTGCACATCAGCGGCGGAGCTAAGAAAGTGATCATTTCCGCACCCCCT
AAGGATGACACACCAATGTTCGTGATGGGCGTGAATCACGAGGAATACCAGCCCACTCTGCAGGTGGTGAGCAAC
GCATCCTGCACCACAAATTGCCTGGCTCCACTGGCAAAGGTGGTGCACGAAAAATTTGGGATTGTGGAGGGCCTG
ATGACTACCGTGCACGCCATGACCGCTAACCAGCTGACAGTGGATGGACCTAGCAAGGGCGGCAAAGATTGGAG
AGCAGGAAGATGCGCTGGGAGCAACATCATTCCTGCCTCCACAGGAGCTGCAAAGGCTGTGGGGAAAGTGATCC
CATCCCTGAATGGCAAGCTGACTGGAATGGCCTTCAGAGTGCCAACCCCCGATGTGAGCGTGGTGGACCTGACCT
GCAGGCTGTCCAAGCCCGCTAAATACGAAGATATCGTGGCCGCTATTAGAGCAGCCAGCGAGGGACCTCTGAAG
GGCATCCTGGGAGTGACAGAGGAAGAGGTGGTGTCCCAGGATTTCTGCGGCGACAAGAGGAGCTCCATCTTTGA
TGTGAAAGCAGGCATCCAGCTGAACGACAGCTTCGTGAAACTGGTGTCCTGGTACGACAACGAATGGGGATACA
GCAATAGACTGGTGGATCTGGCCATCTACATGTCCAAGAAAGACGGCAATGGAGCTGCACACCATCATCACCACC
ACCACCACCACGGAGCAGCTTGGAGCCACCCCCAGTTTGAGAAGGGGGCAGCCTGA
Ea 14-3-3
(SEQ ID NO: 106)
ATGGGAATCGAGGATATTAAGACACTGCGCGAGGAACACGTGTACCGGGCTAAACTGGCAGAGCAGGCCGAACG
CTACGACGAGATGGCAGAAGCCATGAAGAACCTGGTGGAGAATTGCCTGGATCAGAACAGCTCCACACCCGGCG
GAAAAGGGGACGAACTGACTGTGGAGGAACGGAACCTGCTGAGCGTGGCCTACAAGAATGCAGTGGGAGCAAG
AAGGGCATCCTGGAGAATCATTAGCTCCGTGGAGCAGAAGGAAGCAAACCGGAATCACATGACTAATAAAGCCCT
GGCCGCTAGCTACAGACAGAAGGTGGAGAACGAACTGAATAAAATCTGCCAGGAGATTCTGACCCTGCTGACAG
ATAAACTGCTGCCTAGAACCACAGACAGCGAATCCAGGGTGTTCTACTTTAAGATGAAAGGCGATTACTACAGGT
ACATCAGCGAGTTCTCCAACGAGGAAGGAAAGAAAGCTAGCGCAGAACAGGCCGAGGAATCCTACAAGAGAGCT
ACAGACACTGCCGAGGCTGAACTGCCCAGCACTCACCCTATCAGGCTGGGACTGGCCCTGAACTACTCCGTGTTTT
ACTACGAGATTCTGAATCAGCCACAGAAGGCTTGCGAAATGGCTAAACTGGCATTCGATGACGCAATCACCGAGT
TTGATAGCGTGTCCGAGGAAAGCTACAAGGACTCCACACTGATTATGCAGCTGCTGCGCGATAATCTGACTCTGTG
GACCAGCGACCTGCAGACTCAGGAACAGCAGCAGCAGCCAGTGGGAGAGGGAGCAGAAGCACCCAAAGTGGAG
GCTACTACTGAACAGCAGGGAGCAGCACACCATCATCACCACCACCACCACCACGGAGCTGCATGGTCCCACCCTC
AGTTCGAGAAGGGGGCCGCTTGA
EF-1α
(SEQ ID NO: 107)
ATGGGCAAGGAGAAAACACACATTAATCTGGTGGTGATCGGGCACGTGGATAGCGGCAAGTCCACCACAACTGG
ACACCTGATCTACAAGCTGGGCGGAATTGACAAAAGGACTATCGAGAAGTTCGAGAAAGAAAGCTCCGAAATGG
GAAAGGCCTCCTTTAAATACGCTTGGGTGCTGGATAAGCTGAAAGCTGAGCGCGAACGGGGGATCACAATTGAC
ATCGCACTGTGGCAGTTCGAGACTCCCGCCTTTCACTACACCGTGATCGATGCTCCTGGGCACAGAGACTTTATTA
AAAACATGATCACCGGCACATCCCAGGCAGATGTGGCACTGCTGGTGGTGCCTGCAGACCAGGGCGGCTTCGAA
GGAGCCTTTAGCAAAGAGGGCCAGACAAGGGAACACGCTCTGCTGGCATTCACTCTGGGCGTGAAGCAGATGAT
TGTGGGAATCAACAAAATGGATGCCACTTCCCCTGAGAAGTACAGCGAAGCTAGATTTAATGAGATTCAGGCAGA
AGTGTCCAGGTACCTGAAAACCGTGGGCTACAACCCAGAGAAGGTGCCTTTCGTGCCAATCAGCGGGTTTGTGGG
CGATAACATGGTGGAACGCAGCTCCAATATGGGATGGTACAAGGGGAAAACCCTGGTGGAGGCACTGGACAGCG
TGGAACCCCCTAAGCGCCCAGTGGATAAACCCCTGCGGCTGCCTCTGCAGGACGTGTACAAGATTGGAGGGATCG
GAACTGTGCCTGTGGGAAGAGTGGAGACAGGAGTGCTGAAACCTGGAATGGTGGTGACTTTCGCTCCATCCGGG
CTGCAGACCGAGGTGAAGAGCGTGGAAATGCACCACGCACAGCTGGAACAGGCCGTGCCTGGCGATAATGTGGG
ATTTAACGTGAAGAATGTGTCCGTGAAGGACGTGAAAAGAGGACACGTGGCTAGCGATTCCAAGAATGACCCAG
CAAAAGCCGCTGCAAGCTTCCAGGCCCAAGTGATTGTGCTGCACCACCCCGGGCAGATCAACCCAGGCTACACAC
CCGTGCTGGATTGCCACACTGCCCACATTTCCTGCAAGTTTGCTGATCTGGAGAAACGCCTGGACAGAAGGAGCG
GCAAAGCACTGGAAGATAGCCCAAAGTCCATTAAAAGCGGAGACGCCGCTATCGTGCGGATGGAGCCCAGCAAG
CCTATGTGCGTGGAGGCTTTCATCGAATACCCACCCCTGGGGAGATTTGCAGTGAGGGACATGAAACAGACCATT
GCTGTGGGCGTGATCAAGGCAGTGGAGAAGAAAGAAGCCGGCGGAAAGGTGACAAAATCCGCCCAGAAAGCAG
CCGCTAAGAAAGGAGCAGCACACCATCATCACCACCACCACCACCACGGAGCTGCATGGAGCCACCCACAGTTTG
AGAAGGGAGCAGCTTGA
Em surface antigen
(SEQ ID NO: 108)
ATGGATACTATCAGCTCCCCTCTGAGCTGCCTGGAGCAGATGAACAAAGTGCGGGAAGCCGCTGGCCTGCCCAAA
TTCGTGGAGAATACTCAGCTGCTGCCTTCCGAAGCCCCTAAGACCCCAGATGACTTTTGGGAGAAGCTGTGCGCTA
AAGTGAGAGGCGACCCAGCTACAGAAGCAGTGGGCAGCGCCCCTCAGGGAACTTACGCTTTCTACCCTACCTCCG
AGGATGCCGGAAACTGCACTGCAGCCGTGGAGTTTTGGGAAGGCGGATTCAGCCTGTTTAATGACAAGCTGCCAG
AAAAATACATCGCAAGCTCCCCCAACACCTACAGCAATCAGCGCGCTGTGTCCTTCGTGGCACTGTTTAACCCCAA
ACCTGATGCTTCCGTGCACTGCACATTCGCAAAGTGCCCCAAAACTGCCCCAGAACCTGACCTGGGCGGCAGAAG
GCGCCTGGCTGCAGAAACCCTGAATAGCGTGATCTGCATTACATCCCCTGAGGCTCTGGTGAACGAACAGGCACC
ATTTAGCGAGGATGTGTGGAATAAGATTACTGCTGCTCTGACCGGCGGCGTGAAAGCAGTGGGACCAACAGCCCT
GATTGCTCTGGCAGCACTGGCACTGAGCGCATCCGTGCTGATTGGGGCTGCACACCATCATCACCACCACCACCAC
CACGGAGCAGCTTGGAGCCACCCCCAGTTCGAGAAGGGCGCAGCCTGA
EtAMA1 (extracellular domain)
(SEQ ID NO: 109)
ATGGGATCCTGCGCTGGACCTGCTGCTGGAGTGCAGCACAAGCTGCAGCACAGGCAACAACAGCAGCAGCAGCA
TTCCCACGCAAGCACTTCCCACGCAGCAGCTGTGCTGGCAGCAAGCTCCGATGCTAGCACCGACTCCAATCCTTTC
ATGCAGCCCCCTTACGCTGAGTTCATGGCACGCTTTAACATCCCAAAGGTGCACGGGAGCGGCGTGTACGTGGAT
CTGGGGAATGACAAAGAGGTGAAGGGCAAAATGTACAGAGAACCCGGCGGAAGGTGCCCTGTGTTCGGCAAGA
ACATTGAGTTTTACCAGCCCCTGGATAGCGACCTGTACAAAAATGATTTCCTGGAAAACGTGCCTACCGAGGAAGC
TGCTGCTGCTGCAAAGCCTCTGCCAGGGGGCTTCAACAACAACTTTCTGATGAAGGACAAGAAGCCCTTTAGCCCT
ATGTCCGTGGCTCAGCTGAACAGCTACCCCCAGCTGAAGGCACGGACAGGACTGGGGAAATGCGCCGAAATGAG
CTACCTGACCACAGCCGCTGGCAGCTCCTACAGATACCCCTTCGTGTTTGATAGCAAGAAAGACCTGTGCTACCTG
CTGCTGGTGCCTCTGCAGCGCCTGATGGGAGAGCGGTACTGCAGCACTAGAGGGTCCCCACCCGGCCTGTCCCAC
TTCTGCTTTAAGCCACTGAAAAGCGTGTCCCTGAGGCCCCACCTGGTGTACGGAAGCGCATACGTGGGGGAGCGC
CCTGATGACTGGGAAACAAAGTGCCCAAACAAGGCCGTGAAAGATGCAGTGTTTGGAGTGTGGGAGGGAGGGA
GATGCGAGGAACAGAGACTGAGACTGGGAGCTCAGACTGCAGCTGCTGCAGCCAAAGAAGACTGCTGGGCACTG
GCCTTCAATAACCCCTTTGCTGCATCCGATCAGCCTACTAGCCAGGACGAGGCCGCTACCTCCCCAGGATACTACTT
CCCCAGCATCACTCCTAGCCAGCCAAAGAGCGGCGGAGTGGGAGTGAATTTTGCCTCCTACTACCCTAGCGGAGA
GTGCGTGCTGAGCGGAGAAGTGCCTACATGCCTGCTGCCAAGGCAGGGAGCAGCCGCTTTCACTAGCGTGGGGT
CCCTGGAGGAAGAGGAACTGCCACACTGCGATCCTACATTTCCTGCATCCCTGGGGTCCTGCGACCCAAGCTCCTG
CAAGGCAATTCTGACTGAGTGCAGAGGCGGCAGACTGGTGGAACAGCAGACAGATTGCGTGCCAGAGGACGGC
AGCAAGTGCGAAAGCAAGGGCGGCGGCGGCGCCGCACACCATCATCACCACCACCACCACCACGGAGCTGCATG
GAGCCACCCCCAGTTTGAGAAAGGAGCCGCTTGA

### Example 16

### Engineering Bacteria to Include Biosynthetic Gene Clusters (Bcg)

A biosynthetic gene cluster (BGC) is a group of genes in bacteria that work together to produce or generate one or more molecules or proteins, or in some instances a protein complex. that provide one or more activity or related activities and/or serve a related or final function. Clustering of a group of genes can permit or enable timed and coordinated synthesis, for example of proteins involved in a pathway. The proteins can be under the control of multiple promoters or transcribed by a single promoter or group of promoters.

A. Engineering of Polyketide Synthase (PKS) Biosynthetic Cluster from LactoBacillus Reuteri into Baciius Subtilis

Polyketide synthases (PKS) are secondary metabolites produced by biosynthetic gene clusters (BGCs) that assemble simple molecules into complex metabolites that have potential therapeutic value. The gut microbiome encodes for several BGCs that produce secondary metabolites that directly interact with the host immune system. Of particular importance is the BGCs that encode for AhR-activating metabolites. AhR is a ligand-activated transcription factor that recognizes environmental pollutants, dietary compounds (i.e., glucobrassicin and flavonoids), and microbial-derived secondary metabolites (i.e., indole-3-carbinol). Upon ligand binding, AhR translocates into the nucleus to induce target gene expressions. The role of AhR has been extensively studied in relation to metabolism of environmental toxins, but the focus has recently shifted to its role in modulation of the adaptive and innate immune system. AhR is a ligand activated transcription factor that plays a key role in a variety of diseases including amelioration of intestinal inflammation. Ozcam et al have shown that some L. reuteri strains can activate the aryl hydrogen receptor (AhR) and that this activation is associated and correlated with the presence of PKS gene cluster and its metabolite(s) (Ozcam M et al (2019) Appl Environ Microbiology 85(10):e01661-18). Strains that have the PKS biosynthetic gene cluster activate AhR and produce a bright orange pigment. Deletion of the PKS gene cluster results in loss of the abiolity to activate the AhR receptor. AhR activation by L. reuteri has been shown to alleviate E. coli-induced mastitis in mice (Zhao C et al (2021) PLOS Pathogens 17(17):e1009774), and could be an effective approach against mastitis in other animals, particularly lactating animals, such as cattle for example. AhR activity and AhR-expressing micriobiota communications have been multi-factorially implicated, including in modulation of immune tolerance and response, intestinal homeostasis, carcinogenesis and intestinal barrier integrity (Dong F and Perdew G H (2020) Gut Microbes doi.org/10.1080/19490976.202.1859812). AhR has been implicated in various inflammatory- and immune-mediated conditions, such as atopic dermatitis.

The Bacillus subtilis strain 105 is modified to introduce a biosynthetic gene cluster from LactoBacillus reuteri that encodes for a polyketide synthase which provides and acts as an AhR-activating metabolite. In particular, the LactoBacillus reuteri strain is 3632 (ATCC PTA-126788). The L. reuteri metabolite appears to give an orange pigmentation to the strain and is primarily associated with cell envelope. LactoBacillus reuteri strain is 3632 (ATCC PTA-126788) is described as having a characteristic orange pigment, including in Kumar et al, WO 2020/163398A1, published Aug. 13, 2020, and corresponding US publications are US 2022/0088094 published Mar. 24, 2022 and US 2022/0125860 published Apr. 28, 2022, the entire contents of which are incorporated herein by reference. In a particular aspect, Bacillus subtilis strain 105 is modified to introduce the PKS cluster from Lactobacillus reuteri so as to efficiently produce the candidate AhR-activating metabolite. In particular, the LactoBacillus reuteri strain 3632 (ATCC PTA-126788) contains a BGC that encodes for a full suite of proteins required for synthesis and production of the AhR-activating metabolite. LactoBacillus reuteri strain 3632 is detailed and described, including its full genome nucleic acid sequence in Kumar et al, WO 2020/163398A1, published Aug. 13, 2020, and corresponding US publications are US 2022/0088094 published Mar. 24, 2022 and US 2022/0125860 published Apr. 28, 2022, the entire contents of which are incorporated herein by reference.

The PKS gene cluster from LactoBacillus reuteri strain 3632 is encoded on a conjugation plasmid of 165 kb. The biosynthetic gene cluster (BGC) contains 15 genes that encode for a full suite of proteins needed for the synthesis of AhR-activating metabolite (TABLE 22). The gene cluster is introduced into B. subtilis strain 105 to enable synthesis and production of active and effective AhR-activating metabolite by the modified B. subtilis strain.

TABLE 22
Genes involved in the synthesis and secretion of mersacidin

| Locus tag | Gene | Description | Remarks |
|---|---|---|---|
| LREU3632_02405 | emrY/pksA | putative multidrug resistance protein EmrY | Transporter |
| LREU3632_02406 | fabF_2/pksB | 3-oxoacyl-[acyl-carrier-protein] synthase 2 | |
| LREU3632_02407 | pksC | acyl carrier protein | |
| LREU3632_02408 | fabG_2/pksD | 3-oxoacyl-[acyl-carrier-protein] reductase FabG | |
| LREU3632_02409 | pksE | Hypothetical protein | |
| LREU3632_02410 | fabZ 3/pksF | 3-hydroxyacyl-[acyl-carrier-protein] | |
| | | dehydratase FabZ | |
| LREU3632_02411 | pksG | Glyoxylase/metallo-beta-lactamase | |
| | | superfamily protein | |
| LREU3632_02412 | pksH | Hypothetical protein | |
| LREU3632_02413 | pksl | Transcriptional regulator PadR-like family | May not be required |
| | | protein | Deleted from gene cluster |
| | | | for evaluation |
| LREU3632_02414 | baeC/pksJ | Polyketide biosynthesis malonyl | |
| | | CoA-acyl carrierprotein transacylase | |
| | | BaeC | |
| LREU3632_02415 | accB/pksK | Biotin carboxyl carrier protein of | |
| | | acetyl-CoA carboxylase | |
| LREU3632_02416 | cfiB/pksL | 2-oxoglutarate carboxylase small subunit | |
| LREU3632_02417 | accD 2/pksM | Acetyl-coenzyme A carboxylase carboxyl | |
| | | transferase | |
| | | subunit beta | |
| LREU3632_02261 | accA 2/pksN | Acetyl-coenzyme A carboxylase carboxyl | |
| | | transferase subunit alpha | |
| LREU3632_02262 | pksO | bifunctional biotin--[acetyl-CoA-carboxylase] | |
| | | synthetase/biotin operon repressor | |
| LREU3632_02265 | | bifunctional biotin--[acetyl-CoA-carboxylase] | Likely a regulator, |
| | | synthetase/biotin operon repressor | may not be |
| | | | required for |
| | | | producing the |
| | | | metabolite; |
| | | | appears to be |
| | | | located outside the |
| | | | BGC |

Note that LREU3632_02265 indicated in Table 22 may not be required.

The PKS gene cluster from LactoBacillus reuteri 3632 was engineered into Bacillus subtilis #105 to efficiently produce (secrete) AhR-activating metabolite. The BGC cluster was chromosomally integrated and confirmed by PCR and sequencing. The final strain did not contain any antibiotic markers.

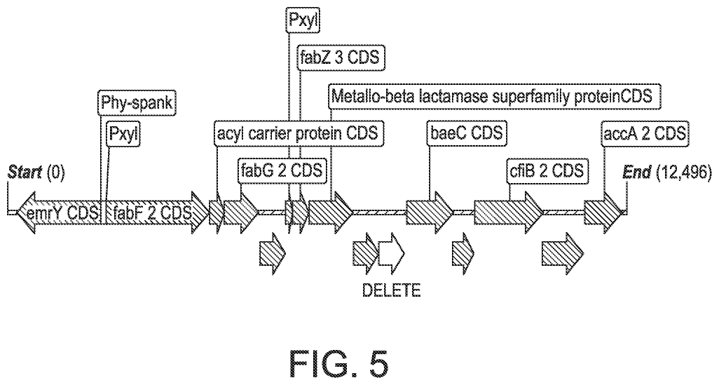

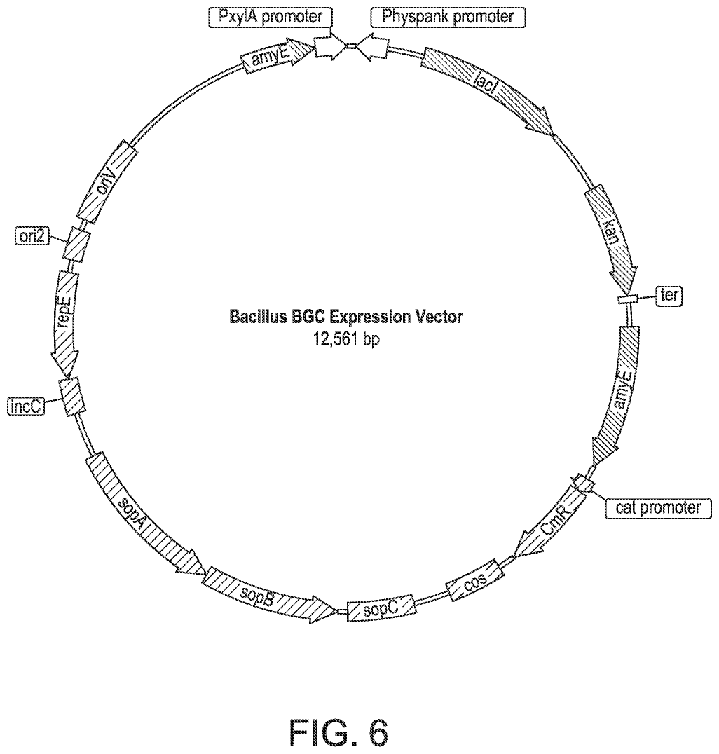

In a first initial step (i) the pksI gene, transcriptional regulator, was deleted from the PKS gene cluster as it may not be required. Then (2) the rest of the gene cluster and pathway gened was cloned as a control. Using the cloned wild type AhR PKS BGC, three promoters—two PxylA promoters and a Physpank promoter were inserted to control gene expression as diagrammed in FIG. 5. The Physpank and pxl promoters were introduced as promoters for the emrY and fabF2 genes (these genes are coded in opposite directions on the gene cluster and the promoters are flanked and promote gene expression in opposite directions). A third promoter, pxlA was introduced in front of and upstream of the fabZ 3 genes. The cloned gene cassette construct was sequenced in its entirety to confirm all of the components and promoters. The cassette was then inserted into a suitable vector, in this instance designated as Bacillus BCG expression vector (FIG. 6). The cassette is flanked by left and right amylase gene amyE arms for homologous recombination and integration at the amylase gene amyE in the Bacillus subtilis strain. B subtilis strain 105 was transformed with the vector under conditions to promote homologous recombination and integration and then screened for full length insertion using PCR to verify the left, right and middle portions of the Ahr PKS BGC.

The PKS encoding BGC was successfully engineered into B. subtilis #105 and confirmed by PCR and sequencing (data not shown). Junctional PCR confirmed the correct and full integration of the PKS gene cluster in the B subtilis 105 genome (data not shown).

B. subtilis 105 is geneticall modified to integrate the PKS gene cassette in its bacterial genome. Extracts and supernatants of the modified strain are evaluated. These are evaluated in concert with native L. reuteri strain 3632, which expresses the Ahr activation product from its native PKS gene cassette. The AhR activation product is evaluated in an in vitro potency assessment against several AhR responsive cell lines such as HepG2-Lucia (human HepG2 hepatoma; Invivogen) and HT-29-Lucia (Human HT29 colon carcinoma; Invivogen).

The engineered strains are evaluated for AhR activity as follows. The strains are grown overnight in LB media. The cell pellet and the filter sterilized culture supernatant is evaluated for AhR activity using HepG2-Lucia^{m} AhR cells (Invivogen, hpgl-ahr). HepG2-Lucia^{m} AhR cells will be grown at 37° C., 5% CO₂ in MEM (Thermo Fisher, 616965-026), with 10% iFCS. For selection purposes, culture medium is supplemented with 100 μg/ml zeocin (Invivogen, ant-zn-5). FICZ (6-Formylindolo[3,2-b]carbazole; AhR Agonist and L-Kynurenine will be used as positive controls. Media control and B sub
105 (ELA191105) parent strain controls are also included. The dilution of test material is performed at 2× of the intended final concentration in 40 ml complete growth medium and added with 40 μl of 2.2×105 cells/ml. Culture supernatants are harvested at different time points (over 48 h) and subjected to Luciferase Assay. For measuring luciferase activity in 384 well, white plate, 10 μl culture supernatant/well sample is added with 15 μl/well of Quanti-Luc/Coelenterazine-utilizing luciferase detection medium (Invivogen rep-qlc) in 50 ml ddH20 (instead 25 ml). The reaction is monitored at room temperature with BioTek plate Reader.

Efficacy of the Bsub integrated PKS cassette produced AhR metabolite evaluation in vivo is assessable using a mouse atopic dermatitits (AD) animal model (Martel B C et al (2017) Yale J Biol and Med 90:389-402). The AhR activator tapinarof is in clinical development for treatment of psoriasis and atopic dermatitits (AD) in humans (Bissonnette R et al (2021) J Am Acad Dermatol 84:1059-1067; Lebwohl M et al (2020) Skin J Cutane Med 4(6):s75). Tapinarof is a secondary metabolite from Photorhabdus luminescens.

The full sequence of the PKS cluster along with 5 kb flanking regions on both sides of the BGC, corresponding in total to 12,969 bp is provided below (SEQ ID NO: 110).

| | |
|---|---|
| 1 | gcaagaattt atcccatagt ttgcgagaaa tccttttaga agagcttgcc taataatagc |
| 61 | ggctcaatat aatttaaatt taaaaccacc taattagcaa tcgtaaaatt gttaattagg |
| 121 | tggttttagt atagaaataa cccctaaatt cagaatacta gaaaatctag tactattatt |
| 181 | ttttcctttt cgggaataat agacatgaaa gacaagaaaa gataagaaat ggtaaggaaa |
| 241 | ctttgtacag tttaataaaa gcagttgagt attgactatt ggcgtaagtt ttgatttttt |
| 301 | taattgtaat attaatagaa ttatttaatc tatgaaatac taatttcaca tttgcttttg |
| 361 | tatgtgaata aatctctttt tcttggatct tagagggttc gtaaggtaag gaattaattt |
| 421 | gtttagtata ttctttatcc gtagtttctt taattaattt cttcgaaaaa tgctttgatg |
| 481 | gtgttcgaga gtttgaattg ttgtttaagc ttttaataga gttatctctt attttatttt |
| 541 | tttgatattt aggaatattt aagttattga cctgctgatt gatgtaacta atagaattat |
| 601 | gctttgcggt atttaaattc ccatatagtc cagttagaaa tattgctaca gctaaggaaa |
| 661 | cccctacttg tcttaaaaca cctgcaacac tttgtgaagc ggttaagaga taaccttcaa |
| 721 | agtttgatgc tgctaaaact gttattggac ctgcaataat cccataccca gttcctaaaa |
| 781 | taatacaagt aaaaataatt aaggtaatat tatccatatc aatatgtgta aataaatagt |
| 841 | aagagatacc cattaataga aaaccagata agataatggc tcgagatcct aatttgtcta |
| 901 | agagaattgc agaaagtggt gacatgataa aaatcattcc tgtaataggc gtgataagaa |
| 961 | gtgcagcttc taattcggtt ctatgttgta tatgggtaaa atatgttggt aaaattactg |
| 1021 | taacagccac cagaaataaa ttacttaaaa ttatagaaac ggcagagccg gtaaattctc |
| 1081 | tattcttaaa taaaacaaga ggaatcattg gtgctttagc ataatgctca gtaattaaga |
| 1141 | atagtatgaa agttaaaatg aaaatgaaaa acagaaggtt gattgcagga cttttccacc |
| 1201 | cccagcttct tccttgagtc aatattagag ttaaagctga aagtaaaatt atacctaaaa |
| 1261 | gggaacctaa ataatcattt ccttcatttg actttggttc gtgaaagttg aaagtagata |
| 1321 | aacaaataac tagtgaaagt atagtaaaag gtatattaac gctaaatata gaatgccagc |
| 1381 | ccataaattg agttagaata cctccgattg aaggaccaag agcagcagct aaaccttgag |
| 1441 | tgattcctaa tgcggcaata actttttttc ttgtctttac tgtaacgata tttattccaa |
| 1501 | tagtcattga aagtggaaaa agaattgcgg ctcctaatga ttgaatgcca cggccaagaa |
| 1561 | ttaaaatagc taggttagga cttataccag aaatacttga tccaataaga aacataatta |
| 1621 | acccaattat atataagaaa ttcataccta atttttctgc aagtttagat agtgggatcg |
| 1681 | ttaaactagc aaaaagtata gtatatatgt ttaatgccca agacaaatta gttagagtga |
| 1741 | cgtgaagttc gttttgaata gctggcaatg caatattcat aacagttgta tctaacatac |
| 1801 | ataagaaaat actaatacac attgcaatta ttattagaaa tttgtttttt cttttggtca |
| 1861 | tacttgtttc ctcactttgt ttatattaac aatgttaata taaacaaaag aaaaataaat |
| 1921 | gtcaacaata ttatcattgt aaatatgaac aatatgtatt aatataaata aaaattagtg |
| 1981 | aggtgatttt atgacagctg taatagttgg aattggtatt actagttcct gtggagagtc |
| 2041 | ctttacagaa attgaacaaa atgtaggtaa aggaaaaaca ggtatttcta atatcgatta |
| 2101 | ttttgataca tctgagttaa catgtggaat tgctggtaac ttatcaaaga aaatatggaa |
| 2161 | agaggttttg caaattgctg ataaaaatga attagattgg agtagtagtc tttcaattta |
| 2221 | tactattcag cgattacttg aatcgtataa tatttcaaaa aaacaaagaa ttggtttatc |
| 2281 | tcttggaaca tgtaatgggg gaattcattc tttagcagaa tatcttgata cttcaaatga |
| 2341 | taaattttta aaaaattatc ctccgtacat tcagagtaaa gatatcgcac attatttcaa |
| 2401 | ctttaacggg ccaaagtatt cttttaattc agcttgtgcg gctagtgcta atgcaatagc |
| 2461 | ttatggagca gaaatgatta ataatagcga tgcagatctt gttgtgacgg gtggttgcga |
| 2521 | tccaatgtca gaatgggttt ttgctgggtt taattcatta agaaccttta atagtaaaaa |
| 2581 | ttgtatgcca tacggtgagg aatatggact taacctagga gaagctgcta catacttctt |
| 2641 | attagaagat aaagataagg caattaaaaa agggcatagg atatatgcgg aaattttagg |
| 2701 | gcacggctta tcaaatgatg cttatcatcc tacagcgcct gataaggatg gttcagggat |
| 2761 | atcctatgca ataaaaatgg ctttaaaaaa ctcgggtctg aagcctgaag atattttata |
| 2821 | tattaactct cacggaacag gaactaaagc caatgatagt gcagaataca gaggatttaa |
| 2881 | aactgttttt agaaatgaga tgccttttat tagctcaatg aaaggatacg taggccataa |
| 2941 | tctgggagca gctgctagta cagaattagc tataagttta atcggtatga atagtcagaa |
| 3001 | agttttgtat cctaacttta atttaaccaa gtatagagaa gattgtaacg atgagcatat |
| 3061 | cttaaaaaag ccttattcat tagatggata tgaagatatc aactttataa acaataatgc |
| 3121 | tgcgtttggt ggacaaaatg ttgctgtaat tttccatgtt aatttagaag gaaaatatgg |
| 3181 | tcatagtgaa aagaaactta aaacacagca acctatatat attaataatt ttggcgttgc |
| 3241 | aagtgataaa gcttatatga caaagcatgg tattggtatt cttgacgatt tacgcccctt |
| 3301 | aaagaaaaaa tatccaaaat tatataagcg acggatgaat atgttaactc aagtaagtat |
| 3361 | tatcgcggct aaacaaacat tgcaggatca gtatagtaat tgtgggcttg tgtatggtac |
| 3421 | gccttttggt agtctttcat caacactaaa atatgttgat tctattcaaa aatatgggtt |
| 3481 | taaaaatgct agtggggcat attttccaga tttagttatt aattcaacga cggggcatat |
| 3541 | ttgtcaagcg ctttcattga agagttatag ttcatctata agttcaggag gtgatgaaga |
| 3601 | cttaagagcc ttaattattg cacataatgc gcttaataaa gggtacgctt caactatgtt |
| 3661 | agttggagca ggccaagaag agacagaact gggcaataaa gtgttgaagc gtgaagttaa |
| 3721 | taatcatgca acatttttaa gccttagtaa caaaaaaatg caagaaacaa tagcagaggt |
| 3781 | tctatcttcc ggtgcaatgg gatttaaaaa taaaaaagag cttttgacca ttatacgatc |
| 3841 | gaaaatagat gagaagttgg ctaatgatgc agatctaaaa gtgataattc aaaataatag |
| 3901 | tgaaattacg aacgacgaat taataagtta tttcaagaat gataacaata tcagtataaa |
| 3961 | ttctgataat tttgcggata gtaattttaa atcatttgtt aatcatagaa atgaaaatag |
| 4021 | gttgttgctt gttggaattt cacaagtcaa tgatgtttcg tttgcagata ttaaaaaaat |
| 4081 | aaaatagaga aagaggtaat actgatgaag catagtattg aagaaattaa ggatatttta |
| 4141 | aaagaaaaag tattaattga acgattagag ttagatgatg tagaacccaa tgatatttca |
| 4201 | gataatgaaa atttatttga tgaagaagga ctagcgttag attccgtgga agcattagat |
| 4261 | atcatgacag gaattagtga agaatttggc attgacacat caatgttagg gcaggaagat |
| 4321 | ataaaccatt ttcaaagcgt taatgatatg gctaaatata tctcagagaa tgaatagagg |
| 4381 | atagttatga aatcagtact tatcacggga ataactggag gaataggaag aaaattaact |
| 4441 | gaagcttatt cttcaaaggg atatcatatt tatggtacgt gtagtagaaa ttcagactct |
| 4501 | ttacaacaat ttaaagagaa atggcctagt gttgaaataa ttcaaataaa tcatgatgac |
| 4561 | ttgatagatg taagtacaga atattccttt ttttttagaa aagtgcaacc agatattgtt |
| 4621 | attaacaatg ctggaatagt gaaagataat tttttagttc aaatgtcagt gaatgatttt |
| 4681 | caagaagtgt tgactactaa tttgatttcg gcttgggtaa tagtcaaaga aatgctttta |
| 4741 | catttaaatg ataataaaat tcataaaatc attaatgtag cttcaatatc tggaattata |
| 4801 | gggcgtgaag gacagtgtaa ctatgcagca actaagggag gattagtagg cttatgtcaa |
| 4861 | ttgatagaac atttagctcc gaaaggcagt aatgttattt cattttcagt tgcaccagga |
| 4921 | ttaattgata cagatataaa gggaaaaatg ccaaagaaaa aaatcgataa tttaaagaag |
| 4981 | gccacattgg caaataggct aggaacacca gaggaagtat caaaatttat ttttaaacta |
| 5041 | tctgaagagg atatttcgta tagtgatgga actttatata ggattgatgg tggagtttta |
| 5101 | aaatgaagat aataaataat acgattcaag taaccgactt gcttcaatca tatggaaaag |
| 5161 | atgaaaaaga catagtaata attggtccca gcccatttaa tgaattagat tgccttaaag |
| 5221 | aaacaaaaat aatcgataaa gttcagctta atttagagga agttttttca tttgtaaaaa |
| 5281 | ataatagcgt tgctttaatg aaaaaaagaa gaggaaccat tgccttttta ttaaatccac |
| 5341 | aaagttttga gggaggcaat aatatctatt ctccaatcta taattcagca attaagagct |
| 5401 | ttttgaaatc cttatcgaag gaaatgaatc cattcagagt taaagttatg gggataatct |
| 5461 | tacctttaac acaagataca aaatcgactc ggaaatatga tttagttaca ttaaagtaca |
| 5521 | aaggaattaa taatgaaaag caggtacaag atattctaag cttacttaag ctttcagaaa |
| 5581 | tactaaacgg acaaattgtc tcgctaggcg ctgaattgaa tctttagtaa aggagaggga |
| 5641 | aatcatttta tttctagatc aaaatgcagt tataaaaatg ttgccgcaga aggagccatt |
| 5701 | caggttctta gatacggttg aatgctttga tagagaaaaa cgtatgatta ctgcattgca |
| 5761 | acaatttggt aatgaggaat tcttctttaa gggtcatttt ccaaataatc caatagttcc |
| 5821 | aggggtactt ctaacagaat cgattgctca agcaggtttg atattgattt ctttattaga |
| 5881 | aggccaaaaa gtgaaaattg gatatctagc ccaaattgaa aagacaaaat ttttcaaaga |
| 5941 | ggtctatccc gacgagcaag taaaggttaa atgttcatta aagaaaaaaa taggtaaata |
| 6001 | ctattacatt gcaggagaag tctactcgca gcaattaaat aaaagatgta tgagagcaac |
| 6061 | agtaatagta tgtatttgat atggtgatgg ttcatgataa aaattactga aaatatagtt |
| 6121 | caaataaaac taaagcaagg caaaaattat ccagacgtta atgtttatgt acttctaaaa |
| 6181 | gaaaaagtgt taattgatat aggccccaaa tcaattaata cacttaacct tttaaaaaaa |
| 6241 | gaattagcta ggttaggatt aagttttgaa acacttaatt taattattct aactcaccat |
| 6301 | catgttgacc atgtaggact tttagagtac cttccctctg ggttacgtat cgttggacct |
| 6361 | gatcatttag acttttatag ttcagatatt tataaaaaaa gtattcaaaa attgttagtt |
| 6421 | gacgataatc tctctattga atttaaaaat gatatagaaa aacaacttac tactgaaata |
| 6481 | attccaagta ttaatagaga aaactacgtt ccttttagtg agtcaaagaa aattttgcaa |
| 6541 | caatttggtc ttacagctgt agagctatca ggtcattcta gtgaagatat tgtaattact |
| 6601 | gattcagaaa ataactgttt tactggtgat attattattc ctaaaatttt ttttaactgt |
| 6661 | atatatgagg ttgataaagt aaggcccaaa catcaacgtt ggtcatatta tcatgagctt |
| 6721 | aattttttag acaggttagt aaacctagta ttgccaggac atggagatat tctaaagtta |
| 6781 | gaagaattaa aaaaggcagt tttggttaat agaaaaagaa tgagacggac agagaaaaaa |
| 6841 | ataataagag agttaaataa agaaacagtt aatggagtgg agaatgtttg tcgaagtgta |
| 6901 | ttccaaagtt ttttgcctta tagcaaattc ttaccgtttt cagaagtagt tagtgttatc |
| 6961 | gagagtaatg atgagagaat taactattga tattatttat ataaatacac taaataataa |
| 7021 | cagctttgca agtttagatt ctagaaaaag aatcaaaaag ttacagcatc agctaggaca |
| 7081 | atatatgctt tctcaaatat cttataaaaa aggttattca atttcccata gtcatatgtg |
| 7141 | tgtagcctta gcaagttatg ttaatagggt tggtattgat attgaactta taaataaaac |
| 7201 | aaagaaggca aggatacagt tcctttcgaa aagcgaaaaa caattagtaa atagatatgg |
| 7261 | ttttacaaga atatggacac taaaggaagc tatagctaag tatcataccg ttgggttacc |
| 7321 | tcggcttaat acagttgaga ttaaagaaat aaatgcttcg aacgttatct attttgttaa |
| 7381 | taaagcacca agaaaattgc agtataaatt tttagatata atcccttctt ataggttgag |
| 7441 | tgtagtggca aaaaaggtat caagtttttg cattagaatc acacaagaag aagatttgaa |
| 7501 | ggggctaata aggagaatgt aattaatgca aggaagagac ataattctag gaatacttga |
| 7561 | acgaaataat agaacaggat atgaaattaa tgatattttg aaaaatcaat tatcttattt |
| 7621 | ctatgacgga acatatggaa tgatatatcc aacattacga aagcttgaaa aggaaggaaa |
| 7681 | aataaaaaag gaaaaaattg tgcaaaatga taagccaaat aagaatgttt attctattac |
| 7741 | agatacggga atagaagaat ttaaagaata cttggattct tctattcaag atgatattta |
| 7801 | taaatcggac tttttaatgc gtctcttttt tggaaactca ttaccaaatc aagaaattat |
| 7861 | tagagctatt aaacaagaga tacaacgtaa aaatgaaaag atagatcaat taactgtaaa |
| 7921 | ttataaaaaa tggaaaaaaa atggaatgag taaaacacaa gaaataaccg tcaaatatgg |
| 7981 | tattgcgcag tataccgcaa ttgttcagat gctgacaaaa gagcttgata cgttacgaag |
| 8041 | aaatgagatg gatgattaat gtcattagga attatattta gtgggcaagg agcacaaaaa |
| 8101 | tctaaaatgg gtcttgattt ttatgaagat ccactatttg ctgaattact taaccacgca |
| 8161 | agtaatatat ctggtctaaa catgttaaaa atcttggaaa acaaaaataa tgagcttaca |
| 8221 | gagactgtta atttacaacc aacattaaca acattgaatt atggcatata tcgaatgctg |
| 8281 | aaaagggata tttttgatat gaaagtaagc tgtatggcag ggctttcttt aggagaatat |
| 8341 | tctgcactga ttgcttctaa tgctttaact tttgaacagg gaatacaatt actagtagac |
| 8401 | cgcggaaaat atatgcagga agcttcgaat agtaatgcag gaaaaatgtt agcacttata |
| 8461 | aaacctaaac taaaagagat aactcaaatt tgtgctttgt gtaaggttga aattgctaat |
| 8521 | tataattctc caaagcagat agtaataggc ggacaaaatt tgcaaattga atttgcaaag |
| 8581 | aaaatgatta tggagcgtaa agctgcatta agaataattg agttagaagt aagtggcgcc |
| 8641 | tttcatactt cgttattttc aaatgttcaa aaacaattgg aaaagcgatt aaaagatgtt |
| 8701 | aaatttgaga atccacaaat tccggtagtt agtaatacta cagttgagga atttcaaaag |
| 8761 | gaaagtctta cagccgtatt atcaaaacaa gttgctaatc ctacatattt tgaaaaagat |
| 8821 | attaagttaa tgaaaaatac ttatggattg acacatatag ttcaaattgg tcctggtaag |
| 8881 | gcattaagta attttgtgaa gcaaatgtca ttaggaatta agacatataa tatttctaat |
| 8941 | ataaaagact atagaaaatt tctgaatagt tatagagata ttaatttgaa aggaaagaaa |
| 9001 | aatggatttt gagaaaattc agcaactaat tcaaatgttt gaaagctcta atacaagaga |
| 9061 | actaaaaata gatgataata actttcacat ttatcttaca aaaaacgtaa gtaaagaacc |
| 9121 | aattcacgat ataaaatttg aatcgaataa aattcagcaa gcctcagagg caaaagcaaa |
| 9181 | caggaaaacg ataacagccc cattggttgg aacagtttat ttagcctctt ctcctacgtc |
| 9241 | taaaccgtat gttcaagtag ggagccatat tgataaagga gatacagtgt gtgtaataga |
| 9301 | ggctatgaag ctaatgacgg aaataaaaag tgaggttacc ggaacaattg aaaaagttaa |
| 9361 | tgtagagaat ggagaattag ttgaagttgg gcaaccgtta ttttcagttt ctggagaaaa |
| 9421 | agaaagttaa tgaaagagtg aggtgtagtg atgttttcta aagtactagt tgctaatcgt |
| 9481 | ggagaaatag ctgttcgtat tattagaact cttcatgaat tgggcattaa agcggtagct |
| 9541 | atttattcga ttgttgatca agaaagtctt catgttcaat tggctgatga agcagtttgt |
| 9601 | gtaggtggag cacgaccaca ggattcgtat ttaaatacta ctaatatttt aacagcagca |
| 9661 | attggaacag gagcacaagc gatacaccct ggctttggat tcctttcaga aaacgctgag |
| 9721 | tttgcaagaa tgtgtgaaaa atgcggaata gtatttatag gtccgcgtgc tgctacgatt |
| 9781 | gatttaatgg gaaataaaga aaatgcgcgc gagactatgc aaaagagagg gatacctgtt |
| 9841 | attcctggaa gtagttccta cataacaaac agttatgatg caaaaagagt tgcagataaa |
| 9901 | ataggatatc ctattttaat aaaggcggca gctgggggtg gaggtaaagg tattagaaga |
| 9961 | gttgtaacgc ctgagcaaat gaaacaagaa tttaataatg cgcaaagaga agctcgtata |
| 10021 | tcatttggtg atgatcgtat gtaccttgaa aaaataatgt gcaatgttaa acatattgaa |
| 10081 | gtacaggtag ttcgtgatag attcggaaat agtgtctatt ttccagaacg cgattgttca |
| 10141 | ctccaacggg ataagcaaaa aattatagaa gagagtccat gctcggttat caattcagaa |
| 10201 | caaagaaaaa ttttaggaag gtatgctatt aaagcaattg aagcagttga ttatcttaat |
| 10261 | acaggaacta ttgaattttt gatggataaa aataataaat tttattttat ggaaatgaat |
| 10321 | actcgtattc aagttgaaca tactgtaact gagatggtaa cgggaataga tctagtaaag |
| 10381 | gtacaactga tgatcgcttc aggtgaggaa cttccttttt cacaaaataa tattaagtta |
| 10441 | aatggggtag ctattgagtg tagaattaat gcagaagatc ctaaaaataa ctttgttcct |
| 10501 | tcaactggaa aaataaatta tttatattta cctgtaggta acttgggtat gagaattgat |
| 10561 | actgcacttt actctggaga aaagataaca ccattttatg attcaatgat tgcaaaagta |
| 10621 | atctcgcatg gccatactcg cgtagaagcg atcaatagaa tgaagaggtt aatgcaagaa |
| 10681 | ctggttataa aaggtgttaa aacaaataaa gatcttcact tatcaatttt gggagattca |
| 10741 | agttttttga aagatacagt aacaactgag tatttagaga aaaacttttt accaatctgg |
| 10801 | aaagagaggg agaaaaatgc agctgtatga aagtgcaacg ttaactaagc aacatgtaaa |
| 10861 | ggcgaatatt aaagcaaatc aaaaagtacc tgatgggatg ttaaaaaaat gtcctaagtg |
| 10921 | tgggaaaatc tttctgtcta cagaatttga taagtatttt tcgtgtcctg gttgtaatta |
| 10981 | tggatttagg attggttcat ggcaaagggt agcctgggcg gttgatgaat tttatgaaga |
| 11041 | ggatggttca cgagaactaa taacctcaga tcctttacat tttcctaatt atgtaaaaaa |
| 11101 | gattcaagga cttcaaaaga ctactaaggt aaatgaagca gttttaacgg gtaaagctaa |
| 11161 | aataaaagac caaatttttg agtgtggcat tatggatcct cactttataa tgggatcatt |
| 11221 | aggaacaatt actggcgaaa agattacgag attatttgaa cgtgctacaa aggagaggtt |
| 11281 | gcctgttgtc ctatggactg cttcaggggg agctagaatg caagaaggaa ttatgtcatt |
| 11341 | aatgcagatg gcaaaaataa gtcaagctat agcacaacat gcatcgaaag gacttttata |
| 11401 | cattgttatt ttaactgatc caacaacagg tggagtaaca gcaagttttg caatgcaagg |
| 11461 | agacattatt ttagcagagc cacatacttt agtaggattt gcagggcggc gagttattga |
| 11521 | gcaaactatt catgagagaa tcccagatac gttacaggat gcggagaatg taataaaaca |
| 11581 | tggattcata gatttaatag tatctcggag tgaagaaaaa tctttacttt ataaattgtt |
| 11641 | aaaatatggt aggtgataat aatggcaact gagaatgcaa tggcaattgt aaaagctgct |
| 11701 | cgaagtgata ataaaataac tgctaaagaa attatagaag aagtattttc agattttatt |
| 11761 | gaattccatg gtgatcgtaa aggaacagat gattcagcaa ttcttggagg attggcaata |
| 11821 | ctttcatcta caccagtaac agtaattgcg actaataggg gggaaacagt tggtgaacat |
| 11881 | ttgagtacac attttgggtg tccaactcca ggtggctatc gtaaagcttt gcgtttagca |
| 11941 | aagcaggctg caaagtttaa tagaccaatt atatttttag taaatactcc tggagcttat |
| 12001 | ccagggaaaa cagctgaaga acaagggcaa ggatctgcga ttgcgcaaaa tattatccaa |
| 12061 | ataagtcaac ttccagttcc aattattact attatttatg gtgaaggtgg cagtggaggt |
| 12121 | gcattagcat tggcttgcgg tgatcaggtg tggatgctag agaatagtac atattcaatt |
| 12181 | ttatcacctg agggttttgc ctctatttta tggaaagatg gatcacgtac agaagaagcc |
| 12241 | gcagaattaa tgcaaatgac cccaaaagcc ttgctaagac ataaggttat tgaaggaata |
| 12301 | attccagaaa gtaaagatca caaagtaaca tgtcgagcaa ttgctaatat tttagataaa |
| 12361 | cagttacaag aattaggaaa actttcagcg gtagatttat tgattcaacg aaaagaaaga |
| 12421 | tatagaaaat tttagttagt atagtaagag gagagaatac attacggtgc acgacttatt |
| 12481 | atcgcttaat agaattaaag aattatataa agagaaagca atgacaaatt gcaacttttt |
| 12541 | ttactttaat aaagtagatt ctactcaaaa aatagcaaaa tattttttag accatttaaa |
| 12601 | tatcaataaa ccatgtacta tttttgctgg aagtcaaacg agaggctatg gtaaaagaaa |
| 12661 | taggctattt tattcaccac aaaattcggg gatttatatg agtataatta ttccacaata |
| 12721 | cgaaatgcaa aatgaggata ttggtttatt aacaatttca ttagggagtg gaatactaga |
| 12781 | agttttaaag aagtattatc ctaaaaaaat tttttattta aaatgggtaa atgatattct |
| 12841 | attaggcaag gtgaactact cggccataaa tgaccgagct tctgggaaca cgaagtagta |
| 12901 | tttaggatat tacacaagta ttcttgctac ctaacttaca gaactccgtt cttttaccaa |
| 12961 | ggctagttc |

The amino acid sequences for the PKS gene cluster proteins noted in Table 22 and encoded by the gene cluster are provided below. Annotations providing their locations in the 12,969 bp cluster sequence referenced above and corresponding to genome map locations are also indicated.

LOCUS PKS_encoding_region 12969 bp DNA

| | |
|---|---|
| DEFINITION | Lactobacillus reuteri strain 3632. |
| SOURCE | Lactobacillus reuteri |
| ORGANISM | Lactobacillus reuteri |
| FEATURES | Location/Qualifiers |
| source | join (11662..>12969,<1..11661) |
| | /organism = ″Lactobacillus reuteri″ |
| | /mol_type = ″genomic DNA″ |
| | /strain = 3632 |
| CDS | complement (176..1861) |
| | /gene = ″emrY″ |
| | /locus_tag = ″LREU3632_02405″ |
| | /inference = ″ab initio prediction: Prodigal: 2.6″ |
| | /inference = ″similar to AA sequence: UniProtKB: P52600″ |
| | /product = ″putative multidrug resistance protein EmrY″ |

/translation = ″MTKRKNKFLIIIAMCISIFLCMLDTTVMNIALPAIQNELHVTLT
NLSWALNIYTILFASLTIPLSKLAEKLGMNFLYIIGLIMFLIGSSISGISPNLAILIL
GRGIQSLGAAILFPLSMTIGINIVTVKTRKKVIAALGITQGLAAALGPSIGGILTQFM
GWHSIFSVNIPFTILSLVICLSTFNFHEPKSNEGNDYLGSLLGIILLSALTLILTQGR
SWGWKSPAINLLFFIFILTFILFLITEHYAKAPMIPLVLFKNREFTGSAVSIILSNLF
LVAVTVILPTYFTHIQHRTELEAALLITPITGMIFIMSPLSAILLDKLGSRAIILSGF
LLMGISYYLFTHIDMDNITLIIFTCIILGTGYGIIAGPITVLAASNFEGYLLTASQSV
AGVLRQVGVSLAVAIFLTGLYGNLNTAKHNSISYINQQVNNLNIPKYQKNKIRDNSIK
SLNNNSNSRTPSKHFSKKLIKETTDKEYTKQINSLPYEPSKIQEKEIYSHTKANVKLV
FHRLNNSINITIKKIKTYANSQYSTAFIKLYKVSLPFLIFSCLSCLLFPKRKK″ (SEQ ID NO: 7)
/label = ″emrY CDS″
CDS 1991..4087

/gene = ″fabF_2″
/locus_tag = ″LREU3632_02406″
/EC_number = ″2.3.1.179″
/inference = ″ab initio prediction:Prodigal: 2.6″
/inference = ″similar to AA sequence: UniProtKB: Q9KQH9″
/product = ″3-oxoacyl-[acyl-carrier-protein] synthase 2″

/translation=″MTAVIVGIGITSSCGESFTEIEQNVGKGKTGISNIDYFDTSELT
CGIAGNLSKKIWKEVLQIADKNELDWSSSLSIYTIQRLLESYNISKKQRIGLSLGTCN
GGIHSLAEYLDTSNDKFLKNYPPYIQSKDIAHYFNFNGPKYSFNSACAASANAIAYGA
EMINNSDADLVVTGGCDPMSEWVFAGFNSLRTFNSKNCMPYGEEYGLNLGEAATYFLL
EDKDKAIKKGHRIYAEILGHGLSNDAYHPTAPDKDGSGISYAIKMALKNSGLKPEDIL
YINSHGTGTKANDSAEYRGFKTVFRNEMPFISSMKGYVGHNLGAAASTELAISLIGMN
SQKVLYPNFNLTKYREDCNDEHILKKPYSLDGYEDINFINNNAAFGGQNVAVIFHVNL
EGKYGHSEKKLKTQQPIYINNFGVASDKAYMTKHGIGILDDLRPLKKKYPKLYKRRMN
MLTQVSIIAAKQTLQDQYSNCGLVYGTPFGSLSSTLKYVDSIQKYGFKNASGAYFPDL
VINSTTGHICQALSLKSYSSSISSGGDEDLRALIIAHNALNKGYASTMLVGAGQEETE
LGNKVLKREVNNHATFLSLSNKKMQETIAEVLSSGAMGFKNKKELLTIIRSKIDEKLA
NDADLKVIIQNNSEITNDELISYFKNDNNISINSDNFADSNFKSFVNHRNENRLLLVG
ISQVNDVSFADIKKIK″ (SEQ ID NO: 8)
/label = ″fabF 2 CDS″
CDS 4105..4377

/locus_tag = ″LREU3632_02407″
/inference = ″ab initio prediction: Prodigal: 2.6″
/inference = ″protein motif: CLUSTERS: PRK09184″
/product = ″acyl carrier protein″

/translation = ″MKHSIEEIKDILKEKVLIERLELDDVEPNDISDNENLFDEEGLA
LDSVEALDIMTGISEEFGIDTSMLGQEDINHFQSVNDMAKYISENE″ (SEQ ID NO: 9)
/label=″acyl carrier protein CDS″
CDS 4387..5106

/gene = ″fabG_2″
/locus_tag = ″LREU3632_02408″
/EC_number = ″1.1.1.100″
/inference = ″ab initio prediction: Prodigal: 2.6″
/inference = ″similar to AA sequence: UniProtKB: 067610″
/product = ″3-oxoacyl-[acyl-carrier-protein] reductase FabG″

/translation = ″MKSVLITGITGGIGRKLTEAYSSKGYHIYGTCSRNSDSLQQFKE
KWPSVEIIQINHDDLIDVSTEYSFFFRKVQPDIVINNAGIVKDNFLVQMSVNDFQEVL
TTNLISAWVIVKEMLLHLNDNKIHKIINVASISGIIGREGQCNYAATKGGLVGLCQLI
EHLAPKGSNVISFSVAPGLIDTDIKGKMPKKKIDNLKKATLANRLGTPEEVSKFIFKL
SEEDISYSDGTLYRIDGGVLK″ (SEQ ID NO: 10)
/label = ″fabG 2 CDS″
CDS 5103..5627

/locus_tag = ″LREU3632_02409″
/inference = ″ab initio prediction: Prodigal: 2.6″
/product = ″hypothetical protein″

/translation = ″MKIINNTIQVTDLLQSYGKDEKDIVIIGPSPFNELDCLKETKII
DKVQLNLEEVFSFVKNNSVALMKKRRGTIAFLLNPQSFEGGNNIYSPIYNSAIKSFLK
SLSKEMNPFRVKVMGIILPLTQDTKSTRKYDLVTLKYKGINNEKQVQDILSLLKLSEI
LNGQIVSLGAELNL″ (SEQ ID NO: 11)
/label = ″hypothetical protein CDS″
CDS 5744..6079

/gene = ″fabZ_3″
/locus_tag = ″LREU3632_02410″
/EC_number = ″4.2.1.59″
/inference = ″ab initio prediction: Prodigal: 2.6″
/inference = ″similar to AA sequence: UniProtKB: P64107″
/product = ″3-hydroxyacyl-[acyl-carrier-protein] dehydrataseFabZ″

/translation = ″MITALQQFGNEEFFFKGHFPNNPIVPGVLLTESIAQAGLILISL
LEGQKVKIGYLAQIEKTKFFKEVYPDEQVKVKCSLKKKIGKYYYIAGEVYSQQLNKRC
MRATVIVCI″ (SEQ ID NO: 12)
/label = ″fabZ 3 CDS″
CDS 6094..6990

/locus_tag = ″LREU3632_02411″
/inference = ″ab initio prediction: Prodigal: 2.6″
/inference = ″protein motif: Pfam: PF00753.21″
/product = ″Metallo-beta-lactamase superfamily protein″

/translation=″MIKITENIVQIKLKQGKNYPDVNVYVLLKEKVLIDIGPKSINTL
NLLKKELARLGLSFETLNLIILTHHHVDHVGLLEYLPSGLRIVGPDHLDFYSSDIYKK
SIQKLLVDDNLSIEFKNDIEKQLTTEIIPSINRENYVPFSESKKILQQFGLTAVELSG
HSSEDIVITDSENNCFTGDIIIPKIFFNCIYEVDKVRPKHQRWSYYHELNFLDRLVNL
VLPGHGDILKLEELKKAVLVNRKRMRRTEKKIIRELNKETVNGVENVCRSVFQSFLPY
SKFLPFSEVVSVIESNDERINY″ (SEQ ID NO: 13)
/label = ″Metallo-beta-lactamase superfamily protein CDS″
CDS 6971..7522

/locus_tag = ″LREU3632_02412″
/inference = ″ab initio prediction: Prodigal:2.6″
/product = ″hypothetical protein″

/translation=″MRELTIDIIYINTLNNNSFASLDSRKRIKKLQHQLGQYMLSQIS
YKKGYSISHSHMCVALASYVNRVGIDIELINKTKKARIQFLSKSEKQLVNRYGFTRIW
TLKEAIAKYHTVGLPRLNTVEIKEINASNVIYFVNKAPRKLQYKFLDIIPSYRLSVVA
KKVSSFCIRITQEEDLKGLIRRM″ (SEQ ID NO: 14)
/label = ″hypothetical protein CDS″
CDS 7526..8059

/locus_tag = ″LREU3632_02413″
/inference = ″ab initio prediction: Prodigal: 2.6″
/inference = ″protein motif: Pfam: PF03551.8″
/product = ″Transcriptional regulator PadR-like family protein″

/translation = ″MQGRDIILGILERNNRTGYEINDILKNQLSYFYDGTYGMIYPTL
RKLEKEGKIKKEKIVQNDKPNKNVYSITDTGIEEFKEYLDSSIQDDIYKSDFLMRLFF
GNSLPNQEIIRAIKQEIQRKNEKIDQLTVNYKKWKKNGMSKTQEITVKYGIAQYTAIV
QMLTKELDTLRRNEMDD″ (SEQ ID NO: 15)
/label = ″Transcriptional regulator PadR-like family protein
CDS 8059..9012

/gene = ″baeC″
/locus_tag = ″LREU3632_02414″
/EC_number = ″2.3.1.39″
/inference = ″ab initio prediction: Prodigal: 2.6″
/inference= ″similar to AA sequence: UniProtKB: A7Z4X8″
/product=″Polyketide biosynthesis malonyl CoA-acyl carrier
protein transacylase BaeC″

/translation = ″MSLGIIFSGQGAQKSKMGLDFYEDPLFAELLNHASNISGLNMLK
ILENKNNELTETVNLQPTLTTLNYGIYRMLKRDIFDMKVSCMAGLSLGEYSALIASNA
LTFEQGIQLLVDRGKYMQEASNSNAGKMLALIKPKLKEITQICALCKVEIANYNSPKQ
IVIGGQNLQIEFAKKMIMERKAALRIIELEVSGAFHTSLFSNVQKQLEKRLKDVKFEN
PQIPVVSNTTVEEFQKESLTAVLSKQVANPTYFEKDIKLMKNTYGLTHIVQIGPGKAL
SNFVKQMSLGIKTYNISNIKDYRKFLNSYRDINLKGKKNGF″ (SEQ ID NO: 16)
/label = ″baeC CDS″
CDS 9002..9430

/gene = ″accB_2″
/locus_tag = ″LREU3632_02415″
/inference = ″ab initio prediction: Prodigal:2.6″
/inference = ″similar to AA sequence: UniProtKB: P0ABD8″
/product=″Biotin carboxyl carrier protein of acetyl-CoA″
carboxylase

/translation = ″MDFEKIQQLIQMFESSNTRELKIDDNNFHIYLTKNVSKEPIHDI
KFESNKIQQASEAKANRKTITAPLVGTVYLASSPTSKPYVQVGSHIDKGDTVCVIEAM
KLMTEIKSEVTGTIEKVNVENGELVEVGQPLFSVSGEKES″ (SEQ ID NO: 17)
/label = ″accB 2 CDS″
CDS 9451..10830

/gene = ″cfiB_2″
/locus_tag = ″LREU3632_02416″
/EC_number = ″6.4.1.7″
/inference = ″ab initio prediction: Prodigal:2.6″
/inference = ″similar to AA sequence: UniProtKB: D3DJ42″
/product = ″2-oxoglutarate carboxylase small subunit″

/translation = ″MFSKVLVANRGEIAVRIIRTLHELGIKAVAIYSIVDQESLHVQL
ADEAVCVGGARPQDSYLNTTNILTAAIGTGAQAIHPGFGFLSENAEFARMCEKCGIVF
IGPRAATIDLMGNKENARETMQKRGIPVIPGSSSYITNSYDAKRVADKIGYPILIKAA
AGGGGKGIRRVVTPEQMKQEFNNAQREARISFGDDRMYLEKIMCNVKHIEVQVVRDRF
GNSVYFPERDCSLQRDKQKIIEESPCSVINSEQRKILGRYAIKAIEAVDYLNTGTIEF
LMDKNNKFYFMEMNTRIQVEHTVTEMVTGIDLVKVQLMIASGEELPFSQNNIKLNGVA
IECRINAEDPKNNFVPSTGKINYLYLPVGNLGMRIDTALYSGEKITPFYDSMIAKVIS
HGHTRVEAINRMKRLMQELVIKGVKTNKDLHLSILGDSSFLKDTVTTEYLEKNFLPIW
KEREKNAAV″ (SEQ ID NO: 18)
/label = ″cfiB 2 CDS″
CDS 10817..11656

/gene = ″accD_2″
/locus_tag = ″LREU3632_02417″
/EC_number = ″6.4.1.2″
/inference = ″ab initio prediction: Prodigal: 2.6″
/inference = ″similar to AA sequence: UniProtKB: P0CC08″
/product=″Acetyl-coenzyme A carboxylase carboxyl transferase subunit beta″

/translation = ″MQLYESATLTKQHVKANIKANQKVPDGMLKKCPKCGKIFLSTEF
DKYFSCPGCNYGFRIGSWQRVAWAVDEFYEEDGSRELITSDPLHFPNYVKKIQGLQKT
TKVNEAVLTGKAKIKDQIFECGIMDPHFIMGSLGTITGEKITRLFERATKERLPVVLW
TASGGARMQEGIMSLMQMAKISQAIAQHASKGLLYIVILTDPTTGGVTASFAMQGDII
LAEPHTLVGFAGRRVIEQTIHERIPDTLQDAENVIKHGFIDLIVSRSEEKSLLYKLLK
YGR″ (SEQ ID NO: 19)
/label = ″accD 2 CDS″
CDS 11680..12435

/gene = ″accA_2″
/locus_tag = ″LREU3632_02261″
/EC_number = ″6.4.1.2″
/inference = ″ab initio prediction: Prodigal: 2.6″
/inference = ″similar to AA sequence: UniProtKB: Q9FBB7″
/product = ″Acetyl-coenzyme A carboxylase carboxyl transferase subunit
alpha″

/translation = ″MAIVKAARSDNKITAKEIIEEVFSDFIEFHGDRKGTDDSAILGG
LAILSSTPVTVIATNRGETVGEHLSTHFGCPTPGGYRKALRLAKQAAKFNRPIIFLVN
TPGAYPGKTAEEQGQGSAIAQNIIQISQLPVPIITIIYGEGGSGGALALACGDQVWML
ENSTYSILSPEGFASILWKDGSRTEEAAELMQMTPKALLRHKVIEGIIPESKDHKVTC
RAIANILDKQLQELGKLSAVDLLIQRKERYRKF″ (SEQ ID NO: 20)
/label = ″accA 2 CDS″
CDS 12467..12898

/locus_tag = ″LREU3632_02262″
/inference = ″ab initio prediction: Prodigal: 2.6″
/inference = ″protein motif: CLUSTERS: PRK11886″
/product = ″bifunctional biotin- [acetyl-CoA-carboxylase] synthetase/biotin
operon repressor″

/translation = ″MHDLLSLNRIKELYKEKAMTNCNFFYFNKVDSTQKIAKYFLDHL
NINKPCTIFAGSQTRGYGKRNRLFYSPQNSGIYMSIIIPQYEMQNEDIGLLTISLGSG
ILEVLKKYYPKKIFYLKWVNDILLGKVNYSAINDRASGNTK″ (SEQ ID NO: 21)
/label = ″bifunctional biotin--[acetyl-CoA-carboxylase]
synthetase/biotin operon repressor CDS″

B. Engineering of Mersacidin Biosynthetic Cluster from LactobaciRus Reuteri into Bacillus Subdlis

The lanthipeptide mersacidin is a ribosomally synthesized and post-translationally modified peptide (RiPP) produced by LactoBacillus reuteri and Bacillus amyloliquefaciens. It has antimicrobial activity against a range of Gram-positive and Gram-negative bacteria, including methicillin resistant Staphylococcus aureus, giving it potential therapeutic relevance. The structure and bioactivity of mersacidin are derived from a unique combination of lanthionine ring structures, which makes mersacidin also interesting from a lantibiotic-engineering point of view. Lantibiotics are a class of polycyclic peptide antibiotics that contain the characteristic thioether amino acids lanthionine or methyllanthionine. as well as the unsaturated amino acids dehydroalanine. and 2-aminoisobutyric acid. They belong to ribosomally synthesized and post-translationally modified peptides. These peptides primarily act by disrupting the membrane integrity of target organisms. The production of active lantibiotics in bacteria is typically mediated via a gene cluster. Production of a lantibiotic by bacteria requires a series of steps, including formation of the prelantibiotic, dehydration and cross-linkage reactions, cleavage of the leader, and secretion, with proteins/enzymes/transporters involved in these necessary steps encoded and/or regulated via the gene cluster.

The Bacillus subtilis strain 105 is modified to introduce the mersacidin cluster from LactoBacillus reuteri so as to efficiently produce mersacidin. In a particular aspect, Bacillus subtilis strain 105 is modified to introduce the mersacidin cluster from LactoBacillus reuteri so as to efficiently secrete mersacidin. In particular, the LactoBacillus reuteri strain 3632 (ATCC PTA-126788) contains a BGC that encodes for a full suite of proteins required for mersacidin production. LactoBacillus reuteri strain 3632 is detailed and described, including its full genome nucleic acid sequence in Kumar et al, WO 2020/163398A1, published Aug. 13, 2020, the entire contents of which is incorporated herein by reference.

LactoBacillus reuteri strain 3632 encodes/produces two mersacidins, denoted Mersacidin 1 and Mersacidin 2, which have the amino acid sequence depicted below:

Mersacidin 1:
(SEQ ID NO: 22)
MEEKELEGVIGNSFESMTVEEMTKIQGMGEYQVDSTPGYFMESAAFSAL
TANITRHAMHHH
Mersacidin 2:
(SEQ ID NO: 23)
MDKEELEKIVGNNFEEMSLQKMTEIQGMGEYQVDSTPAASAISRATIQV
SRASSGKCLSWGSGAAFSAYFTHKRWC

The mersacidin cluster from LactoBacillus reuteri strain 3632 is encoded on a conjugation plasmid of 165 kb. The BGC contains 8 genes that encode for a full suite of proteins for the synthesis of mersacidin (TABLE 23). The gene cluster is introduced into B. subtilis strain 105 to enable synthesis and production of active and effective mersacidin by the modified B. subtilis strain.

TABLE 23
Genes involved in the synthesis and secretion of mersacidin

| Locus tag | Gene | Description | Remarks |
|---|---|---|---|
| LREU3632_02344 | | Sensory histidine kinase DcuS | Regulation of mersacidin production, |
| | | | may not be essential for transferring |
| | | | the cluster to B. subtilis; this was not |
| | | | cloned in the BGC evaluated |
| LREU3632_02343 | | Hypothetical protein | Regulation of mersacidin production, |
| | | | may not be essential for transferring |
| | | | the cluster to B. subtilis; this was not |
| | | | cloned in the BGC evaluated |
| LREU3632_02342 | lagD | Lactococcin-G-processing and | Transport and cleavage of signal |
| | | transport ATP-binding protein | Peptide; integration clones were |
| | | LagD | generated with and without this lagD |
| | | | sequence |
| LREU3632_02341 | | Lanthionine synthetase C-like | Essential |
| | | protein | |
| LREU3632_02340 | | Mersacidin 1 | Essential |
| LREU3632_02339 | | Mersacidin 2 | Essential |
| LREU3632_02338 | MrsD | NADPH-dependent FMN reductase | Essential |
| | | CDS | |
| LREU3632_02337 | | Hypothetical protein | Unknown function |

Genes in the Mersacidin biosynthetic gene cluster (BGC) from Lactobacillus, particularly from LactoBacillus reuteri strain 3632, were engineered into Bacillus subtilis #105 to efficiently produce (secrete) mersacidin. The BGC cluster was chromosomally integrated and confirmed by PCR and sequencing. The final strain did not contain any antibiotic markers.

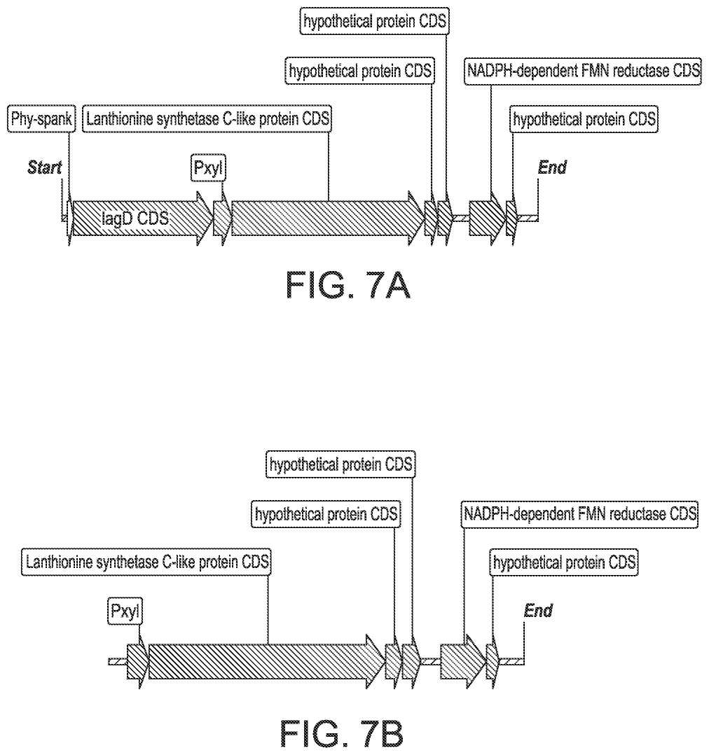

Three constructs were generated for integration in the B sub strain 105 genome. In a first initial step and construct, (i) the mersacidin pathway gene cluster and the wild type mersacidin pathway was cloned without promoters as a control sequence. The sequenced fragment was inserted into a Bacillus BGC genome integration vector (FIG. 6), including amyE sequence in left and right amyE arms for homologous recombination. The cassette is flanked by left and right amylase gene amyE arms for homologous recombination and integration at the amylase gene amyE in the Bacillus subtilis strain. The construct was then transformed into B. subtilis #105 and screened for full length insertion using PCR to verify left, right and middle portions of the mersacidin BGC. Similarly, using the cloned wild type mersacidin BGC, two promoters were inserted. This construct (2) was generated containing: Physpank promoter in front of (upstream of) LagD and a Pxyl promoter in front of the lanthione synthase C-like sequence. The remaining genes in the cassette were retained in series (FIG. 7A). The entire cloned pathway was sequence confirmed. The fragment was then inserted into Bacillus BGC genome integration vector (FIG. 6) using the left and right amyE arms for homologous recombination. In a third step and third construct (3), the lagD sequence was deleted and a single Pxl promoter was coned in front of the lanthione synthase C-like sequence. The remaining genes in the cassette were retained in series (FIG. 7B). The cloned gene cassette constructs was sequenced in its entirety to confirm all of the components and promoters. The cassette was then inserted into a suitable vector, in this instance designated as Bacillus BCG expression vector (FIG. 6). The cassette is flanked by left and right amylase gene amyE arms for homologous recombination and integration at the amylase gene amyE in the Bacillus subtilis strain. B subtilis strain 105 was transformed with the vectors under conditions to promote homologous recombination and integration and then screened for full length insertion using PCR to verify the left, right and middle portions of the Ahr PKS BGC.

The mersacidin encoding BGC was successfully engineered into B. subtilis #105 and confirmed by PCR and sequencing (data not shown). Junctional PCR confirmed the correct integration of mersacidin BGC into B. subtilis #105 genome (data not shown).

The engineered strains are evaluated for mersacidin activity as follows. The strains are grown overnight in Trypticase Soy Broth. The filter sterilized culture supernatant is evaluated for mersacidin activity by using Staphylococcus aureus as an indicator organism and determining inhibition of bacterial growth and/or bacterial killing activity. The culture supernatants are 2-fold serially diluted in 50 ul of Trypticase Soy Broth and added with 50 ul of midlog S. aureus containing approximately 1×105 cells/ml and incubated aerobically at 37° C. for 24-48 hours. Following incubation, minimum inhibitory concentration of the test material is recorded. Culture supernatant from the parent strain and an antibiotic (e.g. oxacillin, vancomycin, linezolid, tetracycline) are included as negative and positive controls, respectively.

The full sequence of the mersacidin cluster along with 5 kb flanking regions on both sides of the BGC, corresponding in total to 8742 bp is provided below (SEQ ID NO: 24).

| | |
|---|---|
| 1 | ctattttgac caatcgacca aagaggaggt tattatccac tattatagtg aatcactcat |
| 61 | gcaacagtat ctgaataaag taatataata tcagaagcaa taaatttatt tttctcagta |
| 121 | tcctgctgca cttctaccag tttacgatat attattgaac cattatctaa gtcatctacc |
| 181 | ttaaaattgt acaagaccca cctgctatgt gggtaggtaa taaaaagtca taaaaaacat |
| 241 | tatggagtaa taagtgtaaa gtaattacag gctaatcaaa aaagcagtaa ataataacta |
| 301 | tttactattc ttaatcatat tcccataaag catagtagag aaaataataa ctatacttgc |
| 361 | aatcattctg taattcatag tttgaattgc gatcattgtc actgccccac ttaaaaataa |
| 421 | caataaataa taaacacttt tctttattat ttctaacata ttagtttcct ttccatgtta |
| 481 | aatattcttg aaatgtttca aaatttatca aattatttgt tttccaatac ataacttcct |
| 541 | cactttcatc accacttttt tctctaagtt caataacctt ttttaagata ataaaaactt |
| 601 | cttcaatagc ggagcttgaa gaatgataaa acaaagactt gtcattgatg cgtttttgaa |
| 661 | tttgctttct aacacttgta acccattttt tatctctcaa ttggtattct aatgcattgc |
| 721 | agtttgcaat aacttttcct cccatacttg taagtacttt acacaaataa cctgttactg |
| 781 | tagtatttcc atttgaatta caagtactta aaactaaaca cggtttacca tctaagcgta |
| 841 | aggtatgtga ccaagtagaa agcttttcta atattaattt taaatctgac gaaatattat |
| 901 | gcatatatac tggagaagca actacaaaac agtctgattt caatatcatt tcttctaact |
| 961 | cgtaaatttt ggaatcacca tttctttgag tatagctaat atcatataac gtattcgggt |
| 1021 | tgagtctcat cttgttaaag tctctacaaa acaaatattg aactttatac tctttgctat |
| 1081 | ctagattgtt aaagaggctt tctaaaaaga attgcgtcga cccttgatat ctacaactgc |
| 1141 | cgttcagtac taatatccgt ttcaaattca tcatcctttc aaaaaagagg ctgggaaaat |
| 1201 | ttttcaccgg cctcattctt tatatcaacc ttgttaagat atcatgggga ttaaccacaa |
| 1261 | ggcggttaat tactctagca ccatctttta tgagtaaaat aagcactaaa tgctgcacca |
| 1321 | ctaccccaac ttagacattt tccagaagat gcacgtgata cttgaattgt tgcccgtgaa |
| 1381 | atcgcagaag ctgctggtgt tgaatccact tggtattcac ccataccttg aatttctgtc |
| 1441 | attttttgta aactcatttc ctcaaagtta ttacctacaa ttttttctaa ttcttctttg |
| 1501 | tccataacaa atacctccct ttaatgatga tgcatagcat gtcttgttat attggctgta |
| 1561 | agagctgaaa aggcagcact ttccataaaa tatccaggcg tcgaatctac ttgatattca |
| 1621 | cccatacctt gaatttttgt catttcctct acagtcatac tttcaaacga attccctatt |
| 1681 | acaccttcta attctttttc ttccataatt ttcaccccct ttcaatttcc aaaagtaatg |
| 1741 | gattagtttt aagcccttta tgacataata aggataaacc gatacccgaa agtccatcaa |
| 1801 | ataagcctaa cgcagaaaac tttccaaatc ttctaataga atagtcctta tttaatatgt |
| 1861 | aatttcctag cataatatta gttagtaagt tggtcttttc cttccattta gtagaacccg |
| 1921 | tctgcataaa aagttcatgc ataatcataa ttattcctgc ttttccgtgg cataatgtat |
| 1981 | cattccttgg cgcattttct aaatcattta atttttcttg tgcgtaatta ttatcaagtt |
| 2041 | gcatataatt acgcgctaaa agcattccag atactccttt acaccatccc atatcattct |
| 2101 | tcacaggagt aatttttttc attaaagcac ttgcttggtc tagctgccca atttcatata |
| 2161 | agcacattgc gtctccacaa ttaccatgcg caacaccgaa tttacctgaa tgcataatac |
| 2221 | aattcataaa ctttttttct aatctattca ttacattttg tgagatgtat ttaaatccaa |
| 2281 | ataatctttt ctgtaaatgt aaaagtttta taattccacc taaaccatta ataaaatcac |
| 2341 | taattagttc gcctttttca atgttttgct caatcttctt ttcaagctgt tttaaataag |
| 2401 | ccacttcttc ataatccaaa gttttataaa gactataatg aaataataat gggtatagtg |
| 2461 | gcgataaaaa tcccttaaag gctccttgta cctccagatc taatgacata ttaatggatg |
| 2521 | cttttaatag tttcttcgca aattctttat atctagcttc gtttttttca tatcctaatt |
| 2581 | gtataaagaa tactgttaca ccactaattc cacaatatag actttcatcc ataggtaaaa |
| 2641 | tatcccaatt gcctgcatta ttgatattta tggtcgaaat aataacacta ttttcatctt |
| 2701 | cgaatgcact attcataata gtatccgcaa tactgcttgc ttgtctcaca taattaaatt |
| 2761 | cttgatctaa caaatctcga cttttctttc ttacatcagt gattctatct cctactgata |
| 2821 | aagaagctaa tagaagtcct ctttcttttt ctatgtttc gtaattgaaa ttattgattc |
| 2881 | tatctaatgc aaggttaagt cctgatttct taaaatatgc atcataattt tttcctctac |
| 2941 | tatcagtaat acttgtcttc ccaactacag cagaaaaata gggaacatcc ccaaacagta |
| 3001 | aatcttcaac ttcactattt actattctct tatcatcata tggataagca gcaatattta |
| 3061 | acattagcct ttcacgatat ttcatttcaa cattataaga tgggtgtgct gcataattta |
| 3121 | acatcgtttc ataacgttct gtactcttcg ttaaaatacg aattctaaag tttgaaaatt |
| 3181 | ctttaacttg tttttttaac tctttaatat tattactaac aaaacttgtc attttgttat |
| 3241 | aaccatctag aatcgctaat ctatattttt ttacattaac ttctttatta ttacaggtaa |
| 3301 | ttggaatatt ttccccgcct ttaacgtatc cttgtcttct catttcaaaa tgaaaatttg |
| 3361 | aattttcaat atttacgggt acgagtactt tatttttcaa taaagtttct cttccttcta |
| 3421 | gtccacttag ttctatcgct tcattatttc ccattggtaa tttcgtgggt aataagcagg |
| 3481 | aaccctttat cgaatctcta ttcaaactca ttaaatattt tccaaacgcg ttttgaggag |
| 3541 | taattaatga attttgaaac atcgtttctc catcaacaag aattggttgg tccttttttg |
| 3601 | caattatatt ctctaaatgc aaatcgttta gccctaataa ataagaaata acaataaggt |
| 3661 | atccataacg ttcataataa tcggcacatt cgtctttact tattacagga acccgtgtta |
| 3721 | caaattcaaa aaaagcataa tctttataaa ataatccttt aggtaaaaaa tctatatcaa |
| 3781 | gtaaagttga actagactta ttaatccaat tacaaaactt ttcaaattgc tgaagaattc |
| 3841 | taagatcacg aggtttatat actatttttt tatgatttaa ttctaagatt ataactgtat |
| 3901 | ttcctttgca atgggaatct cccactccaa tttctatatt agttaagaca atgttttctt |
| 3961 | gtttaagatg taagaaatta caaagatcat ttctatgggt gccaattgcc tcaccaagtt |
| 4021 | ttgataaatt tgtcacaaaa aaattgacgc gcgttattat tattctagat aaggttggat |
| 4081 | actttcttaa aaacgcttta aatctagttt cattacaaaa gttttctttt ataaaagacg |
| 4141 | atagtacatc tgtatcagct tgactaattc ttataccaga ttttattttc tcttttttat |
| 4201 | atttatacaa ctctagtgct agcactttcc cacttttct aaataaatca ttgattagaa |
| 4261 | actcactcat agcttcgctg gctttagtag taattctaat ttgatgattc cacccatcgt |
| 4321 | ctatcctctg cattgcatat tctacaaaag gcttaaaaaa tacatctagc tttaatttat |
| 4381 | gcaaatgtat gttactaaac tgaaattttt caataatttc aaccaattct ttttcgaaag |
| 4441 | ggatctccga atctacttta attaacggtt gtaatgtttt cttaaaatca tctaacagca |
| 4501 | aatgggcatt actacagtac tctgttaaat cctgttcatt aaataaagtg cgtgtgtttt |
| 4561 | tccattcttt ataatattta tcatccagaa acggtttact atatatagca cgttcttcaa |
| 4621 | cggtaaatgc atccgttaaa tttaaggttt tcatattaca gtcctccatt tgtatttata |
| 4681 | ttttaaggat tttctataaa aggaatactt tttttattaa acgcaccata ttatgaataa |
| 4741 | ctgcgtttat agctaacttc aaaagctatt aactgtttaa ggttcattct gtataaacaa |
| 4801 | acatatgaag ttttctaaaa taaaaaccgt cctaaaactg ttagattgta taaccacaat |
| 4861 | tttagggcag ttttttctta aattgtttta ttattcacct attttgaggg ttcatattac |
| 4921 | attttttgtc atcctatgaa tttaaataag tttttttctt tttggttaat tcttcatagg |
| 4981 | taccagcttc aactattttc ccatttttta atacatatac tttattagtg gacttagcaa |
| 5041 | cagctagtct atgagtaata aaaagaattg ttttatgtaa tgtcattaaa tttttaataa |
| 5101 | ctttatgttc agtaatggga tctaggccac tggtagattc atctaatatt aatatttttg |
| 5161 | ctggcgataa aattgcccga gctattgcta agcgttgact ttgaccacca gaaagaatta |
| 5221 | agccaccttc atcgagcttt gtgttgtact ttaagggcat ttcatcaatt tcattgctga |
| 5281 | tacatgctat tttgcatgca tttataattt cttccatact cttttcttta tcacatccaa |
| 5341 | cgatcaaatt atccatgata gatcccgaga aaaggtatgt ttgctgaggc acatagcata |
| 5401 | taaaatttcg taattgataa gtactaagtt gattaatctt aactccatct actaaaatat |
| 5461 | ctccactatt tgattgtaaa aatcctacaa gtaatttagc tattgttgat tttcccgacc |
| 5521 | cactttgccc cagtaatgcc actttctcat catttttaat ccgtaaattt agttttttta |
| 5581 | aaacttcttt attattagca taatgatatg aaacattctt tatcactaat tctcccttgc |
| 5641 | ttaataaatt aagattattt tttctttgtt gtagtgtgat accattttgt tcttttttcg |
| 5701 | aaagcatcac atcatttaat ctatcattcg ctacctgtgc attttgcaat ttgggctgta |
| 5761 | aattaattaa attttgaatt gaactagtaa agtaactaaa taaagcattg aatgcgaaaa |
| 5821 | gttgtcctag tttaaaatca ttattcatta ttagatatgc tgcttgccat attattaaaa |
| 5881 | tatttaacac taactgtaca aaaactttta atgattcttg tatcgaatta gctatgttat |
| 5941 | atttcatcaa agtttttaac aagttagtaa agctattttc gatttttgca taaaattttg |
| 6001 | tttcagaatt taatgattta attacttcta taccttttat actttcaata agtaatgctt |
| 6061 | ctaactctgc attcctgatc attactttct tacttaatct atcgaataac ctagtaaaag |
| 6121 | aaataattat tacaaaataa attggaaaag aaaatactgc tagaaaaaat aaatccttac |
| 6181 | tttgaaaaaa taaagcactt cccaacgtag taacaattat tgtatctaga aaaagtgtaa |
| 6241 | cgactgaacc agctaatgct tctgtaattt tatttgcatc attaaacctt gaaacaatat |
| 6301 | cacctgttgt tctagacgaa aaaaatccta tctgcaaatc aaataatctt tttataaaat |
| 6361 | ttaaaagaat atttttcgat aagttctgtc ccaatactac tagtgcaatt ttttctaaaa |
| 6421 | aagaaaaaat tgcactaaaa aagtatagga ttatcaatcc aaacactatt atagatagta |
| 6481 | agttatattt actttttgga atcaatgaat caattacgaa ctgtaaggat aacgaactaa |
| 6541 | ttatatttat taacaatgta aagattgcaa atagcaaaat ttttattaga agcaatttat |
| 6601 | tactaaaaag ttgcttaaat aaatagttaa aattagattt tctttcatca ataggaataa |
| 6661 | agttgtctgc tttcgaaaaa aatattgctg ctccactcca ttccttaata acttcatcta |
| 6721 | tttctttttt tatggtccct attgttgggt ctggatcagc aatatacact tttttatttt |
| 6781 | taatttttaa aactacataa taatgcaaaa ttttttcttc ttttacaata tgaattataa |
| 6841 | aaggaaatgg taacttttct tttaataaaa agttgttttt tgcttttacc gccaaaggct |
| 6901 | ctaatccata ctttttagaa gcttctacta agccataaat gttcgtacct gaaatactag |
| 6961 | tattagcagc attacgcaaa tgttctagtg aaactcgact accaaattct ttaagtatca |
| 7021 | ttgctaatgc tgccactcca caatcctctt catttacttg aggaatataa ttacgataat |
| 7081 | acatatatct tcctcccacc taatcataac taaaataaat taattcgtat acaattaata |
| 7141 | ttaagcgtat ggcttttcga ttagacgcaa aaaaaggatt ttacaactaa tttagattca |
| 7201 | attataatta actaaatttc aaatttatta tattggagta atgtatatga aagacactaa |
| 7261 | ttatataaaa aaatctctag agaatcgcga aattcaaaaa ctgactgaag atgaaatgaa |
| 7321 | agctttcatt ggtggaacta cttatcatat taactacgat ggatatgcgc caagtaggaa |
| 7381 | tccatgggct tggctaactc actgggggca taaacactaa taatgaacta tataaatcca |
| 7441 | ttttacttat atgggttaga attttttgat ttatttatta acacattgtt aataaataat |
| 7501 | atagctggta aaaggaaaat aaatctactt tcaatctttg aaataggagt tatagtctta |
| 7561 | gtattgggct attatggata tacaatttac attgcatatg cacttctttt cttgtataaa |
| 7621 | aattttaaac ataataagcg ggtcttgaat tttgaagcgc ttattatgtt tgtatgcagt |
| 7681 | ttatttgtct ttcttatctc aaacattaat agtttcattc taaacacctg gtttttacca |
| 7741 | cattatagta acgatgtttt aatacgtttt caaatagtaa tttctgttat tatttctata |
| 7801 | atgttagtta gtatttcgtt tttttataat cctttatttt cgaaaatttc gaacaattta |
| 7861 | tttttaaaat ataaaggtcc aattttaaga aaatttgtaa ttacttatga agtttctttc |
| 7921 | ttctttatta taggtattgc ttctattgct gaatatttaa aaatagaatt taaaattcaa |
| 7981 | tggcttttaa ttttatgctt tctatttctt tcattaagcg taattatact gaccggtatt |
| 8041 | atgtttacat acaatagtaa aatgattcag ctcgaggaga ttagaaatag ttatattaat |
| 8101 | aaacaattgt attggaaaag ctttgaatct aactataaaa agttgagaaa actgcatcat |
| 8161 | gactatacga atcttttaat aacgttaata aggttaattg agaataatga ctttaaacat |
| 8221 | gcaaaatcct atgctaattc cttattagat tacgatagta aagtatattc gcctccttca |
| 8281 | ttcgatatta tacaaatttc tcaaaaaatt tataatcaag aattacgagc attattttta |
| 8341 | gaaaaggtga atataataaa atctcagaga attaaattgc atttggaaat ttctgataag |
| 8401 | atcgctatac cgtctaaatt attaattgat ctattaataa ttattagtaa tttactagat |
| 8461 | aatgctacga gtgcagcttt acagagtgac caaaaatcta tttctataag cttaaaaaaa |
| 8521 | acgctctcta attgttataa atttactata aagaattcaa taaaatctaa aattaatatt |
| 8581 | gaagaattat ttactgaaaa tttttcaacc aagaaaaacc atatgggaat aggacttaat |
| 8641 | aatgttattt caataattga cagttcacca aatttctttt tagaaacaaa atcttttaat |
| 8701 | gaatggatcg agtttgaact aatattaaag aggtgatgtc tt |

The amino acid sequences for cluster proteins noted in Table 23 and encoded by the gene cluster are provided below. Annotations providing their locations in the 8742 bp cluster sequence referenced above and corresponding to map locations are also indicated.

LOCUS Mersacidin_region 8742 bp DNA linear

| | |
|---|---|
| DEFINITION | Lactobacillus reuteri strain 3632. |
| SOURCE | Lactobacillus reuteri |
| ORGANISM | Lactobacillus reuteri |
| FEATURES | Location/Qualifiers |
| source | <1..>8742 |
| | /organism = ″Lactobacillus reuteri″ |
| | /mol_type = ″genomic DNA″ |
| | /strain = 3632 |
| CDS | complement(298 .. 459) |
| | /locus_tag= ″LREU3632_02337″ |
| | /inference = ″ab initio prediction: Prodigal: 2.6″ |
| | /product = ″hypothetical protein″ |

/translation = ″MLEIIKKSVYYLLLFLSGAVTMIAIQTMNYRMIASIVIIFSTML
YGNMIKNSK″ (SEQ ID NO: 25)
/standard_name = ″hypothetical protein CDS″

| | |
|---|---|
| CDS | complement (461..1030) |
| | /locus_tag = ″LREU3632_02338″ |
| | /inference = ″ab initio prediction: Prodigal: 2.6″ |
| | /inference = ″protein motif: Pfam: PF03358.9″ |
| | /product = ″NADPH-dependent FMN reductase″ |

/translation = ″MRLNPNTLYDISYTQRNGDSKIYELEEMILKSDCFVVASPVYMH
NISSDLKLILEKLSTWSHTLRLDGKPCLVLSTCNSNGNTTVTGYLCKVLTSMGGKVIA
NCNALEYQLRDKKWVTSVRKQIQKRINDKSLFYHSSSSAIEEVFIILKKVIELREKSG
DESEEVMYWKTNNLINFETFQEYLTWKGN″ (SEQ ID NO: 26)
/standard_name = ″NADPH-dependent FMN reductase CDS″

| | |
|---|---|
| CDS | complement (1275..1505) |
| | /locus_tag = ″LREU3632_02339″ |
| | /inference = ″ab initio prediction: Prodigal: 2.6″ |
| | /product = ″hypothetical protein″-Mersacidin 2 |

/translation = ″MDKEELEKIVGNNFEEMSLQKMTEIQGMGEYQVDSTPAASAISR
ATIQVSRASSGKCLSWGSGAAFSAYFTHKRWC″ (SEQ ID NO: 27)
/standard_name = ″hypothetical protein CDS″

| | |
|---|---|
| CDS | complement (1521..1706) |
| | /locus_tag = ″LREU3632_02340″ |
| | /inference = ″ab initio prediction: Prodigal: 2.6″ |
| | /product = ″hypothetical protein″-Mersacidin 1 |

/translation = ″MEEKELEGVIGNSFESMTVEEMTKIQGMGEYQVDSTPGYFMESA
AFSALTANITRHAMHHH″ (SEQ ID NO: 28)
/standard_name = ″hypothetical protein CDS″

| | |
|---|---|
| CDS | complement (1712..4654) |
| | /locus_tag = ″LREU3632_02341″ |
| | /inference = ″ab initio prediction: Prodigal: 2.6″ |
| | /inference = ″protein motif: Pfam: PF05147.7″ |
| | /product = ″Lanthionine synthetase C-like protein″-Lanthione |
| | synthetase C-like protein |

/translation = ″MKTLNLTDAFTVEERAIYSKPFLDDKYYKEWKNTRTLFNEQDLT
EYCSNAHLLLDDFKKTLQPLIKVDSEIPFEKELVEIIEKFQFSNIHLHKLKLDVFFKP
FVEYAMQRIDDGWNHQIRITTKASEAMSEFLINDLFRQSGKVLALELYKYKKEKIKSG
IRISQADTDVLSSFIKENFCNETRFKAFLRKYPTLSRIIITRVNFFVTNLSKLGEAIG
THRNDLCNFLHLKQENIVLTNIEIGVGDSHCKGNTVIILELNHKKIVYKPRDLRILQQ
FEKFCNWINKSSSTLLDIDFLPKGLFYKDYAFFEFVTRVPVISKDECADYYERYGYLI
VISYLLGLNDLHLENIIAKKDQPILVDGETMFQNSLITPQNAFGKYLMSLNRDSIKGS
CLLPTKLPMGNNEAIELSGLEGRETLLKNKVLVPVNIENSNFHFEMRRQGYVKGGENI
PITCNNKEVNVKKYRLAILDGYNKMTSFVSNNIKELKKQVKEFSNFRIRILTKSTERY
ETMLNYAAHPSYNVEMKYRERLMLNIAAYPYDDKRIVNSEVEDLLFGDVPYFSAVVGK
TSITDSRGKNYDAYFKKSGLNLALDRINNFNYETIEKERGLLLASLSVGDRITDVRKK
SRDLLDQEFNYVRQASSIADTIMNSAFEDENSVIISTININNAGNWDILPMDESLYCG
ISGVTVFFIQLGYEKNEARYKEFAKKLLKASINMSLDLEVQGAFKGFLSPLYPLLFHY
SLYKTLDYEEVAYLKQLEKKIEQNIEKGELISDFINGLGGIIKLLHLQKRLFGFKYIS
QNVMNRLEKKFMNCIMHSGKFGVAHGNCGDAMCLYEIGQLDQASALMKKITPVKNDMG
WCKGVSGMLLARNYMQLDNNYAQEKLNDLENAPRNDTLCHGKAGIIMIMHELFMQTGS
TKWKEKTNLLTNIMLGNYILNKDYSIRRFGKFSALGLFDGLSGIGLSLLCHKGLKTNP
LLLEIERG″ (SEQ ID NO: 29)
/standard_name = ″Lanthionine synthetase C-like protein CDS″

| | |
|---|---|
| CDS | complement (4934..7084) |
| | /gene = ″lagD″ |
| | /locus_tag = ″LREU3632_02342″ |
| | /EC_number = ″3.4.22.−″ |
| | /inference = ″ab initio prediction: Prodigal: 2.6″ |
| | /inference = ″similar to AA sequence: UniProtKB: P59852″ |
| | /product = ″Lactococcin-G-processing and transport ATP-binding |
| | protein LagD″ |

/translation = ″MYYRNYIPQVNEEDCGVAALAMILKEFGSRVSLEHLRNAANTSI
SGTNIYGLVEASKKYGLEPLAVKAKNNFLLKEKLPFPFIIHIVKEEKILHYYVVLKIK
NKKVYIADPDPTIGTIKKEIDEVIKEWSGAAIFFSKADNFIPIDERKSNFNYLFKQLF
SNKLLLIKILLFAIFTLLINIISSLSLQFVIDSLIPKSKYNLLSIIVFGLIILYFFSA
IFSFLEKIALVVLGQNLSKNILLNFIKRLFDLQIGFFSSRTTGDIVSRFNDANKITEA
LAGSVVTLFLDTIIVTTLGSALFFQSKDLFFLAVFSFPIYFVIIISFTRLFDRLSKKV
MIRNAELEALLIESIKGIEVIKSLNSETKFYAKIENSFTNLLKTLMKYNIANSIQESL
KVFVQLVLNILIIWQAAYLIMNNDFKLGQLFAFNALFSYFTSSIQNLINLQPKLQNAQ
VANDRLNDVMLSKKEQNGITLQQRKNNLNLLSKGELVIKNVSYHYANNKEVLKKLNLR
IKNDEKVALLGQSGSGKSTIAKLLVGFLQSNSGDILVDGVKINQLSTYQLRNFICYVP
QQTYLFSGSIMDNLIVGCDKEKSMEEIINACKIACISNEIDEMPLKYNTKLDEGGLIL
SGGQSQRLAIARAILSPAKILILDESTSGLDPITEHKVIKNLMTLHKTILFITHRLAV
AKSTNKVYVLKNGKIVEAGTYEELTKKKKTYLNS″ (SEQ ID NO: 30)
/standard_name = ″lagD CDS″

| | |
|---|---|
| CDS 7247..7420 | /locus_tag = ″LREU3632_02343″ |
| | /inference = ″ab initio prediction: Prodigal:2.6″ |
| | /product = ″hypothetical protein″ |

/translation = ″MKDTNYIKKSLENREIQKLTEDEMKAFIGGTTYHINYDGYAPSR
NPWAWLTHWGHKH″ (SEQ ID NO: 31)
/standard_name = ″hypothetical protein CDS″

| | |
|---|---|
| CDS 7423..8736 | /locus_tag = ″LREU3632_02344″ |
| | /inference = ″ab initio prediction: Prodigal: 2.6″ |
| | /inference = ″protein motif: CLUSTERS: PRK11086″ |
| | /product = ″sensory histidine kinase DcuS″ |

/translation = ″MNYINPFYLYGLEFFDLFINTLLINNIAGKRKINLLSIFEIGVI
VLVLGYYGYTIYIAYALLFLYKNFKHNKRVLNFEALIMFVCSLFVFLISNINSFILNT
WFLPHYSNDVLIRFQIVISVIISIMLVSISFFYNPLFSKISNNLFLKYKGPILRKFVI
TYEVSFFFIIGIASIAEYLKIEFKIQWLLILCFLFLSLSVIILTGIMFTYNSKMIQLE
EIRNSYINKQLYWKSFESNYKKLRKLHHDYTNLLITLIRLIENNDFKHAKSYANSLLD
YDSKVYSPPSFDIIQISQKIYNQELRALFLEKVNIIKSQRIKLHLEISDKIAIPSKLL
IDLLIIISNLLDNATSAALQSDQKSISISLKKTLSNCYKFTIKNSIKSKINIEELFTE
NFSTKKNHMGIGLNNVISIIDSSPNFFLETKSFNEWIEFELILKR″ (SEQ ID NO: 32)
/standard_name = ″sensory histidine kinase DcuS CDS″

| | |
|---|---|
| misc_feature | /note = ″Geneious type: Editing History Deletion″ |

/standard_name = ″ATGACGTTAGAGATATTATTACTTGAAGATAATCTTTTGCAA
CTTCAAAAATATAAGAAGATAATCCAGAATAGAATTATGATTAATCCTAGTAATAAAT
CTTATGATCTAGAGCTTGCACTCTACTCATCAACTTCAGATGAAATACATCAATACCT
CCATAATAATAGTAAGAAAGATATTTTTGCCTTTCTTGATATTGAAATAGGGTCTACG
TATTCAGGAATAGATATAGCAAAAGAGATTAAAAATACTGAAAGTCAAAATTTTAGAG
AAGTTTGTITTATTTCTACTTATGATAATCTTTTAATAAATATTATTAATGATCATGT
ATCACCTTTTGACTTTATTTTAAAGAATAATGGAATCAATTACGTTACCAAAAAGATT
CAAGAGAATATTGACTGGGCTTATTTAAAATATAAAGATTTAATTACTAAATCTAATC
AAGAATTATTTACATATGAGTCTTTCCCCGGATATATTCATCGCATTCCTAGAGATAA
CATTCTTTTTATATCTACTACTTCTTTGCAACATCGTTTAAGAATAACTTGTAAAGAT
AACGAAATACTTTTTCAGGGGGAATTAAAAAATATTGATTCCAAAAATAATGCATTCT
TTAGAGTAGATAGACAAATTTTAGTAAACTTAAATAATATCGATTATGTCGATTTTCA
TAAAAGAAAAATCTTCTTCAAAAATTCTAAAAACAAAAATTGTAAAATTAGTCTACGT
AAAATCCCTCTTCTTAGAAGGCTATTACAAGAAACGTCGTCGCCGGTCACCCATAAGA
AACAATGATATTACATTATATCAAGAGTTGAAATTTTGCGTATTTATTTAATGATAGA
GCGTAGTAATTCTTCATCCAAAAACCAGCAAGATCTTTAAATTTGCAGAATGATAATT
AGGTGAAGCATAGTAGTATCACTAACACAAACAAAGCCCCTTGGCAATCATAGTCGAT
TGCCAAGGGGCTTTGTTTGTGGGCTACTCTCACGAATT″ (SEQ ID NO: 33)

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A modified Bacillus bacteria for production or live delivery of one or more biomolecule or heterologous protein, wherein the bacteria comprises Bacillus subtilis strain 105 (ELA191105) genetically modified in one or more aspect selected from the following:
(a) genetically modified to increase competency;
(b) genetically modified to reduce or block spore formation;
(c) genetically modified to delete or inactivate one or more native protease; and
(d) genetically modified to include nucleic acid encoding one or more biomolecule or heterologous protein;
wherein the B subtilis strain 105 comprises the nucleic acid sequence set out in SEO ID NO: 1, 2, 3, 4, 5 or 6 or comprises nucleic acid that has at least 90% identity, 95% identity, 97% identity, 98% identity, 99% identity in genomic sequence to SEO ID NO: 1, 2, 3, 4 5 or 6, or
wherein the Bacillus subtilis strain corresponds to ATCC deposit PTA-126786 strain or has at least 95% identity, 97% identity, 98% identity, 99% identity in genomic sequence to the sequence of ELA191105 corresponding to ATCC deposit PTA-126786.

2. (canceled)

3. (canceled)

4. (canceled)

5. The modified Bacillus bacteria of claim 1, wherein in (a) the bacteria is modified to overexpress comK, comS, or comK and comS to increase competency, wherein competency is increased and transformation efficiency of the strain is increased by at least 20 fold, by 50 fold, by 50 fold or greater, by 60 fold, by 80 fold, by 80 fold or greater, by 90 fold, by 100 fold or by 100 fold or greater: wherein in (b) the bacteria is modified to delete or inactivate one or more native gene encoding SpoOA, SpoIVB or SpoA and SpoIVB; and/or wherein in (c) the bacteria is modified to delete or inactivate one or more native protease or the gene encoding one or more native protease selected from NprE, AprE, Epr1, Epr2, Bpr, Mpr, NprB, Vpr, and WprA.

6. The modified Bacillus bacteria of claim 5, wherein a gene cassette encoding comK and comS is integrated in the B subtilis genome.

7. (canceled)

8. (canceled)

9. (canceled)

10. The modified Bacillus bacteria of claim 5, wherein in (c) the bacteria is modified to delete or inactivate the gene encoding native proteases NprE and Vpr, or wherein the bacteria is modified to delete or inactivate the gene encoding native proteases AprE, NprB and WprA.

11. (canceled)

12. The modified Bacillus bacteria of claim 1, further genetically modified to delete or inactivate one or more native lytic enzyme or antibacterial peptide, or further genetically modified to delete or inactivate one or more native gene encoding a virulence factor, toxin or antibacterial resistance (AMR).

13. The modified Bacillus of claim 12, wherein one or more native lytic enzyme or antibacterial peptide selected from xpf, lytC1, lytC2 and sdpC are deleted or inactivated or wherein the one or more virulence factor, toxin or antibacterial resistance (AMR) is selected from macrolide 2′phosphotransferase (mphK), ABC—F type ribosomal protection protein (vmlR), Streptothricin-N-acetyltransferase (satA), tetracyclin efflux protein (tet(L)), aminoglycoside 6-adenylvltransferase (aadK) (29), and rifamycin-inactivating phosphotransferase (rphC), as set out in Table 16.

14. (canceled)

15. (canceled)

16. (canceled)

17. The modified Bacillus bacteria for production or live delivery of one or more biomolecule or heterologous protein of claim 1, wherein the modified Bacillus comprises a B. subtilis strain 105 isolate modified to overexpress comK, comS or comK and comS to increase competency; having at least at least one gene knockout selected from the following genes: spoOA, spoIIIE, spoIVB, NprE, AprE, NprB, Vpr, WprA; and modified to comprise one or more heterologous gene encoding one or more biomolecule or heterologous protein operatively linked to one or more promoter selected from a tuf promoter, sigx promoter, gros promoter, ftsh promoter, a PxylA promoter, a mannose inducible promoter, and a Physpank promoter.

18. The modified Bacillus bacteria of claim 17, wherein the one or more promoter is selected from SEQ ID NO: 66, SEQ ID NO: 67, SEQ ID NO: 68, SEQ ID NO: 69, SEQ ID NO: 40 and SEQ ID NO: 41.

19. The modified Bacillus bacteria of any of claims 1-18, wherein the one or more heterologous gene encoding one or more biomolecule or heterologous protein is integrated in the host B subtilis strain 105 genome, optionally wherein the one ormore heterologous gene encoding one ormore biomolecule or heterologous protein is integrated in the host B subtilis strain 105 genome at one or more gene locations selected from amvE, NprE, AprE, Epr1, Epr2, Bpr, Mpr, NprB, Vpr, and WprA.

20. (canceled)

21. The modified Bacillus bacteria of claim 1, wherein the one or more biomolecule or heterologous protein is selected from an anti-infective agent, anti-bacterial agent, anti-pathogen agent, immunomodulatory factor or agent, antigen, antibody, growth-promoting biomolecule, a probiotic, and a bio-based chemical.

22. (canceled)

23. The modified Bacillus bacteria of claim 21, wherein the one or more biomolecule or heterologous protein is an anti-bacterial agent and wherein the one or more anti-bacterial agent is one or more lysin or lytic peptide.

24. The modified Bacillus bacteria of claim 23, wherein the one or more lysin or lytic peptide is PlyCM, CP025C, lysostaphin or a native B. subtilis 105 lytic enzyme.

25. The modified Bacillus bacteria of claim 2J_2, wherein the one or more anti-bacterial agent is one or more antimicrobial peptide (AMP).

26. The modified Bacillus bacteria of claim 25, wherein the one or more antimicrobial peptide (AMP) is a mersacidin or a cathelicidin peptide, optionally wherein the one or more antimicrobial peptide (AMP) is a CAP18 peptide.

27. (canceled)

28. The modified Bacillus bacteria of claim 21, wherein the one or more biomolecule or heterologous protein is one or more antibody or a fragment thereof, optionally wherein the one or more antibody or fragment thereof is one or more single chain antibody, domain antibody, VHH antibody or nanobody, and optionally wherein the one or more single chain antibody, domain antibody, VHH antibody or nanobody one or more single chain antibody, domain antibody, VHH antibody or nanobody directed against a pathogenic bacteria.

29. (canceled)

30. (canceled)

31. The modified Bacillus bacteria of claim 30, wherein the one or more antibody is one or more VHH antibody or nanobody directed against Clostridium perfringens and wherein the one or more antibody is one or more VHH antibody or nanobody directed against Clostridium perfringens alpha toxin or NetB, optionally wherein the one or more VHH antibody is selected from SEQ ID NO: 97, SEQ ID NO: 98, SEQ ID NO: 101 and SEQ ID NO: 102.

32. (canceled)

33. (canceled)

34. The modified Bacillus bacteria of claim 21, wherein the one or more biomolecule or heterologous protein is one or more antigen and wherein said antigen is capable of stimulating an immune response against a parasite, bacteria, or virus.

35. The modified Bacillus bacteria of claim 34, wherein the one or more biomolecule or heterologous protein is one or more antigen capable of stimulating an immune response against an Eimeria parasite and wherein the one or more antigen is selected from Eimeria tenella elongation factor-1α, EtAMA1, EtAMA2, Eimeria tenella 5401, Eimeria acervuline lactate dehydrogenase antigen gene, Eimeria maxima surface antigen gene, Glyceraldehyde 3-phosphate Dehydregenase (GAPDH) and Eimeria common antigen 14-3-3.

36. (canceled)

37. The modified Bacillus bacteria of claim 36, wherein the one or more antigen is an Eimeria antigen encoded by one or more of SEQ ID NO: 105, SEQ ID NO: 106, SEQ ID NO: 107, SEQ ID NO: 108, or SEQ ID NO: 109.

38. The modified Bacillus bacteria of claim 1, wherein the one or more heterologous gene encoding one or more biomolecule or heterologous protein is provided on a biosynthetic gene cluster (BGC) and wherein the BGC or a portion thereof is integrated in the host B subtilis strain 105 genome.

39. The modified Bacillus bacteria of claim 38, wherein the biosynthetic gene cluster (BGC) is a PKS BGC or a mersacidin BGC, wherein the PKS BGC is capable of producing an AhR-activating metabolite, or wherein the mersacidin BGC is capable of producing one or more mersacidin polypeptide SEQ ID NO: 22 or SEQ ID NO: 23 capable of inhibiting or killing one or more bacteria or virus.

40. (canceled)

41. (canceled)

42. The modified Bacillus bacteria of claim 39, wherein the PKS BGC comprises the nucleic acid set out in SEQ ID NO: 110 or comprises nucleic acid encoding one or more polypeptide selected from SEQ ID NOs: 7-21, or wherein the mersacidin BGC comprises the nucleic acid set out in SEQ ID NO: 24 or comprises nucleic acid encoding one or more polypeptide selected from SEQ ID NOs: 25-32.

43. (canceled)

44. The modified Bacillus bacteria of claim 21, wherein the one or more biomolecule or heterologous protein is a bio-based chemical, optionally wherein the bio-based chemical is gamma polyglutamic acid (γ-PGA).

45. (canceled)

46. The modified Bacillus bacteria of claim 45, wherein the γ-PGA is encoded by the CapABC locus and the B subtilis strain 105 is modified to produce increased amounts of γ-PGA by integrating at least one additional copy of the CapABC locus in B subtilis strain 105 genome, optionally wherein at least one additional copy of the CapABC locus is integrated in B subtilis strain 105 genome at one or more gene locus selected from amvE, nprE, apr and wprA.

47. (canceled)

48. The modified Bacillus bacteria of claim 1, wherein the one or more heterologous gene encoding one or more biomolecule or heterologous protein includes a native B subtilis 105 strain or other bacterial strain signal sequence for secretion of the one or more biomolecule or heterologous protein by the modified bacteria optionally wherein the native B subtilis 105 strain or other bacterial strain signal sequence for secretion is selected from SEO ID NO: 43, SEO ID NO: 44, SEO ID NO: 46, SEO ID NO: 47, SEO ID NO: 49, and SEO ID NOs: 50-64.

49. (canceled)

50. A live delivery platform comprising a genetically-modified Bacillus bacteria for production of one or more biomolecules or heterologous proteins in an animal, wherein the modified Bacillus comprises Bacillus subtilis strain 105 (ELA191105) genetically modified to include nucleic acid encoding one or more biomolecule or heterologous protein which is produced and delivered upon administration of the modified Bacillus bacteria to the animal, wherein the bacteria comprises Bacillus subtilis strain 105 (ELA191105) genetically modified in one or more aspect selected from the following:
(a) genetically modified to increase competency:
(b) genetically modified to reduce or block spore formation:
(c) genetically modified to delete or inactivate one or more native protease; and
(d) genetically modified to include nucleic acid encoding one or more biomolecule or heterologous protein:
wherein the Bacillus subtilis strain 105 comprises the nucleic acid sequence set out in SEQ ID NO: 1 or comprises at least 95% identity, 97% identity, 98% identity, 99% identity in genomic sequence to SEO ID NO: 1, or
wherein the Bacillus subtilis strain corresponds to ATCC deposit PTA-126786 strain or has at least 95% identity, 97% identity, 98% identity, 99% identity in genomic sequence to the sequence of ELA191105 corresponding to ATCC deposit PTA-126786.

51. (canceled)

52. (canceled)

53. (canceled)

54. The live delivery platform of claim 50, wherein the Bacillus subtilis bacteria is genetically modified to include nucleic acid encoding one or more biomolecule or heterologous protein and comprises an expression cassette;
wherein the expression cassette comprises one or more of:
a promoter for transcriptional expression,
a nucleic acid sequence encoding a signal sequence for secretion,
at least one heterologous coding region encoding a desired biomolecule or heterologous protein, and terminators for translation and transcription termination.

55. The live delivery platform of claim 54, wherein the promoter for transcriptional expression is one or more promoter selected from a tuf promoter, sigx promoter, gros promoter, ftsh promoter, a PxylA promoter, a mannose inducible promoter, and a Physpank promoter, optionally wherein the one or more promoter is selected from SEO ID NO: 66, SEO ID NO: 67, SEO ID NO: 68, SEO ID NO: 69, SEO ID NO: 40 and SEO ID NO: 41.

56. (canceled)

57. The live delivery platform of claim 54, wherein the nucleic acid sequence encoding a signal sequence for secretion encodes at least 20 amino acids, at least 25 amino acids, at least 30 amino acids, at least 35 amino acids, at least 40 amino acids, at least 44 amino acids, at least 50 amino acids, at least 55 amino acids, at least 60 amino acids, or at least 65 amino acids, optionally wherein the nucleic acid sequence encoding a signal sequence for secretion encodes a native B subtilis 105 strain or other bacterial strain signal sequence for secretion comprising a sequence selected from SEO ID NO: 43, SEO ID NO: 44, SEO ID NO: 46, SEO ID NO: 47, SEO ID NO: 49, and SEO ID NOs: 50-64.

58. (canceled)

59. The live delivery platform of claim 50, wherein the expression cassette or the at least one heterologous coding region encoding a desired biomolecule or heterologous protein is integrated in the host B subtilis strain 105 genome, optionally wherein the expression cassette or the at least one heterologous coding region encoding a desired biomolecule or heterologous protein is integrated in the host B subtilis strain 105 genome at one or more gene locations selected from amvE, NprE, AprE, Epr1, Epr2, Bpr, Mpr, NprB, Vpr, and WprA.

59. (canceled)

60. The live delivery platform of claim 50, wherein desired biomolecule or heterologous protein is selected from an anti-infective agent, anti-bacterial agent, anti-pathogen agent, immunomodulatory factor or agent, antigen, antibody, growth-promoting biomolecule, a probiotic, and a bio-based chemical.

61. A method of reducing colonization of an animal by a pathogenic bacterium, parasite or virus, the method comprising treating an animal with the modified Bacillus bacteria of claim 1.

62. The method of claim 61, wherein the animal is a bird, a human, or a non-human mammal.

63. The method of claim 61, wherein the pathogenic bacterium is selected from the group consisting of Salmonella, Clostridium, Campylobacter, Staphylococcus, Streptococcus, and an E. coli bacterium, or wherein the pathogenic parasite is Eimeria.

64. (canceled)

65. The method of claim 61, wherein the modified Bacillus bacteria is administered orally, parentally, nasally, or mucosally, or wherein the animal is a bird and wherein treatment is administered in ovo.

66. (canceled)

67. (canceled)

68. (canceled)

69. (canceled)
